(19) United States
(12) Patent Application Publication
(10) Pub. No.: US 2023/0272356 A1
(43) Pub. Date: Aug. 31, 2023

(54) C-TERMINAL PEPTIDE EXTENSIONS WITH INCREASED ACTIVITY

(71) Applicant: Integrated DNA Technologies, Inc., Coralville, IA (US)

(72) Inventors: Sarah Franz Beaudoin, Iowa City, IA (US); Tanner Holden Reeb, Coralville, IA (US); Christopher Anthony Vakulskas, North Liberty, IA (US)

(21) Appl. No.: 18/176,091

(22) Filed: Feb. 28, 2023

Related U.S. Application Data
(60) Provisional application No. 63/314,666, filed on Feb. 28, 2022.

(51) Int. Cl. C12N 9/12; C12Q 1/686

(52) U.S. Cl. CPC C12N 9/1276; C12Y 207/07049; C12Q 1/686

(57) ABSTRACT

The disclosure provides Moloney murine leukemia virus (MMLV) reverse transcriptase (RTase) mutants with C-terminal peptide and/or N-terminal peptide extensions that improve the performance of the MMLV RTase mutants. The disclosure also provides suitable amino acid positions in MMLV RTases for mutagenesis and methods and kits for using MMLV RTase mutants to synthesize cDNA from RNA templates.

### CROSS-REFERENCE TO RELATED APPLICATIONS

This application claims benefit under 35 U.S.C. § 119(e) of U.S. Provisional Application No. 63/314,666 filed Feb. 28, 2022. The above listed application is incorporated by reference herein in its entirety for all purposes

### REFERENCE TO AN ELECTRONIC SEQUENCE LISTING

The instant application contains a Sequence Listing which has been submitted electronically as a text file in .XML format and is hereby incorporated by reference in its entirety. The name of the .XML file is “22-0291-WO_ST26_FINAL.xml”, the file was created on Feb. 28, 2023 and 1,026,058 bytes in size.

### FIELD OF THE DISCLOSURE

The disclosure relates to Moloney murine leukemia virus (MMLV) reverse transcriptase (RTase) mutants with C-terminal and/or N-terminal peptide extensions that improve the performance of the MMLV RTase mutants. The disclosure also relates to suitable amino acid positions in MMLV RTases for mutagenesis and methods and kits for using MMLV RTase mutants to synthesize cDNA from RNA templates.

### BACKGROUND

Reverse transcriptase (RTase) enzymes have revolutionized molecular biology. RTase is a critical component of the reverse transcription polymerase chain reaction (RT-PCR) allowing the production of complementary DNA (cDNA) from RNA. The cDNA produced in reverse transcription reactions can be used in a wide range of downstream applications, including quantitative PCR, gene expression analysis, isolated RNA sequencing, gene cloning, and cDNA library creation.

RTases, first derived from retroviruses, facilitate the reverse transcription of RNA into cDNA by utilizing RNA-dependent polymerase and RNase H, a non-sequence-specific endonuclease enzyme that catalyzes cleavage of RNA in an RNA/DNA duplex. This results in virus replication and integration of the viral sequence into host DNA thereby allowing for the proliferation of the virus along with host DNA. Within the laboratory setting, RTases from Moloney murine leukemia virus (MMLV), avian myeloblastosis virus (AMV), and human immunodeficiency virus type 1 (HIV-1) are the most commonly used RTase for cDNA synthesis.

RTases for research applications are often mutated multi-generational MMLV and AMV RTases that have been optimized for laboratory procedures. Mutations in the RTases alter properties of the enzymes, including thermostability, RTase activity, 5′ mRNA coverage, and RNase H activity.

AMV RTases are thermostable and less sensitive to thermal degradation than MMLV RTase and are preferred for RNA having a strong secondary structure. In addition, AMV RTases are often suitable for use with RNA molecules that are five kilobases or longer because of the heat stability of AMV RTases. However, the high temperatures required to resolve strong secondary structures or long RNA strands can negatively impact RNA integrity and fidelity of transcription. AMV also possess an intrinsic RNase activity that degrades RNA in an RNA/DNA hybrid, which can result in reduced total cDNA and reduced full-length cDNA yield.

MMLV RTase is characterized by low RNase H activity and a higher fidelity as compared to AMV RTase. The reduced RNase H activity allows MMLV RTases to be used for the reverse transcription of long RNAs (>5 kb). However, the RNase H activity of MMLV RTase limits the efficiency of synthesizing long cDNA in vitro. Mutations in MMLV RTase have been introduced to reduce RNase H activity. In addition, because the optimal temperature for MMLV RTase activity is −37° C., the enzyme lacks the ability to effectively reverse transcribe RNAs with strong secondary structures. The use of MMLV RTase at elevated temperatures can compromise cDNA length and yield as a result of lower enzyme activity. MMLV RTase mutants that substitute Mn²⁺ for Mg²⁺ in the reaction mixture attempt to overcome these limitations, but are characterized by inefficiency and error.

Thus, despite the unique properties of AMV and MMLV RTases, there exists a need for an RTase that combines the beneficial attributes of AMV and MMLV RTases. Consistent with this, the present application discloses MMLV RTase mutants containing an unnatural peptide tag on the C-terminal and/or N-terminal end of MMLV RTase that confers increased RTase activity and thermostability as compared to RTases without a C-terminal and/or N-terminal peptide extension.

### SUMMARY

The disclosure provides Moloney murine leukemia virus (MMLV) reverse transcriptase (RTase) mutants with C-terminal and/or N-terminal extensions that improve the performance of the MMLV RTase mutants. The disclosure also provides suitable amino acid positions in MMLV RTases for mutagenesis and methods and kits for using MMLV RTase mutants to synthesize cDNA from RNA templates.

One aspect of the disclosure provides an isolated Moloney murine leukemia virus (MMLV) reverse transcriptase (RTase) mutant comprising a C-terminal peptide extension, an N-terminal peptide extension, or both a C-terminal and an N-terminal peptide extension.

In another aspect, the disclosure provides an isolated Moloney murine leukemia virus (MMLV) reverse transcriptase (RTase) mutant comprising the amino acid sequence of SEQ ID NO: 674 (MMLV-II), wherein the amino acid sequence of the MMLV RTase mutant further comprises a C-terminal peptide extension or N-terminal peptide extension and at least two amino acid substitutions that are: (a) a glutamine to arginine substitution at position 68 (Q68R); (b) a glutamine to arginine substitution at position 79 (Q79R); (c) a leucine to tyrosine at position 82 (L82Y); (d) a leucine to arginine substitution at position 99 (L99R); (e) a leucine to isoluecine at position 280 (L280I); (f) a glutamic acid to aspartic acid substitution at position 282 (E282D); (g) a glutamine to glutamic acid substitution at position 299 (Q299E); (h) threonine to lysine at position 306 (T306K); (i) a valine to asparagine at position 433 (V433N); or (j) an isoleucine to tryptophan at position 593 (I593W).

In yet a further aspect, the disclosure provides a method for synthesizing complementary deoxyribonucleic acid (cDNA) comprising: (a) providing the isolated MMLV RTase mutant of any one of claims 1 to 10; and (b) contacting the isolated MMLV RTase mutant with a nucleic acid template to permit synthesis of cDNA.

In a further aspect, the disclosure provides a method for performing reverse transcription-polymerase chain reaction (RT-PCR) comprising: providing the isolated MMLV RTase mutant of any one of claims 1 to 10; and contacting the isolated MMLV RTase mutant with a nucleic acid template to replicate and amplify the nucleic acid template.

Specific embodiments of the disclosure will become evident from the following more detailed description and the claims.

### BRIEF DESCRIPTION OF THE DRAWINGS

FIGS. 1A-1C are schematics showing reverse transcriptase mutagenesis selection by rational design. Amino acid positions for mutagenesis were chosen at the substrate binding site (FIGS. 1A and 1B) or near the substrate binding site (FIG. 1C).

FIG. 2 shows Western blot analysis of test induction results in in BL21(DE3) cells for MMLV RT in TB medium. Lane 1—Precision Plus Protein Unstained Standards (Bio Rad, Cat #161-0363), Lane 2—Time=0 hour, Lane 3—Time=3 hours after induction at 37° C., Lane 4—Time=0 hour, Lane 5—Time=21 hours after induction at 18° C.

### DETAILED DESCRIPTION

The disclosure relates to C-terminal and/or N-terminal extensions that improve the performance of Moloney murine leukemia virus (MMLV) reverse transcriptase (RTase) mutants. The C-terminal and/or N-terminal peptide extensions of MMLV RTase mutants of the disclosure display increased RTase activity and thermostability as compared with commercially available RTases.

Reference will now be made in detail to exemplary embodiments of the claimed invention. While the claimed invention will be described in conjunction with the exemplary embodiments, it will be understood that it is not intended to limit the claimed invention to those embodiments. To the contrary, it is intended to cover alternatives, modifications, and equivalents, as may be included within the spirit and scope of the claimed invention, as defined by the appended claims.

Those of ordinary skill in the art may make modifications and variations to the embodiments described herein without departing from the spirit or scope of the claimed invention. In addition, although certain methods and materials are described herein, other methods and materials that are similar or equivalent to those described herein can also be used to practice the claimed invention.

In addition, any of the compositions or methods provided, disclosed, or described herein can be combined with one or more of any of the other compositions and methods provided, disclosed, or described herein.

### 1. DEFINITIONS

Unless defined otherwise, all technical and scientific terms used herein have the meaning commonly understood by a person skilled in the art to which the claimed invention belongs. The terminology used herein is for describing particular embodiments only and is not intended to be limiting of the claimed invention. All technical and scientific terms used herein have the same meaning.

The following references provide those of skill in the art with a general understanding of many of the terms used herein (unless defined otherwise herein): Singleton et al., Dictionary of Microbiology and Molecular Biology, 3rd ed. (Wiley, 2006); Walker, The Cambridge Dictionary of Science and Technology (Cambridge University Press, 1990); Rieger et al., Glossary of Genetics: Classical and Molecular, 5th ed. (Springer Verlag, 1991); and Hale et al., Harper Collins Dictionary of Biology (HarperCollins Publishers, 1991). Generally, the procedures or methods described herein and the like are common methods used in the art. Such standard techniques can be found in reference manuals such as, for example, Green et al., Molecular Cloning: A Laboratory Manual, 4th ed. (Cold Spring Harbor Laboratory Press, 2012), and Ausubel, Current Protocols in Molecular Biology (John Wiley & Sons Inc., 2004).

The following terms may have meanings ascribed to them below, unless specified otherwise. However, it should be understood that other meanings known or understood by those having ordinary skill in the art are also possible, and within the scope of the claimed invention. All publications, patent applications, patents, and other references mentioned or discussed herein are expressly incorporated by reference in their entireties. In the case of conflict, the present specification, including definitions, will control. In addition, the materials, methods, and examples are illustrative only and not intended to be limiting.

As used herein, the singular forms “a,” “and,” and “the” include plural references, unless the context clearly dictates otherwise.

As used herein, the term “or” means, and is used interchangeably with, the term “and/or,” unless context clearly indicates otherwise.

As used herein, the term “including” means, and is used interchangeably with, the phrase “including but not limited to.”

As used herein, the term “such as” means, and is used interchangeably with, the phrase “such as, for example” or “such as but not limited.”

Unless specifically stated or obvious from context, as used herein, the term “about” is understood as within a range of normal tolerance in the art, for example, within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “nucleic acid molecule” and “polynucleotide” refer to a polymer or large biomolecule comprised of nucleotides. The term “nucleic acid” includes deoxyribonucleic acid (DNA), ribonucleic acid (RNA), and analogs thereof. Non-limiting examples of nucleic acid molecules include DNA (e.g., genomic DNA, cDNA), RNA molecules (e.g., mRNA, rRNA, cRNA, tRNA), and chimeras thereof. A nucleic acid molecule can be obtained by cloning techniques or synthesized, using techniques that are known to those of skill in the art. DNA can be double-stranded or single-stranded (coding strand or non-coding strand, i.e., antisense). A nucleic acid backbone may comprise a variety of linkages known in the art, including one or more of sugar-phosphodiester linkages, peptide-nucleic acid bonds (referred to as “peptide nucleic acids” (PNA)), phosphorothioate linkages, methylphosphonate linkages, or combinations thereof. Sugar moieties of the nucleic acid may be ribose or deoxyribose, or similar compounds having known substitutions, for example, 2′ methoxy substitutions (containing a 2′-O-methylribofuranosyl moiety) and/or 2′ halide substitutions. Nitrogenous bases may be conventional bases (adenine (A), guanine (G), thymine (T), cytosine (C), and uracil (U)), known analogs thereof (e.g., inosine), known derivatives of purine or pyrimidine bases, or “abasic” residues in which the backbone includes no nitrogenous base for one or more residues. A nucleic acid may comprise only conventional sugars, bases, and linkages, as found in RNA and DNA, or may include both conventional components and substitutions (e.g., conventional bases linked via a methoxy backbone, or a nucleic acid including conventional bases and one or more base analogs). An “isolated nucleic acid molecule,” as is generally understood by those of skill in the art and as used herein, refers to a polymer of nucleotides, and includes but is not limited to DNA and RNA.

As used herein, the term “probe” refers to a nucleic acid oligonucleotide that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. A probe can be produced by molecular cloning of a specific DNA sequence or it can be synthesized. Probes for use in the methods disclosed herein can be readily designed and used by those of skill in the art.

As used herein, the term “primer” refers to a nucleic acid oligonucleotide that hybridizes specifically to a target sequence in a nucleic acid or its complement, and which is capable of priming the synthesis of a nascent nucleic acid in a template-dependent process. Primers may be provided in double-stranded or single-stranded form. Primers for use in the methods disclosed herein can be readily designed and used by those of skill in the art.

Probes or primers for use in the methods disclosed herein may be of any suitable length, depending on the particular assay format and the particular needs and targeted sequences employed. For example, the probes or primers for use in the methods disclosed herein are at least 10 nucleotides in length, or at least 15, 20, 25, 30, or more than 30 nucleotides in length, and they may be adapted to be especially suited for a chosen nucleic acid amplification system and/or hybridization system used. Longer probes and primers are also within the scope of the disclosure.

A “transcribed polynucleotide” or “nucleotide transcript” is a polynucleotide (e.g., mRNA, hnRNA, cDNA, or analog of such RNA or cDNA) that is complementary to or having a high percentage of identity (e.g., at least 80% identity) with all or a portion of a mature mRNA made by transcription of a marker of the disclosure and normal post-transcriptional processing (e.g., splicing), if any, of the RNA transcript, and reverse transcription of the RNA transcript.

As used herein, the terms “reverse transcriptase,” “RTase,” or “RT” refer to an enzyme that is used to generate complementary (cDNA) from an RNA template in a process known as “reverse transcription.” The term reverse transcriptase, as used herein, also refers to any enzyme that exhibits reverse transcription activity. Reverse transcriptases can be derived from a variety of sources including but not limited to viruses including retroviruses and DNA polymerases exhibiting transcriptase activity. Such retroviruses include but are not limited to Moloney murine leukemia virus (MMLV), avian myeloblastosis virus (AMV), and human immunodeficiency virus (HIV).

Reverse transcriptase activity can be measured by incubating an RTase in a buffer containing an RNA template and deoxynucleotides. One of skill in the art will recognize that a wide range of conditions can be used to perform reverse transcription reactions and multiple methods exist for measuring the quantity of cDNA produced during reverse transcription.

Reverse transcriptases of the disclosure include reverse transcriptases having one or a combination of the properties described herein. Such properties include but are not limited to increased activity, enhanced DNA synthesis, enhanced stability or enhanced thermostability, reduced or eliminated RNase H activity, reduced terminal deoxynucleotidyl transferase activity, increased accuracy or increased fidelity, increased specificity, or altered half-life, for example when compared to a base construct. As used herein, the term “base construct” refers to the initial RTase from which the RTase mutants of the disclosure are prepared (e.g. for example a wild-type RTase or a modified wild-type RTase).

As used herein, the terms “accuracy” and “fidelity” are used interchangeably and refer to ability of an RTase to accurately replicate a desired template; i.e., the ability of the RTase to accurately perform cDNA synthesis in a reverse transcription reaction. The “fidelity” or “accuracy” of a reverse transcriptase can be assessed by determining the frequency of incorrect nucleotide incorporation into the synthesized cDNA molecule, which may be referred to as the enzyme's error rate. As used herein, the term “increased fidelity” refers to RTase mutants of the disclosure that exhibit an error rate lower than that of the base construct. For example, the RTase mutants as disclosed herein can exhibit an error rate that is 10%, 15%, 20%, 25%, 30%, 35%, 40%, 45%, 50%, 55%, 60%, 65%, 70%, 75%, 80%, 85%, 90%, 95%, 100%, or 200% lower than, or at least 2-fold, 3-fold, 4-fold, 5-fold, or 10-fold, or more than 10-fold lower than the error rate of the RTase base construct . . . .

As used herein, the term “specificity” refers to a decrease in mis-priming by an RTase during cDNA synthesis. An RTase mutant's specificity can be assessed by performing a reverse transcription reaction at a particular temperature, including higher temperatures, and comparing the amount of mis-priming in that reaction with the amount of mis-priming in a reaction performed with the wild-type RTase (or the RTase base construct) under identical conditions.

As used herein with respect to the RTase molecules of the disclosure, the terms “stable” and “thermostable” are used interchangeably and refer to an enzyme that is resistant to heat inactivation and remains active at temperatures in excess of 37° C. (e.g., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 70° C., or higher temperatures). For example, in one embodiment the disclosure provides an RTase mutant having activity with a longer half-life than that of the base construct RTase at an elevated temperature. Thus, RTase mutants with “enhanced thermostability” can refer to RTase mutants of the disclosure that exhibit an increase in thermostability at temperatures of about 50° C. up to about 90° C. as compared to the base construct RTase. In some embodiments, the thermostability of the RTase mutant is at least 1.5 fold or greater as compared to the thermostability of the base construct RTase. Comparisons of cDNA produced by a base construct and RTase mutant are compared using identical reaction conditions for the base construct and RTase mutant reactions. Reaction conditions can include but are not limited to salt concentration, buffer concentration, pH, divalent metal ion concentration, temperature, nucleoside triphosphate concentration, template concentration, RTase concentration, primer concentration, time, and in one-step PCR, the quantitative PCR primer and probe concentrations.

As used herein, the term “enhanced DNA synthesis” refers to an RTase enzyme that produces more DNA (e.g. cDNA) than the base RTase construct. In some embodiments, DNA synthesis can be measured by quantitative PCR at standard reaction conditions, as compared to the base construct RTase. Consistent with assessments of thermostability, quantitative comparisons are made under similar or the same reaction conditions and the amount of cDNA synthesized using the base construct RTase is compared to the amount of cDNA produced using the RTase mutant (see Tables 4-7). In some embodiments, the RTase mutant of the disclosure with enhanced DNA synthesis may produce about 5% to about 200% more cDNA than the base construct RTase. In some embodiments, the RTase mutant of the disclosure with enhanced DNA synthesis has at least 10%, 15%, 20%, 25%, 30%, 35%, 40%, 45%, 50%, 55%, 60%, 65%, 70%, 75%, 80%, 85%, 90%, 95%, 100%, or 200% more than, or at least 2-fold, 3-fold, 4-fold, 5-fold, or 10-fold, or more than 10-fold more DNA synthesis than the RTase base construct DNA synthesis.

Reverse transcriptase activity, as described herein, was evaluated in a one-step or two-step procedure. The one-step procedure combines reverse transcription and quantitative PCR in a single reaction. The method is performed by including Gene Expression Master Mix, RTase, RNA, a fluorescent probe, and primers and probes as described in Example 3. The two-step procedure comprises reverse transcription followed by quantitative PCR. In the reverse transcription step, RTase is added to a mixture containing RNA, gene specific primers, first strand synthesis buffer, and RNase. The resultant cDNA is then quantified in a second step wherein the cDNA is combined with Gene Expression Master Mix, primers and probes, and a fluorescent marker. The cDNA produced in either the one-step and two-step procedures is quantified, and the mean and standard deviation reported as shown herein in Tables 4-7.

As used herein, “RNase H activity” refers to cleavage of RNA in DNA-RNA duplexes via a hydrolytic mechanism to produce 5′ phosphate terminated oligonucleotides. RNase H activity does not include degradation of single-stranded nucleic acids, duplex DNA, or double-stranded RNA. As used herein, the phrase “substantially lacks RNase H activity” means having less than 10%, 5%, 1%, 0.5%, or 0.1% of the activity of a wild type enzyme. As used herein, the phrase “lacks RNase H activity” means having undetectable RNase H activity or having less than about 1%, 0.5%, or 0.1% of the RNase H activity of a wild type enzyme.

As used herein, the term “mutation” refers to a change introduced into the nucleic acid sequence encoding a protein that changes the amino acid sequence of the protein, including but not limited to substitutions, insertions, deletions, point mutations, transpositions, inversions, frame shifts, nonsense mutations, truncations, or other forms of aberrations. A mutation may produce no discernible changes or result in a new property, function, or trait of the mutated protein. An RTase mutant of the disclosure may have one or more mutations in the nucleic acid sequence encoding the RTase mutant resulting in one or more mutations in the amino acid sequence of the RTase mutant. A mutation can result in one or more amino acids being substituted for an alternate amino acid residue, including Ala, Arg, Asn, Asp, Cys, Gln, Glu, Gly, His, Ile, Leu, Lys, Met, Phe, Pro, Ser, Thr, Trp, Tyr, and/or Val. The resulting amino acid mutations may impart altered functional and biological properties to the RTase mutant including but not limited to increased activity, enhanced DNA synthesis, enhanced stability or enhanced thermostability, reduced or eliminated RNase H activity, reduced terminal deoxynucleotidyl transferase activity, increased accuracy or increased fidelity, increased specificity, or altered half-life.

As used herein, the terms “detecting,” “detection,” “determining,” and the like refer to assays performed for identification of the quantity of cDNA synthesis as a marker of RTase activity. The amount of marker expression or activity detected in the sample can be the same as, decreased, or increased as compared to the amount of marker expression or activity detected using the RTase base construct. One of skill in the art will understand that amount of cDNA can be quantified using multiple techniques.

The term “increased,” as used herein with regard to RTase activity, refers to the level of RTase activity of an RTase mutant as compared to the RTase base construct. An RTase mutant has “increased” RTase activity if the level of its RTase activity, as measured by the quantity of cDNA synthesized or as measured by other methods known in the art, is more than the RTase base construct activity. For example, the RTase activity of the RTase mutant is increased if the RTase activity is at least 10%, 15%, 20%, 25%, 30%, 35%, 40%, 45%, 50%, 55%, 60%, 65%, 70%, 75%, 80%, 85%, 90%, 95%, or 100% more than, or at least 2-fold, 3-fold, 4-fold, 5-fold, or 10-fold, or more than 10-fold more than the RTase base construct activity.

The term “decreased,” as used herein with regard to RTase activity, refers to the level of RTase activity of an RTase mutant as compared to the RTase base construct. An RTase mutant has “decreased” RTase activity if the level of its RTase activity, as measured by the quantity of cDNA synthesized or as measured by other methods known in the art is less than the RTase base construct activity. For example, the RTase activity of the RTase mutant is decreased if the RTase activity is at least 95%, 90%, 85%, 80%, 75%, 70%, 65%, 60%, 55%, 50%, 45%, 40%, 35%, 30%, 25%, 20%, 15%, 10%, or 5% less than, or at least 2-fold, 3-fold, 4-fold, 5-fold, 10-fold, or more than 10-fold less than the RTase base construct activity.

As used herein, the term “amplification” refers to any known in vitro procedure for obtaining multiple copies of a target nucleic acid sequence or its complement or fragments thereof. In vitro amplification refers to production of an amplified nucleic acid that may contain less than the complete target region sequence or its complement. Known in vitro amplification methods include, for example, transcription-mediated amplification, replicase-mediated amplification, polymerase chain reaction (PCR) amplification, ligase chain reaction (LCR) amplification, and strand-displacement amplification (SDA, including multiple strand-displacement amplification method (MSDA)). Replicase-mediated amplification uses self-replicating RNA molecules, and a replicase such as Q-β-replicase. PCR amplification uses DNA polymerase, primers, and thermal cycling to synthesize multiple copies of the two complementary strands of DNA or cDNA. PCR involves denaturation of a double-stranded DNA molecule, followed by annealing of DNA primers directed to the sequence of interest, and amplification/extension of the newly formed DNA strand. LCR amplification uses at least four separate oligonucleotides to amplify a target and its complementary strand by using multiple cycles of hybridization, ligation, and denaturation. SDA is a method in which a primer contains a recognition site for a restriction endonuclease that permits the endonuclease to nick one strand of a hemimodified DNA duplex that includes the target sequence, followed by amplification in a series of primer extension and strand displacement steps. Other strand-displacement amplification methods known in the art (e.g., MSDA) do not require endonuclease nicking. Those of skill in the art will understand that the oligonucleotide primer sequences of the disclosure may be readily used in any in vitro amplification method based on primer extension by a polymerase. As commonly known in the art, oligonucleotides are designed to bind to a complementary sequence under selected conditions.

As used herein, “real time PCR” or “quantitative PCR” refers to a PCR method wherein the amount of product being formed can be monitored using florescent probes and quantified by tracking the fluorescent signal produced, above a threshold level. Real time PCR can be performed in a one-step reaction that includes the reverse transcription step in a simultaneous reaction (i.e., real time PCR or RT-PCR) or in a two-step reaction in which the reverse transcription step and PCR steps are performed consecutively.

As used herein, the term “complementary” refers to the broad concept of sequence complementarity between regions of two nucleic acid strands or between two regions of the same nucleic acid strand. A first region of a nucleic acid is complementary to a second region of the same or a different nucleic acid if, when the two regions are arranged in an antiparallel fashion, at least one nucleotide of the first region is capable of base pairing with a nucleotide of the second region. In some embodiments, the first region comprises a first portion and the second region comprises a second portion, whereby, when the first and second portions are arranged in an antiparallel fashion, at least about 50%, 75%, 80%, 85%, 90%, 95%, 96%, 97%, 98%, or 99% of the nucleotides of the first portion are capable of base pairing with nucleotides in the second portion. In another embodiment, all nucleotides of the first portion are capable of base pairing with nucleotides in the second portion.

Polypeptide and polynucleotide sequences may be aligned, and percentages of identical amino acids or nucleotides in a specified region may be determined against another polypeptide or polynucleotide sequence, using computer algorithms that are publicly available. The percent identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the disclosure; and then multiplying by 100 to determine the percent identity.

As used herein, the terms “sample” and “biological sample” include a specimen or culture obtained from any source. Biological samples can be obtained from cerebrospinal fluid, lacrimal fluid, blood (including any blood product, such as whole blood, plasma, serum, or specific types of cells of the blood), urine, saliva, and the like. Biological samples also include tissue samples, such as biopsy tissues or pathological tissues that have previously been fixed (e.g., formaline snap frozen, cytological processing).

### 2. REVERSE TRANSCRIPTASES

The disclosure relates to novel C-terminal and/or N-terminal peptide extensions of Moloney murine leukemia virus (MMLV) and reverse transcriptase (RTase) mutants. MMLV RTases with C-terminal and/or N-terminal peptide extensions, as summarized in Tables 39 and 42 are prepared by enzyme overexpression in E. coli and purified by affinity, ion exchange, and mixed resin chromatography in order to purify the MMLV Rtase mutants. Purified MMLV RTases were then tested for their ability to synthesize cDNA from isolated total RNA.

The MMLV RTase mutants of the disclosure are prepared by modifying the sequence of an MMLV RTase base construct (SEQ ID NO: 637). In one embodiment, the MMLV RTase mutant of the disclosure comprises the amino acid sequence of SEQ ID NO: 637, wherein the amino acid sequence of the MMLV RTase mutant further comprises at least one amino acid substitution that is: (a) an isoleucine to arginine, lysine, or methionine substitution at position 61 (I61R, I61K, or I61M); (b) a glutamine to arginine, lysine, or isoleucine substitution at position 68 (Q68R, Q68K, or Q68I); (c) a glutamine to arginine, histidine, or isoleucine substitution at position 79 (Q79R, Q79H, or Q79I); (d) a leucine to arginine, lysine, or asparagine substitution at position 99 (L99R, L99K, or L99N); (e) a glutamic acid to aspartic acid, methionine, or tryptophan substitution at position 282 (E282D, E282M, or E282W); and/or (f) an arginine to alanine substitution at position 298 (R298A).

In another embodiment, the MMLV RTase mutant of the disclosure comprises the amino acid sequence of SEQ ID NO: 637, wherein the amino acid sequence of the MMLV RTase mutant further comprises at least two amino acid substitutions that are: (a) an isoleucine to arginine substitution at position 61 and a glutamic acid to aspartic acid substitution at position 282 (I61R/E282D); (b) a leucine to arginine at substitution position 99 and a glutamic acid to aspartic acid substitution at position 282 (L99R/E282D); (c) a glutamine to arginine substitution at position 68 and a glutamic acid to aspartic acid substitution at position 282 (Q68R/E282D); (d) a glutamine to arginine substitution at position 79 and a glutamic acid to aspartic acid substitution at position 282 (Q79R/E282D); (e) a glutamic acid to aspartic acid substitution at position 282 and an arginine to alanine substitution at position 298 (E282D/R298A); (f) an isoleucine to arginine substitution at position 61 and a leucine to arginine substitution at position 99 (I61R/L99R); (g) an isoleucine to arginine substitution at position 61 and a glutamine to arginine substitution at position 68 (I61R/Q68R); (h) an isoleucine to arginine substitution at position 61 and a glutamine to arginine substitution at position 79 (I61R/Q79R); (i) an isoleucine to arginine substitution at position 61 and an arginine to alanine substitution at position 298 (I61R/R298A); (j) a glutamine to arginine substitution at position 68 and a leucine to arginine substitution at position 99 (Q68R/L99R); (k) a glutamine to arginine substitution at position 79 and a leucine to arginine substitution at position 99 (Q79R/L99R); (1) a leucine to arginine at substitution position 99 and an arginine to alanine substitution at position 298 (L99R/R298A); (m) a glutamine to arginine substitution at position 68 and a glutamine to arginine substitution at position 79 (Q68R/Q79R); (n) a glutamine to arginine substitution at position 68 and an arginine to alanine substitution at position 298 (Q68R/R298A); or (o) a glutamine to arginine substitution at position 79 and an arginine to alanine substitution at position 298 (Q79R/R298A).

In another embodiment, the MMLV RTase mutant of the disclosure comprises the amino acid sequence of SEQ ID NO: 637, wherein the amino acid sequence of the MMLV RTase mutant further comprises at least three amino acid substitutions that are: (a) a glutamine to arginine substitution at position 68, a leucine to arginine substitution at position 99, and a glutamic acid to aspartic acid substitution at position 282 (Q68R/L99R/E282D); (b) a glutamine to arginine substitution at position 79, a leucine to arginine substitution at position 99, and a glutamic acid to aspartic acid substitution at position 282 (Q79R/L99R/E282D); (c) a glutamine to arginine substitution at position 68, a glutamine to arginine substitution at position 68, and a glutamic acid to aspartic acid substitution at position 282 (Q68R/Q79R/E282D); or (d) a glutamine to arginine substitution at position 68, a glutamine to arginine substitution at position 68, and a leucine to arginine substitution at position 99 (Q68R/Q79R/L99R).

In another embodiment, the MMLV RTase mutant of the disclosure comprises the amino acid sequence of SEQ ID NO: 637, wherein the amino acid sequence of the MMLV RTase mutant further comprises at least four amino acid substitutions that are: (a) a glutamine to arginine substitution at position 68, a glutamine to arginine substitution at position 79, a leucine to arginine substitution at position 99, and a glutamic acid to aspartic acid substitution at position 282 (Q68R/Q79R/L99R/E282D); (b) a glutamine to arginine substitution at position 68, a glutamine to arginine substitution at position 79, a leucine to lysine substitution at position 99, and a glutamic acid to aspartic acid substitution at position 282 (Q68R/Q79R/L99K/E282D); (c) a glutamine to arginine substitution at position 68, a glutamine to arginine substitution at position 79, a leucine to asparagine substitution at position 99, and a glutamic acid to aspartic acid substitution at position 282 (Q68R/Q79R/L99N/E282D); (d) a glutamine to isoleucine substitution at position 68, a glutamine to arginine substitution at position 79, a leucine to arginine substitution at position 99, and a glutamic acid to aspartic acid substitution at position 282 (Q68I/Q79R/L99R/E282D); (e) a glutamine to lysine substitution at position 68, a glutamine to arginine substitution at position 79, a leucine to arginine substitution at position 99, and a glutamic acid to aspartic acid substitution at position 282 (Q68K/Q79R/L99R/E282D); (f) a glutamine to arginine substitution at position 68, a glutamine to histidine substitution at position 79, a leucine to arginine substitution at position 99, and a glutamic acid to aspartic acid substitution at position 282 (Q68R/Q79H/L99R/E282D); (g) a glutamine to arginine substitution at position 68, a glutamine to isoleucine substitution at position 79, a leucine to arginine substitution at position 99, and a glutamic acid to aspartic acid substitution at position 282 (Q68R/Q79I/L99R/E282D); (h) a glutamine to arginine substitution at position 68, a glutamine to arginine substitution at position 79, a leucine to arginine substitution at position 99, and a glutamic acid to methionine substitution at position 282 (Q68R/Q79R/L99R/E282M); (i) a glutamine to arginine substitution at position 68, a glutamine to arginine substitution at position 79, a leucine to arginine substitution at position 99, and a glutamic acid to tryptophan substitution at position 282 (Q68R/Q79R/L99R/E282W); or (j) a glutamine to isoleucine substitution at position 68, a glutamine to histidine substitution at position 79, a leucine to lysine substitution at position 99, and a glutamic acid to methionine substitution at position 282 (Q68I/Q79H/L99K/E282M).

In another embodiment, the MMLV RTase mutant of the disclosure comprises the amino acid sequence of SEQ ID NO: 637, wherein the amino acid sequence of the MMLV RTase mutant further comprises at least five amino acid substitutions that are: (a) an isoleucine to lysine substitution at position 61, a glutamine to arginine substitution at position 68, a glutamine to arginine substitution at position 79, a leucine to arginine substitution at position 99, and a glutamic acid to aspartic acid substitution at position 282 (I61K/Q68R/Q79R/L99R/E282D); (b) an isoleucine to methionine substitution at position 61, a glutamine to arginine substitution at position 68, a glutamine to arginine substitution at position 79, a leucine to arginine substitution at position 99, and a glutamic acid to aspartic acid substitution at position 282 (I61M/Q68R/Q79R/L99R/E282D); or (c) an isoleucine to methionine substitution at position 61, a glutamine to isoleucine substitution at position 68, a glutamine to histidine substitution at position 79, a leucine to lysine substitution at position 99, and a glutamic acid to methionine substitution at position 282 (161M/Q68IR/Q79H/L99K/E282M).

In another embodiment, the MMLV RTase mutant of the disclosure comprises the amino acid sequence of SEQ ID NO: 637, wherein the amino acid sequence of the MMLV RTase mutant further comprises at least five or more amino acid substitutions that are: (a) a glutamine to arginine substitution at position 68, a glutamine to arginine substitution at position 79, a leucine to arginine substitution at position 99, a glutamic acid to aspartic acid substitution at position 282, a glutamine to glutamic acid substitution at position 299, a valine to arginine substitution at position 433, and a isoleucine to glutamic acid at position 593 (Q68R/Q79R/L99R/E282D/Q299E/V433R/I593E): (b) a glutamine to arginine substitution at position 68, a glutamine to arginine substitution at position 79, a leucine to argine substitution at position 82, a leucine to arginine substitution at position 99, a glutamic acid to aspartic acid substitution at position 282, a glutamine to glutamic acid substitution at position 299, a valine to arginine substitution at position 433, and a isoleucine to glutamic acid at position 593 (Q68R/Q79R/L82R/L99R/E282D/Q299E/V433R/I593E); (c) a glutamine to arginine substitution at position 68, a glutamine to arginine substitution at position 79, a leucine to argine substitution at position 82, a leucine to arginine substitution at position 99, a glutamic acid to aspartic acid substitution at position 282, a glutamine to glutamic acid substitution at position 299, a threonine to glutamic acid substitution at position 332, and a isoleucine to glutamic acid at position 593 (Q68R/Q79R/L82R/L99R/E282D/Q299E/T332E/I593E); (d) a glutamine to arginine substitution at position 68, a glutamine to arginine substitution at position 79, a leucine to argine substitution at position 82, a leucine to arginine substitution at position 99, a glutamic acid to aspartic acid substitution at position 282, a glutamine to glutamic acid substitution at position 299, a threonine to glutamic acid substitution at position 332, a valine to arginine substitution at position 433, and a isoleucine to glutamic acid at position 593 (Q68R/Q79R/L82R/L99R/E282D/Q299E/T332E/V433R/I593E).

In another embodiment, the MMLV RTase mutant of the disclosure comprises the amino acid sequence of SEQ ID NO: 717, wherein the amino acid sequence of the MMLV RTase mutant further comprises at least two amino acid substitutions that are: (a) a glutamine to arginine substitution at position 68 (Q68R); (b) a glutamine to arginine substitution at position 79 (Q79R); (c) a leucine to tyrosine at position 82 (L82Y); (d) a leucine to arginine substitution at position 99 (L99R); (e) a leucine to isoluecine at position 280 (L280I); (f) a glutamic acid to aspartic acid substitution at position 282 (E282D); (g) a glutamine to glutamic acid substitution at position 299 (Q299E); (h) threonine to lysine at position 306 (T306K); (i) a valine to asparagine at position 433 (V433N); (j) a valine to arginine at position 433 (V433R); (k) an isoleucine to glutamic acid at position 593 (I593E); or (1) an isoleucine to tryptophan at position 593 (I593W).

In another embodiment, the MMLV RTase mutant of the disclosure comprises the amino acid sequence of SEQ ID NO: 717, wherein the amino acid sequence of the MMLV RTase mutant further comprises the amino acid substitutions: (a) a glutamine to arginine substitution at position 68 (Q68R); (b) a glutamine to arginine substitution at position 79 (Q79R); (c) a leucine to tyrosine substitution at position 82 (L82Y); (d) a leucine to arginine substitution at position 99 (L99R); (e) a leucine to isoleucine substitution at position 280 (L280I); (f) a glutamic acid to aspartic acid substitution at position 282 (E282D); (g) a glutamine to glutamic acid substitution at position 299 (Q299E); (h) a threonine to lysine substitution at position 306 (T306K); (i) a valine to asparagine substitution at position 433 (V433N); and (j) an isoleucine to tryptophan substitution at position 593 (I593W).

In another embodiment, the MMLV RTase mutant of the disclosure comprises the amino acid sequence of SEQ ID NO: 717, wherein the amino acid sequence of the MMLV RTase mutant further comprises the amino acid substitutions: (a) a glutamine to arginine substitution at position 68 (Q68R); (b) a glutamine to arginine substitution at position 79 (Q79R); (c) a leucine to tyrosine substitution at position 82 (L82Y); (d) a leucine to arginine substitution at position 99 (L99R); (e) a leucine to isoleucine substitution at position 280 (L280I); (f) a glutamic acid to aspartic acid substitution at position 282 (E282D); (g) a glutamine to glutamic acid substitution at position 299 (Q299E); (h) a threonine to lysine substitution at position 306 (T306K); (i) a valine to arginine substitution at position 433 (V433R); and (j) an isoleucine to glutamic acid substitution at position 593 (I593E).

In one embodiment the RTase mutant amino acid sequence comprises a mutant selected from Tables 3, 8, 9, 12, 21, 22, or 38. In one aspect, the RTase mutant amino acid sequence comprises a mutant selected from the amino acid sequences of SEQ ID NO: 638, SEQ ID NO: 639, SEQ ID NO: 640, SEQ ID NO: 641, SEQ ID NO: 642, SEQ ID NO: 643, SEQ ID NO: 644, SEQ ID NO: 645, SEQ ID NO: 646, SEQ ID NO: 647, SEQ ID NO: 648, SEQ ID NO: 649, SEQ ID NO: 650, SEQ ID NO: 651, SEQ ID NO: 652, SEQ ID NO: 653, SEQ ID NO: 654, SEQ ID NO: 655, SEQ ID NO: 656, SEQ ID NO: 657, SEQ ID NO: 658, SEQ ID NO: 659, SEQ ID NO: 660, SEQ ID NO: 661, SEQ ID NO: 662, SEQ ID NO: 663, SEQ ID NO: 664, SEQ ID NO: 665, SEQ ID NO: 666, SEQ ID NO: 667, SEQ ID NO: 668, SEQ ID NO: 669, SEQ ID NO: 679, SEQ ID NO: 671, SEQ ID NO: 672, SEQ ID NO: 673, SEQ ID NO: 674, SEQ ID NO: 675, SEQ ID NO: 676, SEQ ID NO: 677, SEQ ID NO: 678, SEQ ID NO: 679, SEQ ID NO: 670, SEQ ID NO: 671, SEQ ID NO: 672, SEQ ID NO: 673, SEQ ID NO: 674, SEQ ID NO: 675, SEQ ID NO: 676, SEQ ID NO: 677, SEQ ID NO: 678, SEQ ID NO: 679, SEQ ID NO: 680, SEQ ID NO: 681, SEQ ID NO: 682, SEQ ID NO: 683, SEQ ID NO: 684, SEQ ID NO: 685, SEQ ID NO: 686, SEQ ID NO: 687, SEQ ID NO: 688, SEQ ID NO: 689, SEQ ID NO: 690, SEQ ID NO: 691, SEQ ID NO: 692, SEQ ID NO: 693, SEQ ID NO: 694, SEQ ID NO: 695, SEQ ID NO: 696, SEQ ID NO: 697, SEQ ID NO: 698, SEQ ID NO: 699, SEQ ID NO: 716, SEQ ID NO: 717, SEQ ID NO: 718, SEQ ID NO: 719, SEQ ID NO: 720, SEQ ID NO: 721, SEQ ID NO: 722, SEQ ID NO: 723, SEQ ID NO: 724, SEQ ID NO: 725, SEQ ID NO: 726, SEQ ID NO: 727, SEQ ID NO: 728, SEQ ID NO: 729, SEQ ID NO: 730, or SEQ ID NO: 731.

In one embodiment, the RTase mutant amino acid sequence comprises a C-terminal extension. In one aspect, the C-terminal extension comprises a peptide sequence. In another embodiment, an isolated polypeptide encodes a RTase mutant with a C-terminal extension.

In another embodiment, the RTase mutant amino acid sequence comprises an N-terminal extension. In one aspect, the N-terminal extension comprises a peptide sequence. In another embodiment, an isolated polypeptide encodes a RTase mutant with an N-terminal extension.

In another embodiment, the RTase mutant amino acid sequence comprises both a C-terminal extension and an N-terminal extension. In one aspect, the C-terminal extension and the N-terminal extension comprise a peptide sequence. In another embodiment, an isolated polypeptide encodes a RTase mutant with both a C-terminal extension and an N-terminal extension.

The claimed invention is based, at least in part, on the discovery that certain single and double amino acid mutations introduced into an MMLV RTase sequence, as disclosed herein, result in an MMLV RTase with increased or enhanced thermostability and/or RTase activity. Accordingly, methods for synthesizing the MMLV RTase mutants and methods for performing reverse transcription-polymerase chain reaction (RT-PCR) are also provided herein. Further provided are kits comprising the isolated MMLV RTase single, double, triple, or more mutations.

In certain embodiments, the mutated RTase is derived from the retrovirus Moloney murine leukemia virus (MMLV). In other embodiments, a mutated RTase of the disclosure could be derived from the RTase from a retrovirus other than MMLV, such as avian myeloblastosis virus (AMV) or human immunodeficiency virus type 1 (HIV-1), by introducing the same mutations into an RTase base construct obtained from the other retrovirus.

In certain embodiments, the RTase mutants of the disclosure are obtained by genetic engineering techniques that are well known in the art. For example, site-directed and random mutagenesis can be used to generate the RTase mutants of the disclosure.

In one embodiment of the disclosure, an RTase mutant of the disclosure is part of a composition.

### 3. MUTAGENESIS

The RTase mutants of the disclosure can be prepared by standard methods disclosed herein or known in the art. In one embodiment, the nucleic acid sequence of the RTase base construct (SEQ ID NO: 637) is modified to create a nucleic acid sequence encoding an RTase mutant. One of skill in the art will recognize that colonies with the appropriate strains can be used to grow and express an RTase mutant of interest, and following cell harvest and protein isolation, the RTase mutant can be used in cDNA synthesis techniques. Non-limiting examples of mutagenesis and cDNA synthesis are described herein in Examples 1-3.

As used herein, the term “mutagenesis” refers to the introduction of a genetic change in the nucleic acid sequence of a cell, wherein the alteration is then inherited by each cell. One of skill in the art will understand that mutations in a given nucleic acid sequence can be introduced using a variety of methods. One of skill in the art will further recognize that mutagenesis methods seek to mutate a target gene or target polynucleotide. The target gene may encode any one or more desired proteins. Mutagenesis methods commonly use a synthetic oligonucleotide that carries the desired sequence modification. The mutagenic oligonucleotide is incorporated into the DNA sequence using in vitro enzymatic DNA synthesis and is propagated in a mutant or wild-type bacterium.

Site directed mutagenesis, wherein targeted mutations are introduced into one or more desired positions of a template polynucleotide, may be achieved using primer extension mutagenesis. This technique requires the use of a specific primer that contains one or more desired mutations relative to the template polynucleotide. The mutagenesis primer can be a synthetic oligonucleotide or a PCR product. The mutated primer may include one or more substitutions, deletions, additions, or combinations thereof.

Mutated reverse transcriptases may also be generated using random mutagenesis, wherein mutations are introduced into the mutagenesis primer during synthesis. Randomly mutagenized oligonucleotides may also be used as mutagenesis primers.

In another embodiment, the mutated reverse transcriptases of the disclosure can be developed using error-prone rolling circle amplification (RCA). In this technique, the fidelity of a DNA polymerase is decreased by performing the RCA in the presence of MnCl₂ or by decreasing the amount of input DNA.

### 4. CDNA SYNTHESIS

The disclosure also relates to the activity of MMLV RTases, as measured by the quantity of cDNA produced by the MMLV RTases disclosed herein. cDNA can be prepared using one-step or two-step procedures and can be obtained from a variety of template molecules. As used herein, the term “template molecule” refers to a biological molecule that carries the genetic code for use in making a new nucleic acid strand. For example, in DNA replication, the unwound double helix and each single-stranded DNA molecule is used as a template to synthesize a complementary strand. Reverse transcription generates cDNA from RNA. One of skill in the art will understand that cDNA molecules may be prepared from a variety of nucleic acid template molecules. In one embodiment, the nucleic acid template can be single-stranded or double-stranded DNA. In one embodiment, RNA can be used in cDNA synthesis. In certain embodiments, the MMLV RTase mutants of the disclosure exhibit increased or enhanced thermostability and/or RTase activity as compared to an RTase base construct. In other embodiments, the MMLV RTase mutants of the disclosure exhibit altered half-life, reduced or eliminated RNase H activity, reduced terminal deoxynucleotidyl transferase activity, increased accuracy or fidelity, or increased specificity.

The disclosure also provides methods for synthesizing cDNA using the MMLV RTase mutants of the disclosure that have single or double amino acid mutations. The MMLV RTase mutants of the disclosure may be used in methods that produce a first strand cDNA or a first and second strand cDNA. One of skill in the art will understand that first and second strand cDNA may form a double-stranded DNA molecule, which may include a full-length cDNA sequence and cDNA libraries.

The cDNA molecules that have been reverse transcribed by the MMLV RTase mutants of the disclosure may be isolated, or the reaction mixture containing the cDNA molecules may be directly used in downstream applications or for further analysis or manipulation. Amplification methods that may be used to practice the methods of the disclosure are described herein and are well known in the art. Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as AMV RTase or MMLV RTase).

Amplification methods utilize pairs of primers that selectively hybridize to nucleic acids corresponding to a specific nucleotide sequence of interest that are contacted with the isolated nucleic acid under conditions that permit selective hybridization. Once hybridized, the nucleic acid:primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In certain methods, the detection may be performed by visual means. Alternatively, the detection may involve indirect identification of the product via chemiluminescence, radioactive scintigraphy of incorporated radiolabel or fluorescent label, or even via a system using electrical or thermal impulse signals.

Methods based on ligation of two (or more) oligonucleotides in the presence of a nucleic acid having the sequence of the resulting “di-oligonucleotide,” thereby amplifying the di-oligonucleotide, also may be used in the amplification step of the disclosure.

In some embodiments of the disclosure, the detection process can utilize a hybridization technique, for example, wherein a specific primer or probe is selected to anneal to a target biomarker of interest, and thereafter detection of selective hybridization is made. As commonly known in the art, the oligonucleotide probes and primers can be designed by taking into consideration the melting point of hybridization thereof with its targeted sequence.

One of skill in the art will recognize that cDNA molecules made using the MMLV RTase mutants of the disclosure can be used in a variety of additional downstream applications. For example, amplification methods may include one-step PCR, two-step PCR, real-time or quantitative PCR, hot-start PCR, nested PCR, touch down PCR, differential display PCR (DDRT-PCR), microarray technologies, inverse PCR, Rapid amplification of PCR ends (RACE or anchored PCR), multiplex PCR, and site directed PCR mutagenesis. Synthesized cDNA and cDNA libraries created with the MMLV RTase mutants of the disclosure can be used in cloning and/or sequencing for further characterization. One of skill in the art will recognize that nucleic acid amplification using cDNA prepared with the MMLV RTase mutants of the disclosure may include additional techniques not listed herein.

To enable hybridization to occur under the methods presented above, oligonucleotide primers and probes should comprise an oligonucleotide sequence that has at least 70%, 75%, 80%, 85%, 90%, 95%, 96%, 97%, 98%, 99%, or 100% identity to a portion of the sequence of interest.

### 5. C-TERMINAL AND N-TERMINAL EXTENSIONS

The disclosure also relates to C-terminal and/or N-terminal peptide extensions that improve the performance of an MMLV RTase. C-terminal and N-terminal extensions are peptide additions to the C-terminal or N-terminals ends of the MMLV RTase. The MMLV RTase of the current disclosure contains an unnatural peptide tag on the C-terminal end, the N-terminal end, or both the C-terminal and N-terminal ends of the enzyme that improves the performance of the MMLV RTase, including increased RTase activity and thermostability. More specifically, the C-terminal and N-terminal peptide extensions described herein are fusions of domains from known thermostable enzymes to that of the MMLV Rtase. Results disclosed herein were achieved by overexpresseing enzymes in E. coli followed by affinity purification, ion exchange, and mixed resin chromatography to prepare purified protein, and the purified MMLV RTases were tested for their ability to synthesize cDNA from isolated total RNA.

In one embodiment, the C-terminal and/or N-terminal peptide extensions comprise the amino acid sequences of SEQ ID NOs: 732-761. The peptide extensions can reside on either one or both of the C-terminal and N-terminal ends of the MMLV RTase. In other embodiments, the C-terminal or N-terminal peptide extension is the amino acid sequence of SEQ ID NO: 735, SEQ ID NO: 736, SEQ ID NO: 739, SEQ ID NO: 744, or SEQ ID NO: 747.

In one embodiment, the N-terminal or C-terminal peptide extension is added to an MMLV RTase mutant comprising the amino acid sequence of SEQ ID NO: 717, wherein the amino acid sequence of the MMLV RTase mutant further comprises the amino acid substitutions: (a) a glutamine to arginine substitution at position 68 (Q68R); (b) a glutamine to arginine substitution at position 79 (Q79R); (c) a leucine to tyrosine substitution at position 82 (L82Y); (d) a leucine to arginine substitution at position 99 (L99R); (e) a leucine to isoleucine substitution at position 280 (L280I); (f) a glutamic acid to aspartic acid substitution at position 282 (E282D); (g) a glutamine to glutamic acid substitution at position 299 (Q299E); (h) a threonine to lysine substitution at position 306 (T306K); (i) a valine to asparagine substitution at position 433 (V433N); and (j) an isoleucine to tryptophan substitution at position 593 (I593W).

In other embodiments, the C-terminal and N-terminal peptide extensions added to an MMLV TRase mutant are selected from the sequences set forth in Tables 3, 8, 9, 12, 21, 22, or 38. In one aspect, the N-terminal or C-terminal peptide extensions are selected from the amino acid sequences of SEQ ID NO: 732, SEQ ID NO: 733, SEQ ID NO: 734, SEQ ID NO: 735, SEQ ID NO: 736, SEQ ID NO: 737, SEQ ID NO: 738, SEQ ID NO: 739, SEQ ID NO: 740, SEQ ID NO: 741, SEQ ID NO: 742, SEQ ID NO: 743, SEQ ID NO: 744, SEQ ID NO: 745, SEQ ID NO: 746, SEQ ID NO: 747, SEQ ID NO: 748, SEQ ID NO: 749, SEQ ID NO: 750, SEQ ID NO: 751, SEQ ID NO: 752, SEQ ID NO: 753, SEQ ID NO: 754, SEQ ID NO: 755, SEQ ID NO: 756, SEQ ID NO: 757, SEQ ID NO: 758, SEQ ID NO: 759, SEQ ID NO: 760, or SEQ ID NO: 761.

### 6. BIOLOGICAL SAMPLES

The MMLV RTase mutants and associated methods of the disclosure may be practiced with any suitable biological sample from which RNA or DNA can be isolated. In one embodiment of the disclosure, the biological sample may be a bodily fluid or tissue obtained from either a diseased or a healthy subject. In some embodiments of the disclosure, the biological sample may be a bodily fluid, including but not limited to whole blood, plasma, serum, feces, or urine. In another embodiment, the methods of the disclosure may be practiced with any suitable samples that are freshly isolated or that have been frozen or stored after having been collected from a subject, for example, with a known diagnosis, treatment, and/or outcome history. Samples may be collected by any non-invasive means, such as, for example, fine needle aspiration or needle biopsy, or alternatively, by an invasive method, including, for example, surgical biopsy. In such embodiments, RNA or DNA can be extracted from a biological sample (e.g., blood serum) before analysis. Methods of RNA and DNA extraction are well known in the art.

A number of kits for use in extracting RNA (i.e., total RNA or mRNA) from bodily fluids or tissues (e.g., blood serum) and are known in the art and commercially available. One of ordinary skill in the art can easily select an appropriate kit for a particular situation.

In certain embodiments of the disclosure, after extraction, mRNA is amplified, and transcribed into cDNA, which can then serve as template for multiple rounds of transcription by the appropriate RNA polymerase. Amplification methods that may be used to practice the methods of the disclosure are described herein and are well known in the art. Reverse transcription reactions may be carried out using non-specific primers, such as an anchored oligo-dT primer, or random sequence primers, or using a target-specific primer complementary to the RNA for each genetic probe being monitored, or using thermostable DNA polymerases, such as MMLV RTase or the MMLV RTase mutants of the disclosure.

In certain embodiments, the RNA isolated from a biological sample (e.g., after amplification and/or conversion to cDNA or cRNA) is labeled with a detectable agent before being analyzed. The role of a detectable agent is to facilitate detection of RNA or to allow visualization of hybridized nucleic acid fragments (e.g., nucleic acid fragments hybridized to genetic probes in an array-based assay). In some embodiments, the detectable agent is selected such that it generates a signal which can be measured and whose intensity is related to the amount of labeled nucleic acids present in the sample being analyzed.

Methods for labeling nucleic acid molecules are well known in the art. A review of labeling protocols and label detection techniques can be found in Kricka, Ann. Clin. Biochem. 39: 114-29 (2002); van Gijlswijk et al., Expert Rev. Mol. Diagn. 1: 81-91 (2001); and Joos et al., J. Biotechnol. 35: 135-53 (1994). Standard nucleic acid labeling methods include incorporation of radioactive agents; direct attachment of fluorescent dyes or of enzymes; chemical modifications of nucleic acid fragments making them detectable immunochemically or by other affinity reactions; and enzyme-mediated labeling methods, such as random priming, nick translation, PCR, and tailing with terminal transferase.

Any of a wide variety of detectable agents can be used to practice the methods of the disclosure. Suitable detectable agents include but are not limited to various ligands, radionuclides, fluorescent dyes, chemiluminescent agents, microparticles (such as, for example, quantum dots, nanocrystals, and phosphors), enzymes (such as, for example, those used in an ELISA, i.e., horseradish peroxidase, beta-galactosidase, luciferase, and alkaline phosphatase), colorimetric labels, magnetic labels, biotin, dioxigenin, or other haptens and proteins for which antisera or monoclonal antibodies are available.

### 7. KITS

The disclosure also provides kits for use in reverse transcription or related technologies. These kits include one or more of the following: an MMLV RTase mutant enzyme, reagents and buffers for conducting a reverse transcriptase reaction, a box, vial tubes, ampules, and the like. Kits can also include instructions for use of the kit for practicing any of the methods disclosed herein or other methods known to those of skill in the art.

### EXAMPLES

The claimed invention is further illustrated by the following Examples, which should not be construed as limiting. Those of skill in the art will recognize that the claimed invention may be practiced with variations of the disclosed structures, materials, compositions, and methods, and such variations are regarded as within the scope of the claimed invention.

The RTases described herein were overexpressed in E. coli, purified to homogeneity, and tested for their ability to enhance RNA detection in the context of reverse transcriptase quantitative PCR (RT-qPCR).

### Example 1. Preparation of Reverse Transcriptase Mutants by Site Directed Mutagenesis

a. Cloning of MMLV RTase Mutants Created from Base Construct (RNase H Minus Construct)

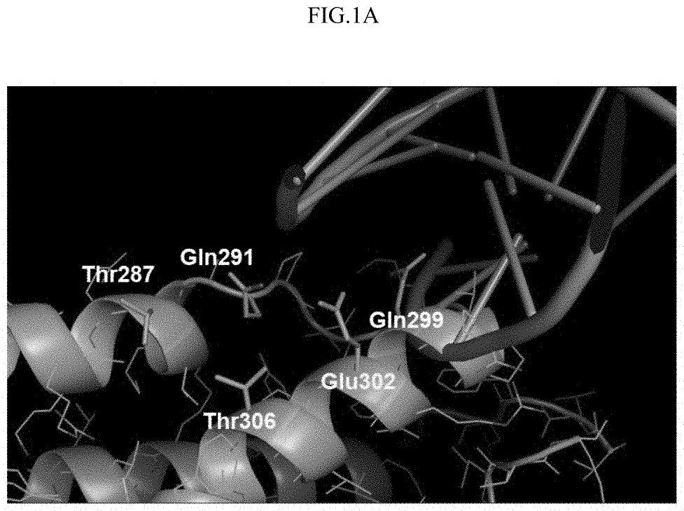

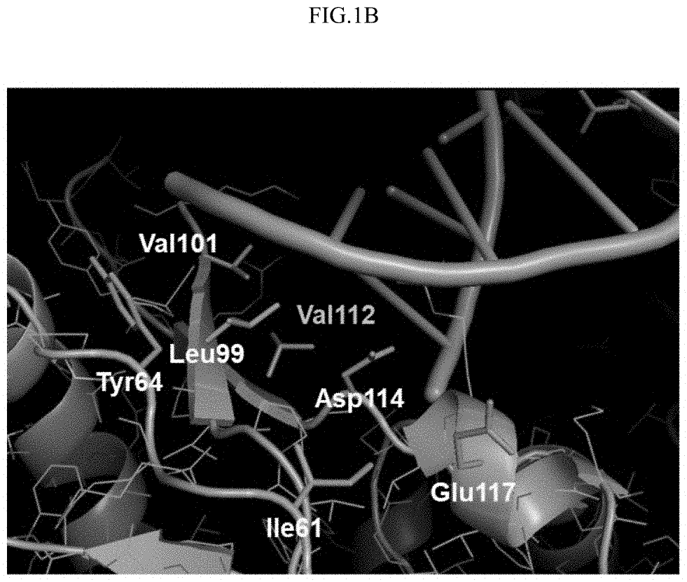

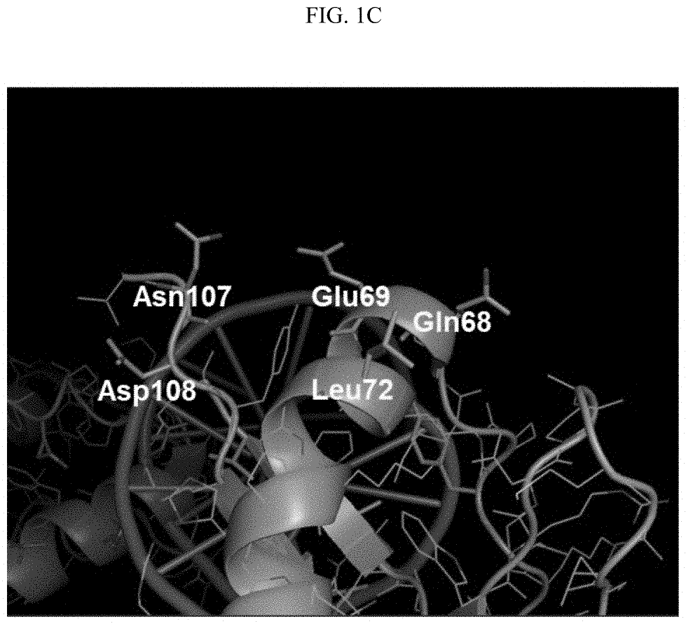

MMLV RTase mutants were prepared by first introducing three mutations (D524G, E562Q, and D583N) into the amino acid sequence of the wild-type, or naturally occurring, MMLV RTase to prepare an MMLV RTase base construct (SEQ ID NO: 637). The three mutations, which are contained in the SuperScript II RTase (Invitrogen), have been shown to reduce RNase H activity (see U.S. Pat. No. 5,405,776). The MMLV RTase base construct was optimized for E. coli expression and obtained as gBlocks® Gene Fragments (Integrated DNA Technologies) or by custom gene synthesis with the appropriate purification tag. Subsequent genes were amplified using standard PCR conditions and primers (see Tables 1 and 21). Amplified DNA was subjected to purification using a QIAquick PCR Purification kit (Qiagen, Catalog #28104), followed by gene fragment assembly into a pET28b expression plasmid. Plasmid DNA was isolated and sequenced to verify the desired sequence following transformation into E. coli cells. MMLV RTase mutations were selected by rational design (FIGS. 1A-1C) and introduced by site-directed mutagenesis, using standard PCR conditions and primers (see Tables 1 and 21). Resulting plasmids were transformed into E. coli BL21(DE3) cells for expression.

TABLE 1
Sequences of primers used for cloning of MMLV RTase base constructs and
mutants into pET28b.

| SEQ | | |
|---|---|---|
| ID NO: | Primer Name | Primer Sequence (5′-3′) |
| 1 | pET28b 5′ Reverse | GGTATATCTCCTTCTTAAAGTTAAACAAAATTATT |
| | | TCTAGAGGGGAAT |
| 2 | pET28b 3′ Forward | GATCCGGCTGCTAACAAAGCC |
| 3 | MMLV 5′ Primer | TTTTGTTTAACTTTAAGAAGGAGATATACCATGGG |
| | | CAGCAGCCATCATCATC |
| 4 | MMLV 3′ Primer | GCAGCCAACTCAGCTTCCTTTCGGGCTTTGTTAAA |
| | | AATGCTCGCTAGTGTAGGGAGAGC |
| 5 | MMLV K53A Top | AAGCACCGTTGATCATCCCGTTAGCGGCAACGTCT |
| | SDM | ACACCTGTCTCTATCAAAC |
| 6 | MMLV K53R Top | AAGCACCGTTGATCATCCCGTTACGTGCAACGTCT |
| | SDM | ACACCTGTCTCTATCAAAC |
| 7 | MMLV K53E Top | AAGCACCGTTGATCATCCCGTTAGAAGCAACGTCT |
| | SDM | ACACCTGTCTCTATCAAAC |
| 8 | MMLV T55A Top | CCGTTGATCATCCCGTTAAAGGCAGCGTCTACACC |
| | SDM | TGTCTCTATCAAACAGTACCCC |
| 9 | MMLV T55R Top | CCGTTGATCATCCCGTTAAAGGCACGTTCTACACC |
| | SDM | TGTCTCTATCAAACAGTACCCC |
| 10 | MMLV T55E Top | CCGTTGATCATCCCGTTAAAGGCAGAATCTACACC |
| | SDM | TGTCTCTATCAAACAGTACCCC |
| 11 | MMLV T57A Top | ATCATCCCGTTAAAGGCAACGTCTGCGCCTGTCTC |
| | SDM | TATCAAACAGTACCCCATGAG |
| 12 | MMLV T57R Top | ATCATCCCGTTAAAGGCAACGTCTCGTCCTGTCTC |
| | SDM | TATCAAACAGTACCCCATGAG |
| 13 | MMLV T57E Top | ATCATCCCGTTAAAGGCAACGTCTGAACCTGTCTC |
| | SDM | TATCAAACAGTACCCCATGAG |
| 14 | MMLV V59A Top | CCGTTAAAGGCAACGTCTACACCTGCGTCTATCAA |
| | SDM | ACAGTACCCCATGAGTCAAGAGG |
| 15 | MMLV V59R Top | CCGTTAAAGGCAACGTCTACACCTCGTTCTATCAA |
| | SDM | ACAGTACCCCATGAGTCAAGAGG |
| 16 | MMLV V59E Top | CCGTTAAAGGCAACGTCTACACCTGAATCTATCAA |
| | SDM | ACAGTACCCCATGAGTCAAGAGG |
| 17 | MMLV 161A Top | TAAAGGCAACGTCTACACCTGTCTCTGCGAAACAG |
| | SDM | TACCCCATGAGTCAAGAGG |
| 18 | MMLV 161R Top | TAAAGGCAACGTCTACACCTGTCTCTCGTAAACAG |
| | SDM | TACCCCATGAGTCAAGAGG |
| 19 | MMLV 161E Top | TAAAGGCAACGTCTACACCTGTCTCTGAAAAACAG |
| | SDM | TACCCCATGAGTCAAGAGG |
| 20 | MMLV K62A Top | GGCAACGTCTACACCTGTCTCTATCGCGCAGTACC |
| | SDM | CCATGAGTCAAGAGGC |
| 21 | MMLV K62R Top | GGCAACGTCTACACCTGTCTCTATCCGTCAGTACC |
| | SDM | CCATGAGTCAAGAGGC |
| 22 | MMLV K62E Top | GGCAACGTCTACACCTGTCTCTATCGAACAGTACC |
| | SDM | CCATGAGTCAAGAGGC |
| 23 | MMLV Q68A Top | CTGTCTCTATCAAACAGTACCCCATGAGTGCGGAG |
| | SDM | GCCCGCCTGGG |
| 24 | MMLV Q68R Top | CTGTCTCTATCAAACAGTACCCCATGAGTCGTGAG |
| | SDM | GCCCGCCTGGG |
| 25 | MMLV Q68E Top | CTGTCTCTATCAAACAGTACCCCATGAGTGAAGAG |
| | SDM | GCCCGCCTGGG |
| 26 | MMLV K75A Top | GGCCCGCCTGGGGATTGCGCCACATATTCAGCGCT |
| | SDM | TGCTGGACCA |
| 27 | MMLV K75R Top | GGCCCGCCTGGGGATTCGTCCACATATTCAGCGCT |
| | SDM | TGCTGGACCA |
| 28 | MMLV K75E Top | GGCCCGCCTGGGGATTGAACCACATATTCAGCGCT |
| | SDM | TGCTGGACCA |
| 29 | MMLV Q79A Top | CGCCTGGGGATTAAGCCACATATTGCGCGCTTGCT |
| | SDM | GGACCAGGGG |
| 30 | MMLV Q79R Top | CGCCTGGGGATTAAGCCACATATTCGTCGCTTGCT |
| | SDM | GGACCAGGGG |
| 31 | MMLV Q79E Top | CGCCTGGGGATTAAGCCACATATTGAACGCTTGCT |
| | SDM | GGACCAGGGG |
| 32 | MMLV L99A Top | CCGTGGAACACCCCCCTTGCGCCCGTGAAAAAGCC |
| | SDM | AGGTACAAAC |
| 33 | MMLV L99R Top | CCGTGGAACACCCCCCTTCGTCCCGTGAAAAAGCC |
| | SDM | AGGTACAAAC |
| 34 | MMLV L99E Top | CCGTGGAACACCCCCCTTGAACCCGTGAAAAAGCC |
| | SDM | AGGTACAAAC |
| 35 | MMLV V101A Top | CACCCCCCTTCTGCCCGCGAAAAAGCCAGGTACAA |
| | SDM | ACGATTATCGTCC |
| 36 | MMLV V101R Top | CACCCCCCTTCTGCCCCGTAAAAAGCCAGGTACAA |
| | SDM | ACGATTATCGTCC |
| 37 | MMLV V101E Top | CACCCCCCTTCTGCCCGAAAAAAAGCCAGGTACAA |
| | SDM | ACGATTATCGTCC |
| 38 | MMLV K102A Top | CCCCCTTCTGCCCGTGGCGAAGCCAGGTACAAACG |
| | SDM | ATTATCGTCC |
| 39 | MMLV K102R Top | CCCCCTTCTGCCCGTGCGTAAGCCAGGTACAAACG |
| | SDM | ATTATCGTCC |
| 40 | MMLV K102E Top | CCCCCTTCTGCCCGTGGAAAAGCCAGGTACAAACG |
| | SDM | ATTATCGTCC |
| 41 | MMLV K103A Top | CCCCCTTCTGCCCGTGAAAGCGCCAGGTACAAACG |
| | SDM | ATTATCGTCCAGTT |
| 42 | MMLV K103R Top | CCCCCTTCTGCCCGTGAAACGTCCAGGTACAAACG |
| | SDM | ATTATCGTCCAGTT |
| 43 | MMLV K103E Top | CCCCCTTCTGCCCGTGAAAGAACCAGGTACAAACG |
| | SDM | ATTATCGTCCAGTT |
| 44 | MMLV T106A Top | GCCCGTGAAAAAGCCAGGTGCGAACGATTATCGTC |
| | SDM | CAGTTCAAGATCTTCG |
| 45 | MMLV T106R Top | GCCCGTGAAAAAGCCAGGTCGTAACGATTATCGTC |
| | SDM | CAGTTCAAGATCTTCG |
| 46 | MMLV T106E Top | GCCCGTGAAAAAGCCAGGTGAAAACGATTATCGTC |
| | SDM | CAGTTCAAGATCTTCG |
| 47 | MMLV N107A Top | CCCGTGAAAAAGCCAGGTACAGCGGATTATCGTCC |
| | SDM | AGTTCAAGATCTTCGCG |
| 48 | MMLV N107R Top | CCCGTGAAAAAGCCAGGTACACGTGATTATCGTCC |
| | SDM | AGTTCAAGATCTTCGCG |
| 49 | MMLV N107E Top | CCCGTGAAAAAGCCAGGTACAGAAGATTATCGTCC |
| | SDM | AGTTCAAGATCTTCGCG |
| 50 | MMLV Y109A Top | CGTGAAAAAGCCAGGTACAAACGATGCGCGTCCAG |
| | SDM | TTCAAGATCTTCGCG |
| 51 | MMLV Y109R Top | CGTGAAAAAGCCAGGTACAAACGATCGTCGTCCAG |
| | SDM | TTCAAGATCTTCGCG |
| 52 | MMLV Y109E Top | CGTGAAAAAGCCAGGTACAAACGATGAACGTCCAG |
| | SDM | TTCAAGATCTTCGCG |
| 53 | MMLV R110A Top | CGTGAAAAAGCCAGGTACAAACGATTATGCGCCAG |
| | SDM | TTCAAGATCTTCGCGAGG |
| 54 | MMLV R110K Top | CGTGAAAAAGCCAGGTACAAACGATTATAAACCAG |
| | SDM | TTCAAGATCTTCGCGAGG |
| 55 | MMLV R110E Top | CGTGAAAAAGCCAGGTACAAACGATTATGAACCAG |
| | SDM | TTCAAGATCTTCGCGAGG |
| 56 | MMLV V112A Top | GCCAGGTACAAACGATTATCGTCCAGCGCAAGATC |
| | SDM | TTCGCGAGGTCAACAAAC |
| 57 | MMLV V112R Top | GCCAGGTACAAACGATTATCGTCCACGTCAAGATC |
| | SDM | TTCGCGAGGTCAACAAAC |
| 58 | MMLV V112E Top | GCCAGGTACAAACGATTATCGTCCAGAACAAGATC |
| | SDM | TTCGCGAGGTCAACAAAC |
| 59 | MMLV K120A Top | AGTTCAAGATCTTCGCGAGGTCAACGCGCGCGTAG |
| | SDM | AAGACATCCATCCGAC |
| 60 | MMLV K120R Top | AGTTCAAGATCTTCGCGAGGTCAACCGTCGCGTAG |
| | SDM | AAGACATCCATCCGAC |
| 61 | MMLV K120E Top | AGTTCAAGATCTTCGCGAGGTCAACGAACGCGTAG |
| | SDM | AAGACATCCATCCGAC |
| 62 | MMLV E123A Top | GCGAGGTCAACAAACGCGTAGCGGACATCCATCCG |
| | SDM | ACTGTACCTAATCC |
| 63 | MMLV E123R Top | GCGAGGTCAACAAACGCGTACGTGACATCCATCCG |
| | SDM | ACTGTACCTAATCC |
| 64 | MMLV E123D Top | GCGAGGTCAACAAACGCGTAGATGACATCCATCCG |
| | SDM | ACTGTACCTAATCC |
| 65 | MMLV T128V Top | ACGCGTAGAAGACATCCATCCGGTGGTACCTAATC |
| | SDM | CTTATAATCTGTTATCAGGCCTGC |
| 66 | MMLV T128R Top | ACGCGTAGAAGACATCCATCCGCGTGTACCTAATC |
| | SDM | CTTATAATCTGTTATCAGGCCTGC |
| 57 | MMLV T128E Top | ACGCGTAGAAGACATCCATCCGGAAGTACCTAATC |
| | SDM | CTTATAATCTGTTATCAGGCCTGC |
| 68 | MMLV K193A Top | CGTCTGCCCCAGGGCTTTGCGAACAGCCCCACATT |
| | SDM | GTTCGATGAA |
| 69 | MMLV K193R Top | CGTCTGCCCCAGGGCTTTCGTAACAGCCCCACATT |
| | SDM | GTTCGATGAA |
| 70 | MMLV K193E Top | CGTCTGCCCCAGGGCTTTGAAAACAGCCCCACATT |
| | SDM | GTTCGATGAA |
| 71 | MMLV E282A Top | AGAAGGTCAACGTTGGCTGACTGCGGCGCGTAAGG |
| | SDM | AGACCGTAATG |
| 72 | MMLV E282R Top | AGAAGGTCAACGTTGGCTGACTCGTGCGCGTAAGG |
| | SDM | AGACCGTAATG |
| 73 | MMLV E282D Top | AGAAGGTCAACGTTGGCTGACTGATGCGCGTAAGG |
| | SDM | AGACCGTAATG |
| 74 | MMLV A283V Top | GAAGGTCAACGTTGGCTGACTGAAGTGCGTAAGGA |
| | SDM | GACCGTAATGGGGC |
| 75 | MMLV A283R Top | GAAGGTCAACGTTGGCTGACTGAACGTCGTAAGGA |
| | SDM | GACCGTAATGGGGC |
| 76 | MMLV A283E Top | GAAGGTCAACGTTGGCTGACTGAAGAACGTAAGGA |
| | SDM | GACCGTAATGGGGC |
| 77 | MMLV Q291A Top | GCGTAAGGAGACCGTAATGGGGGCGCCTACGCCTA |
| | SDM | AGACGCCACG |
| 78 | MMLV Q291R Top | GCGTAAGGAGACCGTAATGGGGCGTCCTACGCCTA |
| | SDM | AGACGCCACG |
| 79 | MMLV Q291E Top | GCGTAAGGAGACCGTAATGGGGGAACCTACGCCTA |
| | SDM | AGACGCCACG |
| 80 | MMLV T293A Top | GAGACCGTAATGGGGCAGCCTGCGCCTAAGACGCC |
| | SDM | ACGCCAGTTG |
| 81 | MMLV T293R Top | GAGACCGTAATGGGGCAGCCTCGTCCTAAGACGCC |
| | SDM | ACGCCAGTTG |
| 82 | MMLV T293E Top | GAGACCGTAATGGGGCAGCCTGAACCTAAGACGCC |
| | SDM | ACGCCAGTTG |
| 83 | MMLV K295A Top | GTAATGGGGCAGCCTACGCCTGCGACGCCACGCCA |
| | SDM | GTTGCGTGAA |
| 84 | MMLV K295R Top | GTAATGGGGCAGCCTACGCCTCGTACGCCACGCCA |
| | SDM | GTTGCGTGAA |
| 85 | MMLV K295E Top | GTAATGGGGCAGCCTACGCCTGAAACGCCACGCCA |
| | SDM | GTTGCGTGAA |
| 86 | MMLV T296A Top | TGGGGCAGCCTACGCCTAAGGCGCCACGCCAGTTG |
| | SDM | CGTGAATTTT |
| 87 | MMLV T296R Top | TGGGGCAGCCTACGCCTAAGCGTCCACGCCAGTTG |
| | SDM | CGTGAATTTT |
| 88 | MMLV T296E Top | TGGGGCAGCCTACGCCTAAGGAACCACGCCAGTTG |
| | SDM | CGTGAATTTT |
| 89 | MMLV R298A Top | GCCTACGCCTAAGACGCCAGCGCAGTTGCGTGAAT |
| | SDM | TTTTGGGCACAG |
| 90 | MMLV R298K Top | GCCTACGCCTAAGACGCCAAAACAGTTGCGTGAAT |
| | SDM | TTTTGGGCACAG |
| 91 | MMLV R298E Top | GCCTACGCCTAAGACGCCAGAACAGTTGCGTGAAT |
| | SDM | TTTTGGGCACAG |
| 92 | MMLV R301A Top | CCTAAGACGCCACGCCAGTTGGCGGAATTTTTGGG |
| | SDM | CACAGCGGGA |
| 93 | MMLV R301K Top | CCTAAGACGCCACGCCAGTTGAAAGAATTTTTGGG |
| | SDM | CACAGCGGGA |
| 94 | MMLV R301E Top | CCTAAGACGCCACGCCAGTTGGAAGAATTTTTGGG |
| | SDM | CACAGCGGGA |
| 95 | MMLV K329A Top | GCACCCCTGTACCCCTTAACAGCGACAGGGACGCT |
| | SDM | TTTCAACTGG |
| 96 | MMLV K329R Top | GCACCCCTGTACCCCTTAACACGTACAGGGACGCT |
| | SDM | TTTCAACTGG |
| 97 | MMLV K329E Top | GCACCCCTGTACCCCTTAACAGAAACAGGGACGCT |
| | SDM | TTTCAACTGG |
| 98 | MMLV K53A Btm | GTTTGATAGAGACAGGTGTAGACGTTGCCGCTAAC |
| | SDM | GGGATGATCAACGGTGCTT |
| 99 | MMLV K53R Btm | GTTTGATAGAGACAGGTGTAGACGTTGCACGTAAC |
| | SDM | GGGATGATCAACGGTGCTT |
| 100 | MMLV K53E Btm | GTTTGATAGAGACAGGTGTAGACGTTGCTTCTAAC |
| | SDM | GGGATGATCAACGGTGCTT |
| 101 | MMLV T55A Btm | GGGGTACTGTTTGATAGAGACAGGTGTAGACGCTG |
| | SDM | CCTTTAACGGGATGATCAACGG |
| 102 | MMLV T55R Btm | GGGGTACTGTTTGATAGAGACAGGTGTAGAACGTG |
| | SDM | CCTTTAACGGGATGATCAACGG |
| 103 | MMLV T55E Btm | GGGGTACTGTTTGATAGAGACAGGTGTAGATTCTG |
| | SDM | CCTTTAACGGGATGATCAACGG |
| 104 | MMLV T57A Btm | CTCATGGGGTACTGTTTGATAGAGACAGGCGCAGA |
| | SDM | CGTTGCCTTTAACGGGATGAT |
| 105 | MMLV T57R Btm | CTCATGGGGTACTGTTTGATAGAGACAGGACGAGA |
| | SDM | CGTTGCCTTTAACGGGATGAT |
| 106 | MMLV T57E Btm | CTCATGGGGTACTGTTTGATAGAGACAGGTTCAGA |
| | SDM | CGTTGCCTTTAACGGGATGAT |
| 107 | MMLV V59A Btm | CCTCTTGACTCATGGGGTACTGTTTGATAGACGCA |
| | SDM | GGTGTAGACGTTGCCTTTAACGG |
| 108 | MMLV V59R Btm | CCTCTTGACTCATGGGGTACTGTTTGATAGAACGA |
| | SDM | GGTGTAGACGTTGCCTTTAACGG |
| 109 | MMLV V59E Btm | CCTCTTGACTCATGGGGTACTGTTTGATAGATTCA |
| | SDM | GGTGTAGACGTTGCCTTTAACGG |
| 110 | MMLV I61A Btm | CCTCTTGACTCATGGGGTACTGTTTCGCAGAGACA |
| | SDM | GGTGTAGACGTTGCCTTTA |
| 111 | MMLV 161R Btm | CCTCTTGACTCATGGGGTACTGTTTACGAGAGACA |
| | SDM | GGTGTAGACGTTGCCTTTA |
| 112 | MMLV 161E Btm | CCTCTTGACTCATGGGGTACTGTTTTTCAGAGACA |
| | SDM | GGTGTAGACGTTGCCTTTA |
| 113 | MMLV K62A Btm | GCCTCTTGACTCATGGGGTACTGCGCGATAGAGAC |
| | SDM | AGGTGTAGACGTTGCC |
| 114 | MMLV K62R Btm | GCCTCTTGACTCATGGGGTACTGACGGATAGAGAC |
| | SDM | AGGTGTAGACGTTGCC |
| 115 | MMLV K62E Btm | GCCTCTTGACTCATGGGGTACTGTTCGATAGAGAC |
| | SDM | AGGTGTAGACGTTGCC |
| 116 | MMLV Q68A Btm | CTGTCTCTATCAAACAGTACCCCATGAGTGCGGAG |
| | SDM | GCCCGCCTGGG |
| 117 | MMLV Q68R Btm | CTGTCTCTATCAAACAGTACCCCATGAGTCGTGAG |
| | SDM | GCCCGCCTGGG |
| 118 | MMLV Q68E Btm | CTGTCTCTATCAAACAGTACCCCATGAGTGAAGAG |
| | SDM | GCCCGCCTGGG |
| 119 | MMLV K75A Btm | TGGTCCAGCAAGCGCTGAATATGTGGCGCAATCCC |
| | SDM | CAGGCGGGCC |
| 120 | MMLV K75R Btm | TGGTCCAGCAAGCGCTGAATATGTGGACGAATCCC |
| | SDM | CAGGCGGGCC |
| 121 | MMLV K75E Btm | TGGTCCAGCAAGCGCTGAATATGTGGTTCAATCCC |
| | SDM | CAGGCGGGCC |
| 122 | MMLV Q79A Btm | CCCCTGGTCCAGCAAGCGCGCAATATGTGGCTTAA |
| | SDM | TCCCCAGGCG |
| 123 | MMLV Q79R Btm | CCCCTGGTCCAGCAAGCGACGAATATGTGGCTTAA |
| | SDM | TCCCCAGGCG |
| 124 | MMLV Q79E Btm | CCCCTGGTCCAGCAAGCGTTCAATATGTGGCTTAA |
| | SDM | TCCCCAGGCG |
| 125 | MMLV L99A Btm | GTTTGTACCTGGCTTTTTCACGGGCGCAAGGGGGG |
| | SDM | TGTTCCACGG |
| 126 | MMLV L99R Btm | GTTTGTACCTGGCTTTTTCACGGGACGAAGGGGGG |
| | SDM | TGTTCCACGG |
| 127 | MMLV L99E Btm | GTTTGTACCTGGCTTTTTCACGGGTTCAAGGGGGG |
| | SDM | TGTTCCACGG |
| 128 | MMLV V101A Btm | GGACGATAATCGTTTGTACCTGGCTTTTTCGCGGG |
| | SDM | CAGAAGGGGGGTG |
| 129 | MMLV V101R Btm | GGACGATAATCGTTTGTACCTGGCTTTTTACGGGG |
| | SDM | CAGAAGGGGGGTG |
| 130 | MMLV V101E Btm | GGACGATAATCGTTTGTACCTGGCTTTTTTTCGGG |
| | SDM | CAGAAGGGGGGTG |
| 131 | MMLV K102A Btm | GGACGATAATCGTTTGTACCTGGCTTCGCCACGGG |
| | SDM | CAGAAGGGGG |
| 132 | MMLV K102R Btm | GGACGATAATCGTTTGTACCTGGCTTACGCACGGG |
| | SDM | CAGAAGGGGG |
| 133 | MMLV K102E Btm | GGACGATAATCGTTTGTACCTGGCTTTTCCACGGG |
| | SDM | CAGAAGGGGG |
| 134 | MMLV K103A Btm | AACTGGACGATAATCGTTTGTACCTGGCGCTTTCA |
| | SDM | CGGGCAGAAGGGGG |
| 135 | MMLV K103R Btm | AACTGGACGATAATCGTTTGTACCTGGACGTTTCA |
| | SDM | CGGGCAGAAGGGGG |
| 136 | MMLV K103E Btm | AACTGGACGATAATCGTTTGTACCTGGTTCTTTCA |
| | SDM | CGGGCAGAAGGGGG |
| 137 | MMLV T106A Btm | CGAAGATCTTGAACTGGACGATAATCGTTCGCACC |
| | SDM | TGGCTTTTTCACGGGC |
| 138 | MMLV T106R Btm | CGAAGATCTTGAACTGGACGATAATCGTTACGACC |
| | SDM | TGGCTTTTTCACGGGC |
| 139 | MMLV T106E Btm | CGAAGATCTTGAACTGGACGATAATCGTTTTCACC |
| | SDM | TGGCTTTTTCACGGGC |
| 140 | MMLV N107A Btm | CGCGAAGATCTTGAACTGGACGATAATCCGCTGTA |
| | SDM | CCTGGCTTTTTCACGGG |
| 141 | MMLV N107R Btm | CGCGAAGATCTTGAACTGGACGATAATCACGTGTA |
| | SDM | CCTGGCTTTTTCACGGG |
| 142 | MMLV N107E Btm | CGCGAAGATCTTGAACTGGACGATAATCTTCTGTA |
| | SDM | CCTGGCTTTTTCACGGG |
| 143 | MMLV Y109A Btm | CGCGAAGATCTTGAACTGGACGCGCATCGTTTGTA |
| | SDM | CCTGGCTTTTTCACG |
| 144 | MMLV Y109R Btm | CGCGAAGATCTTGAACTGGACGACGATCGTTTGTA |
| | SDM | CCTGGCTTTTTCACG |
| 145 | MMLV Y109E Btm | CGCGAAGATCTTGAACTGGACGTTCATCGTTTGTA |
| | SDM | CCTGGCTTTTTCACG |
| 146 | MMLV R110A Btm | CCTCGCGAAGATCTTGAACTGGCGCATAATCGTTT |
| | SDM | GTACCTGGCTTTTTCACG |
| 147 | MMLV R110K Btm | CCTCGCGAAGATCTTGAACTGGTTTATAATCGTTT |
| | SDM | GTACCTGGCTTTTTCACG |
| 148 | MMLV R110E Btm | CCTCGCGAAGATCTTGAACTGGTTCATAATCGTTT |
| | SDM | GTACCTGGCTTTTTCACG |
| 149 | MMLV V112A Btm | GTTTGTTGACCTCGCGAAGATCTTGCGCTGGACGA |
| | SDM | TAATCGTTTGTACCTGGC |
| 150 | MMLV V112R Btm | GTTTGTTGACCTCGCGAAGATCTTGACGTGGACGA |
| | SDM | TAATCGTTTGTACCTGGC |
| 151 | MMLV V112E Btm | GTTTGTTGACCTCGCGAAGATCTTGTTCTGGACGA |
| | SDM | TAATCGTTTGTACCTGGC |
| 152 | MMLV K120A Btm | GTCGGATGGATGTCTTCTACGCGCGCGTTGACCTC |
| | SDM | GCGAAGATCTTGAACT |
| 153 | MMLV K120R Btm | GTCGGATGGATGTCTTCTACGCGACGGTTGACCTC |
| | SDM | GCGAAGATCTTGAACT |
| 154 | MMLV K120E Btm | GTCGGATGGATGTCTTCTACGCGTTCGTTGACCTC |
| | SDM | GCGAAGATCTTGAACT |
| 155 | MMLV E123A Btm | GGATTAGGTACAGTCGGATGGATGTCCGCTACGCG |
| | SDM | TTTGTTGACCTCGC |
| 156 | MMLV E123R Btm | GGATTAGGTACAGTCGGATGGATGTCACGTACGCG |
| | SDM | TTTGTTGACCTCGC |
| 157 | MMLV E123D Btm | GGATTAGGTACAGTCGGATGGATGTCATCTACGCG |
| | SDM | TTTGTTGACCTCGC |
| 158 | MMLV T128V Btm | GCAGGCCTGATAACAGATTATAAGGATTAGGTACC |
| | SDM | ACCGGATGGATGTCTTCTACGCGT |
| 159 | MMLV T128R Btm | GCAGGCCTGATAACAGATTATAAGGATTAGGTACA |
| | SDM | CGCGGATGGATGTCTTCTACGCGT |
| 160 | MMLV T128E Btm | GCAGGCCTGATAACAGATTATAAGGATTAGGTACT |
| | SDM | TCCGGATGGATGTCTTCTACGCGT |
| 161 | MMLV K193A Btm | TTCATCGAACAATGTGGGGCTGTTCGCAAAGCCCT |
| | SDM | GGGGCAGACG |
| 162 | MMLV K193R Btm | TTCATCGAACAATGTGGGGCTGTTACGAAAGCCCT |
| | SDM | GGGGCAGACG |
| 163 | MMLV K193E Btm | TTCATCGAACAATGTGGGGCTGTTTTCAAAGCCCT |
| | SDM | GGGGCAGACG |
| 164 | MMLV E282A Btm | CATTACGGTCTCCTTACGCGCCGCAGTCAGCCAAC |
| | SDM | GTTGACCTTCT |
| 165 | MMLV E282R Btm | CATTACGGTCTCCTTACGCGCACGAGTCAGCCAAC |
| | SDM | GTTGACCTTCT |
| 166 | MMLV E282D Btm | CATTACGGTCTCCTTACGCGCATCAGTCAGCCAAC |
| | SDM | GTTGACCTTCT |
| 167 | MMLV A283V Btm | GCCCCATTACGGTCTCCTTACGCACTTCAGTCAGC |
| | SDM | CAACGTTGACCTTC |
| 168 | MMLV A 283R Btm | GCCCCATTACGGTCTCCTTACGACGTTCAGTCAGC |
| | SDM | CAACGTTGACCTTC |
| 169 | MMLV A283E Btm | GCCCCATTACGGTCTCCTTACGTTCTTCAGTCAGC |
| | SDM | CAACGTTGACCTTC |
| 170 | MMLV Q291A Btm | CGTGGCGTCTTAGGCGTAGGCGCCCCCATTACGGT |
| | SDM | CTCCTTACGC |
| 171 | MMLV Q291R Btm | CGTGGCGTCTTAGGCGTAGGACGCCCCATTACGGT |
| | SDM | CTCCTTACGC |
| 172 | MMLV Q291E Btm | CGTGGCGTCTTAGGCGTAGGTTCCCCCATTACGGT |
| | SDM | CTCCTTACGC |
| 173 | MMLV T293A Btm | CAACTGGCGTGGCGTCTTAGGCGCAGGCTGCCCCA |
| | SDM | TTACGGTCTC |
| 174 | MMLV T293R Btm | CAACTGGCGTGGCGTCTTAGGACGAGGCTGCCCCA |
| | SDM | TTACGGTCTC |
| 175 | MMLV T293E Btm | CAACTGGCGTGGCGTCTTAGGTTCAGGCTGCCCCA |
| | SDM | TTACGGTCTC |
| 176 | MMLV K295A Btm | TTCACGCAACTGGCGTGGCGTCGCAGGCGTAGGCT |
| | SDM | GCCCCATTAC |
| 177 | MMLV K295R Btm | TTCACGCAACTGGCGTGGCGTACGAGGCGTAGGCT |
| | SDM | GCCCCATTAC |
| 178 | MMLV K295E Btm | TTCACGCAACTGGCGTGGCGTTTCAGGCGTAGGCT |
| | SDM | GCCCCATTAC |
| 179 | MMLV T296A Btm | AAAATTCACGCAACTGGCGTGGCGCCTTAGGCGTA |
| | SDM | GGCTGCCCCA |
| 180 | MMLV T296R Btm | AAAATTCACGCAACTGGCGTGGACGCTTAGGCGTA |
| | SDM | GGCTGCCCCA |
| 181 | MMLV T296E Btm | AAAATTCACGCAACTGGCGTGGTTCCTTAGGCGTA |
| | SDM | GGCTGCCCCA |
| 182 | MMLV R298A Btm | CTGTGCCCAAAAATTCACGCAACTGCGCTGGCGTC |
| | SDM | TTAGGCGTAGGC |
| 183 | MMLV R298K Btm | CTGTGCCCAAAAATTCACGCAACTGTTTTGGCGTC |
| | SDM | TTAGGCGTAGGC |
| 184 | MMLV R298E Btm | CTGTGCCCAAAAATTCACGCAACTGTTCTGGCGTC |
| | SDM | TTAGGCGTAGGC |
| 185 | MMLV R301A Btm | TCCCGCTGTGCCCAAAAATTCCGCCAACTGGCGTG |
| | SDM | GCGTCTTAGG |
| 186 | MMLV R301K Btm | TCCCGCTGTGCCCAAAAATTCTTTCAACTGGCGTG |
| | SDM | GCGTCTTAGG |
| 187 | MMLV R301E Btm | TCCCGCTGTGCCCAAAAATTCTTCCAACTGGCGTG |
| | SDM | GCGTCTTAGG |
| 188 | MMLV K329A Btm | CCAGTTGAAAAGCGTCCCTGTCGCTGTTAAGGGGT |
| | SDM | ACAGGGGTGC |
| 189 | MMLV K329R Btm | CCAGTTGAAAAGCGTCCCTGTACGTGTTAAGGGGT |
| | SDM | ACAGGGGTGC |
| 190 | MMLV K329E Btm | CCAGTTGAAAAGCGTCCCTGTTTCTGTTAAGGGGT |
| | SDM | ACAGGGGTGC |
| 191 | MMLV 161G Top | TAAAGGCAACGTCTACACCTGTCTCTGGCAAACAG |
| | SDM | TACCCCATGAGTCAAGAGG |
| 192 | MMLV 161G Btm | CCTCTTGACTCATGGGGTACTGTTTGCCAGAGACA |
| | SDM | GGTGTAGACGTTGCCTTTA |
| 193 | MMLV 161L Top | TAAAGGCAACGTCTACACCTGTCTCTCTGAAACAG |
| | SDM | TACCCCATGAGTCAAGAGG |
| 194 | MMLV I61L Btm | CCTCTTGACTCATGGGGTACTGTTTCAGAGAGACA |
| | SDM | GGTGTAGACGTTGCCTTTA |
| 195 | MMLV 161V Top | TAAAGGCAACGTCTACACCTGTCTCTGTGAAACAG |
| | SDM | TACCCCATGAGTCAAGAGG |
| 196 | MMLV I61V Btm | CCTCTTGACTCATGGGGTACTGTTTCACAGAGACA |
| | SDM | GGTGTAGACGTTGCCTTTA |
| 197 | MMLV 161P Top | TAAAGGCAACGTCTACACCTGTCTCTCCGAAACAG |
| | SDM | TACCCCATGAGTCAAGAGG |
| 198 | MMLV 161P Btm | CCTCTTGACTCATGGGGTACTGTTTCGGAGAGACA |
| | SDM | GGTGTAGACGTTGCCTTTA |
| 199 | MMLV 161M Top | TAAAGGCAACGTCTACACCTGTCTCTATGAAACAG |
| | SDM | TACCCCATGAGTCAAGAGG |
| 200 | MMLV I61M Btm | CCTCTTGACTCATGGGGTACTGTTTCATAGAGACA |
| | SDM | GGTGTAGACGTTGCCTTTA |
| 201 | MMLV 161S Top | TAAAGGCAACGTCTACACCTGTCTCTAGCAAACAG |
| | SDM | TACCCCATGAGTCAAGAGG |
| 202 | MMLV 161S Btm | CCTCTTGACTCATGGGGTACTGTTTGCTAGAGACA |
| | SDM | GGTGTAGACGTTGCCTTTA |
| 203 | MMLV 161T Top | TAAAGGCAACGTCTACACCTGTCTCTACCAAACAG |
| | SDM | TACCCCATGAGTCAAGAGG |
| 204 | MMLV 161T Btm | CCTCTTGACTCATGGGGTACTGTTTGGTAGAGACA |
| | SDM | GGTGTAGACGTTGCCTTTA |
| 205 | MMLV 161C Top | TAAAGGCAACGTCTACACCTGTCTCTTGCAAACAG |
| | SDM | TACCCCATGAGTCAAGAGG |
| 206 | MMLV I61C Btm | CCTCTTGACTCATGGGGTACTGTTTGCAAGAGACA |
| | SDM | GGTGTAGACGTTGCCTTTA |
| 207 | MMLV 161F Top | TAAAGGCAACGTCTACACCTGTCTCTTTTAAACAG |
| | SDM | TACCCCATGAGTCAAGAGG |
| 208 | MMLV 161F Btm | CCTCTTGACTCATGGGGTACTGTTTAAAAGAGACA |
| | SDM | GGTGTAGACGTTGCCTTTA |
| 209 | MMLV 161Y Top | TAAAGGCAACGTCTACACCTGTCTCTTATAAACAG |
| | SDM | TACCCCATGAGTCAAGAGG |
| 210 | MMLV I61Y Btm | CCTCTTGACTCATGGGGTACTGTTTATAAGAGACA |
| | SDM | GGTGTAGACGTTGCCTTTA |
| 211 | MMLV 161H Top | TAAAGGCAACGTCTACACCTGTCTCTCATAAACAG |
| | SDM | TACCCCATGAGTCAAGAGG |
| 212 | MMLV I61H Btm | CCTCTTGACTCATGGGGTACTGTTTATGAGAGACA |
| | SDM | GGTGTAGACGTTGCCTTTA |
| 213 | MMLV 161W Top | TAAAGGCAACGTCTACACCTGTCTCTTGGAAACAG |
| | SDM | TACCCCATGAGTCAAGAGG |
| 214 | MMLV I61W Btm | CCTCTTGACTCATGGGGTACTGTTTCCAAGAGACA |
| | SDM | GGTGTAGACGTTGCCTTTA |
| 215 | MMLV 161D Top | TAAAGGCAACGTCTACACCTGTCTCTGATAAACAG |
| | SDM | TACCCCATGAGTCAAGAGG |
| 216 | MMLV I61D Btm | CCTCTTGACTCATGGGGTACTGTTTATCAGAGACA |
| | SDM | GGTGTAGACGTTGCCTTTA |
| 217 | MMLV 161N Top | TAAAGGCAACGTCTACACCTGTCTCTAACAAACAG |
| | SDM | TACCCCATGAGTCAAGAGG |
| 218 | MMLV I61N Btm | CCTCTTGACTCATGGGGTACTGTTTGTTAGAGACA |
| | SDM | GGTGTAGACGTTGCCTTTA |
| 219 | MMLV 161Q Top | TAAAGGCAACGTCTACACCTGTCTCTCAGAAACAG |
| | SDM | TACCCCATGAGTCAAGAGG |
| 220 | MMLV I61Q Btm | CCTCTTGACTCATGGGGTACTGTTTCTGAGAGACA |
| | SDM | GGTGTAGACGTTGCCTTTA |
| 221 | MMLV 161K Top | TAAAGGCAACGTCTACACCTGTCTCTAAAAAACAG |
| | SDM | TACCCCATGAGTCAAGAGG |
| 222 | MMLV 161K Btm | CCTCTTGACTCATGGGGTACTGTTTTTTAGAGACA |
| | SDM | GGTGTAGACGTTGCCTTTA |
| 223 | MMLV Q68G Top | CTGTCTCTATCAAACAGTACCCCATGAGTGGCGAG |
| | SDM | GCCCGCCTGGG |
| 224 | MMLV Q68G Btm | CCCAGGCGGGCCTCGCCACTCATGGGGTACTGTTT |
| | SDM | GATAGAGACAG |
| 225 | MMLV Q68L Top | CTGTCTCTATCAAACAGTACCCCATGAGTCTGGAG |
| | SDM | GCCCGCCTGGG |
| 226 | MMLV Q68L Btm | CCCAGGCGGGCCTCCAGACTCATGGGGTACTGTTT |
| | SDM | GATAGAGACAG |
| 227 | MMLV Q68I Top | CTGTCTCTATCAAACAGTACCCCATGAGTATTGAG |
| | SDM | GCCCGCCTGGG |
| 228 | MMLV Q68I Btm | CCCAGGCGGGCCTCAATACTCATGGGGTACTGTTT |
| | SDM | GATAGAGACAG |
| 229 | MMLV Q68V Top | CTGTCTCTATCAAACAGTACCCCATGAGTGTGGAG |
| | SDM | GCCCGCCTGGG |
| 230 | MMLV Q68V Btm | CCCAGGCGGGCCTCCACACTCATGGGGTACTGTTT |
| | SDM | GATAGAGACAG |
| 231 | MMLV Q68P Top | CTGTCTCTATCAAACAGTACCCCATGAGTCCGGAG |
| | SDM | GCCCGCCTGGG |
| 232 | MMLV Q68P Btm | CCCAGGCGGGCCTCCGGACTCATGGGGTACTGTTT |
| | SDM | GATAGAGACAG |
| 233 | MMLV Q68M Top | CTGTCTCTATCAAACAGTACCCCATGAGTATGGAG |
| | SDM | GCCCGCCTGGG |
| 234 | MMLV Q68M Btm | CCCAGGCGGGCCTCCATACTCATGGGGTACTGTTT |
| | SDM | GATAGAGACAG |
| 235 | MMLV Q68S Top | CTGTCTCTATCAAACAGTACCCCATGAGTAGCGAG |
| | SDM | GCCCGCCTGGG |
| 236 | MMLV Q68S Btm | CCCAGGCGGGCCTCGCTACTCATGGGGTACTGTTT |
| | SDM | GATAGAGACAG |
| 237 | MMLV Q68T Top | CTGTCTCTATCAAACAGTACCCCATGAGTACCGAG |
| | SDM | GCCCGCCTGGG |
| 238 | MMLV Q68T Btm | CCCAGGCGGGCCTCGGTACTCATGGGGTACTGTTT |
| | SDM | GATAGAGACAG |
| 239 | MMLV Q68C Top | CTGTCTCTATCAAACAGTACCCCATGAGTTGCGAG |
| | SDM | GCCCGCCTGGG |
| 240 | MMLV Q68C Btm | CCCAGGCGGGCCTCGCAACTCATGGGGTACTGTTT |
| | SDM | GATAGAGACAG |
| 241 | MMLV Q68F Top | CTGTCTCTATCAAACAGTACCCCATGAGTTTTGAG |
| | SDM | GCCCGCCTGGG |
| 242 | MMLV Q68F Btm | CCCAGGCGGGCCTCAAAACTCATGGGGTACTGTTT |
| | SDM | GATAGAGACAG |
| 243 | MMLV Q68Y Top | CTGTCTCTATCAAACAGTACCCCATGAGTTATGAG |
| | SDM | GCCCGCCTGGG |
| 244 | MMLV Q68Y Btm | CCCAGGCGGGCCTCATAACTCATGGGGTACTGTTT |
| | SDM | GATAGAGACAG |
| 245 | MMLV Q68H Top | CTGTCTCTATCAAACAGTACCCCATGAGTCATGAG |
| | SDM | GCCCGCCTGGG |
| 246 | MMLV Q68H Btm | CCCAGGCGGGCCTCATGACTCATGGGGTACTGTTT |
| | SDM | GATAGAGACAG |
| 247 | MMLV Q68W Top | CTGTCTCTATCAAACAGTACCCCATGAGTTGGGAG |
| | SDM | GCCCGCCTGGG |
| 248 | MMLV Q68W Btm | CCCAGGCGGGCCTCCCAACTCATGGGGTACTGTTT |
| | SDM | GATAGAGACAG |
| 249 | MMLV Q68D Top | CTGTCTCTATCAAACAGTACCCCATGAGTGATGAG |
| | SDM | GCCCGCCTGGG |
| 250 | MMLV Q68D Btm | CCCAGGCGGGCCTCATCACTCATGGGGTACTGTTT |
| | SDM | GATAGAGACAG |
| 251 | MMLV Q68N Top | CTGTCTCTATCAAACAGTACCCCATGAGTAACGAG |
| | SDM | GCCCGCCTGGG |
| 252 | MMLV Q68N Btm | CCCAGGCGGGCCTCGTTACTCATGGGGTACTGTTT |
| | SDM | GATAGAGACAG |
| 253 | MMLV Q68K Top | CTGTCTCTATCAAACAGTACCCCATGAGTAAAGAG |
| | SDM | GCCCGCCTGGG |
| 254 | MMLV Q68K Btm | CCCAGGCGGGCCTCTTTACTCATGGGGTACTGTTT |
| | SDM | GATAGAGACAG |
| 255 | MMLV Q79G Top | CGCCTGGGGATTAAGCCACATATTGGCCGCTTGCT |
| | SDM | GGACCAGGGG |
| 256 | MMLV Q79G Btm | CCCCTGGTCCAGCAAGCGGCCAATATGTGGCTTAA |
| | SDM | TCCCCAGGCG |
| 257 | MMLV Q79L Top | CGCCTGGGGATTAAGCCACATATTCTGCGCTTGCT |
| | SDM | GGACCAGGGG |
| 258 | MMLV Q79L Btm | CCCCTGGTCCAGCAAGCGCAGAATATGTGGCTTAA |
| | SDM | TCCCCAGGCG |
| 259 | MMLV Q79I Top | CGCCTGGGGATTAAGCCACATATTATTCGCTTGCT |
| | SDM | GGACCAGGGG |
| 260 | MMLV Q79I Btm | CCCCTGGTCCAGCAAGCGAATAATATGTGGCTTAA |
| | SDM | TCCCCAGGCG |
| 261 | MMLV Q79V Top | CGCCTGGGGATTAAGCCACATATTGTGCGCTTGCT |
| | SDM | GGACCAGGGG |
| 262 | MMLV Q79V Btm | CCCCTGGTCCAGCAAGCGCACAATATGTGGCTTAA |
| | SDM | TCCCCAGGCG |
| 263 | MMLV Q79P Top | CGCCTGGGGATTAAGCCACATATTCCGCGCTTGCT |
| | SDM | GGACCAGGGG |
| 264 | MMLV Q79P Btm | CCCCTGGTCCAGCAAGCGCGGAATATGTGGCTTAA |
| | SDM | TCCCCAGGCG |
| 265 | MMLV Q79M Top | CGCCTGGGGATTAAGCCACATATTATGCGCTTGCT |
| | SDM | GGACCAGGGG |
| 266 | MMLV Q79M Btm | CCCCTGGTCCAGCAAGCGCATAATATGTGGCTTAA |
| | SDM | TCCCCAGGCG |
| 267 | MMLV Q79S Top | CGCCTGGGGATTAAGCCACATATTAGCCGCTTGCT |
| | SDM | GGACCAGGGG |
| 268 | MMLV Q79S Btm | CCCCTGGTCCAGCAAGCGGCTAATATGTGGCTTAA |
| | SDM | TCCCCAGGCG |
| 269 | MMLV Q79T Top | CGCCTGGGGATTAAGCCACATATTACCCGCTTGCT |
| | SDM | GGACCAGGGG |
| 270 | MMLV Q79T Btm | CCCCTGGTCCAGCAAGCGGGTAATATGTGGCTTAA |
| | SDM | TCCCCAGGCG |
| 271 | MMLV Q79C Top | CGCCTGGGGATTAAGCCACATATTTGCCGCTTGCT |
| | SDM | GGACCAGGGG |
| 272 | MMLV Q79C Btm | CCCCTGGTCCAGCAAGCGGCAAATATGTGGCTTAA |
| | SDM | TCCCCAGGCG |
| 273 | MMLV Q79F Top | CGCCTGGGGATTAAGCCACATATTTTTCGCTTGCT |
| | SDM | GGACCAGGGG |
| 274 | MMLV Q79F Btm | CCCCTGGTCCAGCAAGCGAAAAATATGTGGCTTAA |
| | SDM | TCCCCAGGCG |
| 275 | MMLV Q79Y Top | CGCCTGGGGATTAAGCCACATATTTATCGCTTGCT |
| | SDM | GGACCAGGGG |
| 276 | MMLV Q79Y Btm | CCCCTGGTCCAGCAAGCGATAAATATGTGGCTTAA |
| | SDM | TCCCCAGGCG |
| 277 | MMLV Q79H Top | CGCCTGGGGATTAAGCCACATATTCATCGCTTGCT |
| | SDM | GGACCAGGGG |
| 278 | MMLV Q79H Btm | CCCCTGGTCCAGCAAGCGATGAATATGTGGCTTAA |
| | SDM | TCCCCAGGCG |
| 279 | MMLV Q79W Top | CGCCTGGGGATTAAGCCACATATTTGGCGCTTGCT |
| | SDM | GGACCAGGGG |
| 280 | MMLV Q79W Btm | CCCCTGGTCCAGCAAGCGCCAAATATGTGGCTTAA |
| | SDM | TCCCCAGGCG |
| 281 | MMLV Q79D Top | CGCCTGGGGATTAAGCCACATATTGATCGCTTGCT |
| | SDM | GGACCAGGGG |
| 282 | MMLV Q79D Btm | CCCCTGGTCCAGCAAGCGATCAATATGTGGCTTAA |
| | SDM | TCCCCAGGCG |
| 283 | MMLV Q79N Top | CGCCTGGGGATTAAGCCACATATTAACCGCTTGCT |
| | SDM | GGACCAGGGG |
| 284 | MMLV Q79N Btm | CCCCTGGTCCAGCAAGCGGTTAATATGTGGCTTAA |
| | SDM | TCCCCAGGCG |
| 285 | MMLV Q79K Top | CGCCTGGGGATTAAGCCACATATTAAACGCTTGCT |
| | SDM | GGACCAGGGG |
| 286 | MMLV Q79K Btm | CCCCTGGTCCAGCAAGCGTTTAATATGTGGCTTAA |
| | SDM | TCCCCAGGCG |
| 287 | MMLV L99G Top | CCGTGGAACACCCCCCTTGGCCCCGTGAAAAAGCC |
| | SDM | AGGTACAAAC |
| 288 | MMLV L99G Btm | GTTTGTACCTGGCTTTTTCACGGGGCCAAGGGGGG |
| | SDM | TGTTCCACGG |
| 289 | MMLV L99I Top | CCGTGGAACACCCCCCTTATTCCCGTGAAAAAGCC |
| | SDM | AGGTACAAAC |
| 290 | MMLV L99I Btm | GTTTGTACCTGGCTTTTTCACGGGAATAAGGGGGG |
| | SDM | TGTTCCACGG |
| 291 | MMLV L99V Top | CCGTGGAACACCCCCCTTGTGCCCGTGAAAAAGCC |
| | SDM | AGGTACAAAC |
| 292 | MMLV L99V Btm | GTTTGTACCTGGCTTTTTCACGGGCACAAGGGGGG |
| | SDM | TGTTCCACGG |
| 293 | MMLV L99P Top | CCGTGGAACACCCCCCTTCCGCCCGTGAAAAAGCC |
| | SDM | AGGTACAAAC |
| 294 | MMLV L99P Btm | GTTTGTACCTGGCTTTTTCACGGGCGGAAGGGGGG |
| | SDM | TGTTCCACGG |
| 295 | MMLV L99M Top | CCGTGGAACACCCCCCTTATGCCCGTGAAAAAGCC |
| | SDM | AGGTACAAAC |
| 296 | MMLV L99M Btm | GTTTGTACCTGGCTTTTTCACGGGCATAAGGGGGG |
| | SDM | TGTTCCACGG |
| 297 | MMLV L99S Top | CCGTGGAACACCCCCCTTAGCCCCGTGAAAAAGCC |
| | SDM | AGGTACAAAC |
| 298 | MMLV L99S Btm | GTTTGTACCTGGCTTTTTCACGGGGCTAAGGGGGG |
| | SDM | TGTTCCACGG |
| 299 | MMLV L99T Top | CCGTGGAACACCCCCCTTACCCCCGTGAAAAAGCC |
| | SDM | AGGTACAAAC |
| 300 | MMLV L99T Btm | GTTTGTACCTGGCTTTTTCACGGGGGTAAGGGGGG |
| | SDM | TGTTCCACGG |
| 301 | MMLV L99C Top | CCGTGGAACACCCCCCTTTGCCCCGTGAAAAAGCC |
| | SDM | AGGTACAAAC |
| 302 | MMLV L99C Btm | GTTTGTACCTGGCTTTTTCACGGGGCAAAGGGGGG |
| | SDM | TGTTCCACGG |
| 303 | MMLV L99F Top | CCGTGGAACACCCCCCTTTTTCCCGTGAAAAAGCC |
| | SDM | AGGTACAAAC |
| 304 | MMLV L99F Btm | GTTTGTACCTGGCTTTTTCACGGGAAAAAGGGGGG |
| | SDM | TGTTCCACGG |
| 305 | MMLV L99Y Top | CCGTGGAACACCCCCCTTTATCCCGTGAAAAAGCC |
| | SDM | AGGTACAAAC |
| 306 | MMLV L99Y Btm | GTTTGTACCTGGCTTTTTCACGGGATAAAGGGGGG |
| | SDM | TGTTCCACGG |
| 307 | MMLV L99H Top | CCGTGGAACACCCCCCTTCATCCCGTGAAAAAGCC |
| | SDM | AGGTACAAAC |
| 308 | MMLV L99H Btm | GTTTGTACCTGGCTTTTTCACGGGATGAAGGGGGG |
| | SDM | TGTTCCACGG |
| 309 | MMLV L99W Top | CCGTGGAACACCCCCCTTTGGCCCGTGAAAAAGCC |
| | SDM | AGGTACAAAC |
| 310 | MMLV L99W Btm | GTTTGTACCTGGCTTTTTCACGGGCCAAAGGGGGG |
| | SDM | TGTTCCACGG |
| 311 | MMLV L99D Top | CCGTGGAACACCCCCCTTGATCCCGTGAAAAAGCC |
| | SDM | AGGTACAAAC |
| 312 | MMLV L99D Btm | GTTTGTACCTGGCTTTTTCACGGGATCAAGGGGGG |
| | SDM | TGTTCCACGG |
| 313 | MMLV L99N Top | CCGTGGAACACCCCCCTTAACCCCGTGAAAAAGCC |
| | SDM | AGGTACAAAC |
| 314 | MMLV L99N Btm | GTTTGTACCTGGCTTTTTCACGGGGTTAAGGGGGG |
| | SDM | TGTTCCACGG |
| 315 | MMLV L99Q Top | CCGTGGAACACCCCCCTTCAGCCCGTGAAAAAGCC |
| | SDM | AGGTACAAAC |
| 316 | MMLV L99Q Btm | GTTTGTACCTGGCTTTTTCACGGGCTGAAGGGGGG |
| | SDM | TGTTCCACGG |
| 317 | MMLV L99K Top | CCGTGGAACACCCCCCTTAAACCCGTGAAAAAGCC |
| | SDM | AGGTACAAAC |
| 318 | MMLV L99K Btm | GTTTGTACCTGGCTTTTTCACGGGTTTAAGGGGGG |
| | SDM | TGTTCCACGG |
| 319 | MMLV E282G Top | AGAAGGTCAACGTTGGCTGACTGGCGCGCGTAAGG |
| | SDM | AGACCGTAATG |
| 320 | MMLV E282G Btm | CATTACGGTCTCCTTACGCGCGCCAGTCAGCCAAC |
| | SDM | GTTGACCTTCT |
| 321 | MMLV E282L Top | AGAAGGTCAACGTTGGCTGACTCTGGCGCGTAAGG |
| | SDM | AGACCGTAATG |
| 322 | MMLV E282L Btm | CATTACGGTCTCCTTACGCGCCAGAGTCAGCCAAC |
| | SDM | GTTGACCTTCT |
| 323 | MMLV E282I Top | AGAAGGTCAACGTTGGCTGACTATTGCGCGTAAGG |
| | SDM | AGACCGTAATG |
| 324 | MMLV E282I Btm | CATTACGGTCTCCTTACGCGCAATAGTCAGCCAAC |
| | SDM | GTTGACCTTCT |
| 325 | MMLV E282V Top | AGAAGGTCAACGTTGGCTGACTGTGGCGCGTAAGG |
| | SDM | AGACCGTAATG |
| 326 | MMLV E282V Btm | CATTACGGTCTCCTTACGCGCCACAGTCAGCCAAC |
| | SDM | GTTGACCTTCT |
| 327 | MMLV E282P Top | AGAAGGTCAACGTTGGCTGACTCCGGCGCGTAAGG |
| | SDM | AGACCGTAATG |
| 328 | MMLV E282P Btm | CATTACGGTCTCCTTACGCGCCGGAGTCAGCCAAC |
| | SDM | GTTGACCTTCT |
| 329 | MMLV E282M Top | AGAAGGTCAACGTTGGCTGACTATGGCGCGTAAGG |
| | SDM | AGACCGTAATG |
| 330 | MMLV E282M Btm | CATTACGGTCTCCTTACGCGCCATAGTCAGCCAAC |
| | SDM | GTTGACCTTCT |
| 331 | MMLV E282S Top | AGAAGGTCAACGTTGGCTGACTAGCGCGCGTAAGG |
| | SDM | AGACCGTAATG |
| 332 | MMLV E282S Btm | CATTACGGTCTCCTTACGCGCGCTAGTCAGCCAAC |
| | SDM | GTTGACCTTCT |
| 333 | MMLV E282T Top | AGAAGGTCAACGTTGGCTGACTACCGCGCGTAAGG |
| | SDM | AGACCGTAATG |
| 334 | MMLV E282T Btm | CATTACGGTCTCCTTACGCGCGGTAGTCAGCCAAC |
| | SDM | GTTGACCTTCT |
| 335 | MMLV E282C Top | AGAAGGTCAACGTTGGCTGACTTGCGCGCGTAAGG |
| | SDM | AGACCGTAATG |
| 336 | MMLV E282C Btm | CATTACGGTCTCCTTACGCGCGCAAGTCAGCCAAC |
| | SDM | GTTGACCTTCT |
| 337 | MMLV E282F Top | AGAAGGTCAACGTTGGCTGACTTTTGCGCGTAAGG |
| | SDM | AGACCGTAATG |
| 338 | MMLV E282F Btm | CATTACGGTCTCCTTACGCGCAAAAGTCAGCCAAC |
| | SDM | GTTGACCTTCT |
| 339 | MMLV E282Y Top | AGAAGGTCAACGTTGGCTGACTTATGCGCGTAAGG |
| | SDM | AGACCGTAATG |
| 340 | MMLV E282Y Btm | CATTACGGTCTCCTTACGCGCATAAGTCAGCCAAC |
| | SDM | GTTGACCTTCT |
| 341 | MMLV E282H Top | AGAAGGTCAACGTTGGCTGACTCATGCGCGTAAGG |
| | SDM | AGACCGTAATG |
| 342 | MMLV E282H Btm | CATTACGGTCTCCTTACGCGCATGAGTCAGCCAAC |
| | SDM | GTTGACCTTCT |
| 343 | MMLV E282W Top | AGAAGGTCAACGTTGGCTGACTTGGGCGCGTAAGG |
| | SDM | AGACCGTAATG |
| 344 | MMLV E282W Btm | CATTACGGTCTCCTTACGCGCCCAAGTCAGCCAAC |
| | SDM | GTTGACCTTCT |
| 345 | MMLV E282N Top | AGAAGGTCAACGTTGGCTGACTAACGCGCGTAAGG |
| | SDM | AGACCGTAATG |
| 346 | MMLV E282N Btm | CATTACGGTCTCCTTACGCGCGTTAGTCAGCCAAC |
| | SDM | GTTGACCTTCT |
| 347 | MMLV E282Q Top | AGAAGGTCAACGTTGGCTGACTCAGGCGCGTAAGG |
| | SDM | AGACCGTAATG |
| 348 | MMLV E282Q Btm | CATTACGGTCTCCTTACGCGCCTGAGTCAGCCAAC |
| | SDM | GTTGACCTTCT |
| 349 | MMLV E282K Top | AGAAGGTCAACGTTGGCTGACTAAAGCGCGTAAGG |
| | SDM | AGACCGTAATG |
| 350 | MMLV E282K Btm | CATTACGGTCTCCTTACGCGCTTTAGTCAGCCAAC |
| | SDM | GTTGACCTTCT |
| 351 | MMLV R298G Top | GCCTACGCCTAAGACGCCAGGCCAGTTGCGTGAAT |
| | SDM | TTTTGGGCACAG |
| 352 | MMLV R298G Btm | CTGTGCCCAAAAATTCACGCAACTGGCCTGGCGTC |
| | SDM | TTAGGCGTAGGC |
| 353 | MMLV R298L Top | GCCTACGCCTAAGACGCCACTGCAGTTGCGTGAAT |
| | SDM | TTTTGGGCACAG |
| 354 | MMLV R298L Btm | CTGTGCCCAAAAATTCACGCAACTGCAGTGGCGTC |
| | SDM | TTAGGCGTAGGC |
| 355 | MMLV R298I Top | GCCTACGCCTAAGACGCCAATTCAGTTGCGTGAAT |
| | SDM | TTTTGGGCACAG |
| 356 | MMLV R298I Btm | CTGTGCCCAAAAATTCACGCAACTGAATTGGCGTC |
| | SDM | TTAGGCGTAGGC |
| 357 | MMLV R298V Top | GCCTACGCCTAAGACGCCAGTGCAGTTGCGTGAAT |
| | SDM | TTTTGGGCACAG |
| 358 | MMLV R298V Btm | CTGTGCCCAAAAATTCACGCAACTGCACTGGCGTC |
| | SDM | TTAGGCGTAGGC |
| 359 | MMLV R298P Top | GCCTACGCCTAAGACGCCACCGCAGTTGCGTGAAT |
| | SDM | TTTTGGGCACAG |
| 360 | MMLV R298P Btm | CTGTGCCCAAAAATTCACGCAACTGCGGTGGCGTC |
| | SDM | TTAGGCGTAGGC |
| 361 | MMLV R298M Top | GCCTACGCCTAAGACGCCAATGCAGTTGCGTGAAT |
| | SDM | TTTTGGGCACAG |
| 362 | MMLV R298M Btm | CTGTGCCCAAAAATTCACGCAACTGCATTGGCGTC |
| | SDM | TTAGGCGTAGGC |
| 363 | MMLV R298S Top | GCCTACGCCTAAGACGCCAAGCCAGTTGCGTGAAT |
| | SDM | TTTTGGGCACAG |
| 364 | MMLV R298S Btm | CTGTGCCCAAAAATTCACGCAACTGGCTTGGCGTC |
| | SDM | TTAGGCGTAGGC |
| 365 | MMLV R298T Top | GCCTACGCCTAAGACGCCAACCCAGTTGCGTGAAT |
| | SDM | TTTTGGGCACAG |
| 366 | MMLV R298T Btm | CTGTGCCCAAAAATTCACGCAACTGGGTTGGCGTC |
| | SDM | TTAGGCGTAGGC |
| 367 | MMLV R298C Top | GCCTACGCCTAAGACGCCATGCCAGTTGCGTGAAT |
| | SDM | TTTTGGGCACAG |
| 368 | MMLV R298C Btm | CTGTGCCCAAAAATTCACGCAACTGGCATGGCGTC |
| | SDM | TTAGGCGTAGGC |
| 369 | MMLV R298F Top | GCCTACGCCTAAGACGCCATTTCAGTTGCGTGAAT |
| | SDM | TTTTGGGCACAG |
| 370 | MMLV R298F Btm | CTGTGCCCAAAAATTCACGCAACTGAAATGGCGTC |
| | SDM | TTAGGCGTAGGC |
| 371 | MMLV R298Y Top | GCCTACGCCTAAGACGCCATATCAGTTGCGTGAAT |
| | SDM | TTTTGGGCACAG |
| 372 | MMLV R298Y Btm | CTGTGCCCAAAAATTCACGCAACTGATATGGCGTC |
| | SDM | TTAGGCGTAGGC |
| 373 | MMLV R298H Top | GCCTACGCCTAAGACGCCACATCAGTTGCGTGAAT |
| | SDM | TTTTGGGCACAG |
| 374 | MMLV R298H Btm | CTGTGCCCAAAAATTCACGCAACTGATGTGGCGTC |
| | SDM | TTAGGCGTAGGC |
| 375 | MMLV R298W Top | GCCTACGCCTAAGACGCCATGGCAGTTGCGTGAAT |
| | SDM | TTTTGGGCACAG |
| 376 | MMLV R298W Btm | CTGTGCCCAAAAATTCACGCAACTGCCATGGCGTC |
| | SDM | TTAGGCGTAGGC |
| 377 | MMLV R298D Top | GCCTACGCCTAAGACGCCAGATCAGTTGCGTGAAT |
| | SDM | TTTTGGGCACAG |
| 378 | MMLV R298D Btm | CTGTGCCCAAAAATTCACGCAACTGATCTGGCGTC |
| | SDM | TTAGGCGTAGGC |
| 379 | MMLV R298N Top | GCCTACGCCTAAGACGCCAAACCAGTTGCGTGAAT |
| | SDM | TTTTGGGCACAG |
| 380 | MMLV R298N Btm | CTGTGCCCAAAAATTCACGCAACTGGTTTGGCGTC |
| | SDM | TTAGGCGTAGGC |
| 381 | MMLV R298Q Top | GCCTACGCCTAAGACGCCACAGCAGTTGCGTGAAT |
| | SDM | TTTTGGGCACAG |
| 382 | MMLV R298Q Btm | CTGTGCCCAAAAATTCACGCAACTGCTGTGGCGTC |
| | SDM | TTAGGCGTAGGC |
| 383 | MMLV I61R/Q68R | AGGCAACGTCTACACCTGTCTCTCGTAAACAGTAC |
| | Top SDM | CCCATGAGTCGTGAGGCCCGCCTGGGG |
| 384 | MMLV I61R/Q68R | CCCCAGGCGGGCCTCACGACTCATGGGGTACTGTT |
| | Btm SDM | TACGAGAGACAGGTGTAGACGTTGCCT |
| 385 | MMLV I61K/Q68R | AGGCAACGTCTACACCTGTCTCTAAAAAACAGTAC |
| | Top SDM | CCCATGAGTCGTGAGG |
| 386 | MMLV I61K/Q68R | CCTCACGACTCATGGGGTACTGTTTTTTAGAGACA |
| | Btm SDM | GGTGTAGACGTTGCCT |
| 387 | MMLV I61M/Q68R | AGGCAACGTCTACACCTGTCTCTATGAAACAGTAC |
| | Top SDM | CCCATGAGTCGTGAGG |
| 388 | MMLV I61M/Q68R | CCTCACGACTCATGGGGTACTGTTTCATAGAGACA |
| | Btm SDM | GGTGTAGACGTTGCCT |
| 389 | MMLV I61M/Q68I | AGGCAACGTCTACACCTGTCTCTATGAAACAGTAC |
| | Top SDM | CCCATGAGTATTGAGGCC |
| 390 | MMLV I61M/Q68I | GGCCTCAATACTCATGGGGTACTGTTTCATAGAGA |
| | Btm SDM | CAGGTGTAGACGTTGCCT |
| 393 | MMLV 5′ Primer | GTCTCTATCAAACAGTACCCCATGGCGCAAGAGGC |
| | | CCGCCTGGG |
| 394 | MMLV 3′ Primer | GTCTCTATCAAACAGTACCCCATGCGTCAAGAGGC |
| | | CCGCCTGGG |
| 395 | MMLV G73A Top | CATGAGTCAAGAGGCCCGCGAGGGGATTAAGCCAC |
| | SDM | ATATTCAGCG |
| 396 | MMLV G73R Top | GAGTCAAGAGGCCCGCCTGGCGATTAAGCCACATA |
| | SDM | TTCAGCGCTTGC |
| 397 | MMLV G73E Top | GAGTCAAGAGGCCCGCCTGCGTATTAAGCCACATA |
| | SDM | TTCAGCGCTTGC |
| 398 | MMLV P76A Top | GAGTCAAGAGGCCCGCCTGGAGATTAAGCCACATA |
| | SDM | TTCAGCGCTTGC |
| 399 | MMLV P76R Top | GGCCCGCCTGGGGATTAAGGCGCATATTCAGCGCT |
| | SDM | TGCTGGACC |
| 400 | MMLV P76E Top | GGCCCGCCTGGGGATTAAGCGTCATATTCAGCGCT |
| | SDM | TGCTGGACC |
| 401 | MMLV H77A Top | GGCCCGCCTGGGGATTAAGGAGCATATTCAGCGCT |
| | SDM | TGCTGGACC |
| 402 | MMLV H77R Top | CCGCCTGGGGATTAAGCCAGCGATTCAGCGCTTGC |
| | SDM | TGGACCAG |
| 403 | MMLV H77E Top | CCGCCTGGGGATTAAGCCACGTATTCAGCGCTTGC |
| | SDM | TGGACCAG |
| 404 | MMLV L82A Top | CCGCCTGGGGATTAAGCCAGAGATTCAGCGCTTGC |
| | SDM | TGGACCAG |
| 405 | MMLV L82R Top | GATTAAGCCACATATTCAGCGCTTGGCGGACCAGG |
| | SDM | GGATCTTGGTCC |
| 406 | MMLV L82E Top | GATTAAGCCACATATTCAGCGCTTGCGTGACCAGG |
| | SDM | GGATCTTGGTCC |
| 407 | MMLV D83A Top | GATTAAGCCACATATTCAGCGCTTGGAGGACCAGG |
| | SDM | GGATCTTGGTCC |
| 408 | MMLV D83R Top | GCCACATATTCAGCGCTTGCTGGCGCAGGGGATCT |
| | SDM | TGGTCCCATG |
| 409 | MMLV D83E Top | GCCACATATTCAGCGCTTGCTGCGTCAGGGGATCT |
| | SDM | TGGTCCCATG |
| 410 | MMLV I125A Top | GCCACATATTCAGCGCTTGCTGGAGCAGGGGATCT |
| | SDM | TGGTCCCATG |
| 411 | MMLV I125R Top | AGGTCAACAAACGCGTAGAAGACGCGCATCCGACT |
| | SDM | GTACCTAATCCTTATAAT |
| 412 | MMLV I125E Top | AGGTCAACAAACGCGTAGAAGACCGTCATCCGACT |
| | SDM | GTACCTAATCCTTATAAT |
| 413 | MMLV V129A Top | AGGTCAACAAACGCGTAGAAGACGAGCATCCGACT |
| | SDM | GTACCTAATCCTTATAAT |
| 414 | MMLV V129R Top | GCGTAGAAGACATCCATCCGACTGCGCCTAATCCT |
| | SDM | TATAATCTGTTATCAGGC |
| 415 | MMLV V129E Top | GCGTAGAAGACATCCATCCGACTCGTCCTAATCCT |
| | SDM | TATAATCTGTTATCAGGC |
| 416 | MMLV L198A Top | GCGTAGAAGACATCCATCCGACTGAGCCTAATCCT |
| | SDM | TATAATCTGTTATCAGGC |
| 417 | MMLV L198R Top | AGGGCTTTAAAAACAGCCCCACAGCGTTCGATGAA |
| | SDM | GCACTTCACCGTGA |
| 418 | MMLV L198E Top | AGGGCTTTAAAAACAGCCCCACACGTTTCGATGAA |
| | SDM | GCACTTCACCGTGA |
| 419 | MMLV E201A Top | AGGGCTTTAAAAACAGCCCCACAGAGTTCGATGAA |
| | SDM | GCACTTCACCGTGA |
| 420 | MMLV E201R Top | TTTAAAAACAGCCCCACATTGTTCGATGCGGCACT |
| | SDM | TCACCGTGACTTAGCAG |
| 421 | MMLV E201D Top | TTTAAAAACAGCCCCACATTGTTCGATCGTGCACT |
| | SDM | TCACCGTGACTTAGCAG |
| 422 | MMLV R205A Top | TTTAAAAACAGCCCCACATTGTTCGATGATGCACT |
| | SDM | TCACCGTGACTTAGCAG |
| 423 | MMLV R205K | CACATTGTTCGATGAAGCACTTCACGCGGACTTAG |
| | Top SDM | CAGACTTCCGTATCCA |
| 424 | MMLV R205E Top | CACATTGTTCGATGAAGCACTTCACAAAGACTTAG |
| | SDM | CAGACTTCCGTATCCA |
| 425 | MMLV D209A Top | GATGAAGCACTTCACCGTGACTTAGAGGACTTCCG |
| | SDM | TATCCAACACCCAG |
| 426 | MMLV D209R Top | AAGCACTTCACCGTGACTTAGCAGCGTTCCGTATC |
| | SDM | CAACACCCAGACTT |
| 427 | MMLV D209E Top | AAGCACTTCACCGTGACTTAGCACGTTTCCGTATC |
| | SDM | CAACACCCAGACTT |
| 428 | MMLV F210A Top | AAGCACTTCACCGTGACTTAGCAGAGTTCCGTATC |
| | SDM | CAACACCCAGACTT |
| 429 | MMLV F210R Top | CACTTCACCGTGACTTAGCAGACGCGCGTATCCAA |
| | SDM | CACCCAGACTTAATTC |
| 430 | MMLV F210E Top | CACTTCACCGTGACTTAGCAGACCGTCGTATCCAA |
| | SDM | CACCCAGACTTAATTC |
| 431 | MMLV R211A Top | CACTTCACCGTGACTTAGCAGACGAGCGTATCCAA |
| | SDM | CACCCAGACTTAATTC |
| 432 | MMLV R211K | TTCACCGTGACTTAGCAGACTTCGCGATCCAACAC |
| | Top SDM | CCAGACTTAATTCTGTTA |
| 433 | MMLV R211E Top | TTCACCGTGACTTAGCAGACTTCAAAATCCAACAC |
| | SDM | CCAGACTTAATTCTGTTA |
| 434 | MMLV I212A Top | TTCACCGTGACTTAGCAGACTTCGAGATCCAACAC |
| | SDM | CCAGACTTAATTCTGTTA |
| 435 | MMLV I212R Top | CCGTGACTTAGCAGACTTCCGTGCGCAACACCCAG |
| | SDM | ACTTAATTCTGTTACAG |
| 436 | MMLV I212E Top | CCGTGACTTAGCAGACTTCCGTCGTCAACACCCAG |
| | SDM | ACTTAATTCTGTTACAG |
| 437 | MMLV Q213A | CCGTGACTTAGCAGACTTCCGTGAGCAACACCCAG |
| | Top SDM | ACTTAATTCTGTTACAG |
| 438 | MMLV Q213R | GTGACTTAGCAGACTTCCGTATCGCGCACCCAGAC |
| | Top SDM | TTAATTCTGTTACAGTAT |
| 439 | MMLV Q213E Top | GTGACTTAGCAGACTTCCGTATCCGTCACCCAGAC |
| | SDM | TTAATTCTGTTACAGTAT |
| 440 | MMLV K348A | GTGACTTAGCAGACTTCCGTATCGAGCACCCAGAC |
| | Top SDM | TTAATTCTGTTACAGTAT |
| 441 | MMLV K348R | AGCAAAAGGCGTATCAGGAGATCGCGCAAGCTTTG |
| | Top SDM | TTGACCGCACCC |
| 442 | MMLV K348E Top | AGCAAAAGGCGTATCAGGAGATCCGTCAAGCTTTG |
| | SDM | TTGACCGCACCC |
| 443 | MMLV L352A Top | AGCAAAAGGCGTATCAGGAGATCGAGCAAGCTTTG |
| | SDM | TTGACCGCACCC |
| 444 | MMLV L352R Top | CGTATCAGGAGATCAAACAAGCTTTGGCGACCGCA |
| | SDM | CCCGCGTTGGG |
| 445 | MMLV L352E Top | CGTATCAGGAGATCAAACAAGCTTTGCGTACCGCA |
| | SDM | CCCGCGTTGGG |
| 446 | MMLV K285A | CGTATCAGGAGATCAAACAAGCTTTGGAGACCGCA |
| | Top SDM | CCCGCGTTGGG |
| 447 | MMLV K285R | GTTGGCTGACTGAAGCGCGTGCGGAGACCGTAATG |
| | Top SDM | GGGCAGC |
| 448 | MMLV K285E Top | GTTGGCTGACTGAAGCGCGTCGTGAGACCGTAATG |
| | SDM | GGGCAGC |
| 449 | MMLV Q299A | GTTGGCTGACTGAAGCGCGTGAGGAGACCGTAATG |
| | Top SDM | GGGCAGC |
| 450 | MMLV Q299R | TACGCCTAAGACGCCACGCGCGTTGCGTGAATTTT |
| | Top SDM | TGGGCACAGC |
| 451 | MMLV Q299E Top | TACGCCTAAGACGCCACGCCGTTTGCGTGAATTTT |
| | SDM | TGGGCACAGC |
| 452 | MMLV G308A | TACGCCTAAGACGCCACGCGAGTTGCGTGAATTTT |
| | Top SDM | TGGGCACAGC |
| 453 | MMLV G308R | GCGTGAATTTTTGGGCACAGCGGCGTTCTGTCGTT |
| | Top SDM | TATGGATTCCTGGG |
| 454 | MMLV G308E Top | GCGTGAATTTTTGGGCACAGCGCGTTTCTGTCGTT |
| | SDM | TATGGATTCCTGGG |
| 455 | MMLV R311A Top | GCGTGAATTTTTGGGCACAGCGGAGTTCTGTCGTT |
| | SDM | TATGGATTCCTGGG |
| 456 | MMLV R311K | GGGCACAGCGGGATTCTGTGCGTTATGGATTCCTG |
| | Top SDM | GGTTCGCTGA |
| 457 | MMLV R311E Top | GGGCACAGCGGGATTCTGTAAATTATGGATTCCTG |
| | SDM | GGTTCGCTGA |
| 458 | MMLV Y271A Top | GGGCACAGCGGGATTCTGTGAGTTATGGATTCCTG |
| | SDM | GGTTCGCTGA |
| 459 | MMLV Y271R Top | GTCAAAAACAGGTAAAGTACCTTGGGGCGTTGCTG |
| | SDM | AAAGAAGGTCAACGTTGG |
| 460 | MMLV Y271E Top | GTCAAAAACAGGTAAAGTACCTTGGGCGTTTGCTG |
| | SDM | AAAGAAGGTCAACGTTGG |
| 461 | MMLV L280A Top | GTCAAAAACAGGTAAAGTACCTTGGGGAGTTGCTG |
| | SDM | AAAGAAGGTCAACGTTGG |
| 462 | MMLV L280R Top | TGCTGAAAGAAGGTCAACGTTGGGCGACTGAAGCG |
| | SDM | CGTAAGGAGACC |
| 463 | MMLV L280E Top | TGCTGAAAGAAGGTCAACGTTGGCGTACTGAAGCG |
| | SDM | CGTAAGGAGACC |
| 464 | MMLV L357A Top | TGCTGAAAGAAGGTCAACGTTGGGAGACTGAAGCG |
| | SDM | CGTAAGGAGACC |
| 465 | MMLV L357R Top | TTTGTTGACCGCACCCGCGGCGGGTCTTCCGGATT |
| | SDM | TAACCAAGCC |
| 466 | MMLV L357E Top | TTTGTTGACCGCACCCGCGCGTGGTCTTCCGGATT |
| | SDM | TAACCAAGCC |
| 467 | MMLV T328A Top | TTTGTTGACCGCACCCGCGGAGGGTCTTCCGGATT |
| | SDM | TAACCAAGCC |
| 468 | MMLV T328R Top | CTGCACCCCTGTACCCCTTAGCGAAAACAGGGACG |
| | SDM | CTTTTCAACTGG |
| 469 | MMLV T328E Top | CTGCACCCCTGTACCCCTTACGTAAAACAGGGACG |
| | SDM | CTTTTCAACTGG |
| 470 | MMLV G331A | CTGCACCCCTGTACCCCTTAGAGAAAACAGGGACG |
| | Top SDM | CTTTTCAACTGG |
| 471 | MMLV G331R | CCCCTGTACCCCTTAACAAAAACAGCGACGCTTTT |
| | Top SDM | CAACTGGGGGCC |
| 472 | MMLV G331E Top | CCCCTGTACCCCTTAACAAAAACACGTACGCTTTT |
| | SDM | CAACTGGGGGCC |
| 473 | MMLV T332A Top | CCCCTGTACCCCTTAACAAAAACAGAGACGCTTTT |
| | SDM | CAACTGGGGGCC |
| 474 | MMLV T332R Top | CTGTACCCCTTAACAAAAACAGGGGCGCTTTTCAA |
| | SDM | CTGGGGGCCAGAC |
| 475 | MMLV T332E Top | CTGTACCCCTTAACAAAAACAGGGCGTCTTTTCAA |
| | SDM | CTGGGGGCCAGAC |
| 476 | MMLV N335A Top | CTGTACCCCTTAACAAAAACAGGGGAGCTTTTCAA |
| | SDM | CTGGGGGCCAGAC |
| 477 | MMLV N335R Top | CCTTAACAAAAACAGGGACGCTTTTCGCGTGGGGG |
| | SDM | CCAGACCAGCAAA |
| 478 | MMLV N335E Top | CCTTAACAAAAACAGGGACGCTTTTCCGTTGGGGG |
| | SDM | CCAGACCAGCAAA |
| 479 | MMLV E367A Top | CTTCCGGATTTAACCAAGCCCTTTGCGCTGTTCGT |
| | SDM | TGATGAAAAACAGGGATAT |
| 480 | MMLV E367R Top | CTTCCGGATTTAACCAAGCCCTTTCGTCTGTTCGT |
| | SDM | TGATGAAAAACAGGGATAT |
| 481 | MMLV E367D Top | CTTCCGGATTTAACCAAGCCCTTTGATCTGTTCGT |
| | SDM | TGATGAAAAACAGGGATAT |
| 482 | MMLV F369A Top | GATTTAACCAAGCCCTTTGAGCTGGCGGTTGATGA |
| | SDM | AAAACAGGGATATGCAAAAG |
| 483 | MMLV F369R Top | GATTTAACCAAGCCCTTTGAGCTGCGTGTTGATGA |
| | SDM | AAAACAGGGATATGCAAAAG |
| 484 | MMLV F369E Top | GATTTAACCAAGCCCTTTGAGCTGGAGGTTGATGA |
| | SDM | AAAACAGGGATATGCAAAAG |
| 485 | MMLV R389A Top | CCCAAAAGTTAGGCCCGTGGGCGCGCCCTGTTGCT |
| | SDM | TACTTGAGTAA |
| 486 | MMLV R389K | CCCAAAAGTTAGGCCCGTGGAAACGCCCTGTTGCT |
| | Top SDM | TACTTGAGTAA |
| 487 | MMLV R389E Top | CCCAAAAGTTAGGCCCGTGGGAGCGCCCTGTTGCT |
| | SDM | TACTTGAGTAA |
| 488 | MMLV V433A Top | AGTTGACGATGGGTCAACCCTTAGCGATCTTGGCT |
| | SDM | CCACATGCTGTAGA |
| 489 | MMLV V433R Top | AGTTGACGATGGGTCAACCCTTACGTATCTTGGCT |
| | SDM | CCACATGCTGTAGA |
| 490 | MMLV V433E Top | AGTTGACGATGGGTCAACCCTTAGAGATCTTGGCT |
| | SDM | CCACATGCTGTAGA |
| 491 | MMLV V476A Top | GGATCGTGTACAATTTGGACCAGTTGCGGCTTTGA |
| | SDM | ATCCAGCTACTTTGCTTC |
| 492 | MMLV V476R Top | GGATCGTGTACAATTTGGACCAGTTCGTGCTTTGA |
| | SDM | ATCCAGCTACTTTGCTTC |
| 493 | MMLV V476E Top | GGATCGTGTACAATTTGGACCAGTTGAGGCTTTGA |
| | SDM | ATCCAGCTACTTTGCTTC |
| 494 | MMLV I593A Top | CGTTATGCTTTTGCAACAGCGCATGCGCATGGCGA |
| | SDM | AATTTACCGCCGC |
| 495 | MMLV I593R Top | CGTTATGCTTTTGCAACAGCGCATCGTCATGGCGA |
| | SDM | AATTTACCGCCGC |
| 496 | MMLV I593E Top | CGTTATGCTTTTGCAACAGCGCATGAGCATGGCGA |
| | SDM | AATTTACCGCCGC |
| 497 | MMLV E596A Top | GCAACAGCGCATATCCATGGCGCGATTTACCGCCG |
| | SDM | CCGTGGTC |
| 498 | MMLV E596R Top | GCAACAGCGCATATCCATGGCCGTATTTACCGCCG |
| | SDM | CCGTGGTC |
| 499 | MMLV E596D Top | GCAACAGCGCATATCCATGGCGATATTTACCGCCG |
| | SDM | CCGTGGTC |
| 500 | MMLV I597A Top | CAACAGCGCATATCCATGGCGAAGCGTACCGCCGC |
| | SDM | CGTGGTCTG |
| 501 | MMLV I597R Top | CAACAGCGCATATCCATGGCGAACGTTACCGCCGC |
| | SDM | CGTGGTCTG |
| 502 | MMLV I597E Top | CAACAGCGCATATCCATGGCGAAGAGTACCGCCGC |
| | SDM | CGTGGTCTG |
| 503 | MMLV R650A Top | AGCGGAGGCTCGTGGAAACGCGATGGCGGACCAAG |
| | SDM | CTGCCC |
| 504 | MMLV R650K | AGCGGAGGCTCGTGGAAACAAAATGGCGGACCAAG |
| | Top SDM | CTGCCC |
| 505 | MMLV R650E Top | AGCGGAGGCTCGTGGAAACGAGATGGCGGACCAAG |
| | SDM | CTGCCC |
| 506 | MMLV Q654A | GTGGAAACCGTATGGCGGACGCGGCTGCCCGTAAG |
| | Top SDM | GCGGC |
| 507 | MMLV Q654R | GTGGAAACCGTATGGCGGACCGTGCTGCCCGTAAG |
| | Top SDM | GCGGC |
| 508 | MMLV Q654E Top | GTGGAAACCGTATGGCGGACGAGGCTGCCCGTAAG |
| | SDM | GCGGC |
| 509 | MMLV R657A Top | TATGGCGGACCAAGCTGCCGCGAAGGCGGCGATCA |
| | SDM | CAGAGAC |
| 510 | MMLV R657K | TATGGCGGACCAAGCTGCCAAAAAGGCGGCGATCA |
| | Top SDM | CAGAGAC |
| 511 | MMLV R657E Top | TATGGCGGACCAAGCTGCCGAGAAGGCGGCGATCA |
| | SDM | CAGAGAC |
| 512 | MMLV G73A Btm | GCAAGCGCTGAATATGTGGCTTAATCGCCAGGCGG |
| | SDM | GCCTCTTGACTC |
| 513 | MMLV G73R Btm | GCAAGCGCTGAATATGTGGCTTAATACGCAGGCGG |
| | SDM | GCCTCTTGACTC |
| 514 | MMLV G73E Btm | GCAAGCGCTGAATATGTGGCTTAATCTCCAGGCGG |
| | SDM | GCCTCTTGACTC |
| 515 | MMLV P76A Btm | GGTCCAGCAAGCGCTGAATATGCGCCTTAATCCCC |
| | SDM | AGGCGGGCC |
| 516 | MMLV P76R Btm | GGTCCAGCAAGCGCTGAATATGACGCTTAATCCCC |
| | SDM | AGGCGGGCC |
| 517 | MMLV P76E Btm | GGTCCAGCAAGCGCTGAATATGCTCCTTAATCCCC |
| | SDM | AGGCGGGCC |
| 518 | MMLV H77A Btm | CTGGTCCAGCAAGCGCTGAATCGCTGGCTTAATCC |
| | SDM | CCAGGCGG |
| 519 | MMLV H77R Btm | CTGGTCCAGCAAGCGCTGAATACGTGGCTTAATCC |
| | SDM | CCAGGCGG |
| 520 | MMLV H77E Btm | CTGGTCCAGCAAGCGCTGAATCTCTGGCTTAATCC |
| | SDM | CCAGGCGG |
| 521 | MMLV L82A Btm | GGACCAAGATCCCCTGGTCCGCCAAGCGCTGAATA |
| | SDM | TGTGGCTTAATC |
| 522 | MMLV L82R Btm | GGACCAAGATCCCCTGGTCACGCAAGCGCTGAATA |
| | SDM | TGTGGCTTAATC |
| 523 | MMLV L82E Btm | GGACCAAGATCCCCTGGTCCTCCAAGCGCTGAATA |
| | SDM | TGTGGCTTAATC |
| 524 | MMLV D83A Btm | CATGGGACCAAGATCCCCTGCGCCAGCAAGCGCTG |
| | SDM | AATATGTGGC |
| 525 | MMLV D83R Btm | CATGGGACCAAGATCCCCTGACGCAGCAAGCGCTG |
| | SDM | AATATGTGGC |
| 526 | MMLV D83E Btm | CATGGGACCAAGATCCCCTGCTCCAGCAAGCGCTG |
| | SDM | AATATGTGGC |
| 527 | MMLV I125A Btm | ATTATAAGGATTAGGTACAGTCGGATGCGCGTCTT |
| | SDM | CTACGCGTTTGTTGACCT |
| 528 | MMLV I125R Btm | ATTATAAGGATTAGGTACAGTCGGATGACGGTCTT |
| | SDM | CTACGCGTTTGTTGACCT |
| 529 | MMLV I125E Btm | ATTATAAGGATTAGGTACAGTCGGATGCTCGTCTT |
| | SDM | CTACGCGTTTGTTGACCT |
| 530 | MMLV V129A | GCCTGATAACAGATTATAAGGATTAGGCGCAGTCG |
| | Btm SDM | GATGGATGTCTTCTACGC |
| 531 | MMLV V129R | GCCTGATAACAGATTATAAGGATTAGGACGAGTCG |
| | Btm SDM | GATGGATGTCTTCTACGC |
| 532 | MMLV V129E | GCCTGATAACAGATTATAAGGATTAGGCTCAGTCG |
| | Btm SDM | GATGGATGTCTTCTACGC |
| 533 | MMLV L198A | TCACGGTGAAGTGCTTCATCGAACGCTGTGGGGCT |
| | Btm SDM | GTTTTTAAAGCCCT |
| 534 | MMLV L198R | TCACGGTGAAGTGCTTCATCGAAACGTGTGGGGCT |
| | Btm SDM | GTTTTTAAAGCCCT |
| 535 | MMLV L198E Btm | TCACGGTGAAGTGCTTCATCGAACTCTGTGGGGCT |
| | SDM | GTTTTTAAAGCCCT |
| 536 | MMLV E201A | CTGCTAAGTCACGGTGAAGTGCCGCATCGAACAAT |
| | Btm SDM | GTGGGGCTGTTTTTAAA |
| 537 | MMLV E201R | CTGCTAAGTCACGGTGAAGTGCACGATCGAACAAT |
| | Btm SDM | GTGGGGCTGTTTTTAAA |
| 538 | MMLV E201D | CTGCTAAGTCACGGTGAAGTGCATCATCGAACAAT |
| | Btm SDM | GTGGGGCTGTTTTTAAA |
| 539 | MMLV R205A | TGGATACGGAAGTCTGCTAAGTCCGCGTGAAGTGC |
| | Btm SDM | TTCATCGAACAATGTG |
| 540 | MMLV R205K | TGGATACGGAAGTCTGCTAAGTCTTTGTGAAGTGC |
| | Btm SDM | TTCATCGAACAATGTG |
| 541 | MMLV R205E | TGGATACGGAAGTCTGCTAAGTCCTCGTGAAGTGC |
| | Btm SDM | TTCATCGAACAATGTG |
| 542 | MMLV D209A | AAGTCTGGGTGTTGGATACGGAACGCTGCTAAGTC |
| | Btm SDM | ACGGTGAAGTGCTT |
| 543 | MMLV D209R | AAGTCTGGGTGTTGGATACGGAAACGTGCTAAGTC |
| | Btm SDM | ACGGTGAAGTGCTT |
| 544 | MMLV D209E | AAGTCTGGGTGTTGGATACGGAACTCTGCTAAGTC |
| | Btm SDM | ACGGTGAAGTGCTT |
| 545 | MMLV F210A Btm | GAATTAAGTCTGGGTGTTGGATACGCGCGTCTGCT |
| | SDM | AAGTCACGGTGAAGTG |
| 546 | MMLV F210R Btm | GAATTAAGTCTGGGTGTTGGATACGACGGTCTGCT |
| | SDM | AAGTCACGGTGAAGTG |
| 547 | MMLV F210E Btm | GAATTAAGTCTGGGTGTTGGATACGCTCGTCTGCT |
| | SDM | AAGTCACGGTGAAGTG |
| 548 | MMLV R211A | TAACAGAATTAAGTCTGGGTGTTGGATCGCGAAGT |
| | Btm SDM | CTGCTAAGTCACGGTGAA |
| 549 | MMLV R211K | TAACAGAATTAAGTCTGGGTGTTGGATTTTGAAGT |
| | Btm SDM | CTGCTAAGTCACGGTGAA |
| 550 | MMLV R211E | TAACAGAATTAAGTCTGGGTGTTGGATCTCGAAGT |
| | Btm SDM | CTGCTAAGTCACGGTGAA |
| 551 | MMLV I212A Btm | CTGTAACAGAATTAAGTCTGGGTGTTGCGCACGGA |
| | SDM | AGTCTGCTAAGTCACGG |
| 552 | MMLV I212R Btm | CTGTAACAGAATTAAGTCTGGGTGTTGACGACGGA |
| | SDM | AGTCTGCTAAGTCACGG |
| 553 | MMLV I212E Btm | CTGTAACAGAATTAAGTCTGGGTGTTGCTCACGGA |
| | SDM | AGTCTGCTAAGTCACGG |
| 554 | MMLV Q213A | ATACTGTAACAGAATTAAGTCTGGGTGCGCGATAC |
| | Btm SDM | GGAAGTCTGCTAAGTCAC |
| 555 | MMLV Q213R | ATACTGTAACAGAATTAAGTCTGGGTGACGGATAC |
| | Btm SDM | GGAAGTCTGCTAAGTCAC |
| 556 | MMLV Q213E | ATACTGTAACAGAATTAAGTCTGGGTGCTCGATAC |
| | Btm SDM | GGAAGTCTGCTAAGTCAC |
| 557 | MMLV K348A | GGGTGCGGTCAACAAAGCTTGCGCGATCTCCTGAT |
| | Btm SDM | ACGCCTTTTGCT |
| 558 | MMLV K348R | GGGTGCGGTCAACAAAGCTTGACGGATCTCCTGAT |
| | Btm SDM | ACGCCTTTTGCT |
| 559 | MMLV K348E | GGGTGCGGTCAACAAAGCTTGCTCGATCTCCTGAT |
| | Btm SDM | ACGCCTTTTGCT |
| 560 | MMLV L352A | CCCAACGCGGGTGCGGTCGCCAAAGCTTGTTTGAT |
| | Btm SDM | CTCCTGATACG |
| 561 | MMLV L352R | CCCAACGCGGGTGCGGTACGCAAAGCTTGTTTGAT |
| | Btm SDM | CTCCTGATACG |
| 562 | MMLV L352E Btm | CCCAACGCGGGTGCGGTCTCCAAAGCTTGTTTGAT |
| | SDM | CTCCTGATACG |
| 563 | MMLV K285A | GCTGCCCCATTACGGTCTCCGCACGCGCTTCAGTC |
| | Btm SDM | AGCCAAC |
| 564 | MMLV K285R | GCTGCCCCATTACGGTCTCACGACGCGCTTCAGTC |
| | Btm SDM | AGCCAAC |
| 565 | MMLV K285E | GCTGCCCCATTACGGTCTCCTCACGCGCTTCAGTC |
| | Btm SDM | AGCCAAC |
| 566 | MMLV Q299A | GCTGTGCCCAAAAATTCACGCAACGCGCGTGGCGT |
| | Btm SDM | CTTAGGCGTA |
| 567 | MMLV Q299R | GCTGTGCCCAAAAATTCACGCAAACGGCGTGGCGT |
| | Btm SDM | CTTAGGCGTA |
| 568 | MMLV Q299E | GCTGTGCCCAAAAATTCACGCAACTCGCGTGGCGT |
| | Btm SDM | CTTAGGCGTA |
| 569 | MMLV G308A | CCCAGGAATCCATAAACGACAGAACGCCGCTGTGC |
| | Btm SDM | CCAAAAATTCACGC |
| 570 | MMLV G308R | CCCAGGAATCCATAAACGACAGAAACGCGCTGTGC |
| | Btm SDM | CCAAAAATTCACGC |
| 571 | MMLV G308E | CCCAGGAATCCATAAACGACAGAACTCCGCTGTGC |
| | Btm SDM | CCAAAAATTCACGC |
| 572 | MMLV R311A | TCAGCGAACCCAGGAATCCATAACGCACAGAATCC |
| | Btm SDM | CGCTGTGCCC |
| 573 | MMLV R311K | TCAGCGAACCCAGGAATCCATAATTTACAGAATCC |
| | Btm SDM | CGCTGTGCCC |
| 574 | MMLV R311E | TCAGCGAACCCAGGAATCCATAACTCACAGAATCC |
| | Btm SDM | CGCTGTGCCC |
| 575 | MMLV Y271A | CCAACGTTGACCTTCTTTCAGCAACGCCCCAAGGT |
| | Btm SDM | ACTTTACCTGTTTTTGAC |
| 576 | MMLV Y271R | CCAACGTTGACCTTCTTTCAGCAAACGCCCAAGGT |
| | Btm SDM | ACTTTACCTGTTTTTGAC |
| 577 | MMLV Y271E | CCAACGTTGACCTTCTTTCAGCAACTCCCCAAGGT |
| | Btm SDM | ACTTTACCTGTTTTTGAC |
| 578 | MMLV L280A | GGTCTCCTTACGCGCTTCAGTCGCCCAACGTTGAC |
| | Btm SDM | CTTCTTTCAGCA |
| 579 | MMLV L280R | GGTCTCCTTACGCGCTTCAGTACGCCAACGTTGAC |
| | Btm SDM | CTTCTTTCAGCA |
| 580 | MMLV L280E Btm | GGTCTCCTTACGCGCTTCAGTCTCCCAACGTTGAC |
| | SDM | CTTCTTTCAGCA |
| 581 | MMLV L357A | GGCTTGGTTAAATCCGGAAGACCCGCCGCGGGTGC |
| | Btm SDM | GGTCAACAAA |
| 582 | MMLV L357R | GGCTTGGTTAAATCCGGAAGACCACGCGCGGGTGC |
| | Btm SDM | GGTCAACAAA |
| 583 | MMLV L357E Btm | GGCTTGGTTAAATCCGGAAGACCCTCCGCGGGTGC |
| | SDM | GGTCAACAAA |
| 584 | MMLV T328A | CCAGTTGAAAAGCGTCCCTGTTTTCGCTAAGGGGT |
| | Btm SDM | ACAGGGGTGCAG |
| 585 | MMLV T328R | CCAGTTGAAAAGCGTCCCTGTTTTACGTAAGGGGT |
| | Btm SDM | ACAGGGGTGCAG |
| 586 | MMLV T328E Btm | CCAGTTGAAAAGCGTCCCTGTTTTCTCTAAGGGGT |
| | SDM | ACAGGGGTGCAG |
| 587 | MMLV G331A | GGCCCCCAGTTGAAAAGCGTCGCTGTTTTTGTTAA |
| | Btm SDM | GGGGTACAGGGG |
| 588 | MMLV G331R | GGCCCCCAGTTGAAAAGCGTACGTGTTTTTGTTAA |
| | Btm SDM | GGGGTACAGGGG |
| 589 | MMLV G331E | GGCCCCCAGTTGAAAAGCGTCTCTGTTTTTGTTAA |
| | Btm SDM | GGGGTACAGGGG |
| 590 | MMLV T332A | GTCTGGCCCCCAGTTGAAAAGCGCCCCTGTTTTTG |
| | Btm SDM | TTAAGGGGTACAG |
| 591 | MMLV T332R | GTCTGGCCCCCAGTTGAAAAGACGCCCTGTTTTTG |
| | Btm SDM | TTAAGGGGTACAG |
| 592 | MMLV T332E Btm | GTCTGGCCCCCAGTTGAAAAGCTCCCCTGTTTTTG |
| | SDM | TTAAGGGGTACAG |
| 593 | MMLV N335A | TTTGCTGGTCTGGCCCCCACGCGAAAAGCGTCCCT |
| | Btm SDM | GTTTTTGTTAAGG |
| 594 | MMLV N335R | TTTGCTGGTCTGGCCCCCAACGGAAAAGCGTCCCT |
| | Btm SDM | GTTTTTGTTAAGG |
| 595 | MMLV N335E | TTTGCTGGTCTGGCCCCCACTCGAAAAGCGTCCCT |
| | Btm SDM | GTTTTTGTTAAGG |
| 596 | MMLV E367A | ATATCCCTGTTTTTCATCAACGAACAGCGCAAAGG |
| | Btm SDM | GCTTGGTTAAATCCGGAAG |
| 597 | MMLV E367R | ATATCCCTGTTTTTCATCAACGAACAGACGAAAGG |
| | Btm SDM | GCTTGGTTAAATCCGGAAG |
| 598 | MMLV E367D | ATATCCCTGTTTTTCATCAACGAACAGATCAAAGG |
| | Btm SDM | GCTTGGTTAAATCCGGAAG |
| 599 | MMLV F369A Btm | CTTTTGCATATCCCTGTTTTTCATCAACCGCCAGC |
| | SDM | TCAAAGGGCTTGGTTAAATC |
| 600 | MMLV F369R Btm | CTTTTGCATATCCCTGTTTTTCATCAACACGCAGC |
| | SDM | TCAAAGGGCTTGGTTAAATC |
| 601 | MMLV F369E Btm | CTTTTGCATATCCCTGTTTTTCATCAACCTCCAGC |
| | SDM | TCAAAGGGCTTGGTTAAATC |
| 602 | MMLV R389A | TTACTCAAGTAAGCAACAGGGCGCGCCCACGGGCC |
| | Btm SDM | TAACTTTTGGG |
| 603 | MMLV R389K | TTACTCAAGTAAGCAACAGGGCGTTTCCACGGGCC |
| | Btm SDM | TAACTTTTGGG |
| 604 | MMLV R389E | TTACTCAAGTAAGCAACAGGGCGCTCCCACGGGCC |
| | Btm SDM | TAACTTTTGGG |
| 605 | MMLV V433A | TCTACAGCATGTGGAGCCAAGATCGCTAAGGGTTG |
| | Btm SDM | ACCCATCGTCAACT |
| 606 | MMLV V433R | TCTACAGCATGTGGAGCCAAGATACGTAAGGGTTG |
| | Btm SDM | ACCCATCGTCAACT |
| 607 | MMLV V433E | TCTACAGCATGTGGAGCCAAGATCTCTAAGGGTTG |
| | Btm SDM | ACCCATCGTCAACT |
| 608 | MMLV V476A | GAAGCAAAGTAGCTGGATTCAAAGCCGCAACTGGT |
| | Btm SDM | CCAAATTGTACACGATCC |
| 609 | MMLV V476R | GAAGCAAAGTAGCTGGATTCAAAGCACGAACTGGT |
| | Btm SDM | CCAAATTGTACACGATCC |
| 610 | MMLV V476E | GAAGCAAAGTAGCTGGATTCAAAGCCTCAACTGGT |
| | Btm SDM | CCAAATTGTACACGATCC |
| 611 | MMLV I593A Btm | GCGGCGGTAAATTTCGCCATGCGCATGCGCTGTTG |
| | SDM | CAAAAGCATAACG |
| 612 | MMLV I593R Btm | GCGGCGGTAAATTTCGCCATGACGATGCGCTGTTG |
| | SDM | CAAAAGCATAACG |
| 613 | MMLV I593E Btm | GCGGCGGTAAATTTCGCCATGCTCATGCGCTGTTG |
| | SDM | CAAAAGCATAACG |
| 614 | MMLV E596A | GACCACGGCGGCGGTAAATCGCGCCATGGATATGC |
| | Btm SDM | GCTGTTGC |
| 615 | MMLV E596R | GACCACGGCGGCGGTAAATACGGCCATGGATATGC |
| | Btm SDM | GCTGTTGC |
| 616 | MMLV E596D | GACCACGGCGGCGGTAAATATCGCCATGGATATGC |
| | Btm SDM | GCTGTTGC |
| 617 | MMLV I597A Btm | CAGACCACGGCGGCGGTACGCTTCGCCATGGATAT |
| | SDM | GCGCTGTTG |
| 618 | MMLV I597R Btm | CAGACCACGGCGGCGGTAACGTTCGCCATGGATAT |
| | SDM | GCGCTGTTG |
| 619 | MMLV I597E Btm | CAGACCACGGCGGCGGTACTCTTCGCCATGGATAT |
| | SDM | GCGCTGTTG |
| 620 | MMLV R650A | GGGCAGCTTGGTCCGCCATCGCGTTTCCACGAGCC |
| | Btm SDM | TCCGCT |
| 621 | MMLV R650K | GGGCAGCTTGGTCCGCCATTTTGTTTCCACGAGCC |
| | Btm SDM | TCCGCT |
| 622 | MMLV R650E | GGGCAGCTTGGTCCGCCATCTCGTTTCCACGAGCC |
| | Btm SDM | TCCGCT |
| 623 | MMLV Q654A | GCCGCCTTACGGGCAGCCGCGTCCGCCATACGGTT |
| | Btm SDM | TCCAC |
| 624 | MMLV Q654R | GCCGCCTTACGGGCAGCACGGTCCGCCATACGGTT |
| | Btm SDM | TCCAC |
| 625 | MMLV Q654E | GCCGCCTTACGGGCAGCCTCGTCCGCCATACGGTT |
| | Btm SDM | TCCAC |
| 626 | MMLV R657A | GTCTCTGTGATCGCCGCCTTCGCGGCAGCTTGGTC |
| | Btm SDM | CGCCATA |
| 627 | MMLV R657K | GTCTCTGTGATCGCCGCCTTTTTGGCAGCTTGGTC |
| | Btm SDM | CGCCATA |
| 628 | MMLV R657E | GTCTCTGTGATCGCCGCCTTCTCGGCAGCTTGGTC |
| | Btm SDM | CGCCATA |
| 629 | MMLV L280R Top | ATTTGCTGAAAGAAGGTCAACGTTGGCGTACTGAT |
| | SDM V2 | GCGCGTAAGGAGACC |
| 630 | MMLV L280R | GGTCTCCTTACGCGCATCAGTACGCCAACGTTGAC |
| | Btm SDM V2 | CTTCTTTCAGCAAAT |
| 631 | MMLV L82R Top | GGGATTAAGCCACATATTCGTCGCTTGCGTGACCA |
| | SDM V2 | GGGGATCTTGGTCCC |
| 632 | MMLV L82R Btm | GGGACCAAGATCCCCTGGTCACGCAAGCGACGAAT |
| | SDM V2 | ATGTGGCTTAATCCC |

### Example 2: Preparation of Reverse Transcriptase Mutants for Screening Increased Activity and Thermostability

a. Overexpression of MMLV RTase and Mutant Variants

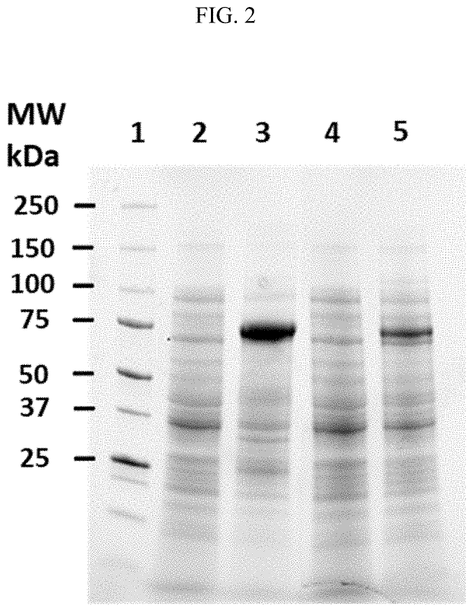

A test induction was used to determine optimum growing conditions. A colony, with the appropriate strain, was used to inoculate Terrific Broth (TB) media (50 mL) with kanamycin (0.05 mg/mL) and grown at 37° C. until an OD of approximately 0.9 was reached. The 50 mL culture was divided in half to accommodate two induction temperatures. IPTG (1M; 12.5 μL) was used to induce protein expression, followed by growth at two induction temperatures for 21 hours. Aliquots (normalized to an OD of 1.25) were taken at 3 and 21 hours, cells were harvested at 13,000×g for one minute, and harvested cells were stored at −20° C. Cells were resuspended in 1×SDS-PAGE running buffer (270 μL) and 5×SDS-PAGE loading dye (70 μL). Samples were boiled for 5 minutes, sonicated, and loaded (15 μL) onto a 4-20% Mini-PROTEAN® TGX Stain-Free™ Protein Gel (Bio Rad, Cat #4568094). SDS-PAGE images are shown in FIG. 2.

b. Expression and Purification of MMLV RTase and Mutant Variants

A colony with the appropriate strain was used to inoculate TB media (1 mL, in a 96-well deep well plate) with kanamycin (0.05 mg/mL) and grown at 37° C. until an OD of approximately 0.9 was achieved followed by cooling of the plate on ice for 5 minutes. Protein expression was induced by the addition of 100 mM IPTG (5 μL), followed by growth at 18° C. for 21 hours. Cells were harvested by spinning samples at 4,700×g for 10 minutes.

Cell pellets were re-suspended in a lysis buffer (50 mM NaPO₄, pH 7.8, 5% glycerol, 300 mM NaCl, and 10 mM imidazole) and lysed by the addition of 1×BugBuster® (Millipore Sigma, Cat #70921) and incubation on an end-over-end mixer for 15 minutes at room temperature. Cell debris was removed by centrifuging the lysate at 16,000×g for 20 minutes at 4° C.

Cleared lysates were applied to a HisPur™ Ni-NTA spin plate (ThermoFisher, Cat #88230). Resin was equilibrated with Screening His-Bind buffer (50 mM NaPO₄, pH 7.8, 5% glycerol, 300 mM NaCl, and 10 mM imidazole) and samples loaded. Samples were washed three times with Screening His-Wash buffer (50 mM NaPO₄, pH 7.8, 5% glycerol, 300 mM NaCl, and 25 mM imidazole) and eluted using Screening His-Elution buffer (50 mM NaPO₄, pH 7.8, 5% glycerol, 300 mM NaCl, and 250 mM imidazole). Purified proteins were normalized to a set concentration (100 nM) for testing purposes.

### Example 3: Evaluation of Reverse Transcriptase Mutants

a. Evaluation of Ability of RTase Mutants to Synthesize DNA

The ability of mutant RTase to synthesize cDNA from purified total RNA (DNased, isolated from HeLa cells) was compared to an MMLV RTase base construct (RNase H minus construct). Mutant MMLV RTases were tested in two formats: (1) standard two-step cDNA synthesis with gene specific primers, followed by qPCR, and (2) one-step addition of the RTase in Integrated DNA Technologies PrimeTime® Gene Expression Master Mix (GEM).

b. Standard Two-Step Procedure

RTases (2 μL, 100 nM) were added to a reaction mixture containing RNA (50 ng), dNTPs (100 μM), gene specific primer set (500 nM; see Table 2), first strand synthesis buffer (1×, 50 mM Tris-HCl, pH 8.3, 75 mM KCl, 3 mM MgCl₂, 10 mM DTT), and SuperaseIN (0.17 U/μL) in a 50 μL volume. The reaction was allowed to proceed at 50° C. for 15 minutes, followed by incubation at 80° C. for 10 minutes.

cDNA synthesized by RTase mutants was quantified by qPCR amplification using an assay that identified the SFRS9 gene in human cells. The assay master mix composition included GEM (1×), ROX (50 nM), SFRS9 primer set (500 nM; see Table 2), and SFRS9 probe (250 nM; see Table 2). Assay master mix and synthesized cDNA were mixed at a 4:1 ratio for a final volume of 20 μL. The reaction was run on qPCR (QuantStudio) for 40 cycles under the following cycle conditions: 95° C. hold for 3 minutes, 95° C. for 15 seconds, and 60° C. for one minute.

TABLE 2
Sequences of primers and probes used for qPCR assays.

| SEQ ID NO: | Primer Name | Primer Sequence (5′-3′) |
|---|---|---|
| 633 | Hs SFRS9 | GTCGAGTATCTCAGAAAAGAAGACA |
| | Forward Primer | |
| 634 | Hs SFRS9 | CTCGGATGTAGGAAGTTTCACC |
| | Reverse Primer | |
| 635 | Hs SFRS9 Probe- | /5SUN/ATGCCCTGC/ZEN/GTAAACTGGATGACA |
| | SUN | /3IABKFQ/ |

c. One-Step Procedure in GEM

RTases (1 μL, 100 nM) were added to a reaction mixture containing RNA (10 ng), GEM (1×), ROX (50 nM), SFRS9 primer set (500 nM; see Table 2), and SFRS9 probe (250 nM; see Table 2) in a final volume of 20 μL. The reaction was run on a qPCR machine (QuantStudio) for 40 cycles using the following cycle conditions: 60° C. hold for 15 minutes, 95° C. hold for 3 minutes, 95° C. for 15 seconds, and 60° C. for one minute.

d. MMLV RTase Base Construct and Single Mutant Variants

As described in Example 1, MMLV RTase single mutant variants were prepared by introducing selected mutations into the MMLV RTase base construct by site-directed mutagenesis, using standard PCR conditions and primers. The sequences of the MMLV RTase base construct and single mutant variants are shown in Table 3. One of skill in the art will understand that the MMLV RTase amino acid sequences of SEQ ID NO: 637 and SEQ ID NO: 717 (the latter of which is described in Example 6 below) are truncated forms of the full-length amino acid sequence of wild-type, or naturally occurring, MMLV RTase. In addition, a person having ordinary skill in the art will understand that a methionine residue is required to recombinantly produce the MMLV RTase base construct and mutants of the disclosure, and as such, that the MMLV RTase sequences disclosed herein (see, e.g., Table 3 below, Table 8 in Example 4, Tables 9 and 12 in Example 5, Table 22 in Example 6, and Table 38 in Example 9) include a methionine residue at the N-terminal end of the amino acid sequence. However, with respect to the present disclosure and for the purpose of identifying and numbering residues in the MMLV RTase amino acid sequence where mutations have been introduced, this methionine residue is considered to be amino acid residue 0 (i.e., is not counted) and the second amino acid residue (e.g., threonine in the MMLV RTase base construct set forth in SEQ ID NO: 637 and SEQ ID NO: 717) is considered to be amino acid residue 1.

TABLE 3
Sequences of MMLV RTase base construct and single mutant MMLV
RTase constructs.

| SEQ ID NO: | Construct | Construct Sequence (DNA: 5′-3′ or AA) |
|---|---|---|
| 636 | MMLV RTase | ATGACTTTAAATATTGAGGATGAGCATCGTTTA |
| | | CATGAGACATCAAAAGAACCCGACGTGAGCTTA |
| | | GGGTCAACGTGGCTTTCTGACTTCCCCCAGGCG |
| | | TGGGCGGAGACTGGCGGAATGGGGTTAGCTGTC |
| | | CGCCAAGCACCGTTGATCATCCCGTTAAAGGCA |
| | | ACGTCTACACCTGTCTCTATCAAACAGTACCCC |
| | | ATGAGTCAAGAGGCCCGCCTGGGGATTAAGCCA |
| | | CATATTCAGCGCTTGCTGGACCAGGGGATCTTG |
| | | GTCCCATGTCAATCTCCGTGGAACACCCCCCTT |
| | | CTGCCCGTGAAAAAGCCAGGTACAAACGATTAT |
| | | CGTCCAGTTCAAGATCTTCGCGAGGTCAACAAA |
| | | CGCGTAGAAGACATCCATCCGACTGTACCTAAT |
| | | CCTTATAATCTGTTATCAGGCCTGCCCCCATCG |
| | | CACCAATGGTATACAGTATTAGACTTGAAAGAC |
| | | GCGTTCTTTTGCCTGCGTCTGCACCCAACGTCT |
| | | CAGCCGCTGTTTGCGTTCGAATGGCGTGATCCT |
| | | GAAATGGGAATTTCGGGTCAGTTAACCTGGACT |
| | | CGTCTGCCCCAGGGCTTTAAAAACAGCCCCACA |
| | | TTGTTCGATGAAGCACTTCACCGTGACTTAGCA |
| | | GACTTCCGTATCCAACACCCAGACTTAATTCTG |
| | | TTACAGTATGTTGACGACCTTTTGTTGGCGGCA |
| | | ACGTCTGAACTTGACTGTCAGCAAGGCACACGC |
| | | GCGTTATTACAAACGTTAGGTAACTTAGGATAT |
| | | CGTGCGTCCGCGAAAAAGGCGCAAATTTGTCAA |
| | | AAACAGGTAAAGTACCTTGGGTATTTGCTGAAA |
| | | GAAGGTCAACGTTGGCTGACTGAAGCGCGTAAG |
| | | GAGACCGTAATGGGGCAGCCTACGCCTAAGACG |
| | | CCACGCCAGTTGCGTGAATTTTTGGGCACAGCG |
| | | GGATTCTGTCGTTTATGGATTCCTGGGTTCGCT |
| | | GAAATGGCTGCACCCCTGTACCCCTTAACAAAA |
| | | ACAGGGACGCTTTTCAACTGGGGGCCAGACCAG |
| | | CAAAAGGCGTATCAGGAGATCAAACAAGCTTTG |
| | | TTGACCGCACCCGCGTTGGGTCTTCCGGATTTA |
| | | ACCAAGCCCTTTGAGCTGTTCGTTGATGAAAAA |
| | | CAGGGATATGCAAAAGGAGTATTAACCCAAAAG |
| | | TTAGGCCCGTGGCGTCGCCCTGTTGCTTACTTG |
| | | AGTAAAAAATTGGATCCTGTCGCAGCAGGATGG |
| | | CCACCGTGCTTGCGTATGGTCGCGGCAATTGCC |
| | | GTTTTGACAAAGGATGCAGGTAAGTTGACGATG |
| | | GGTCAACCCTTAGTAATCTTGGCTCCACATGCT |
| | | GTAGAAGCGTTAGTAAAGCAGCCCCCAGACCGC |
| | | TGGCTTTCTAATGCGCGCATGACCCACTATCAG |
| | | GCGCTTCTGCTTGATACGGATCGTGTACAATTT |
| | | GGACCAGTTGTAGCTTTGAATCCAGCTACTTTG |
| | | CTTCCCCTTCCAGAAGAAGGACTTCAGCACAAT |
| | | TGTTTAGATATTCTGGCCGAGGCACATGGGACG |
| | | CGCCCTGATTTGACGGATCAGCCACTGCCTGAT |
| | | GCCGACCATACATGGTATACTGGCGGCAGTAGT |
| | | CTTCTTCAAGAGGGGCAACGCAAGGCGGGAGCA |
| | | GCCGTCACTACGGAGACCGAAGTTATCTGGGCC |
| | | AAAGCGTTACCCGCGGGAACATCCGCGCAACGT |
| | | GCACAGTTAATCGCTCTGACACAGGCCCTGAAG |
| | | ATGGCAGAGGGCAAAAAGTTGAATGTCTACACC |
| | | AACTCACGTTATGCTTTTGCAACAGCGCATATC |
| | | CATGGCGAAATTTACCGCCGCCGTGGTCTGCTG |
| | | ACTAGTGAGGGTAAGGAAATTAAAAATAAAGAT |
| | | GAGATTCTTGCGTTGTTAAAAGCTTTATTCTTA |
| | | CCAAAACGCCTTTCGATCATTCATTGCCCGGGG |
| | | CATCAAAAGGGTCACTCAGCGGAGGCTCGTGGA |
| | | AACCGTATGGCGGACCAAGCTGCCCGTAAGGCG |
| | | GCGATCACAGAGACCCCGGATACATCAACGCTG |
| | | TTGATCGAAAACAGCTCTCCCTACACTAGCGAG |
| | | CATTTTTAA |
| 637 | MMLV RTase | MTLNIEDEHRLHETSKEPDVSLGSTWLSDFPQA |
| | | WAETGGMGLAVROAPLIIPLKATSTPVSIKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 638 | MMLV RTase with | MTLNIEDEHRLHETSKEPDVSLGSTWLSDFPQA |
| | I61R mutation | WAETGGMGLAVROAPLIIPLKATSTPVSRKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 639 | MMLV RTase with | MTLNIEDEHRLHETSKEPDVSLGSTWLSDFPQA |
| | Q68R mutation | WAETGGMGLAVROAPLIIPLKATSTPVSIKQYP |
| | | MSREARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 640 | MMLV RTase with | MTLNIEDEHRLHETSKEPDVSLGSTWLSDFPQA |
| | Q79R mutation | WAETGGMGLAVRQAPLIIPLKATSTPVSIKQYP |
| | | MSQEARLGIKPHIRRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 641 | MMLV RTase with | MTLNIEDEHRLHETSKEPDVSLGSTWLSDFPQA |
| | L99R mutation | WAETGGMGLAVRQAPLIIPLKATSTPVSIKQYP |
| | | MSQEARLGIKPHIQRLLDQGILVPCQSPWNTPR |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKOPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 642 | MMLV RTase with | MTLNIEDEHRLHETSKEPDVSLGSTWLSDFPQA |
| | E282D mutation | WAETGGMGLAVROAPLIIPLKATSTPVSIKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGOLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTOALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 643 | MMLV RTase with | MTLNIEDEHRLHETSKEPDVSLGSTWLSDFPQA |
| | R298A mutation | WAETGGMGLAVROAPLIIPLKATSTPVSIKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGOLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PAQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |

e. Experimental Results

The two-step and one-step reactions for MMLV RTase base construct and MMLV RTase single mutant variants were analyzed and reported by copy number output based on a standard curve (see Tables 4 and 5). Six single mutant MMLV RTase variants were found to exhibit an increase in the overall activity and thermostability as compared to the MMLV RTase base construct. The six single mutant MMLV RTase variants were as follows: I61R, Q68R, Q79R, L99R, E282D, and R298A.

TABLE 4
Two-step cDNA synthesis by MMLV RT single mutants.
Data was generated via qPCR human
normalizer assay and translated by copy number.

| MMLV RT Variant | Quantity Mean | Quantity Standard Deviation |
|---|---|---|
| MMLV-II | 21,046.784 | 954.827 |
| MMLV-II A283V | 280.423 | 50.910 |
| MMLV-II A283R | 10,390.819 | 340.236 |
| MMLV-II A283E | 7,378.705 | 122.716 |
| MMLV-II E123A | 15,059.791 | 556.095 |
| MMLV-II E123R | 19,043.292 | 415.522 |
| MMLV-II E123D | 3,619.959 | 243.766 |
| MMLV-II E282A | 19,939.551 | 1,645.246 |
| MMLV-II E282R | 15,588.940 | 546.467 |
| MMLV-II E282D | 24,282.327 | 2,259.264 |
| MMLV-II I61A | 648.252 | 45.640 |
| MMLV-II I61R | 26,280.811 | 549.417 |
| MMLV-II I61E | 10,966.741 | 469.747 |
| MMLV-II K102A | 98.438 | 12.778 |
| MMLV-II K102R | 780.114 | 90.331 |
| MMLV-II K102E | 1,674.854 | 157.485 |
| MMLV-II K103A | 359.984 | 67.322 |
| MMLV-II K103R | 206.765 | 20.758 |
| MMLV-II K103E | 200.883 | 16.719 |
| MMLV-II K120A | 217.787 | 72.696 |
| MMLV-II K120R | 3,619.338 | 100.478 |
| MMLV-II K120E | 2,230.375 | 210.050 |
| MMLV-II K193A | 2,736.271 | 162.383 |
| MMLV-II K193R | 11,496.935 | 193.681 |
| MMLV-II K193E | 325.109 | 50.932 |
| MMLV-II K295A | 8,101.927 | 348.373 |
| MMLV-II K295R | 6,879.112 | 131.993 |
| MMLV-II K295E | 9,673.612 | 351.106 |
| MMLV-II K329A | 3,199.167 | 212.003 |
| MMLV-II K329R | 10,387.670 | 330.429 |
| MMLV-II K329E | 18,306.813 | 1,167.600 |
| MMLV-II K53A | 474.465 | 62.390 |
| MMLV-II K53R | 369.020 | 49.436 |
| MMLV-II K53E | 5,308.165 | 104.585 |
| MMLV-II K62A | 2,102.396 | 64.197 |
| MMLV-II K62R | 4,920.330 | 251.414 |
| MMLV-II K62E | 71.723 | 11.419 |
| MMLV-II K75A | 76.659 | 24.657 |
| MMLV-II K75R | 2,842.314 | 77.212 |
| MMLV-II K75E | 1,697.887 | 158.946 |
| MMLV-II L99A | 1,576.246 | 213.455 |
| MMLV-II L99R | 37,070.048 | 1,531.910 |
| MMLV-II L99E | 195.448 | 22.530 |
| MMLV-II N107A | 3,354.325 | 176.385 |
| MMLV-II N107R | 41.532 | 24.527 |
| MMLV-II N107E | 8,523.285 | 353.411 |
| MMLV-II Q291A | 14,093.444 | 576.318 |
| MMLV-II Q291R | 15,736.443 | 566.630 |
| MMLV-II Q291E | 1,480.309 | 93.187 |
| MMLV-II Q68A | n.d. | n.d. |
| MMLV-II Q68R | 20,158.035 | 722.022 |
| MMLV-II Q68E | 2,263.714 | 150.236 |
| MMLV-II Q79A | 2,317.484 | 43.518 |
| MMLV-II Q79R | 37,480.443 | 1,268.309 |
| MMLV-II Q79E | 489.184 | 39.449 |
| MMLV-II R110A | 1,815.710 | 7.917 |
| MMLV-II R110K | 502.172 | 38.619 |
| MMLV-II R110E | 383.331 | 38.162 |
| MMLV-II R298A | 44,477.013 | 3,036.502 |
| MMLV-II R298K | 14,925.202 | 186.581 |
| MMLV-II R298E | 1,150.932 | 56.107 |
| MMLV-II R301A | 2,745.075 | 82.646 |
| MMLV-II R301K | 12,813.899 | 568.898 |
| MMLV-II R301E | 1,583.826 | 198.913 |
| MMLV-II T106A | 16,641.642 | 179.631 |
| MMLV-II T106R | 2,248.217 | 71.295 |
| MMLV-II T106E | 10,302.113 | 250.531 |
| MMLV-II T128V | 7,034.032 | 351.446 |
| MMLV-II T128R | 3,465.069 | 143.456 |
| MMLV-II T128E | 10,709.019 | 110.124 |
| MMLV-II T293A | 4,612.880 | 167.335 |
| MMLV-II T293R | 13,753.879 | 319.851 |
| MMLV-II T293E | 12,893.457 | 223.100 |
| MMLV-II T296A | 2,192.531 | 76.071 |
| MMLV-II T296R | 893.449 | 51.913 |
| MMLV-II T296E | 473.936 | 102.414 |
| MMLV-II T55A | 5,774.471 | 223.173 |
| MMLV-II T55R | 3,284.089 | 314.651 |
| MMLV-II T55E | 6,143.058 | 429.507 |
| MMLV-II T57A | 6,129.791 | 285.070 |
| MMLV-II T57R | 888.244 | 11.952 |
| MMLV-II T57E | 1,487.448 | 71.681 |
| MMLV-II V101A | 552.130 | 98.391 |
| MMLV-II V101R | 4,754.017 | 107.434 |
| MMLV-II V101E | 1,388.699 | 87.091 |
| MMLV-II V112A | 2,085.594 | 72.265 |
| MMLV-II V112R | 377.194 | 41.722 |
| MMLV-II V112E | 210.825 | 17.715 |
| MMLV-II V59A | 628.779 | 15.216 |
| MMLV-II V59R | 6,662.173 | 210.234 |
| MMLV-II V59E | 3,249.465 | 79.848 |
| MMLV-II Y109A | 101.656 | 6.717 |
| MMLV-II Y109R | 349.373 | 27.171 |
| MMLV-II Y109E | 1,029.589 | 45.189 |
| MMLV-IV | 71,572.714 | 4,656.679 |

TABLE 5
One-step cDNA synthesis by MMLV RT single
mutants. Data was generated via qPCR human
normalizer assay and data is translated by copy number.

| MMLV RT Variant | Quantity Mean | Quantity Standard Deviation |
|---|---|---|
| MMLV-II | 20,638.973 | 614.785 |
| MMLV-II A283V | 8,802.753 | 220.902 |
| MMLV-II A283R | 14,379.575 | 337.562 |
| MMLV-II A283E | 16,396.614 | 203.476 |
| MMLV-II E123A | 17,975.218 | 259.986 |
| MMLV-II E123R | 20,652.508 | 515.600 |
| MMLV-II E123D | 14,452.672 | 242.000 |
| MMLV-II E282A | 19,017.751 | 827.419 |
| MMLV-II E282R | 17,180.421 | 204.739 |
| MMLV-II E282D | 20,735.271 | 420.881 |
| MMLV-II I61A | 7,450.147 | 348.788 |
| MMLV-II I61R | 25,123.507 | 2,977.836 |
| MMLV-II I61E | 17,441.860 | 1,662.749 |
| MMLV-II K102A | 9,342.754 | 120.846 |
| MMLV-II K102R | 10,563.589 | 255.139 |
| MMLV-II K102E | 13,925.008 | 307.601 |
| MMLV-II K103A | 9,429.555 | 437.351 |
| MMLV-II K103R | 9,009.846 | 155.888 |
| MMLV-II K103E | 7,985.278 | 189.792 |
| MMLV-II K120A | 8,593.433 | 438.722 |
| MMLV-II K120R | 12,558.793 | 407.946 |
| MMLV-II K120E | 12,268.574 | 303.495 |
| MMLV-II K193A | 12,977.263 | 537.992 |
| MMLV-II K193R | 13,446.766 | 2,337.906 |
| MMLV-II K193E | 8,536.558 | 182.514 |
| MMLV-II K295A | 13,506.491 | 1,613.467 |
| MMLV-II K295R | 13,944.407 | 1,839.608 |
| MMLV-II K295E | 15,021.823 | 650.111 |
| MMLV-II K329A | 13,284.541 | 246.298 |
| MMLV-II K329R | 15,935.899 | 970.971 |
| MMLV-II K329E | 20,628.859 | 884.254 |
| MMLV-II K53A | 10,868.676 | 161.435 |
| MMLV-II K53R | 9,908.252 | 632.663 |
| MMLV-II K53E | 20,666.775 | 518.895 |
| MMLV-II K62A | 9,454.043 | 732.242 |
| MMLV-II K62R | 14,532.171 | 63.450 |
| MMLV-II K62E | 8,341.361 | 436.076 |
| MMLV-II K75A | 9,084.502 | 113.100 |
| MMLV-II K75R | 13,106.462 | 331.663 |
| MMLV-II K75E | 11,191.849 | 565.160 |
| MMLV-II L99A | 12,876.076 | 49.507 |
| MMLV-II L99R | 27,167.197 | 142.371 |
| MMLV-II L99E | 6,534.199 | 2,730.598 |
| MMLV-II N107A | 13,563.421 | 349.378 |
| MMLV-II N107R | 8,654.167 | 497.167 |
| MMLV-II N107E | 16,675.075 | 172.596 |
| MMLV-II Q291A | 20,957.729 | 150.006 |
| MMLV-II Q291R | 17,980.723 | 346.436 |
| MMLV-II Q291E | 11,025.722 | 407.116 |
| MMLV-II Q68A | n.d. | n.d. |
| MMLV-II Q68R | 24,925.791 | 937.265 |
| MMLV-II Q68E | 12,844.484 | 165.039 |
| MMLV-II Q79A | 12,038.975 | 482.596 |
| MMLV-II Q79R | 28,458.521 | 296.595 |
| MMLV-II Q79E | 10,358.863 | 309.043 |
| MMLV-II R110A | 11,517.764 | 562.094 |
| MMLV-II R110K | 8,112.167 | 76.742 |
| MMLV-II R110E | 8,809.423 | 290.785 |
| MMLV-II R298A | 27,817.905 | 172.690 |
| MMLV-II R298K | 18,222.660 | 825.743 |
| MMLV-II R298E | 10,783.790 | 783.279 |
| MMLV-II R301A | 11,344.854 | 63.499 |
| MMLV-II R301K | 17,584.850 | 445.587 |
| MMLV-II R301E | 10,146.906 | 1,879.902 |
| MMLV-II T106A | 17,717.520 | 215.965 |
| MMLV-II T106R | 11,680.187 | 148.213 |
| MMLV-II T106E | 21,203.557 | 366.469 |
| MMLV-II T128V | 14,384.970 | 355.754 |
| MMLV-II T128R | 12,938.223 | 464.841 |
| MMLV-II T128E | 14,781.394 | 1,930.931 |
| MMLV-II T293A | 15,658.189 | 347.640 |
| MMLV-II T293R | 19,976.165 | 253.604 |
| MMLV-II T293E | 17,580.335 | 404.397 |
| MMLV-II T296A | 10,312.142 | 159.775 |
| MMLV-II T296R | 8,482.071 | 92.806 |
| MMLV-II T296E | 7,687.972 | 112.884 |
| MMLV-II T55A | 18,073.262 | 618.174 |
| MMLV-II T55R | 11,546.179 | 138.906 |
| MMLV-II T55E | 12,299.658 | 815.911 |
| MMLV-II T57A | 14,700.042 | 2,916.521 |
| MMLV-II T57R | 11,195.901 | 145.433 |
| MMLV-II T57E | 11,958.503 | 605.445 |
| MMLV-II V101A | 10,697.751 | 269.696 |
| MMLV-II V101R | 8,934.765 | 53.924 |
| MMLV-II V101E | 11,295.874 | 296.506 |
| MMLV-II V112A | 12,854.738 | 356.724 |
| MMLV-II V112R | 6,331.802 | 303.453 |
| MMLV-II V112E | 7,643.184 | 448.446 |
| MMLV-II V59A | 9,520.143 | 339.954 |
| MMLV-II V59R | 18,523.053 | 499.377 |
| MMLV-II V59E | 16,029.631 | 137.454 |
| MMLV-II Y109A | 8,421.361 | 185.196 |
| MMLV-II Y109R | 8,581.961 | 129.732 |
| MMLV-II Y109E | 10,216.473 | 416.388 |
| MMLV-IV | 65,726.159 | 1,811.314 |

### Example 4: Extension of Reverse Transcriptase Single Mutants

The amino acid positions that enclosed the MMLV RTase single mutants identified in Example 3 were further evaluated to include all possible amino acid substitutions at that position. The single mutants were cloned, overexpressed, and purified as described in Examples 1 and 2, and evaluated as described in Example 3. The two-step and one-step reactions for MMLV RTase base construct and MMLV RTase double mutant variants were analyzed and reported by copy number output based on a standard curve (see Tables 6 and 7). Ten single mutant MMLV RTase variants (see Table 8) were found to exhibit an increase in the overall activity and thermostability as compared to the MMLV RTase base construct. The ten single mutant MMLV RTase variants were as follows: I61K, I61M, Q68I, Q68K, Q79H, Q79I, L99K, L99N, E282M and E282W.

TABLE 6
Two-step cDNA synthesis by MMLV RT single mutants.
Data was generated via qPCR human
normalizer assay and translated by copy number.

| MMLV RT Variant | Quantity Mean | Quantity Standard Deviation |
|---|---|---|
| MMLV-II | 1,484.121 | 125.278 |
| MMLV-II E282C | 749.332 | 37.947 |
| MMLV-II E282F | 968.042 | 28.112 |
| MMLV-II E282G | 841.839 | 30.618 |
| MMLV-II E282H | 936.562 | 64.904 |
| MMLV-II E282I | 1,418.551 | 8.682 |
| MMLV-II E282K | 2,399.973 | 50.862 |
| MMLV-II E282L | 1,778.903 | 134.133 |
| MMLV-II E282M | 2,115.328 | 125.477 |
| MMLV-II E282N | 1,175.130 | 79.221 |
| MMLV-II E282P | 1,529.331 | 61.525 |
| MMLV-II E282Q | 1,856.418 | 24.118 |
| MMLV-II E282S | 673.670 | 44.770 |
| MMLV-II E282T | 994.318 | 24.066 |
| MMLV-II E282V | 748.877 | 29.053 |
| MMLV-II E282W | 2,469.404 | 141.080 |
| MMLV-II E282Y | 1,360.706 | 338.309 |
| MMLV-II I61C | 283.240 | 11.244 |
| MMLV-II I61D | 349.008 | 10.979 |
| MMLV-II I61F | 784.163 | 22.643 |
| MMLV-II I61G | 395.348 | 21.967 |
| MMLV-II I61H | 736.015 | 30.271 |
| MMLV-II I61K | 4,479.606 | 62.627 |
| MMLV-II I61L | 1,106.547 | 38.553 |
| MMLV-II I61M | 4,198.088 | 93.025 |
| MMLV-II I61N | 709.752 | 29.312 |
| MMLV-II I61P | 32.935 | 16.814 |
| MMLV-II I61Q | 1,311.695 | 145.810 |
| MMLV-II I61S | 797.783 | 50.626 |
| MMLV-II I61T | 628.173 | 33.371 |
| MMLV-II I61V | 1,439.915 | 27.490 |
| MMLV-II I61W | 442.039 | 29.310 |
| MMLV-II I61Y | 534.249 | 26.831 |
| MMLV-II L99C | 3,109.142 | 80.016 |
| MMLV-II L99D | 83.653 | 3.432 |
| MMLV-II L99F | 2,811.513 | 79.584 |
| MMLV-II L99G | 908.041 | 16.157 |
| MMLV-II L99H | 4,881.196 | 390.497 |
| MMLV-II L99I | 910.072 | 71.671 |
| MMLV-II L99K | 6,410.818 | 127.262 |
| MMLV-II L99M | 976.548 | 65.154 |
| MMLV-II L99N | 4,974.458 | 162.464 |
| MMLV-II L99P | 6.416 | 1.820 |
| MMLV-II L99Q | 3,908.473 | 337.167 |
| MMLV-II L99S | 3,793.955 | 86.959 |
| MMLV-II L99T | 4,189.211 | 27.640 |
| MMLV-II L99V | 964.081 | 48.105 |
| MMLV-II L99W | 1,614.660 | 40.442 |
| MMLV-II L99Y | 2,123.406 | 181.945 |
| MMLV-II Q68A | 1,184.702 | 7.676 |
| MMLV-II Q68C | 2,038.167 | 36.463 |
| MMLV-II Q68D | 1,613.880 | 77.796 |
| MMLV-II Q68F | 1,805.647 | 62.456 |
| MMLV-II Q68G | 2,262.873 | 69.688 |
| MMLV-II Q68H | 106.421 | 9.860 |
| MMLV-II Q681 | 2,675.446 | 73.874 |
| MMLV-II Q68K | 1,042.979 | 70.081 |
| MMLV-II Q68L | 1,070.742 | 57.215 |
| MMLV-II Q68M | 1,342.806 | 58.349 |
| MMLV-II Q68N | 1,993.946 | 65.808 |
| MMLV-II Q68P | 2,025.753 | 25.540 |
| MMLV-II Q68S | 1,895.984 | 26.959 |
| MMLV-II Q68T | 431.442 | 22.751 |
| MMLV-II Q68V | 1,534.710 | 110.794 |
| MMLV-II Q68W | 1,790.706 | 124.583 |
| MMLV-II Q79C | 2,477.812 | 107.510 |
| MMLV-II Q79D | 627.902 | 11.073 |
| MMLV-II Q79F | 1,786.571 | 126.904 |
| MMLV-II Q79G | 2,702.985 | 83.998 |
| MMLV-II Q79H | 2,851.710 | 57.501 |
| MMLV-II Q791 | 2,967.710 | 57.440 |
| MMLV-II Q79K | 1,346.751 | 64.513 |
| MMLV-II Q79L | 2,214.615 | 67.622 |
| MMLV-II Q79M | 1,847.181 | 31.384 |
| MMLV-II Q79N | 1,365.563 | 54.775 |
| MMLV-II Q79P | 674.074 | 42.100 |
| MMLV-II Q79S | 2,199.353 | 52.958 |
| MMLV-II Q79T | 1,523.163 | 77.025 |
| MMLV-II Q79V | 1,704.661 | 77.643 |
| MMLV-II Q79W | 2,186.489 | 31.470 |
| MMLV-II Q79Y | 2,326.023 | 123.508 |
| MMLV-II R298C | 79.970 | 9.815 |
| MMLV-II R298D | 0.000 | 0.000 |
| MMLV-II R298F | 84.760 | 9.362 |
| MMLV-II R298G | 357.027 | 15.726 |
| MMLV-II R298H | 269.257 | 20.814 |
| MMLV-II R298I | 130.983 | 5.364 |
| MMLV-II R298L | 199.612 | 5.843 |
| MMLV-II R298M | 172.013 | 18.710 |
| MMLV-II R298N | 199.678 | 2.660 |
| MMLV-II R298P | 122.098 | 5.900 |
| MMLV-II R298Q | 118.092 | 40.694 |
| MMLV-II R298S | 406.112 | 7.695 |
| MMLV-II R298T | 618.616 | 20.023 |
| MMLV-II R298V | 136.498 | 13.297 |
| MMLV-II R298W | 68.096 | 7.016 |
| MMLV-II R298Y | 162.713 | 7.854 |
| MMLV-IV | 6,830.294 | 376.878 |

TABLE 7
One-step cDNA synthesis by MMLV RT single mutants.
Data was generated via qPCR human normalizer
assay and data is translated by copy number.

| | | Quantity Standard |
|---|---|---|
| MMLV RT Variant | Quantity Mean | Deviation |
| MMLV-II | 408.018 | 8.693 |
| MMLV-II E282C | 175.083 | 7.005 |
| MMLV-II E282F | 1,043.025 | 16.137 |
| MMLV-II E282G | 635.037 | 13.293 |
| MMLV-II E282H | 656.956 | 10.018 |
| MMLV-II E282I | 1,033.125 | 44.996 |
| MMLV-II E282K | 751.309 | 17.611 |
| MMLV-II E282L | 1,072.350 | 80.365 |
| MMLV-II E282M | 1,318.072 | 51.735 |
| MMLV-II E282N | 539.305 | 10.767 |
| MMLV-II E282P | 725.869 | 92.685 |
| MMLV-II E282Q | 626.674 | 12.129 |
| MMLV-II E282S | 354.956 | 34.850 |
| MMLV-II E282T | 485.477 | 45.783 |
| MMLV-II E282V | 594.047 | 27.898 |
| MMLV-II E282W | 913.290 | 61.145 |
| MMLV-II E282Y | 759.920 | 34.784 |
| MMLV-II I61C | 219.438 | 18.403 |
| MMLV-II I61D | 347.020 | 13.303 |
| MMLV-II I61F | 428.623 | 25.316 |
| MMLV-II I61G | 389.503 | 21.764 |
| MMLV-II I61H | 514.330 | 18.416 |
| MMLV-II I61K | 2,343.894 | 67.214 |
| MMLV-II I61L | 621.572 | 14.892 |
| MMLV-II I61M | 2,536.807 | 150.371 |
| MMLV-II I61N | 538.519 | 20.736 |
| MMLV-II I61P | 61.683 | 18.802 |
| MMLV-II I61Q | 701.471 | 32.487 |
| MMLV-II I61S | 611.977 | 30.430 |
| MMLV-II I61T | 534.254 | 31.643 |
| MMLV-II I61V | 881.608 | 20.662 |
| MMLV-II I61W | 428.440 | 17.964 |
| MMLV-II I61Y | 347.930 | 4.412 |
| MMLV-II L99C | 2,390.104 | 35.867 |
| MMLV-II L99D | 185.044 | 6.975 |
| MMLV-II L99F | 1,577.767 | 7.757 |
| MMLV-II L99G | 987.225 | 9.718 |
| MMLV-II L99H | 3,886.372 | 111.670 |
| MMLV-II L99I | 613.648 | 46.303 |
| MMLV-II L99K | 7,597.650 | 321.753 |
| MMLV-II L99M | 934.817 | 52.006 |
| MMLV-II L99N | 4,689.222 | 160.641 |
| MMLV-II L99P | 18.537 | 1.131 |
| MMLV-II L99Q | 2,394.744 | 64.077 |
| MMLV-II L99S | 3,293.831 | 111.802 |
| MMLV-II L99T | 3,505.113 | 101.670 |
| MMLV-II L99V | 677.756 | 49.356 |
| MMLV-II L99W | 839.088 | 50.301 |
| MMLV-II L99Y | 1,127.536 | 19.074 |
| MMLV-II Q68A | 827.617 | 30.689 |
| MMLV-II Q68C | 1,110.680 | 45.944 |
| MMLV-II Q68D | 1,045.802 | 25.488 |
| MMLV-II Q68F | 1,210.166 | 120.899 |
| MMLV-II Q68G | 907.279 | 30.688 |
| MMLV-II Q68H | 150.384 | 6.867 |
| MMLV-II Q68I | 1,550.372 | 76.712 |
| MMLV-II Q68K | 1,712.176 | 47.342 |
| MMLV-II Q68L | 651.039 | 51.426 |
| MMLV-II Q68M | 1,395.463 | 34.805 |
| MMLV-II Q68N | 1,241.364 | 25.780 |
| MMLV-II Q68P | 1,249.444 | 13.709 |
| MMLV-II Q68S | 1,125.260 | 21.324 |
| MMLV-II Q68T | 792.901 | 31.513 |
| MMLV-II Q68V | 1,026.654 | 24.972 |
| MMLV-II Q68W | 1,594.175 | 101.221 |
| MMLV-II Q79C | 1,948.151 | 87.341 |
| MMLV-II Q79D | 458.131 | 10.763 |
| MMLV-II Q79F | 1,623.675 | 50.723 |
| MMLV-II Q79G | 1,885.097 | 20.190 |
| MMLV-II Q79H | 2,508.763 | 149.926 |
| MMLV-II Q79I | 2,329.030 | 76.545 |
| MMLV-II Q79K | 1,861.302 | 24.320 |
| MMLV-II Q79L | 1,496.247 | 30.399 |
| MMLV-II Q79M | 1,496.469 | 38.178 |
| MMLV-II Q79N | 995.813 | 42.279 |
| MMLV-II Q79P | 526.914 | 23.216 |
| MMLV-II Q79S | 1,685.124 | 42.694 |
| MMLV-II Q79T | 966.505 | 8.377 |
| MMLV-II Q79V | 1,218.191 | 21.512 |
| MMLV-II Q79W | 1,962.326 | 37.135 |
| MMLV-II Q79Y | 2,218.504 | 56.938 |
| MMLV-II R298C | 45.500 | 1.456 |
| MMLV-II R298D | 0.000 | 0.000 |
| MMLV-II R298F | 104.825 | 5.133 |
| MMLV-II R298G | 323.542 | 14.052 |
| MMLV-II R298H | 253.202 | 47.711 |
| MMLV-II R298I | 205.982 | 8.304 |
| MMLV-II R298L | 213.674 | 15.199 |
| MMLV-II R298M | 176.347 | 12.484 |
| MMLV-II R298N | 142.969 | 39.198 |
| MMLV-II R298P | 188.995 | 3.689 |
| MMLV-II R298Q | 95.525 | 44.292 |
| MMLV-II R298S | 307.614 | 9.962 |
| MMLV-II R298T | 487.828 | 3.480 |
| MMLV-II R298V | 255.828 | 12.902 |
| MMLV-II R298W | 37.872 | 8.482 |
| MMLV-II R298Y | 153.333 | 25.137 |
| MMLV-IV | 19,407.721 | 466.310 |

TABLE 8
Sequences of single mutant MMLV RTase variants.

| SEQ ID NO: | Construct | Construct Sequence (AA) |
|---|---|---|
| 644 | MMLV RTase with | MTLNIEDEHRLHETSKEPDVSLGSTWLSDFPQA |
| | I61K mutation | WAETGGMGLAVRQAPLIIPLKATSTPVSKKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 645 | MMLV RTase with | MTLNIEDEHRLHETSKEPDVSLGSTWLSDFPQA |
| | I61M mutation | WAETGGMGLAVRQAPLIIPLKATSTPVSMKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 646 | MMLV RTase with | MTLNIEDEHRLHETSKEPDVSLGSTWLSDFPQA |
| | Q68I mutation | WAETGGMGLAVRQAPLIIPLKATSTPVSIKQYP |
| | | MSIEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 647 | MMLV RTase with | MTLNIEDEHRLHETSKEPDVSLGSTWLSDFPQA |
| | Q68K mutation | WAETGGMGLAVRQAPLIIPLKATSTPVSIKQYP |
| | | MSKEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 648 | MMLV RTase with | MTLNIEDEHRLHETSKEPDVSLGSTWLSDFPQA |
| | Q79H mutation | WAETGGMGLAVRQAPLIIPLKATSTPVSIKQYP |
| | | MSQEARLGIKPHIHRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 649 | MMLV RTase with | MTLNIEDEHRLHETSKEPDVSLGSTWLSDFPQA |
| | Q79I mutation | WAETGGMGLAVRQAPLIIPLKATSTPVSIKQYP |
| | | MSQEARLGIKPHIIRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 650 | MMLV RTase with | MTLNIEDEHRLHETSKEPDVSLGSTWLSDFPQA |
| | L99K mutation | WAETGGMGLAVRQAPLIIPLKATSTPVSIKQYP |
| | | MSQEARLGIKPHIQRLLDQGILVPCQSPWNTPL |
| | | KPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 651 | MMLV RTase with | MTLNIEDEHRLHETSKEPDVSLGSTWLSDFPQA |
| | L99N mutation | WAETGGMGLAVRQAPLIIPLKATSTPVSIKQYP |
| | | MSQEARLGIKPHIQRLLDQGILVPCQSPWNTPL |
| | | NPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 652 | MMLV RTase with | MTLNIEDEHRLHETSKEPDVSLGSTWLSDFPQA |
| | E282M mutation | WAETGGMGLAVRQAPLIIPLKATSTPVSIKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTMARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 653 | MMLV RTase with | MTLNIEDEHRLHETSKEPDVSLGSTWLSDFPQA |
| | E282W mutation | WAETGGMGLAVRQAPLIIPLKATSTPVSIKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTWARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |

### Example 5: Stacking of Reverse Transcriptase Mutants with Enhanced Activity

a. MMLV RTase Double Mutants

The MMLV RTase single mutants identified in Example 3 were stacked to further improve the ability of MMLV RTase to synthesize cDNA from purified total RNA (DNased, isolated from HeLa cells) as compared to the MMLV RTase base construct (RNase H minus construct). Fifteen MMLV RTase double mutant variants (see Table 9) were cloned, overexpressed, and purified as described in Examples 1 and 2, and evaluated as described in Example 3. The two-step and one-step reactions for MMLV RTase base construct and MMLV RTase double mutant variants were analyzed and reported by copy number output based on a standard curve (see Tables 10 and 11).

Four of the fifteen MMLV RTase double mutant variants were found to exhibit increased overall activity and thermostability as compared to the other MMLV RTase double mutant variants, and almost all of the MMLV RTase double mutant variants exhibited increased overall activity and thermostability as compared to the MMLV RTase base construct. The four MMLV RTase double mutant variants that were found to exhibit the highest overall activity were E282D/L99R, L99R/Q68R, L99R/Q79R, and Q68R/Q79R.

TABLE 9
Sequences of double mutant MMLV RTase variants.

| SEQ ID NO: | Construct | Construct Sequence (AA) |
|---|---|---|
| 654 | MMLV RTase with | MTLNIEDEHRLHETSKEPDVSLGSTWLSDFPQA |
| | I61R/E282D mutations | WAETGGMGLAVRQAPLIIPLKATSTPVSRKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 655 | MMLV RTase with | MTLNIEDEHRLHETSKEPDVSLGSTWLSDFPQA |
| | L99R/E282D mutations | WAETGGMGLAVRQAPLIIPLKATSTPVSIKQYP |
| | | MSQEARLGIKPHIQRLLDQGILVPCQSPWNTPR |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 656 | MMLV RTase with | MTLNIEDEHRLHETSKEPDVSLGSTWLSDFPQA |
| | Q68R/E282D | WAETGGMGLAVRQAPLIIPLKATSTPVSIKQYP |
| | mutations | MSREARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 657 | MMLV RTase with | MTLNIEDEHRLHETSKEPDVSLGSTWLSDFPQA |
| | Q79R/E282D | WAETGGMGLAVRQAPLIIPLKATSTPVSIKQYP |
| | mutations | MSQEARLGIKPHIRRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 658 | MMLV RTase with | MTLNIEDEHRLHETSKEPDVSLGSTWLSDFPQA |
| | E282D/R298A | WAETGGMGLAVRQAPLIIPLKATSTPVSIKQYP |
| | mutations | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PAQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 659 | MMLV RTase with | MTLNIEDEHRLHETSKEPDVSLGSTWLSDFPQA |
| | I61R/L99R mutations | WAETGGMGLAVRQAPLIIPLKATSTPVSRKQYP |
| | | MSQEARLGIKPHIQRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 660 | MMLV RTase with | MTLNIEDEHRLHETSKEPDVSLGSTWLSDFPQA |
| | I61R/Q68R mutations | WAETGGMGLAVRQAPLIIPLKATSTPVSRKQYP |
| | | MSREARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEH |
| 661 | MMLV RTase with | MTLNIEDEHRLHETSKEPDVSLGSTWLSDFPQA |
| | I61R/Q79R mutations | WAETGGMGLAVRQAPLIIPLKATSTPVSRKQYP |
| | | MSQEARLGIKPHIRRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 662 | MMLV RTase with | MTLNIEDEHRLHETSKEPDVSLGSTWLSDFPQA |
| | Q68R/L99R mutations | WAETGGMGLAVRQAPLIIPLKATSTPVSRKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PAQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 663 | MMLV RTase with | MTLNIEDEHRLHETSKEPDVSLGSTWLSDFPQA |
| | I61R/R298A mutations | WAETGGMGLAVRQAPLIIPLKATSTPVSIKQYP |
| | | MSREARLGIKPHIQRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 664 | MMLV RTase with | MTLNIEDEHRLHETSKEPDVSLGSTWLSDFPQA |
| | Q79R/L99R mutations | WAETGGMGLAVRQAPLIIPLKATSTPVSIKQYP |
| | | MSQEARLGIKPHIRRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 665 | MMLV RTase with | MTLNIEDEHRLHETSKEPDVSLGSTWLSDFPQA |
| | L99R/R298A | WAETGGMGLAVRQAPLIIPLKATSTPVSIKQYP |
| | mutations | MSQEARLGIKPHIQRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PAQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 666 | MMLV RTase with | MTLNIEDEHRLHETSKEPDVSLGSTWLSDFPQA |
| | Q68R/Q79R mutations | WAETGGMGLAVRQAPLIIPLKATSTPVSIKQYP |
| | | MSREARLGIKPHIRRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 667 | MMLV RTase with | MTLNIEDEHRLHETSKEPDVSLGSTWLSDFPQA |
| | Q68R/R298A | WAETGGMGLAVRQAPLIIPLKATSTPVSIKQYP |
| | mutations | MSREARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PAQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 668 | MMLV RTase with | MTLNIEDEHRLHETSKEPDVSLGSTWLSDFPQA |
| | Q79R/R298A | WAETGGMGLAVRQAPLIIPLKATSTPVSIKQYP |
| | mutations | MSQEARLGIKPHIRRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PAQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |

TABLE 10
Two-Step cDNA synthesis by MMLV RT double mutants.
Data was generated via qPCR human normalizer
assay and data is translated by copy number.

| | | Quantity Standard |
|---|---|---|
| MMLV RT Variant | Quantity Mean | Deviation |
| MMLV-II | 1,773.623 | 5.057 |
| MMLV-II E282D/I61R | 4,810.277 | 143.422 |
| MMLV-II E282D/L99R | 7,266.281 | 50.730 |
| MMLV-II E282D/Q68R | 5,186.392 | 69.563 |
| MMLV-II E282D/Q79R | 4,311.403 | 95.402 |
| MMLV-II E282D/R298A | 1,366.524 | 16.429 |
| MMLV-II I61R/L99R | 6,061.812 | 174.619 |
| MMLV-II I61R/Q68R | 5,899.316 | 39.879 |
| MMLV-II I61R/Q79R | 5,257.089 | 98.378 |
| MMLV-II I61R/R298A | 2,661.223 | 68.948 |
| MMLV-II L99R/Q68R | 7,750.519 | 94.408 |
| MMLV-II L99R/Q79R | 7,455.203 | 124.095 |
| MMLV-II L99R/R298A | 5,351.021 | 179.558 |
| MMLV-II Q68R/Q79R | 7,178.681 | 86.595 |
| MMLV-II Q68R/R298A | 4,524.340 | 84.703 |
| MMLV-II Q79R/R298A | 3,739.608 | 58.621 |
| MMLV-IV | 8,258.715 | 79.458 |

TABLE 11
One-Step cDNA synthesis by MMLV RT double mutants.
Data was generated via qPCR human normalizer
assay and data is translated by copy number.

| | | Quantity Standard |
|---|---|---|
| MMLV-RT Variant | Quantity Mean | Deviation |
| MMLV-II | 859.127 | 24.795 |
| MMLV-II E282D/I61R | 2,948.906 | 49.177 |
| MMLV-II E282D/L99R | 4,814.957 | 239.110 |
| MMLV-II E282D/Q68R | 3,709.046 | 131.434 |
| MMLV-II E282D/Q79R | 3,694.187 | 98.772 |
| MMLV-II E282D/R298A | 794.643 | 39.913 |
| MMLV-II I61R/L99R | 3,443.713 | 180.210 |
| MMLV-II I61R/Q68R | 3,525.138 | 112.288 |
| MMLV-II I61R/Q79R | 3,125.990 | 120.996 |
| MMLV-II I61R/R298A | 2,006.208 | 83.559 |
| MMLV-II L99R/Q68R | 6,755.852 | 102.788 |
| MMLV-II L99R/Q79R | 6,709.502 | 35.997 |
| MMLV-II L99R/R298A | 2,128.451 | 55.565 |
| MMLV-II Q68R/Q79R | 6,343.821 | 140.779 |
| MMLV-II Q68R/R298A | 2,406.470 | 74.117 |
| MMLV-II Q79R/R298A | 2,301.759 | 22.849 |
| MMLV-IV | 15,411.857 | 333.388 |

b. Cloning of MMLV RTase Triple and More Mutants

Following the double mutant variants, MMLV RTase single mutants were stacked further to improve the ability of MMLV RTase to synthesize cDNA from purified total RNA (DNased, isolated from HeLa cells) as compared to the MMLV RTase base construct (RNase H minus construct). Seventeen MMLV RTase triple or more mutant variants (see Table 12) were cloned as described in Example 1.

TABLE 12
Sequences of triple or more mutant MMLV RTase variants.

| SEQ ID | | |
|---|---|---|
| NO: | Construct | Construct Sequence (AA) |
| 669 | MMLV RTase with | MTLNIEDEHRLHETSKEPDVSLGSTWLSDFPQA |
| | Q68R/L99R/E282D | WAETGGMGLAVRQAPLIIPLKATSTPVSIKQYP |
| | mutations | MSREARLGIKPHIQRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 670 | MMLV RTase with | MTLNIEDEHRLHETSKEPDVSLGSTWLSDFPQA |
| | Q79R/L99R/E282D | WAETGGMGLAVRQAPLIIPLKATSTPVSIKQYP |
| | mutations | MSQEARLGIKPHIRRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 671 | MMLV RTase with | MTLNIEDEHRLHETSKEPDVSLGSTWLSDFPQA |
| | Q68R/Q79R/L99R | WAETGGMGLAVRQAPLIIPLKATSTPVSIKQYP |
| | mutations | MSREARLGIKPHIRRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 672 | MMLV RTase with | MTLNIEDEHRLHETSKEPDVSLGSTWLSDFPQA |
| | Q68R/Q79R/E282D | WAETGGMGLAVRQAPLIIPLKATSTPVSIKQYP |
| | mutations | MSREARLGIKPHIRRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 673 | MMLV RTase with | MTLNIEDEHRLHETSKEPDVSLGSTWLSDFPQA |
| | Q68R/Q79R/L99R/E282D | WAETGGMGLAVRQAPLIIPLKATSTPVSIKQYP |
| | mutations | MSREARLGIKPHIRRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 674 | MMLV RTase with | MTLNIEDEHRLHETSKEPDVSLGSTWLSDFPQA |
| | Q68R/Q79R/L99K/E282D | WAETGGMGLAVRQAPLIIPLKATSTPVSIKQYP |
| | mutations | MSREARLGIKPHIRRLLDQGILVPCQSPWNTPL |
| | | KPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 675 | MMLV RTase with | MTLNIEDEHRLHETSKEPDVSLGSTWLSDFPQA |
| | Q68R/Q79R/L99N/E282D | WAETGGMGLAVRQAPLIIPLKATSTPVSIKQYP |
| | mutations | MSREARLGIKPHIRRLLDQGILVPCQSPWNTPL |
| | | NPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 676 | MMLV RTase with | MTLNIEDEHRLHETSKEPDVSLGSTWLSDFPQA |
| | Q68I/Q79R/L99R/E282D | WAETGGMGLAVRQAPLIIPLKATSTPVSIKQYP |
| | mutations | MSIEARLGIKPHIRRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 677 | MMLV RTase with | MTLNIEDEHRLHETSKEPDVSLGSTWLSDFPQA |
| | Q68K/Q79R/L99R/E282D | WAETGGMGLAVRQAPLIIPLKATSTPVSIKQYP |
| | mutations | MSKEARLGIKPHIRRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 678 | MMLV RTase with | MTLNIEDEHRLHETSKEPDVSLGSTWLSDFPQA |
| | Q68R/Q79H/L99R/E282D | WAETGGMGLAVRQAPLIIPLKATSTPVSIKQYP |
| | mutations | MSREARLGIKPHIHRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 679 | MMLV RTase with | MTLNIEDEHRLHETSKEPDVSLGSTWLSDFPQA |
| | Q68R/Q79I/L99R/E282D | WAETGGMGLAVRQAPLIIPLKATSTPVSIKQYP |
| | mutations | MSREARLGIKPHIIRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 680 | MMLV RTase with | MTLNIEDEHRLHETSKEPDVSLGSTWLSDFPQA |
| | Q68R/Q79R/L99R/E282M | WAETGGMGLAVRQAPLIIPLKATSTPVSIKQYP |
| | mutations | MSREARLGIKPHIRRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTMARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 681 | MMLV RTase with | MTLNIEDEHRLHETSKEPDVSLGSTWLSDFPQA |
| | Q68R/Q79R/L99R/E282W | WAETGGMGLAVRQAPLIIPLKATSTPVSIKQYP |
| | mutations | MSREARLGIKPHIRRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTWARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 682 | MMLV RTase with | MTLNIEDEHRLHETSKEPDVSLGSTWLSDFPQA |
| | I61K/Q68R/Q79R/L99R/ | WAETGGMGLAVRQAPLIIPLKATSTPVSKKQYP |
| | E282D mutations | MSREARLGIKPHIRRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 683 | MMLV RTase with | MTLNIEDEHRLHETSKEPDVSLGSTWLSDFPQA |
| | I61M/Q68R/Q79R/L99R/ | WAETGGMGLAVRQAPLIIPLKATSTPVSMKQYP |
| | E282D mutations | MSREARLGIKPHIRRLLDQGILVPCQSPWNTPL |
| | | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 684 | MMLV RTase with | MTLNIEDEHRLHETSKEPDVSLGSTWLSDFPQA |
| | Q68I/Q79H/L99K/E282M | WAETGGMGLAVRQAPLIIPLKATSTPVSIKQYP |
| | mutations | MSIEARLGIKPHIHRLLDQGILVPCQSPWNTPL |
| | | KPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTMARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 685 | MMLV RTase with | MTLNIEDEHRLHETSKEPDVSLGSTWLSDFPQA |
| | I61M/Q68I/Q79H/L99K/ | WAETGGMGLAVRQAPLIIPLKATSTPVSMKQYP |
| | E282M mutations | MSIEARLGIKPHIHRLLDQGILVPCQSPWNTPL |
| | | KPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTMARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |

c. Expression and Purification of MMLV RTase and Mutant Variants

A colony with the appropriate strain was used to inoculate TB media (200 mL) with kanamycin (0.05 mg/mL) and grown at 37° C. until an OD of approximately 0.9 was achieved followed by cooling of the flask for 30 minutes at 4° C. Protein expression was induced by the addition of 1 M IPTG (100 μL), followed by growth at 18° C. for 21 hours. Cells were harvested by spinning samples at 4,700×g for 10 minutes.

Cell pellets were re-suspended in a lysis buffer (50 mM NaPO₄, pH 7.8, 5% glycerol, 300 mM NaCl, 10 mM imidazole, 5 mM DTT, 0.01% n-ocyl-β-D-glucopyranoside, DNaseI, 10 mM CaCl2, lysozyme (1 mg/mL), and protease inhibitor). The sample was lysed on an Avestin Emulsiflex C3 pre-chilled to 4° C. at 15-20 kpsi with three passes. Cell debris was removed by centrifuging the lysate at 16,000×g for 30 minutes at 4° C.

Cleared lysates were applied to a HisTrap HP column (Cytiva Life Sciences, Cat #17524701). The resin was equilibrated with MMLV His-Bind buffer (50 mM NaPO4, pH 7.8, 5% glycerol, 0.3 M NaCl, 10 mM imidazole, 1 mM DTT and 0.01% IGEPAL-CA), followed by sample loading. The samples were washed with MMLV His-Bind buffer, followed by a 25% B wash (B=MMLV His Elution buffer=50 mM NaPO₄, pH 7.8, 5% glycerol, 0.3 M NaCl, 250 mM imidazole, 1 mM DTT and 0.01% IGEPAL-CA). The sample was eluted with 100% B for 10 CVs in 45 mL fractions.

Purified proteins were applied to a HiTrap Heparin HP column (Cytiva Life Sciences, Cat #17040601). The resin was equilibrated with MMLV Heparin-Bind buffer (50 mM Tris HCl pH 8.5, 75 mM NaCl, 1 mM DTT, 5% glycerol and 0.01% IGEPAL-CA), followed by sample loading. The sample was washed with MLV Heparin Bind buffer, followed by a 25% B wash (B=MLV Heparin Elution Buffer). The sample was eluted with 60% B for 10 CVs in 45 mL fractions.

Purified proteins were applied to a Bio-Scale™ Mini CHT™ Cartridge (Bio-Rad Laboratories, Cat #7324322). The resin was washed with 1 M NaOH, followed by equilibration with MMLV Heparin-Bind buffer and sample loading. The sample was washed with MLV Heparin Elution buffer, followed by MMLV Heparin Bind buffer. The sample was linearly eluted to 100% B2 (B2=MMLV HA Elution Buffer=250 mM KPO4 pH 7.5, 1 mM DTT, 5% glycerol and 0.01% IGEPAL-CA) for 15 CVs in 5 mL fractions.

Fractions containing purified protein were pooled and dialyzed in MMLV Storage Buffer (50 mM Tris-HCl (pH 7.5), 100 mM NaCl, 1 mM DTT, 50% (v/v) glycerol).

d. Evaluation of Ability of Purified MMLV RTase Mutant Variants to Synthesize DNA by Gene Specific Priming

MMLV RTase base construct and MMLV RTase mutant variants evaluated as described in Example 3. Temperatures were adjusted for both two-step and one-step reactions to 55 and 60° C., respectively. The two-step and one-step reactions for MMLV RTase base construct and MMLV RTase mutant variants were analyzed and reported by Ct output from the qPCR (see Tables 13 and 14).

Six of the seventeen MMLV RTase triple or more mutant variants were found to exhibit increased overall activity and thermostability as compared to the other MMLV RTase stacked mutant variants, and almost all of the MMLV RTase stacked mutant variants exhibited increased overall activity and thermostability as compared to the MMLV RTase base construct. The six MMLV RTase mutant variants that were found to exhibit the highest overall activity were Q68R/L99R, Q68R/Q79R/L99R, Q68R/Q79R/L99R/E282D, Q68R/Q79R/L99K/E282D, Q68R/Q79R/L99R/E282W, I61M/Q68R/Q79R/L99R/E282D and Q68I/Q79H/L99K/E282M.

TABLE 13
Two-Step cDNA synthesis by MMLV RT triple and more mutants.
Data was generated via qPCR human normalizer
assay and data is reported by Ct value.

| | Concentration | | Ct Standard |
|---|---|---|---|
| MMLV RT Variant | of RTase (nM) | Ct Mean | Deviation |
| MMLV-II | 0.625 | 25.520 | 0.047 |
| MMLV-II L99R/E282D | 0.625 | 24.332 | 0.060 |
| MMLV-II Q68R/L99R | 0.625 | 22.207 | 0.097 |
| MMLV-II Q79R/L99R | 0.625 | 23.789 | 0.012 |
| MMLV-II Q68R/Q79R | 0.625 | 23.629 | 0.038 |
| MMLV-II Q68R/L99R/E282D | 0.625 | 22.855 | 0.079 |
| MMLV-II Q79R/L99R/E282D | 0.625 | 23.095 | 0.035 |
| MMLV-II Q68R/Q79R/E282D | 0.625 | 22.526 | 0.027 |
| MMLV-II Q68R/Q79R/L99R | 0.625 | 22.099 | 0.018 |
| MMLV-II | 0.625 | 21.056 | 0.023 |
| Q68R/Q79R/L99R/E282D | | | |
| MMLV-II | 0.625 | 21.833 | 0.031 |
| Q68R/Q79R/L99K/E282D | | | |
| MMLV-II | 0.625 | 23.607 | 0.031 |
| Q68R/Q79R/L99N/E282D | | | |
| MMLV-II | 0.625 | 23.858 | 0.029 |
| Q68I/Q79R/L99R/E282D | | | |
| MMLV-II | 0.625 | 22.615 | 0.054 |
| Q68K/Q79R/L99R/E282D | | | |
| MMLV-II | 0.625 | 28.866 | 0.008 |
| Q68R/Q79H/L99R/E282D | | | |
| MMLV-II | 0.625 | 23.283 | 0.085 |
| Q68R/Q79I/L99R/E282D | | | |
| MMLV-II | 0.625 | 25.073 | 0.097 |
| Q68R/Q79R/L99R/E282M | | | |
| MMLV-II | 0.625 | 22.331 | 0.048 |
| Q68R/Q79R/L99R/E282W | | | |
| MMLV-II | 0.625 | 23.271 | 0.065 |
| I61K/Q68R/Q79R/L99R/E282D | | | |
| MMLV-II | 0.625 | 22.133 | 0.018 |
| I61M/Q68R/Q79R/L99R/E282D | | | |
| MMLV-II | 0.625 | 23.344 | 0.037 |
| Q68I/Q79H/L99K/E282M | | | |
| MMLV-II | 0.625 | 25.255 | 0.058 |
| I61M/Q68I/Q79H/L99K/E282M | | | |
| MMLV-II | 2.5 | 22.154 | 0.052 |
| MMLV-II L99R/E282D | 2.5 | 21.501 | 0.054 |
| MMLV-II Q68R/L99R | 2.5 | 21.151 | 0.048 |
| MMLV-II Q79R/L99R | 2.5 | 21.229 | 0.163 |
| MMLV-II Q68R/Q79R | 2.5 | 21.228 | 0.054 |
| MMLV-II Q68R/L99R/E282D | 2.5 | 21.126 | 0.030 |
| MMLV-II Q79R/L99R/E282D | 2.5 | 21.418 | 0.033 |
| MMLV-II Q68R/Q79R/E282D | 2.5 | 21.011 | 0.052 |
| MMLV-II Q68R/Q79R/L99R | 2.5 | 20.953 | 0.041 |
| MMLV-II | 2.5 | 21.113 | 0.108 |
| Q68R/Q79R/L99R/E282D | | | |
| MMLV-II | 2.5 | 20.906 | 0.081 |
| Q68R/Q79R/L99K/E282D | | | |
| MMLV-II | 2.5 | 21.196 | 0.029 |
| Q68R/Q79R/L99N/E282D | | | |
| MMLV-II | 2.5 | 21.369 | 0.009 |
| Q68I/Q79R/L99R/E282D | | | |
| MMLV-II | 2.5 | 20.960 | 0.030 |
| Q68K/Q79R/L99R/E282D | | | |
| MMLV-II | 2.5 | 26.167 | 0.038 |
| Q68R/Q79H/L99R/E282D | | | |
| MMLV-II | 2.5 | 21.012 | 0.056 |
| Q68R/Q79I/L99R/E282D | | | |
| MMLV-II | 2.5 | 21.277 | 0.036 |
| Q68R/Q79R/L99R/E282M | | | |
| MMLV-II | 2.5 | 20.944 | 0.020 |
| Q68R/Q79R/L99R/E282W | | | |
| MMLV-II | 2.5 | 21.320 | 0.009 |
| I61K/Q68R/Q79R/L99R/E282D | | | |
| MMLV-II | 2.5 | 21.095 | 0.013 |
| I61M/Q68R/Q79R/L99R/E282D | | | |
| MMLV-II | 2.5 | 21.329 | 0.047 |
| Q68I/Q79H/L99K/E282M | | | |
| MMLV-II | 2.5 | 22.159 | 0.031 |
| I61M/Q68I/Q79H/L99K/E282M | | | |
| MMLV-II | 10 | 21.575 | 0.101 |
| MMLV-II L99R/E282D | 10 | 21.546 | 0.041 |
| MMLV-II Q68R/L99R | 10 | 21.343 | 0.021 |
| MMLV-II Q79R/L99R | 10 | 21.387 | 0.016 |
| MMLV-II Q68R/Q79R | 10 | 21.147 | 0.032 |
| MMLV-II Q68R/L99R/E282D | 10 | 21.265 | 0.076 |
| MMLV-II Q79R/L99R/E282D | 10 | 21.250 | 0.036 |
| MMLV-II Q68R/Q79R/E282D | 10 | 21.135 | 0.015 |
| MMLV-II Q68R/Q79R/L99R | 10 | 21.051 | 0.036 |
| MMLV-II | 10 | 21.159 | 0.065 |
| Q68R/Q79R/L99R/E282D | | | |
| MMLV-II | 10 | 21.056 | 0.032 |
| Q68R/Q79R/L99K/E282D | | | |
| MMLV-II | 10 | 21.180 | 0.052 |
| Q68R/Q79R/L99N/E282D | | | |
| MMLV-II | 10 | 21.068 | 0.069 |
| Q68I/Q79R/L99R/E282D | | | |
| MMLV-II | 10 | 21.065 | 0.053 |
| Q68K/Q79R/L99R/E282D | | | |
| MMLV-II | 10 | 21.683 | 0.075 |
| Q68R/Q79H/L99R/E282D | | | |
| MMLV-II | 10 | 21.152 | 0.064 |
| Q68R/Q79I/L99R/E282D | | | |
| MMLV-II | 10 | 21.029 | 0.055 |
| Q68R/Q79R/L99R/E282M | | | |
| MMLV-II | 10 | 21.214 | 0.052 |
| Q68R/Q79R/L99R/E282W | | | |
| MMLV-II | 10 | 21.391 | 0.051 |
| I61K/Q68R/Q79R/L99R/E282D | | | |
| MMLV-II | 10 | 21.307 | 0.038 |
| I61M/Q68R/Q79R/L99R/E282D | | | |
| MMLV-II | 10 | 21.583 | 0.019 |
| Q68I/Q79H/L99K/E282M | | | |
| MMLV-II | 10 | 21.759 | 0.029 |
| I61M/Q68I/Q79H/L99K/E282M | | | |

TABLE 14
One-Step cDNA synthesis by MMLV RT triple and more mutants.
Data was generated via qPCR human normalizer
assay and data is reported by Ct value.

| | Concentration | | Ct Standard |
|---|---|---|---|
| MMLV RT Variant | of RTase (nM) | Ct Mean | Deviation |
| MMLV-II | 0.625 | 22.153 | 0.122 |
| MMLV-II L99R/E282D | 0.625 | 21.713 | 0.111 |
| MMLV-II Q68R/L99R | 0.625 | 21.334 | 0.167 |
| MMLV-II Q79R/L99R | 0.625 | 21.398 | 0.069 |
| MMLV-II Q68R/Q79R | 0.625 | 21.546 | 0.096 |
| MMLV-II Q68R/L99R/E282D | 0.625 | 21.112 | 0.149 |
| MMLV-II Q79R/L99R/E282D | 0.625 | 21.260 | 0.104 |
| MMLV-II Q68R/Q79R/E282D | 0.625 | 21.014 | 0.102 |
| MMLV-II Q68R/Q79R/L99R | 0.625 | 20.338 | 0.042 |
| MMLV-II | 0.625 | 19.537 | 0.120 |
| Q68R/Q79R/L99R/E282D | | | |
| MMLV-II | 0.625 | 20.516 | 0.131 |
| Q68R/Q79R/L99K/E282D | | | |
| MMLV-II | 0.625 | 20.960 | 0.023 |
| Q68R/Q79R/L99N/E282D | | | |
| MMLV-II | 0.625 | 21.325 | 0.088 |
| Q68I/Q79R/L99R/E282D | | | |
| MMLV-II | 0.625 | 20.602 | 0.038 |
| Q68K/Q79R/L99R/E282D | | | |
| MMLV-II | 0.625 | 23.889 | 0.042 |
| Q68R/Q79H/L99R/E282D | | | |
| MMLV-II | 0.625 | 21.375 | 0.035 |
| Q68R/Q79I/L99R/E282D | | | |
| MMLV-II | 0.625 | 21.805 | 0.054 |
| Q68R/Q79R/L99R/E282M | | | |
| MMLV-II | 0.625 | 20.229 | 0.085 |
| Q68R/Q79R/L99R/E282W | | | |
| MMLV-II | 0.625 | 20.972 | 0.037 |
| I61K/Q68R/Q79R/L99R/E282D | | | |
| MMLV-II | 0.625 | 20.225 | 0.042 |
| I61M/Q68R/Q79R/L99R/E282D | | | |
| MMLV-II | 0.625 | 20.578 | 0.061 |
| Q68I/Q79H/L99K/E282M | | | |
| MMLV-II | 0.625 | 21.107 | 0.101 |
| I61M/Q68I/Q79H/L99K/E282M | | | |
| MMLV-II | 2.5 | 20.874 | 0.042 |
| MMLV-II L99R/E282D | 2.5 | 19.679 | 0.047 |
| MMLV-II Q68R/L99R | 2.5 | 19.152 | 0.024 |
| MMLV-II Q79R/L99R | 2.5 | 19.202 | 0.091 |
| MMLV-II Q68R/Q79R | 2.5 | 19.506 | 0.010 |
| MMLV-II Q68R/L99R/E282D | 2.5 | 19.142 | 0.060 |
| MMLV-II Q79R/L99R/E282D | 2.5 | 19.301 | 0.004 |
| MMLV-II Q68R/Q79R/E282D | 2.5 | 19.023 | 0.041 |
| MMLV-II Q68R/Q79R/L99R | 2.5 | 18.312 | 0.041 |
| MMLV-II | 2.5 | 17.867 | 0.099 |
| Q68R/Q79R/L99R/E282D | | | |
| MMLV-II | 2.5 | 18.591 | 0.036 |
| Q68R/Q79R/L99K/E282D | | | |
| MMLV-II | 2.5 | 19.123 | 0.097 |
| Q68R/Q79R/L99N/E282D | | | |
| MMLV-II | 2.5 | 19.553 | 0.076 |
| Q68I/Q79R/L99R/E282D | | | |
| MMLV-II | 2.5 | 18.771 | 0.113 |
| Q68K/Q79R/L99R/E282D | | | |
| MMLV-II | 2.5 | 21.911 | 0.048 |
| Q68R/Q79H/L99R/E282D | | | |
| MMLV-II | 2.5 | 19.298 | 0.146 |
| Q68R/Q79I/L99R/E282D | | | |
| MMLV-II | 2.5 | 19.621 | 0.027 |
| Q68R/Q79R/L99R/E282M | | | |
| MMLV-II | 2.5 | 18.219 | 0.103 |
| Q68R/Q79R/L99R/E282W | | | |
| MMLV-II | 2.5 | 18.846 | 0.056 |
| I61K/Q68R/Q79R/L99R/E282D | | | |
| MMLV-II | 2.5 | 18.500 | 0.042 |
| I61M/Q68R/Q79R/L99R/E282D | | | |
| MMLV-II | 2.5 | 18.752 | 0.148 |
| Q68I/Q79H/L99K/E282M | | | |
| MMLV-II | 2.5 | 19.445 | 0.098 |
| I61M/Q68I/Q79H/L99K/E282M | | | |
| MMLV-II | 10 | 18.239 | 0.025 |
| MMLV-II L99R/E282D | 10 | 17.293 | 0.021 |
| MMLV-II Q68R/L99R | 10 | 17.144 | 0.032 |
| MMLV-II Q79R/L99R | 10 | 17.324 | 0.016 |
| MMLV-II Q68R/Q79R | 10 | 17.123 | 0.072 |
| MMLV-II Q68R/L99R/E282D | 10 | 17.082 | 0.088 |
| MMLV-II Q79R/L99R/E282D | 10 | 17.353 | 0.068 |
| MMLV-II Q68R/Q79R/E282D | 10 | 17.111 | 0.036 |
| MMLV-II Q68R/Q79R/L99R | 10 | 16.562 | 0.101 |
| MMLV-II | 10 | 16.492 | 0.066 |
| Q68R/Q79R/L99R/E282D | | | |
| MMLV-II | 10 | 17.027 | 0.054 |
| Q68R/Q79R/L99K/E282D | | | |
| MMLV-II | 10 | 17.335 | 0.080 |
| Q68R/Q79R/L99N/E282D | | | |
| MMLV-II | 10 | 17.726 | 0.055 |
| Q68I/Q79R/L99R/E282D | | | |
| MMLV-II | 10 | 17.144 | 0.140 |
| Q68K/Q79R/L99R/E282D | | | |
| MMLV-II | 10 | 19.772 | 0.064 |
| Q68R/Q79H/L99R/E282D | | | |
| MMLV-II | 10 | 17.424 | 0.020 |
| Q68R/Q79I/L99R/E282D | | | |
| MMLV-II | 10 | 17.624 | 0.014 |
| Q68R/Q79R/L99R/E282M | | | |
| MMLV-II | 10 | 16.629 | 0.080 |
| Q68R/Q79R/L99R/E282W | | | |
| MMLV-II | 10 | 16.903 | 0.022 |
| I61K/Q68R/Q79R/L99R/E282D | | | |
| MMLV-II | 10 | 16.803 | 0.028 |
| I61M/Q68R/Q79R/L99R/E282D | | | |
| MMLV-II | 10 | 16.894 | 0.056 |
| Q68I/Q79H/L99K/E282M | | | |
| MMLV-II | 10 | 17.509 | 0.058 |
| I61M/Q68I/Q79H/L99K/E282M | | | |

e. Evaluation of Ability of Purified MMLV RTase Mutant Variants to Synthesize DNA by Oligo-dT or Random Priming

MMLV RTase base construct and MMLV RTase mutant variants evaluated as described in Example 3. Oligo-dT or random hexamer priming conditions were adjusted for the two-step reactions and RTase concentration was normalized to 31 nM. The two-step reactions for MMLV RTase base construct and MMLV RTase mutant variants were analyzed and reported by Ct output from the qPCR (see Tables 15 and 16).

Nine of the seventeen MMLV RTase triple or more mutant variants were found to exhibit increased overall activity and thermostability as compared to the other MMLV RTase stacked mutant variants, and almost all of the MMLV RTase stacked mutant variants exhibited increased overall activity and thermostability as compared to the MMLV RTase base construct. The nine MMLV RTase mutant variants that were found to exhibit the highest overall activity were Q79R/L99R/E282D, Q68R/Q79R/L99R, Q68R/Q79R/L99R/E282D, Q68R/Q79R/L99K/E282D, Q68R/Q79R/L99N/E282D, Q68K/Q79R/L99R/E282D, Q68R/Q79R/L99R/E282M, I61K/Q68R/Q79R/L99R/E282D and 161M/Q68R/Q79R/L99R/E282D.

TABLE 15
Two-Step cDNA synthesis by MMLV RT triple and more mutants
by Oligo-dT priming. Data was generated via qPCR human
normalizer assay and data is reported by Ct value.

| | Temperature | | |
|---|---|---|---|
| | of Reaction | | Ct Standard |
| MMLV RT Variant | (° C.) | Ct Mean | Deviation |
| MMLV-II | 42 | 25.165 | 0.057 |
| MMLV-II L99R/E282D | 42 | 25.287 | 0.062 |
| MMLV-II Q68R/L99R | 42 | 25.026 | 0.035 |
| MMLV-II Q79R/L99R | 42 | 24.932 | 0.032 |
| MMLV-II Q68R/Q79R | 42 | 25.002 | 0.076 |
| MMLV-II Q68R/L99R/E282D | 42 | 24.964 | 0.068 |
| MMLV-II Q79R/L99R/E282D | 42 | 24.822 | 0.106 |
| MMLV-II Q68R/Q79R/E282D | 42 | 24.905 | 0.134 |
| MMLV-II Q68R/Q79R/L99R | 42 | 24.673 | 0.131 |
| MMLV-II | 42 | 24.523 | 0.111 |
| Q68R/Q79R/L99R/E282D | | | |
| MMLV-II | 42 | 24.677 | 0.076 |
| Q68R/Q79R/L99K/E282D | | | |
| MMLV-II | 42 | 24.635 | 0.087 |
| Q68R/Q79R/L99N/E282D | | | |
| MMLV-II | 42 | 25.010 | 0.074 |
| Q68I/Q79R/L99R/E282D | | | |
| MMLV-II | 42 | 24.676 | 0.066 |
| Q68K/Q79R/L99R/E282D | | | |
| MMLV-II | 42 | 28.929 | 0.021 |
| Q68R/Q79H/L99R/E282D | | | |
| MMLV-II | 42 | 24.932 | 0.039 |
| Q68R/Q79I/L99R/E282D | | | |
| MMLV-II | 42 | 24.900 | 0.113 |
| Q68R/Q79R/L99R/E282M | | | |
| MMLV-II | 42 | 24.967 | 0.091 |
| Q68R/Q79R/L99R/E282W | | | |
| MMLV-II | 42 | 24.597 | 0.076 |
| I61K/Q68R/Q79R/L99R/E282D | | | |
| MMLV-II | 42 | 24.833 | 0.007 |
| I61M/Q68R/Q79R/L99R/E282D | | | |
| MMLV-II | 42 | 25.440 | 0.048 |
| Q68I/Q79H/L99K/E282M | | | |
| MMLV-II | 42 | 25.679 | 0.050 |
| I61M/Q68I/Q79H/L99K/E282M | | | |
| MMLV-II | 55 | 34.223 | 0.406 |
| MMLV-II L99R/E282D | 55 | 34.732 | 3.729 |
| MMLV-II Q68R/L99R | 55 | 31.509 | 0.169 |
| MMLV-II Q79R/L99R | 55 | 31.831 | 0.019 |
| MMLV-II Q68R/Q79R | 55 | 32.633 | 1.094 |
| MMLV-II Q68R/L99R/E282D | 55 | 32.089 | 0.075 |
| MMLV-II Q79R/L99R/E282D | 55 | 32.134 | 0.081 |
| MMLV-II Q68R/Q79R/E282D | 55 | 34.639 | 3.791 |
| MMLV-II Q68R/Q79R/L99R | 55 | 29.559 | 0.029 |
| MMLV-II | 55 | 28.013 | 0.136 |
| Q68R/Q79R/L99R/E282D | | | |
| MMLV-II | 55 | 29.712 | 0.090 |
| Q68R/Q79R/L99K/E282D | | | |
| MMLV-II | 55 | 30.442 | 0.224 |
| Q68R/Q79R/L99N/E282D | | | |
| MMLV-II | 55 | 32.857 | 0.378 |
| Q68I/Q79R/L99R/E282D | | | |
| MMLV-II | 55 | 31.186 | 0.630 |
| Q68K/Q79R/L99R/E282D | | | |
| MMLV-II | 55 | 37.338 | 1.882 |
| Q68R/Q79H/L99R/E282D | | | |
| MMLV-II | 55 | 31.830 | 0.120 |
| Q68R/Q79I/L99R/E282D | | | |
| MMLV-II | 55 | 31.682 | 0.181 |
| Q68R/Q79R/L99R/E282M | | | |
| MMLV-II | 55 | 32.256 | 0.228 |
| Q68R/Q79R/L99R/E282W | | | |
| MMLV-II | 55 | 30.362 | 0.129 |
| I61K/Q68R/Q79R/L99R/E282D | | | |
| MMLV-II | 55 | 31.473 | 0.070 |
| I61M/Q68R/Q79R/L99R/E282D | | | |
| MMLV-II | 55 | 32.892 | 0.286 |
| Q68I/Q79H/L99K/E282M | | | |
| MMLV-II | 55 | 33.872 | 0.131 |
| I61M/Q68I/Q79H/L99K/E282M | | | |

TABLE 16
Two-Step cDNA synthesis by MMLV RT triple and more mutants
by random hexamer priming. Data was generated via qPCR
human normalizer assay and data is reported by Ct value.

| | Temperature | | |
|---|---|---|---|
| | of Reaction | | Ct Standard |
| MMLV RT Variant | (° C.) | Ct Mean | Deviation |
| MMLV-II | 42 | 24.675 | 0.054 |
| MMLV-II L99R/E282D | 42 | 24.864 | 0.043 |
| MMLV-II Q68R/L99R | 42 | 24.577 | 0.066 |
| MMLV-II Q79R/L99R | 42 | 24.630 | 0.103 |
| MMLV-II Q68R/Q79R | 42 | 24.496 | 0.050 |
| MMLV-II Q68R/L99R/E282D | 42 | 24.549 | 0.059 |
| MMLV-II Q79R/L99R/E282D | 42 | 24.625 | 0.013 |
| MMLV-II Q68R/Q79R/E282D | 42 | 24.623 | 0.083 |
| MMLV-II Q68R/Q79R/L99R | 42 | 24.494 | 0.070 |
| MMLV-II | 42 | 24.422 | 0.035 |
| Q68R/Q79R/L99R/E282D | | | |
| MMLV-II | 42 | 24.517 | 0.066 |
| Q68R/Q79R/L99K/E282D | | | |
| MMLV-II | 42 | 24.324 | 0.059 |
| Q68R/Q79R/L99N/E282D | | | |
| MMLV-II | 42 | 24.488 | 0.070 |
| Q68I/Q79R/L99R/E282D | | | |
| MMLV-II | 42 | 24.501 | 0.041 |
| Q68K/Q79R/L99R/E282D | | | |
| MMLV-II | 42 | 26.574 | 0.029 |
| Q68R/Q79H/L99R/E282D | | | |
| MMLV-II | 42 | 24.496 | 0.055 |
| Q68R/Q79I/L99R/E282D | | | |
| MMLV-II | 42 | 24.382 | 0.043 |
| Q68R/Q79R/L99R/E282M | | | |
| MMLV-II | 42 | 24.617 | 0.109 |
| Q68R/Q79R/L99R/E282W | | | |
| MMLV-II | 42 | 24.391 | 0.045 |
| I61K/Q68R/Q79R/L99R/E282D | | | |
| MMLV-II | 42 | 24.426 | 0.028 |
| I61M/Q68R/Q79R/L99R/E282D | | | |
| MMLV-II | 42 | 24.660 | 0.027 |
| Q68I/Q79H/L99K/E282M | | | |
| MMLV-II | 42 | 24.949 | 0.052 |
| I61M/Q68I/Q79H/L99K/E282M | | | |
| MMLV-II | 55 | 32.082 | 0.095 |
| MMLV-II L99R/E282D | 55 | 31.612 | 0.190 |
| MMLV-II Q68R/L99R | 55 | 30.349 | 0.041 |
| MMLV-II Q79R/L99R | 55 | 30.494 | 0.094 |
| MMLV-II Q68R/Q79R | 55 | 29.735 | 0.153 |
| MMLV-II Q68R/L99R/E282D | 55 | 30.724 | 0.045 |
| MMLV-II Q79R/L99R/E282D | 55 | 30.774 | 0.152 |
| MMLV-II Q68R/Q79R/E282D | 55 | 30.232 | 0.079 |
| MMLV-II Q68R/Q79R/L99R | 55 | 28.270 | 0.340 |
| MMLV-II | 55 | 26.673 | 0.143 |
| Q68R/Q79R/L99R/E282D | | | |
| MMLV-II | 55 | 28.258 | 0.018 |
| Q68R/Q79R/L99K/E282D | | | |
| MMLV-II | 55 | 28.973 | 0.116 |
| Q68R/Q79R/L99N/E282D | | | |
| MMLV-II | 55 | 31.617 | 0.071 |
| Q68I/Q79R/L99R/E282D | | | |
| MMLV-II | 55 | 28.994 | 0.110 |
| Q68K/Q79R/L99R/E282D | | | |
| MMLV-II | 55 | 35.664 | 0.695 |
| Q68R/Q79H/L99R/E282D | | | |
| MMLV-II | 55 | 30.265 | 0.116 |
| Q68R/Q79I/L99R/E282D | | | |
| MMLV-II | 55 | 29.765 | 0.059 |
| Q68R/Q79R/L99R/E282M | | | |
| MMLV-II | 55 | 30.535 | 0.424 |
| Q68R/Q79R/L99R/E282W | | | |
| MMLV-II | 55 | 28.878 | 0.038 |
| I61K/Q68R/Q79R/L99R/E282D | | | |
| MMLV-II | 55 | 29.778 | 0.081 |
| I61M/Q68R/Q79R/L99R/E282D | | | |
| MMLV-II | 55 | 31.836 | 0.222 |
| Q68I/Q79H/L99K/E282M | | | |
| MMLV-II | 55 | 31.984 | 0.223 |
| I61M/Q68I/Q79H/L99K/E282M | | | |

f. Evaluation of Ability of Purified MMLV RTase Mutant Variants to Synthesize DNA Over a Wide Range of Temperatures

MMLV RTase base construct and MMLV RTase mutant variants evaluated as described in Example 3. Oligo-dT or random hexamer priming conditions and reaction temperatures were adjusted for the two-step reactions and RTase concentration was normalized to 31 nM. The two-step reactions for MMLV RTase base construct and MMLV RTase mutant variants were analyzed and reported by Ct output from the qPCR (see Tables 17 and 18).

Six of the nine MMLV RTase triple or more mutant variants were found to exhibit high overall activity as compared to the other MMLV RTase stacked mutant variants over a wide range of temperatures, spanning from 37.0 to 65° C., regardless of which priming method used. All of the MMLV RTase stacked mutant variants exhibited increased overall activity and thermostability as compared to the MMLV RTase base construct. The six MMLV RTase mutant variants that were found to exhibit the highest overall activity at a wide range of temperatures were Q68R/Q79R/L99R, Q68R/Q79R/L99R/E282D, Q68R/Q79R/L99K/E282D, Q68R/Q79R/L99N/E282D, I61K/Q68R/Q79R/L99R/E282D and I61M/Q68R/Q79R/L99R/E282D.

TABLE 17
Two-Step cDNA synthesis by MMLV RT triple and more mutants
by Oligo-dT priming. Data was generated via qPCR human
normalizer assay and data is reported by Ct value.

| | Temperature | | |
|---|---|---|---|
| | of Reaction | | Ct Standard |
| MMLV RT Variant | (° C.) | Ct Mean | Deviation |
| MMLV-II | 37.0 | 26.593 | 0.020 |
| MMLV-II Q79R/L99R/E282D | 37.0 | 25.713 | 0.024 |
| MMLV-II Q68R/Q79R/L99R | 37.0 | 25.164 | 0.059 |
| MMLV-II | 37.0 | 25.163 | 0.035 |
| Q68R/Q79R/L99R/E282D | | | |
| MMLV-II | 37.0 | 25.135 | 0.078 |
| Q68R/Q79R/L99K/E282D | | | |
| MMLV-II | 37.0 | 25.693 | 0.048 |
| Q68R/Q79R/L99N/E282D | | | |
| MMLV-II | 37.0 | 25.491 | 0.062 |
| Q68K/Q79R/L99R/E282D | | | |
| MMLV-II | 37.0 | 25.450 | 0.083 |
| Q68R/Q79R/L99R/E282M | | | |
| MMLV-II | 37.0 | 25.094 | 0.071 |
| I61K/Q68R/Q79R/L99R/E282D | | | |
| MMLV-II | 37.0 | 25.356 | 0.034 |
| I61M/Q68R/Q79R/L99R/E282D | | | |
| MMLV-II | 37.8 | 26.623 | 0.062 |
| MMLV-II Q79R/L99R/E282D | 37.8 | 25.516 | 0.078 |
| MMLV-II Q68R/Q79R/L99R | 37.8 | 25.251 | 0.094 |
| MMLV-II | 37.8 | 24.987 | 0.050 |
| Q68R/Q79R/L99R/E282D | | | |
| MMLV-II | 37.8 | 25.093 | 0.084 |
| Q68R/Q79R/L99K/E282D | | | |
| MMLV-II | 37.8 | 25.273 | 0.095 |
| Q68R/Q79R/L99N/E282D | | | |
| MMLV-II | 37.8 | 25.310 | 0.079 |
| Q68K/Q79R/L99R/E282D | | | |
| MMLV-II | 37.8 | 25.545 | 0.044 |
| Q68R/Q79R/L99R/E282M | | | |
| MMLV-II | 37.8 | 25.144 | 0.196 |
| I61K/Q68R/Q79R/L99R/E282D | | | |
| MMLV-II | 37.8 | 25.302 | 0.035 |
| I61M/Q68R/Q79R/L99R/E282D | | | |
| MMLV-II | 39.5 | 26.430 | 0.074 |
| MMLV-II Q79R/L99R/E282D | 39.5 | 25.067 | 0.026 |
| MMLV-II Q68R/Q79R/L99R | 39.5 | 25.138 | 0.050 |
| MMLV-II | 39.5 | 24.788 | 0.022 |
| Q68R/Q79R/L99R/E282D | | | |
| MMLV-II | 39.5 | 24.842 | 0.071 |
| Q68R/Q79R/L99K/E282D | | | |
| MMLV-II | 39.5 | 24.892 | 0.042 |
| Q68R/Q79R/L99N/E282D | | | |
| MMLV-II | 39.5 | 25.047 | 0.038 |
| Q68K/Q79R/L99R/E282D | | | |
| MMLV-II | 39.5 | 25.249 | 0.081 |
| Q68R/Q79R/L99R/E282M | | | |
| MMLV-II | 39.5 | 24.845 | 0.130 |
| I61K/Q68R/Q79R/L99R/E282D | | | |
| MMLV-II | 39.5 | 25.130 | 0.072 |
| I61M/Q68R/Q79R/L99R/E282D | | | |
| MMLV-II | 42.0 | 25.485 | 0.052 |
| MMLV-II Q79R/L99R/E282D | 42.0 | 24.941 | 0.024 |
| MMLV-II Q68R/Q79R/L99R | 42.0 | 24.848 | 0.101 |
| MMLV-II | 42.0 | 24.802 | 0.009 |
| Q68R/Q79R/L99R/E282D | | | |
| MMLV-II | 42.0 | 24.805 | 0.008 |
| Q68R/Q79R/L99K/E282D | | | |
| MMLV-II | 42.0 | 24.744 | 0.076 |
| Q68R/Q79R/L99N/E282D | | | |
| MMLV-II | 42.0 | 24.893 | 0.073 |
| Q68K/Q79R/L99R/E282D | | | |
| MMLV-II | 42.0 | 24.968 | 0.031 |
| Q68R/Q79R/L99R/E282M | | | |
| MMLV-II | 42.0 | 24.933 | 0.088 |
| I61K/Q68R/Q79R/L99R/E282D | | | |
| MMLV-II | 42.0 | 24.821 | 0.045 |
| I61M/Q68R/Q79R/L99R/E282D | | | |
| MMLV-II | 45.2 | 25.776 | 0.028 |
| MMLV-II Q79R/L99R/E282D | 45.2 | 24.902 | 0.034 |
| MMLV-II Q68R/Q79R/L99R | 45.2 | 24.792 | 0.055 |
| MMLV-II | 45.2 | 24.705 | 0.092 |
| Q68R/Q79R/L99R/E282D | | | |
| MMLV-II | 45.2 | 24.791 | 0.009 |
| Q68R/Q79R/L99K/E282D | | | |
| MMLV-II | 45.2 | 24.890 | 0.071 |
| Q68R/Q79R/L99N/E282D | | | |
| MMLV-II | 45.2 | 25.420 | 0.101 |
| Q68K/Q79R/L99R/E282D | | | |
| MMLV-II | 45.2 | 25.196 | 0.086 |
| Q68R/Q79R/L99R/E282M | | | |
| MMLV-II | 45.2 | 24.823 | 0.079 |
| I61K/Q68R/Q79R/L99R/E282D | | | |
| MMLV-II | 45.2 | 24.720 | 0.006 |
| I61M/Q68R/Q79R/L99R/E282D | | | |
| MMLV-II | 47.8 | 27.932 | 0.049 |
| MMLV-II Q79R/L99R/E282D | 47.8 | 24.858 | 0.063 |
| MMLV-II Q68R/Q79R/L99R | 47.8 | 24.685 | 0.095 |
| MMLV-II | 47.8 | 24.689 | 0.067 |
| Q68R/Q79R/L99R/E282D | | | |
| MMLV-II | 47.8 | 24.620 | 0.072 |
| Q68R/Q79R/L99K/E282D | | | |
| MMLV-II | 47.8 | 24.780 | 0.039 |
| Q68R/Q79R/L99N/E282D | | | |
| MMLV-II | 47.8 | 24.855 | 0.018 |
| Q68K/Q79R/L99R/E282D | | | |
| MMLV-II | 47.8 | 24.961 | 0.040 |
| Q68R/Q79R/L99R/E282M | | | |
| MMLV-II | 47.8 | 24.681 | 0.076 |
| I61K/Q68R/Q79R/L99R/E282D | | | |
| MMLV-II | 47.8 | 24.759 | 0.055 |
| I61M/Q68R/Q79R/L99R/E282D | | | |
| MMLV-II | 49.2 | 30.393 | 0.118 |
| MMLV-II Q79R/L99R/E282D | 49.2 | 24.974 | 0.090 |
| MMLV-II Q68R/Q79R/L99R | 49.2 | 24.794 | 0.056 |
| MMLV-II | 49.2 | 24.720 | 0.100 |
| Q68R/Q79R/L99R/E282D | | | |
| MMLV-II | 49.2 | 25.007 | 0.096 |
| Q68R/Q79R/L99K/E282D | | | |
| MMLV-II | 49.2 | 25.304 | 0.147 |
| Q68R/Q79R/L99N/E282D | | | |
| MMLV-II | 49.2 | 25.273 | 0.066 |
| Q68K/Q79R/L99R/E282D | | | |
| MMLV-II | 49.2 | 25.560 | 0.019 |
| Q68R/Q79R/L99R/E282M | | | |
| MMLV-II | 49.2 | 24.719 | 0.177 |
| I61K/Q68R/Q79R/L99R/E282D | | | |
| MMLV-II | 49.2 | 25.123 | 0.034 |
| I61M/Q68R/Q79R/L99R/E282D | | | |
| MMLV-II | 50.0 | 30.870 | 0.210 |
| MMLV-II Q79R/L99R/E282D | 50.0 | 26.677 | 0.090 |
| MMLV-II Q68R/Q79R/L99R | 50.0 | 25.381 | 0.049 |
| MMLV-II | 50.0 | 24.820 | 0.064 |
| Q68R/Q79R/L99R/E282D | | | |
| MMLV-II | 50.0 | 25.348 | 0.098 |
| Q68R/Q79R/L99K/E282D | | | |
| MMLV-II | 50.0 | 25.287 | 0.064 |
| Q68R/Q79R/L99N/E282D | | | |
| MMLV-II | 50.0 | 25.208 | 0.085 |
| Q68K/Q79R/L99R/E282D | | | |
| MMLV-II | 50.0 | 25.790 | 0.051 |
| Q68R/Q79R/L99R/E282M | | | |
| MMLV-II | 50.0 | 24.840 | 0.071 |
| I61K/Q68R/Q79R/L99R/E282D | | | |
| MMLV-II | 50.0 | 25.317 | 0.042 |
| I61M/Q68R/Q79R/L99R/E282D | | | |
| MMLV-II | 51.0 | 27.914 | 0.002 |
| MMLV-II Q79R/L99R/E282D | 51.0 | 25.561 | 0.069 |
| MMLV-II Q68R/Q79R/L99R | 51.0 | 25.225 | 0.069 |
| MMLV-II | 51.0 | 24.726 | 0.034 |
| Q68R/Q79R/L99R/E282D | | | |
| MMLV-II | 51.0 | 25.324 | 0.071 |
| Q68R/Q79R/L99K/E282D | | | |
| MMLV-II | 51.0 | 25.157 | 0.062 |
| Q68R/Q79R/L99N/E282D | | | |
| MMLV-II | 51.0 | 25.275 | 0.039 |
| Q68K/Q79R/L99R/E282D | | | |
| MMLV-II | 51.0 | 25.938 | 0.095 |
| Q68R/Q79R/L99R/E282M | | | |
| MMLV-II | 51.0 | 25.821 | 0.072 |
| I61K/Q68R/Q79R/L99R/E282D | | | |
| MMLV-II | 51.0 | 25.053 | 0.044 |
| I61M/Q68R/Q79R/L99R/E282D | | | |
| MMLV-II | 51.9 | 28.602 | 0.059 |
| MMLV-II Q79R/L99R/E282D | 51.9 | 25.975 | 0.024 |
| MMLV-II Q68R/Q79R/L99R | 51.9 | 25.256 | 0.075 |
| MMLV-II | 51.9 | 24.903 | 0.050 |
| Q68R/Q79R/L99R/E282D | | | |
| MMLV-II | 51.9 | 25.163 | 0.169 |
| Q68R/Q79R/L99K/E282D | | | |
| MMLV-II | 51.9 | 25.272 | 0.011 |
| Q68R/Q79R/L99N/E282D | | | |
| MMLV-II | 51.9 | 25.491 | 0.075 |
| Q68K/Q79R/L99R/E282D | | | |
| MMLV-II | 51.9 | 25.878 | 0.038 |
| Q68R/Q79R/L99R/E282M | | | |
| MMLV-II | 51.9 | 26.071 | 0.044 |
| I61K/Q68R/Q79R/L99R/E282D | | | |
| MMLV-II | 51.9 | 25.419 | 0.067 |
| I61M/Q68R/Q79R/L99R/E282D | | | |
| MMLV-II | 53.8 | 26.412 | 0.082 |
| MMLV-II Q79R/L99R/E282D | 53.8 | 25.558 | 0.063 |
| MMLV-II Q68R/Q79R/L99R | 53.8 | 24.969 | 0.065 |
| MMLV-II | 53.8 | 25.356 | 0.063 |
| Q68R/Q79R/L99R/E282D | | | |
| MMLV-II | 53.8 | 25.460 | 0.056 |
| Q68R/Q79R/L99K/E282D | | | |
| MMLV-II | 53.8 | 25.769 | 0.118 |
| Q68R/Q79R/L99N/E282D | | | |
| MMLV-II | 53.8 | 26.251 | 0.103 |
| Q68K/Q79R/L99R/E282D | | | |
| MMLV-II | 53.8 | 26.310 | 0.174 |
| Q68R/Q79R/L99R/E282M | | | |
| MMLV-II | 53.8 | 25.701 | 0.106 |
| I61K/Q68R/Q79R/L99R/E282D | | | |
| MMLV-II | 53.8 | 26.412 | 0.082 |
| I61M/Q68R/Q79R/L99R/E282D | | | |
| MMLV-II | 56.5 | 29.343 | 0.085 |
| MMLV-II Q79R/L99R/E282D | 56.5 | 26.885 | 0.083 |
| MMLV-II Q68R/Q79R/L99R | 56.5 | 25.736 | 0.015 |
| MMLV-II | 56.5 | 25.223 | 0.016 |
| Q68R/Q79R/L99R/E282D | | | |
| MMLV-II | 56.5 | 25.900 | 0.039 |
| Q68R/Q79R/L99K/E282D | | | |
| MMLV-II | 56.5 | 25.930 | 0.031 |
| Q68R/Q79R/L99N/E282D | | | |
| MMLV-II | 56.5 | 25.869 | 0.204 |
| Q68K/Q79R/L99R/E282D | | | |
| MMLV-II | 56.5 | 26.622 | 0.067 |
| Q68R/Q79R/L99R/E282M | | | |
| MMLV-II | 56.5 | 25.817 | 0.089 |
| I61K/Q68R/Q79R/L99R/E282D | | | |
| MMLV-II | 56.5 | 26.290 | 0.009 |
| I61M/Q68R/Q79R/L99R/E282D | | | |
| MMLV-II | 59.9 | 29.693 | 0.047 |
| MMLV-II Q79R/L99R/E282D | 59.9 | 27.820 | 0.014 |
| MMLV-II Q68R/Q79R/L99R | 59.9 | 26.069 | 0.057 |
| MMLV-II | 59.9 | 25.374 | 0.061 |
| Q68R/Q79R/L99R/E282D | | | |
| MMLV-II | 59.9 | 26.066 | 0.053 |
| Q68R/Q79R/L99K/E282D | | | |
| MMLV-II | 59.9 | 25.873 | 0.018 |
| Q68R/Q79R/L99N/E282D | | | |
| MMLV-II | 59.9 | 26.278 | 0.073 |
| Q68K/Q79R/L99R/E282D | | | |
| MMLV-II | 59.9 | 27.068 | 0.075 |
| Q68R/Q79R/L99R/E282M | | | |
| MMLV-II | 59.9 | 26.863 | 0.025 |
| I61K/Q68R/Q79R/L99R/E282D | | | |
| MMLV-II | 59.9 | 26.176 | 0.072 |
| I61M/Q68R/Q79R/L99R/E282D | | | |
| MMLV-II | 62.6 | 29.731 | 0.092 |
| MMLV-II Q79R/L99R/E282D | 62.6 | 27.161 | 0.035 |
| MMLV-II Q68R/Q79R/L99R | 62.6 | 25.929 | 0.026 |
| MMLV-II | 62.6 | 25.303 | 0.074 |
| Q68R/Q79R/L99R/E282D | | | |
| MMLV-II | 62.6 | 25.907 | 0.003 |
| Q68R/Q79R/L99K/E282D | | | |
| MMLV-II | 62.6 | 26.145 | 0.053 |
| Q68R/Q79R/L99N/E282D | | | |
| MMLV-II | 62.6 | 26.181 | 0.056 |
| Q68K/Q79R/L99R/E282D | | | |
| MMLV-II | 62.6 | 27.134 | 0.015 |
| Q68R/Q79R/L99R/E282M | | | |
| MMLV-II | 62.6 | 26.025 | 0.178 |
| I61K/Q68R/Q79R/L99R/E282D | | | |
| MMLV-II | 62.6 | 26.304 | 0.041 |
| I61M/Q68R/Q79R/L99R/E282D | | | |
| MMLV-II | 64.2 | 26.809 | 0.080 |
| MMLV-II Q79R/L99R/E282D | 64.2 | 27.325 | 0.038 |
| MMLV-II Q68R/Q79R/L99R | 64.2 | 26.131 | 0.018 |
| MMLV-II | 64.2 | 25.542 | 0.135 |
| Q68R/Q79R/L99R/E282D | | | |
| MMLV-II | 64.2 | 26.408 | 0.093 |
| Q68R/Q79R/L99K/E282D | | | |
| MMLV-II | 64.2 | 26.734 | 0.040 |
| Q68R/Q79R/L99N/E282D | | | |
| MMLV-II | 64.2 | 30.589 | 0.128 |
| Q68K/Q79R/L99R/E282D | | | |
| MMLV-II | 64.2 | 26.262 | 0.090 |
| Q68R/Q79R/L99R/E282M | | | |
| MMLV-II | 64.2 | 27.594 | 0.118 |
| I61K/Q68R/Q79R/L99R/E282D | | | |
| MMLV-II | 64.2 | 27.062 | 0.051 |
| I61M/Q68R/Q79R/L99R/E282D | | | |
| MMLV-II | 65.0 | 30.277 | 0.050 |
| MMLV-II Q79R/L99R/E282D | 65.0 | 27.119 | 0.065 |
| MMLV-II Q68R/Q79R/L99R | 65.0 | 26.078 | 0.025 |
| MMLV-II | 65.0 | 25.583 | 0.068 |
| Q68R/Q79R/L99R/E282D | | | |
| MMLV-II | 65.0 | 25.906 | 0.080 |
| Q68R/Q79R/L99K/E282D | | | |
| MMLV-II | 65.0 | 26.943 | 0.058 |
| Q68R/Q79R/L99N/E282D | | | |
| MMLV-II | 65.0 | 26.413 | 0.067 |
| Q68K/Q79R/L99R/E282D | | | |
| MMLV-II | 65.0 | 28.233 | 0.075 |
| Q68R/Q79R/L99R/E282M | | | |
| MMLV-II | 65.0 | 25.778 | 0.129 |
| I61K/Q68R/Q79R/L99R/E282D | | | |
| MMLV-II | 65.0 | 27.345 | 0.015 |
| I61M/Q68R/Q79R/L99R/E282D | | | |

TABLE 18
Two-Step cDNA synthesis by MMLV RT triple and more mutants
by random hexamer priming. Data was generated via qPCR
human normalizer assay and data is reported by Ct value.

| | Temperature | | |
|---|---|---|---|
| | of Reaction | | Ct Standard |
| MMLV RT Variant | (° C.) | Ct Mean | Deviation |
| MMLV-II | 37.0 | 25.827 | 0.120 |
| MMLV-II Q79R/L99R/E282D | 37.0 | 25.616 | 0.094 |
| MMLV-II Q68R/Q79R/L99R | 37.0 | 24.747 | 0.041 |
| MMLV-II | 37.0 | 24.595 | 0.034 |
| Q68R/Q79R/L99R/E282D | | | |
| MMLV-II | 37.0 | 24.917 | 0.078 |
| Q68R/Q79R/L99K/E282D | | | |
| MMLV-II | 37.0 | 24.817 | 0.024 |
| Q68R/Q79R/L99N/E282D | | | |
| MMLV-II | 37.0 | 24.757 | 0.032 |
| Q68K/Q79R/L99R/E282D | | | |
| MMLV-II | 37.0 | 24.754 | 0.062 |
| Q68R/Q79R/L99R/E282M | | | |
| MMLV-II | 37.0 | 24.883 | 0.106 |
| I61K/Q68R/Q79R/L99R/E282D | | | |
| MMLV-II | 37.0 | 24.776 | 0.028 |
| I61M/Q68R/Q79R/L99R/E282D | | | |
| MMLV-II | 37.8 | 25.609 | 0.038 |
| MMLV-II Q79R/L99R/E282D | 37.8 | 25.300 | 0.061 |
| MMLV-II Q68R/Q79R/L99R | 37.8 | 24.822 | 0.037 |
| MMLV-II | 37.8 | 24.690 | 0.044 |
| Q68R/Q79R/L99R/E282D | | | |
| MMLV-II | 37.8 | 24.884 | 0.033 |
| Q68R/Q79R/L99K/E282D | | | |
| MMLV-II | 37.8 | 24.665 | 0.022 |
| Q68R/Q79R/L99N/E282D | | | |
| MMLV-II | 37.8 | 24.846 | 0.021 |
| Q68K/Q79R/L99R/E282D | | | |
| MMLV-II | 37.8 | 24.882 | 0.043 |
| Q68R/Q79R/L99R/E282M | | | |
| MMLV-II | 37.8 | 24.846 | 0.059 |
| I61K/Q68R/Q79R/L99R/E282D | | | |
| MMLV-II | 37.8 | 24.723 | 0.023 |
| I61M/Q68R/Q79R/L99R/E282D | | | |
| MMLV-II | 39.5 | 25.455 | 0.020 |
| MMLV-II Q79R/L99R/E282D | 39.5 | 24.790 | 0.109 |
| MMLV-II Q68R/Q79R/L99R | 39.5 | 24.712 | 0.050 |
| MMLV-II | 39.5 | 24.543 | 0.005 |
| Q68R/Q79R/L99R/E282D | | | |
| MMLV-II | 39.5 | 24.714 | 0.035 |
| Q68R/Q79R/L99K/E282D | | | |
| MMLV-II | 39.5 | 24.520 | 0.084 |
| Q68R/Q79R/L99N/E282D | | | |
| MMLV-II | 39.5 | 24.752 | 0.047 |
| Q68K/Q79R/L99R/E282D | | | |
| MMLV-II | 39.5 | 24.850 | 0.054 |
| Q68R/Q79R/L99R/E282M | | | |
| MMLV-II | 39.5 | 24.698 | 0.059 |
| I61K/Q68R/Q79R/L99R/E282D | | | |
| MMLV-II | 39.5 | 24.682 | 0.024 |
| I61M/Q68R/Q79R/L99R/E282D | | | |
| MMLV-II | 42.0 | 25.136 | 0.034 |
| MMLV-II Q79R/L99R/E282D | 42.0 | 24.760 | 0.052 |
| MMLV-II Q68R/Q79R/L99R | 42.0 | 24.637 | 0.037 |
| MMLV-II | 42.0 | 24.449 | 0.008 |
| Q68R/Q79R/L99R/E282D | | | |
| MMLV-II | 42.0 | 24.650 | 0.068 |
| Q68R/Q79R/L99K/E282D | | | |
| MMLV-II | 42.0 | 24.477 | 0.055 |
| Q68R/Q79R/L99N/E282D | | | |
| MMLV-II | 42.0 | 24.624 | 0.029 |
| Q68K/Q79R/L99R/E282D | | | |
| MMLV-II | 42.0 | 24.627 | 0.044 |
| Q68R/Q79R/L99R/E282M | | | |
| MMLV-II | 42.0 | 24.718 | 0.083 |
| I61K/Q68R/Q79R/L99R/E282D | | | |
| MMLV-II | 42.0 | 24.532 | 0.021 |
| I61M/Q68R/Q79R/L99R/E282D | | | |
| MMLV-II | 45.2 | 25.079 | 0.017 |
| MMLV-II Q79R/L99R/E282D | 45.2 | 24.624 | 0.026 |
| MMLV-II Q68R/Q79R/L99R | 45.2 | 24.525 | 0.021 |
| MMLV-II | 45.2 | 24.430 | 0.014 |
| Q68R/Q79R/L99R/E282D | | | |
| MMLV-II | 45.2 | 24.525 | 0.037 |
| Q68R/Q79R/L99K/E282D | | | |
| MMLV-II | 45.2 | 34.853 | 0.705 |
| Q68R/Q79R/L99N/E282D | | | |
| MMLV-II | 45.2 | 24.653 | 0.055 |
| Q68K/Q79R/L99R/E282D | | | |
| MMLV-II | 45.2 | 24.552 | 0.060 |
| Q68R/Q79R/L99R/E282M | | | |
| MMLV-II | 45.2 | 24.595 | 0.027 |
| I61K/Q68R/Q79R/L99R/E282D | | | |
| MMLV-II | 45.2 | 24.493 | 0.016 |
| I61M/Q68R/Q79R/L99R/E282D | | | |
| MMLV-II | 47.8 | 25.346 | 0.007 |
| MMLV-II Q79R/L99R/E282D | 47.8 | 24.521 | 0.097 |
| MMLV-II Q68R/Q79R/L99R | 47.8 | 24.605 | 0.018 |
| MMLV-II | 47.8 | 24.333 | 0.107 |
| Q68R/Q79R/L99R/E282D | | | |
| MMLV-II | 47.8 | 24.516 | 0.043 |
| Q68R/Q79R/L99K/E282D | | | |
| MMLV-II | 47.8 | 24.527 | 0.026 |
| Q68R/Q79R/L99N/E282D | | | |
| MMLV-II | 47.8 | 24.539 | 0.064 |
| Q68K/Q79R/L99R/E282D | | | |
| MMLV-II | 47.8 | 24.631 | 0.019 |
| Q68R/Q79R/L99R/E282M | | | |
| MMLV-II | 47.8 | 24.227 | 0.260 |
| I61K/Q68R/Q79R/L99R/E282D | | | |
| MMLV-II | 47.8 | 24.441 | 0.030 |
| I61M/Q68R/Q79R/L99R/E282D | | | |
| MMLV-II | 49.2 | 25.791 | 0.064 |
| MMLV-II Q79R/L99R/E282D | 49.2 | 24.700 | 0.033 |
| MMLV-II Q68R/Q79R/L99R | 49.2 | 24.658 | 0.008 |
| MMLV-II | 49.2 | 24.471 | 0.069 |
| Q68R/Q79R/L99R/E282D | | | |
| MMLV-II | 49.2 | 24.590 | 0.024 |
| Q68R/Q79R/L99K/E282D | | | |
| MMLV-II | 49.2 | 24.482 | 0.099 |
| Q68R/Q79R/L99N/E282D | | | |
| MMLV-II | 49.2 | 24.549 | 0.028 |
| Q68K/Q79R/L99R/E282D | | | |
| MMLV-II | 49.2 | 24.753 | 0.030 |
| Q68R/Q79R/L99R/E282M | | | |
| MMLV-II | 49.2 | 24.499 | 0.157 |
| I61K/Q68R/Q79R/L99R/E282D | | | |
| MMLV-II | 49.2 | 24.559 | 0.033 |
| I61M/Q68R/Q79R/L99R/E282D | | | |
| MMLV-II | 50.0 | 26.267 | 0.025 |
| MMLV-II Q79R/L99R/E282D | 50.0 | 24.729 | 0.047 |
| MMLV-II Q68R/Q79R/L99R | 50.0 | 24.462 | 0.040 |
| MMLV-II | 50.0 | 24.412 | 0.035 |
| Q68R/Q79R/L99R/E282D | | | |
| MMLV-II | 50.0 | 24.438 | 0.090 |
| Q68R/Q79R/L99K/E282D | | | |
| MMLV-II | 50.0 | 24.509 | 0.050 |
| Q68R/Q79R/L99N/E282D | | | |
| MMLV-II | 50.0 | 24.405 | 0.059 |
| Q68K/Q79R/L99R/E282D | | | |
| MMLV-II | 50.0 | 24.547 | 0.041 |
| Q68R/Q79R/L99R/E282M | | | |
| MMLV-II | 50.0 | 24.504 | 0.005 |
| I61K/Q68R/Q79R/L99R/E282D | | | |
| MMLV-II | 50.0 | 24.481 | 0.009 |
| I61M/Q68R/Q79R/L99R/E282D | | | |
| MMLV-II | 51.0 | 27.277 | 0.058 |
| MMLV-II Q79R/L99R/E282D | 51.0 | 25.694 | 0.104 |
| MMLV-II Q68R/Q79R/L99R | 51.0 | 24.579 | 0.037 |
| MMLV-II | 51.0 | 24.364 | 0.019 |
| Q68R/Q79R/L99R/E282D | | | |
| MMLV-II | 51.0 | 24.849 | 0.041 |
| Q68R/Q79R/L99K/E282D | | | |
| MMLV-II | 51.0 | 24.899 | 0.121 |
| Q68R/Q79R/L99N/E282D | | | |
| MMLV-II | 51.0 | 24.980 | 0.048 |
| Q68K/Q79R/L99R/E282D | | | |
| MMLV-II | 51.0 | 25.292 | 0.065 |
| Q68R/Q79R/L99R/E282M | | | |
| MMLV-II | 51.0 | 25.147 | 0.100 |
| I61K/Q68R/Q79R/L99R/E282D | | | |
| MMLV-II | 51.0 | 25.034 | 0.075 |
| I61M/Q68R/Q79R/L99R/E282D | | | |
| MMLV-II | 51.9 | 28.797 | 0.055 |
| MMLV-II Q79R/L99R/E282D | 51.9 | 26.585 | 0.011 |
| MMLV-II Q68R/Q79R/L99R | 51.9 | 25.021 | 0.036 |
| MMLV-II | 51.9 | 24.763 | 0.028 |
| Q68R/Q79R/L99R/E282D | | | |
| MMLV-II | 51.9 | 25.392 | 0.012 |
| Q68R/Q79R/L99K/E282D | | | |
| MMLV-II | 51.9 | 25.543 | 0.087 |
| Q68R/Q79R/L99N/E282D | | | |
| MMLV-II | 51.9 | 25.549 | 0.058 |
| Q68K/Q79R/L99R/E282D | | | |
| MMLV-II | 51.9 | 26.025 | 0.065 |
| Q68R/Q79R/L99R/E282M | | | |
| MMLV-II | 51.9 | 26.087 | 0.024 |
| I61K/Q68R/Q79R/L99R/E282D | | | |
| MMLV-II | 51.9 | 25.756 | 0.054 |
| I61M/Q68R/Q79R/L99R/E282D | | | |
| MMLV-II | 53.8 | 30.985 | 0.073 |
| MMLV-II Q79R/L99R/E282D | 53.8 | 29.356 | 0.044 |
| MMLV-II Q68R/Q79R/L99R | 53.8 | 26.370 | 0.041 |
| MMLV-II | 53.8 | 25.580 | 0.049 |
| Q68R/Q79R/L99R/E282D | | | |
| MMLV-II | 53.8 | 26.682 | 0.029 |
| Q68R/Q79R/L99K/E282D | | | |
| MMLV-II | 53.8 | 26.438 | 0.031 |
| Q68R/Q79R/L99N/E282D | | | |
| MMLV-II | 53.8 | 27.024 | 0.042 |
| Q68K/Q79R/L99R/E282D | | | |
| MMLV-II | 53.8 | 28.314 | 0.051 |
| Q68R/Q79R/L99R/E282M | | | |
| MMLV-II | 53.8 | 27.489 | 0.025 |
| I61K/Q68R/Q79R/L99R/E282D | | | |
| MMLV-II | 53.8 | 27.871 | 0.118 |
| I61M/Q68R/Q79R/L99R/E282D | | | |
| MMLV-II | 56.5 | 33.313 | 0.164 |
| MMLV-II Q79R/L99R/E282D | 56.5 | 32.626 | 0.113 |
| MMLV-II Q68R/Q79R/L99R | 56.5 | 30.047 | 0.089 |
| MMLV-II | 56.5 | 29.183 | 0.155 |
| Q68R/Q79R/L99R/E282D | | | |
| MMLV-II | 56.5 | 30.750 | 0.051 |
| Q68R/Q79R/L99K/E282D | | | |
| MMLV-II | 56.5 | 30.403 | 0.095 |
| Q68R/Q79R/L99N/E282D | | | |
| MMLV-II | 56.5 | 31.707 | 0.111 |
| Q68K/Q79R/L99R/E282D | | | |
| MMLV-II | 56.5 | 31.878 | 0.093 |
| Q68R/Q79R/L99R/E282M | | | |
| MMLV-II | 56.5 | 32.235 | 0.291 |
| I61K/Q68R/Q79R/L99R/E282D | | | |
| MMLV-II | 56.5 | 32.395 | 0.105 |
| I61M/Q68R/Q79R/L99R/E282D | | | |
| MMLV-II | 59.9 | 34.408 | 0.498 |
| MMLV-II Q79R/L99R/E282D | 59.9 | 36.798 | 2.131 |
| MMLV-II Q68R/Q79R/L99R | 59.9 | 33.997 | 0.035 |
| MMLV-II | 59.9 | 32.009 | 0.051 |
| Q68R/Q79R/L99R/E282D | | | |
| MMLV-II | 59.9 | 33.685 | 0.317 |
| Q68R/Q79R/L99K/E282D | | | |
| MMLV-II | 59.9 | 33.083 | 0.163 |
| Q68R/Q79R/L99N/E282D | | | |
| MMLV-II | 59.9 | 34.160 | 0.066 |
| Q68K/Q79R/L99R/E282D | | | |
| MMLV-II | 59.9 | 33.650 | 0.161 |
| Q68R/Q79R/L99R/E282M | | | |
| MMLV-II | 59.9 | 33.341 | 0.096 |
| I61K/Q68R/Q79R/L99R/E282D | | | |
| MMLV-II | 59.9 | 34.439 | 0.222 |
| I61M/Q68R/Q79R/L99R/E282D | | | |
| MMLV-II | 62.6 | 35.163 | 0.447 |
| MMLV-II Q79R/L99R/E282D | 62.6 | 37.138 | 1.603 |
| MMLV-II Q68R/Q79R/L99R | 62.6 | 34.108 | 0.604 |
| MMLV-II | 62.6 | 32.539 | 0.060 |
| Q68R/Q79R/L99R/E282D | | | |
| MMLV-II | 62.6 | 34.175 | 0.421 |
| Q68R/Q79R/L99K/E282D | | | |
| MMLV-II | 62.6 | 33.726 | 0.622 |
| Q68R/Q79R/L99N/E282D | | | |
| MMLV-II | 62.6 | 34.376 | 0.408 |
| Q68K/Q79R/L99R/E282D | | | |
| MMLV-II | 62.6 | 33.792 | 0.231 |
| Q68R/Q79R/L99R/E282M | | | |
| MMLV-II | 62.6 | 33.768 | 0.387 |
| I61K/Q68R/Q79R/L99R/E282D | | | |
| MMLV-II | 62.6 | 34.428 | 0.085 |
| I61M/Q68R/Q79R/L99R/E282D | | | |
| MMLV-II | 64.2 | 37.284 | 0.764 |
| MMLV-II Q79R/L99R/E282D | 64.2 | 36.661 | 0.192 |
| MMLV-II Q68R/Q79R/L99R | 64.2 | 34.463 | 0.213 |
| MMLV-II | 64.2 | 32.992 | 0.023 |
| Q68R/Q79R/L99R/E282D | | | |
| MMLV-II | 64.2 | 34.805 | 0.472 |
| Q68R/Q79R/L99K/E282D | | | |
| MMLV-II | 64.2 | 34.060 | 0.043 |
| Q68R/Q79R/L99N/E282D | | | |
| MMLV-II | 64.2 | 34.508 | 0.302 |
| Q68K/Q79R/L99R/E282D | | | |
| MMLV-II | 64.2 | 34.481 | 0.078 |
| Q68R/Q79R/L99R/E282M | | | |
| MMLV-II | 64.2 | 34.231 | 0.253 |
| I61K/Q68R/Q79R/L99R/E282D | | | |
| MMLV-II | 64.2 | 35.049 | 0.885 |
| I61M/Q68R/Q79R/L99R/E282D | | | |
| MMLV-II | 65.0 | 35.809 | 0.511 |
| MMLV-II Q79R/L99R/E282D | 65.0 | 35.932 | 0.372 |
| MMLV-II Q68R/Q79R/L99R | 65.0 | 34.979 | 0.856 |
| MMLV-II | 65.0 | 33.293 | 0.319 |
| Q68R/Q79R/L99R/E282D | | | |
| MMLV-II | 65.0 | 34.974 | 0.536 |
| Q68R/Q79R/L99K/E282D | | | |
| MMLV-II | 65.0 | 34.862 | 0.268 |
| Q68R/Q79R/L99N/E282D | | | |
| MMLV-II | 65.0 | 34.363 | 0.201 |
| Q68K/Q79R/L99R/E282D | | | |
| MMLV-II | 65.0 | 34.687 | 0.666 |
| Q68R/Q79R/L99R/E282M | | | |
| MMLV-II | 65.0 | 34.246 | 0.563 |
| I61K/Q68R/Q79R/L99R/E282D | | | |
| MMLV-II | 65.0 | 34.872 | 0.467 |
| I61M/Q68R/Q79R/L99R/E282D | | | |

### Example 6: Reverse Transcriptase Mutant Evaluation by Oligo dT or Random Priming

This example demonstrates the procedure used to evaluate each mutant RTase's ability to synthesize cDNA from purified total RNA (DNased, isolated from HeLa cells) compared to the base construct of MMLV RTase. The mutant MMLV RTases were tested by two priming conditions: Oligo dT only and random hexamer priming using a standard two-step cDNA synthesis as described in Example 5.

The reactions were analyzed and reported by Ct value (Tables 19 and 20). Four mutant variants of MMLV RTase showed an increase in the overall activity using oligo dT priming compared to the base construct, Q299E, T332E and V433R. Eight mutant variants of MMLV RTase showed an increase in the overall activity using random priming compared to the base construct, P76R, L82R, I125R, Y271A, L280A, L280R, T328R and V433R.

TABLE 19
Two-Step cDNA Synthesis by MMLV-RT single mutants
using oligo dT priming. The data was generated via qPCR
human normalizer assay and data is reported by Ct value.

| MMLV-RT Variant | Ct Mean | Ct Standard Deviation |
|---|---|---|
| MMLV-II | 40.000 | 0.000 |
| MMLV-II D209A | 40.000 | 0.000 |
| MMLV-II D209E | 40.000 | 0.000 |
| MMLV-II D209R | 40.000 | 0.000 |
| MMLV-II D83A | 40.000 | 0.000 |
| MMLV-II D83E | 40.000 | 0.000 |
| MMLV-II D83R | 40.000 | 0.000 |
| MMLV-II E201A | 40.000 | 0.000 |
| MMLV-II E201D | 40.000 | 0.000 |
| MMLV-II E201R | 40.000 | 0.000 |
| MMLV-II E367A | 40.000 | 0.000 |
| MMLV-II E367D | 40.000 | 0.000 |
| MMLV-II E367R | 40.000 | 0.000 |
| MMLV-II E596A | 40.000 | 0.000 |
| MMLV-II E596D | 40.000 | 0.000 |
| MMLV-II E596R | 40.000 | 0.000 |
| MMLV-II F210A | 40.000 | 0.000 |
| MMLV-II F210E | 40.000 | 0.000 |
| MMLV-II F210R | 40.000 | 0.000 |
| MMLV-II F369A | 40.000 | 0.000 |
| MMLV-II F369E | 40.000 | 0.000 |
| MMLV-II F369R | 40.000 | 0.000 |
| MMLV-II G308A | 40.000 | 0.000 |
| MMLV-II G308E | 40.000 | 0.000 |
| MMLV-II G308R | 40.000 | 0.000 |
| MMLV-II G331A | 40.000 | 0.000 |
| MMLV-II G331E | 40.000 | 0.000 |
| MMLV-II G331R | 40.000 | 0.000 |
| MMLV-II G73A | 40.000 | 0.000 |
| MMLV-II G73E | 40.000 | 0.000 |
| MMLV-II G73R | 40.000 | 0.000 |
| MMLV-II H77A | 40.000 | 0.000 |
| MMLV-II H77E | 40.000 | 0.000 |
| MMLV-II H77R | 40.000 | 0.000 |
| MMLV-II I125A | 40.000 | 0.000 |
| MMLV-II I125E | 40.000 | 0.000 |
| MMLV-II I125R | 40.000 | 0.000 |
| MMLV-II I212A | 40.000 | 0.000 |
| MMLV-II I212E | 40.000 | 0.000 |
| MMLV-II I212R | 40.000 | 0.000 |
| MMLV-II I593A | 40.000 | 0.000 |
| MMLV-II I593E | 40.000 | 0.000 |
| MMLV-II I593R | 40.000 | 0.000 |
| MMLV-II I597A | 40.000 | 0.000 |
| MMLV-II I597E | 40.000 | 0.000 |
| MMLV-II I597R | 40.000 | 0.000 |
| MMLV-II K285A | 40.000 | 0.000 |
| MMLV-II K285E | 40.000 | 0.000 |
| MMLV-II K285R | 40.000 | 0.000 |
| MMLV-II K348A | 40.000 | 0.000 |
| MMLV-II K348E | 40.000 | 0.000 |
| MMLV-II K348R | 40.000 | 0.000 |
| MMLV-II L198A | 40.000 | 0.000 |
| MMLV-II L198E | 40.000 | 0.000 |
| MMLV-II L198R | 40.000 | 0.000 |
| MMLV-II L280A | 40.000 | 0.000 |
| MMLV-II L280E | 40.000 | 0.000 |
| MMLV-II L280R | 40.000 | 0.000 |
| MMLV-II L352A | 40.000 | 0.000 |
| MMLV-II L352E | 40.000 | 0.000 |
| MMLV-II L352R | 40.000 | 0.000 |
| MMLV-II L357A | 40.000 | 0.000 |
| MMLV-II L357E | 40.000 | 0.000 |
| MMLV-II L357R | 40.000 | 0.000 |
| MMLV-II L82A | 40.000 | 0.000 |
| MMLV-II L82E | 40.000 | 0.000 |
| MMLV-II L82R | 40.000 | 0.000 |
| MMLV-II N335A | 39.787 | 0.302 |
| MMLV-II N335E | 40.000 | 0.000 |
| MMLV-II N335R | 40.000 | 0.000 |
| MMLV-II P76A | 40.000 | 0.000 |
| MMLV-II P76E | 40.000 | 0.000 |
| MMLV-II P76R | 40.000 | 0.000 |
| MMLV-II Q213A | 40.000 | 0.000 |
| MMLV-II Q213E | 40.000 | 0.000 |
| MMLV-II Q213R | 40.000 | 0.000 |
| MMLV-II Q299A | 40.000 | 0.000 |
| MMLV-II Q299E | 37.177 | 3.993 |
| MMLV-II Q299R | 40.000 | 0.000 |
| MMLV-II Q654A | 40.000 | 0.000 |
| MMLV-II Q654E | 40.000 | 0.000 |
| MMLV-II Q654R | 40.000 | 0.000 |
| MMLV-II R205A | 40.000 | 0.000 |
| MMLV-II R205E | 39.947 | 0.075 |
| MMLV-II R205K | 40.000 | 0.000 |
| MMLV-II R211A | 40.000 | 0.000 |
| MMLV-II R211E | 40.000 | 0.000 |
| MMLV-II R211K | 40.000 | 0.000 |
| MMLV-II R311A | 40.000 | 0.000 |
| MMLV-II R311E | 40.000 | 0.000 |
| MMLV-II R311K | 40.000 | 0.000 |
| MMLV-II R389A | 40.000 | 0.000 |
| MMLV-II R389E | 40.000 | 0.000 |
| MMLV-II R389K | 40.000 | 0.000 |
| MMLV-II R650A | 40.000 | 0.000 |
| MMLV-II R650E | 40.000 | 0.000 |
| MMLV-II R650K | 40.000 | 0.000 |
| MMLV-II R657A | 40.000 | 0.000 |
| MMLV-II R657E | 39.965 | 0.050 |
| MMLV-II R657K | 40.000 | 0.000 |
| MMLV-II S67A | 40.000 | 0.000 |
| MMLV-II S67E | 40.000 | 0.000 |
| MMLV-II S67R | 36.816 | 0.703 |
| MMLV-II T328A | 40.000 | 0.000 |
| MMLV-II T328E | 40.000 | 0.000 |
| MMLV-II T328R | 40.000 | 0.000 |
| MMLV-II T332A | 39.750 | 0.354 |
| MMLV-II T332E | 38.461 | 2.177 |
| MMLV-II T332R | 40.000 | 0.000 |
| MMLV-II V129A | 40.000 | 0.000 |
| MMLV-II V129E | 40.000 | 0.000 |
| MMLV-II V129R | 40.000 | 0.000 |
| MMLV-II V433A | 40.000 | 0.000 |
| MMLV-II V433E | 40.000 | 0.000 |
| MMLV-II V433R | 38.884 | 0.806 |
| MMLV-II V476A | 40.000 | 0.000 |
| MMLV-II V476E | 40.000 | 0.000 |
| MMLV-II V476R | 40.000 | 0.000 |
| MMLV-II Y271A | 40.000 | 0.000 |
| MMLV-II Y271E | 40.000 | 0.000 |
| MMLV-II Y271R | 40.000 | 0.000 |
| MMLV-IV | 31.467 | 0.190 |

TABLE 20
Two-Step cDNA Synthesis by MMLV-RT single mutants
using random priming. The data was generated via qPCR
human normalizer assay and data is reported by Ct value.

| MMLV-RT Variant | Ct Mean | Ct Standard Deviation |
|---|---|---|
| MMLV-II | 40.000 | 0.000 |
| MMLV-II D209A | 40.000 | 0.000 |
| MMLV-II D209E | 40.000 | 0.000 |
| MMLV-II D209R | 40.000 | 0.000 |
| MMLV-II D83A | 40.000 | 0.000 |
| MMLV-II D83E | 40.000 | 0.000 |
| MMLV-II D83R | 40.000 | 0.000 |
| MMLV-II E201A | 40.000 | 0.000 |
| MMLV-II E201D | 40.000 | 0.000 |
| MMLV-II E201R | 40.000 | 0.000 |
| MMLV-II E367A | 40.000 | 0.000 |
| MMLV-II E367D | 40.000 | 0.000 |
| MMLV-II E367R | 40.000 | 0.000 |
| MMLV-II E596A | 40.000 | 0.000 |
| MMLV-II E596D | 40.000 | 0.000 |
| MMLV-II E596R | 40.000 | 0.000 |
| MMLV-II F210A | 40.000 | 0.000 |
| MMLV-II F210E | 40.000 | 0.000 |
| MMLV-II F210R | 40.000 | 0.000 |
| MMLV-II F369A | 40.000 | 0.000 |
| MMLV-II F369E | 40.000 | 0.000 |
| MMLV-II F369R | 40.000 | 0.000 |
| MMLV-II G308A | 40.000 | 0.000 |
| MMLV-II G308E | 40.000 | 0.000 |
| MMLV-II G308R | 40.000 | 0.000 |
| MMLV-II G331A | 40.000 | 0.000 |
| MMLV-II G331E | 40.000 | 0.000 |
| MMLV-II G331R | 40.000 | 0.000 |
| MMLV-II G73A | 40.000 | 0.000 |
| MMLV-II G73E | 40.000 | 0.000 |
| MMLV-II G73R | 40.000 | 0.000 |
| MMLV-II H77A | 39.708 | 0.412 |
| MMLV-II H77E | 40.000 | 0.000 |
| MMLV-II H77R | 40.000 | 0.000 |
| MMLV-II I125A | 40.000 | 0.000 |
| MMLV-II I125E | 40.000 | 0.000 |
| MMLV-II I125R | 39.449 | 0.779 |
| MMLV-II I212A | 40.000 | 0.000 |
| MMLV-II I212E | 40.000 | 0.000 |
| MMLV-II I212R | 40.000 | 0.000 |
| MMLV-II I593A | 40.000 | 0.000 |
| MMLV-II I593E | 40.000 | 0.000 |
| MMLV-II I593R | 40.000 | 0.000 |
| MMLV-II I597A | 40.000 | 0.000 |
| MMLV-II I597E | 40.000 | 0.000 |
| MMLV-II I597R | 40.000 | 0.000 |
| MMLV-II K285A | 40.000 | 0.000 |
| MMLV-II K285E | 40.000 | 0.000 |
| MMLV-II K285R | 39.783 | 0.308 |
| MMLV-II K348A | 40.000 | 0.000 |
| MMLV-II K348E | 40.000 | 0.000 |
| MMLV-II K348R | 40.000 | 0.000 |
| MMLV-II L198A | 40.000 | 0.000 |
| MMLV-II L198E | 40.000 | 0.000 |
| MMLV-II L198R | 40.000 | 0.000 |
| MMLV-II L280A | 39.503 | 0.703 |
| MMLV-II L280E | 40.000 | 0.000 |
| MMLV-II L280R | 38.762 | 1.751 |
| MMLV-II L352A | 39.778 | 0.313 |
| MMLV-II L352E | 40.000 | 0.000 |
| MMLV-II L352R | 40.000 | 0.000 |
| MMLV-II L357A | 40.000 | 0.000 |
| MMLV-II L357E | 40.000 | 0.000 |
| MMLV-II L357R | 40.000 | 0.000 |
| MMLV-II L82A | 40.000 | 0.000 |
| MMLV-II L82E | 39.673 | 0.462 |
| MMLV-II L82R | 38.926 | 1.518 |
| MMLV-II N335A | 39.876 | 0.175 |
| MMLV-II N335E | 40.000 | 0.000 |
| MMLV-II N335R | 39.861 | 0.196 |
| MMLV-II P76A | 40.000 | 0.000 |
| MMLV-II P76E | 40.000 | 0.000 |
| MMLV-II P76R | 39.535 | 0.658 |
| MMLV-II Q213A | 40.000 | 0.000 |
| MMLV-II Q213E | 40.000 | 0.000 |
| MMLV-II Q213R | 40.000 | 0.000 |
| MMLV-II Q299A | 40.000 | 0.000 |
| MMLV-II Q299E | 40.000 | 0.000 |
| MMLV-II Q299R | 40.000 | 0.000 |
| MMLV-II Q654A | 40.000 | 0.000 |
| MMLV-II Q654E | 40.000 | 0.000 |
| MMLV-II Q654R | 40.000 | 0.000 |
| MMLV-II R205A | 39.811 | 0.267 |
| MMLV-II R205E | 40.000 | 0.000 |
| MMLV-II R205K | 40.000 | 0.000 |
| MMLV-II R211A | 40.000 | 0.000 |
| MMLV-II R211E | 40.000 | 0.000 |
| MMLV-II R211K | 40.000 | 0.000 |
| MMLV-II R311A | 40.000 | 0.000 |
| MMLV-II R311E | 40.000 | 0.000 |
| MMLV-II R311K | 40.000 | 0.000 |
| MMLV-II R389A | 40.000 | 0.000 |
| MMLV-II R389E | 40.000 | 0.000 |
| MMLV-II R389K | 40.000 | 0.000 |
| MMLV-II R650A | 40.000 | 0.000 |
| MMLV-II R650E | 40.000 | 0.000 |
| MMLV-II R650K | 40.000 | 0.000 |
| MMLV-II R657A | 40.000 | 0.000 |
| MMLV-II R657E | 40.000 | 0.000 |
| MMLV-II R657K | 40.000 | 0.000 |
| MMLV-II S67A | 40.000 | 0.000 |
| MMLV-II S67E | 39.435 | 0.800 |
| MMLV-II S67R | 38.209 | 0.977 |
| MMLV-II T328A | 40.000 | 0.000 |
| MMLV-II T328E | 40.000 | 0.000 |
| MMLV-II T328R | 39.478 | 0.739 |
| MMLV-II T332A | 40.000 | 0.000 |
| MMLV-II T332E | 40.000 | 0.000 |
| MMLV-II T332R | 40.000 | 0.000 |
| MMLV-II V129A | 40.000 | 0.000 |
| MMLV-II V129E | 40.000 | 0.000 |
| MMLV-II V129R | 40.000 | 0.000 |
| MMLV-II V433A | 40.000 | 0.000 |
| MMLV-II V433E | 40.000 | 0.000 |
| MMLV-II V433R | 38.071 | 1.452 |
| MMLV-II V476A | 40.000 | 0.000 |
| MMLV-II V476E | 40.000 | 0.000 |
| MMLV-II V476R | 40.000 | 0.000 |
| MMLV-II Y271A | 39.466 | 0.755 |
| MMLV-II Y271E | 40.000 | 0.000 |
| MMLV-II Y271R | 40.000 | 0.000 |
| MMLV-IV | 31.850 | 0.183 |

In addition to the increased activity demonstrated in the MMLV RTase mutations Q299E, T332E, and V433R (Table 19), and the MMLV RTase mutations P76R, L82R, I125R, Y271A, L280A, L280R, T328R, and V433R (Table 20), further MMLV RTase mutations were selected by rational design and introduced by site-directed mutagenesis using standard PCR conditions and primers (Table 21).

TABLE 21
Sequences of primers used for cloning of MMLV
RTase base construct and mutants into pET28b. All
primers were ordered as DNA oligos from Integrated
DNA Technologies.

| SEQ | | |
|---|---|---|
| ID | | |
| NO: | Primer Name | Primer Sequence (5′ - 3′) |
| 700 | MMLV V433R | AGTTGACGATGGGTCAACCCTTACGTATCTTGGCT |
| | SDM F | CCACATGCTGTAGA |
| 701 | MMLV V433R | TCTACAGCATGTGGAGCCAAGATACGTAAGGGTTG |
| | SDM R | ACCCATCGTCAACT |
| 702 | MMLV I593E | CGTTATGCTTTTGCAACAGCGCATGAGCATGGCGA |
| | SDM F | AATTTACCGCCGC |
| 703 | MMLV I593E | GCGGCGGTAAATTTCGCCATGCTCATGCGCTGTTG |
| | SDM R | CAAAAGCATAACG |
| 704 | MMLV Q299E | TACGCCTAAGACGCCACGCGAGTTGCGTGAATTTT |
| | SDM F | TGGGCACAGC |
| 705 | MMLV Q299E | GCTGTGCCCAAAAATTCACGCAACTCGCGTGGCGT |
| | SDM R | CTTAGGCGTA |
| 706 | MMLV L82Y | GATTAAGCCACATATTCAGCGCTTGTATGACCAGG |
| | SDM F | GGATCTTGGTCC |
| 707 | MMLV L82Y | GGACCAAGATCCCCTGGTCATACAAGCGCTGAATA |
| | SDM R | TGTGGCTTAATC |
| 708 | MMLV L280I | TGCTGAAAGAAGGTCAACGTTGGATCACTGAAGCG |
| | SDM F | CGTAAGGAGACC |
| 709 | MMLV L280I | GGTCTCCTTACGCGCTTCAGTGATCCAACGTTGACC |
| | SDM R | TTCTTTCAGCA |
| 710 | MMLV V433N | AGTTGACGATGGGTCAACCCTTAAACATCTTGGCT |
| | SDM F | CCACATGCTGTAGA |
| 711 | MMLV V433N | TCTACAGCATGTGGAGCCAAGATGTTTAAGGGTTG |
| | SDM R | ACCCATCGTCAACT |
| 712 | MMLV I593W | CGTTATGCTTTTGCAACAGCGCATTGGCATGGCGA |
| | SDM F | AATTTACCGCCGC |
| 713 | MMLV I593W | GCGGCGGTAAATTTCGCCATGCCAATGCGCTGTTG |
| | SDM R | CAAAAGCATAACG |
| 714 | MMLV T306K | GCCAGTTGCGTGAATTTTTGGGCAAAGCGGGATTC |
| | TQP | TGTCGTTTATGGATTCC |
| 715 | MMLV T306K | GGAATCCATAAACGACAGAATCCCGCTTTGCCCAA |
| | BTM | AAATTCACGCAACTGGC |

The resulting plasmids were transformed into E. coli BL21(DE3) cells for protein expression and proteins isolated through affinity and ion exchange chromatography (Table 22).

TABLE 22
Sequences of MMLV RTase base construct and mutant MMLV RTase

| SEQ ID NO: | Construct | Construct Sequence (DNA: 5′-3′ or AA) |
|---|---|---|
| 716 | MMLV-II RTase | ATGACTTTAAATATTGAGGATGAGCATCGTTTA |
| | | CATGAGACATCAAAAGAACCCGACGTGAGCTTA |
| | | GGGTCAACGTGGCTTTCTGACTTCCCCCAGGCG |
| | | TGGGCGGAGACTGGCGGAATGGGGTTAGCTGTC |
| | | CGCCAAGCACCGTTGATCATCCCGTTAAAGGCA |
| | | ACGTCTACACCTGTCTCTATCAAACAGTACCCC |
| | | ATGAGTCAAGAGGCCCGCCTGGGGATTAAGCCA |
| | | CATATTCAGCGCTTGCTGGACCAGGGGATCTTG |
| | | GTCCCATGTCAATCTCCGTGGAACACCCCCCTT |
| | | CTGCCCGTGAAAAAGCCAGGTACAAACGATTAT |
| | | CGTCCAGTTCAAGATCTTCGCGAGGTCAACAAA |
| | | CGCGTAGAAGACATCCATCCGACTGTACCTAAT |
| | | CCTTATAATCTGTTATCAGGCCTGCCCCCATCG |
| | | CACCAATGGTATACAGTATTAGACTTGAAAGAC |
| | | GCGTTCTTTTGCCTGCGTCTGCACCCAACGTCT |
| | | CAGCCGCTGTTTGCGTTCGAATGGCGTGATCCT |
| | | GAAATGGGAATTTCGGGTCAGTTAACCTGGACT |
| | | CGTCTGCCCCAGGGCTTTAAAAACAGCCCCACA |
| | | TTGTTCGATGAAGCACTTCACCGTGACTTAGCA |
| | | GACTTCCGTATCCAACACCCAGACTTAATTCTG |
| | | TTACAGTATGTTGACGACCTTTTGTTGGCGGCA |
| | | ACGTCTGAACTTGACTGTCAGCAAGGCACACGC |
| | | GCGTTATTACAAACGTTAGGTAACTTAGGATAT |
| | | CGTGCGTCCGCGAAAAAGGCGCAAATTTGTCAA |
| | | AAACAGGTAAAGTACCTTGGGTATTTGCTGAAA |
| | | GAAGGTCAACGTTGGCTGACTGAAGCGCGTAAG |
| | | GAGACCGTAATGGGGCAGCCTACGCCTAAGACG |
| | | CCACGCCAGTTGCGTGAATTTTTGGGCACAGCG |
| | | GGATTCTGTCGTTTATGGATTCCTGGGTTCGCT |
| | | GAAATGGCTGCACCCCTGTACCCCTTAACAAAA |
| | | ACAGGGACGCTTTTCAACTGGGGGCCAGACCAG |
| | | CAAAAGGCGTATCAGGAGATCAAACAAGCTTTG |
| | | TTGACCGCACCCGCGTTGGGTCTTCCGGATTTA |
| | | ACCAAGCCCTTTGAGCTGTTCGTTGATGAAAAA |
| | | CAGGGATATGCAAAAGGAGTATTAACCCAAAAG |
| | | TTAGGCCCGTGGCGTCGCCCTGTTGCTTACTTG |
| | | AGTAAAAAATTGGATCCTGTCGCAGCAGGATGG |
| | | CCACCGTGCTTGCGTATGGTCGCGGCAATTGCC |
| | | GTTTTGACAAAGGATGCAGGTAAGTTGACGATG |
| | | GGTCAACCCTTAGTAATCTTGGCTCCACATGCT |
| | | GTAGAAGCGTTAGTAAAGCAGCCCCCAGACCGC |
| | | TGGCTTTCTAATGCGCGCATGACCCACTATCAG |
| | | GCGCTTCTGCTTGATACGGATCGTGTACAATTT |
| | | GGACCAGTTGTAGCTTTGAATCCAGCTACTTTG |
| | | CTTCCCCTTCCAGAAGAAGGACTTCAGCACAAT |
| | | TGTTTAGATATTCTGGCCGAGGCACATGGGACG |
| | | CGCCCTGATTTGACGGATCAGCCACTGCCTGAT |
| | | GCCGACCATACATGGTATACTGGCGGCAGTAGT |
| | | CTTCTTCAAGAGGGGCAACGCAAGGCGGGAGCA |
| | | GCCGTCACTACGGAGACCGAAGTTATCTGGGCC |
| | | AAAGCGTTACCCGCGGGAACATCCGCGCAACGT |
| | | GCACAGTTAATCGCTCTGACACAGGCCCTGAAG |
| | | ATGGCAGAGGGCAAAAAGTTGAATGTCTACACC |
| | | AACTCACGTTATGCTTTTGCAACAGCGCATATC |
| | | CATGGCGAAATTTACCGCCGCCGTGGTCTGCTG |
| | | ACTAGTGAGGGTAAGGAAATTAAAAATAAAGAT |
| | | GAGATTCTTGCGTTGTTAAAAGCTTTATTCTTA |
| | | CCAAAACGCCTTTCGATCATTCATTGCCCGGGG |
| | | CATCAAAAGGGTCACTCAGCGGAGGCTCGTGGA |
| | | AACCGTATGGCGGACCAAGCTGCCCGTAAGGCG |
| | | GCGATCACAGAGACCCCGGATACATCAACGCTG |
| | | TTGATCGAAAACAGCTCTCCCTACACTAGCGAG |
| | | CATTTTTAA |
| 717 | MMLV-II RTase | MTLNIEDEHRLHETSKEPDVSLGSTWLSDFPQA |
| | | WAETGGMGLAVRQAPLIIPLKATSTPVSIKQYP |
| | | MSQEARLGIKPHIQRLLDQGILVPCQSPWNTPL |
| | | LPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTEARKETVMGQPTPKT |
| | | PRQLREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLVILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHI |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 718 | MMLV-II | ATGACTTTAAATATTGAGGATGAGCATCGTTTA |
| | Q68R/Q79R/L99R/ | CATGAGACATCAAAAGAACCCGACGTGAGCTTA |
| | E282D/Q299E/V433N/ | GGGTCAACGTGGCTTTCTGACTTCCCCCAGGCG |
| | I593W | TGGGCGGAGACTGGCGGAATGGGGTTAGCTGTC |
| | | CGCCAAGCACCGTTGATCATCCCGTTAAAGGCA |
| | | ACGTCTACACCTGTCTCTATCAAACAGTACCCC |
| | | ATGAGTCGTGAGGCCCGCCTGGGGATTAAGCCA |
| | | CATATTCGTCGCTTGCTGGACCAGGGGATCTTG |
| | | GTCCCATGTCAATCTCCGTGGAACACCCCCCTT |
| | | CGTCCCGTGAAAAAGCCAGGTACAAACGATTAT |
| | | CGTCCAGTTCAAGATCTTCGCGAGGTCAACAAA |
| | | CGCGTAGAAGACATCCATCCGACTGTACCTAAT |
| | | CCTTATAATCTGTTATCAGGCCTGCCCCCATCG |
| | | CACCAATGGTATACAGTATTAGACTTGAAAGAC |
| | | GCGTTCTTTTGCCTGCGTCTGCACCCAACGTCT |
| | | CAGCCGCTGTTTGCGTTCGAATGGCGTGATCCT |
| | | GAAATGGGAATTTCGGGTCAGTTAACCTGGACT |
| | | CGTCTGCCCCAGGGCTTTAAAAACAGCCCCACA |
| | | TTGTTCGATGAAGCACTTCACCGTGACTTAGCA |
| | | GACTTCCGTATCCAACACCCAGACTTAATTCTG |
| | | TTACAGTATGTTGACGACCTTTTGTTGGCGGCA |
| | | ACGTCTGAACTTGACTGTCAGCAAGGCACACGC |
| | | GCGTTATTACAAACGTTAGGTAACTTAGGATAT |
| | | CGTGCGTCCGCGAAAAAGGCGCAAATTTGTCAA |
| | | AAACAGGTAAAGTACCTTGGGTATTTGCTGAAA |
| | | GAAGGTCAACGTTGGCTGACTGATGCGCGTAAG |
| | | GAGACCGTAATGGGGCAGCCTACGCCTAAGACG |
| | | CCACGCGAATTGCGTGAATTTTTGGGCACAGCG |
| | | GGATTCTGTCGTTTATGGATTCCTGGGTTCGCT |
| | | GAAATGGCTGCACCCCTGTACCCCTTAACAAAA |
| | | ACAGGGACGCTTTTCAACTGGGGGCCAGACCAG |
| | | CAAAAGGCGTATCAGGAGATCAAACAAGCTTTG |
| | | TTGACCGCACCCGCGTTGGGTCTTCCGGATTTA |
| | | ACCAAGCCCTTTGAGCTGTTCGTTGATGAAAAA |
| | | CAGGGATATGCAAAAGGAGTATTAACCCAAAAG |
| | | TTAGGCCCGTGGCGTCGCCCTGTTGCTTACTTG |
| | | AGTAAAAAATTGGATCCTGTCGCAGCAGGATGG |
| | | CCACCGTGCTTGCGTATGGTCGCGGCAATTGCC |
| | | GTTTTGACAAAGGATGCAGGTAAGTTGACGATG |
| | | GGTCAACCCTTACGTATCTTGGCTCCACATGCT |
| | | GTAGAAGCGTTAGTAAAGCAGCCCCCAGACCGC |
| | | TGGCTTTCTAATGCGCGCATGACCCACTATCAG |
| | | GCGCTTCTGCTTGATACGGATCGTGTACAATTT |
| | | GGACCAGTTGTAGCTTTGAATCCAGCTACTTTG |
| | | CTTCCCCTTCCAGAAGAAGGACTTCAGCACAAT |
| | | TGTTTAGATATTCTGGCCGAGGCACATGGGACG |
| | | CGCCCTGATTTGACGGATCAGCCACTGCCTGAT |
| | | GCCGACCATACATGGTATACTGGCGGCAGTAGT |
| | | CTTCTTCAAGAGGGGCAACGCAAGGCGGGAGCA |
| | | GCCGTCACTACGGAGACCGAAGTTATCTGGGCC |
| | | AAAGCGTTACCCGCGGGAACATCCGCGCAACGT |
| | | GCACAGTTAATCGCTCTGACACAGGCCCTGAAG |
| | | ATGGCAGAGGGCAAAAAGTTGAATGTCTACACC |
| | | AACTCACGTTATGCTTTTGCAACAGCGCATTGG |
| | | CATGGCGAAATTTACCGCCGCCGTGGTCTGCTG |
| | | ACTAGTGAGGGTAAGGAAATTAAAAATAAAGAT |
| | | GAGATTCTTGCGTTGTTAAAAGCTTTATTCTTA |
| | | CCAAAACGCCTTTCGATCATTCATTGCCCGGGG |
| | | CATCAAAAGGGTCACTCAGCGGAGGCTCGTGGA |
| | | AACCGTATGGCGGACCAAGCTGCCCGTAAGGCG |
| | | GCGATCACAGAGACCCCGGATACATCAACGCTG |
| | | TTGATCGAAAACAGCTCTCCCTACACTAGCGAG |
| | | CATTTT |
| 719 | MMLV-II | MTLNIEDEHRLHETSKEPDVSLGSTWLSDFPQA |
| | Q68R/Q79R/L99R/ | WAETGGMGLAVRQAPLIIPLKATSTPVSIKQYP |
| | E282D/Q299E/V433N/ | MSREARLGIKPHIRRLLDQGILVPCQSPWNTPL |
| | I593W | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRELREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLRILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHW |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 720 | MMLV-II | ATGACTTTAAATATTGAGGATGAGCATCGTTTA |
| | Q68R/Q79R/L99R/ | CATGAGACATCAAAAGAACCCGACGTGAGCTTA |
| | L280I/E282D/Q299E/ | GGGTCAACGTGGCTTTCTGACTTCCCCCAGGCG |
| | V433N/I593W | TGGGCGGAGACTGGCGGAATGGGGTTAGCTGTC |
| | | CGCCAAGCACCGTTGATCATCCCGTTAAAGGCA |
| | | ACGTCTACACCTGTCTCTATCAAACAGTACCCC |
| | | ATGAGTCGTGAGGCCCGCCTGGGGATTAAGCCA |
| | | CATATTCGTCGCTTGCTGGACCAGGGGATCTTG |
| | | GTCCCATGTCAATCTCCGTGGAACACCCCCCTT |
| | | CGTCCCGTGAAAAAGCCAGGTACAAACGATTAT |
| | | CGTCCAGTTCAAGATCTTCGCGAGGTCAACAAA |
| | | CGCGTAGAAGACATCCATCCGACTGTACCTAAT |
| | | CCTTATAATCTGTTATCAGGCCTGCCCCCATCG |
| | | CACCAATGGTATACAGTATTAGACTTGAAAGAC |
| | | GCGTTCTTTTGCCTGCGTCTGCACCCAACGTCT |
| | | CAGCCGCTGTTTGCGTTCGAATGGCGTGATCCT |
| | | GAAATGGGAATTTCGGGTCAGTTAACCTGGACT |
| | | CGTCTGCCCCAGGGCTTTAAAAACAGCCCCACA |
| | | TTGTTCGATGAAGCACTTCACCGTGACTTAGCA |
| | | GACTTCCGTATCCAACACCCAGACTTAATTCTG |
| | | TTACAGTATGTTGACGACCTTTTGTTGGCGGCA |
| | | ACGTCTGAACTTGACTGTCAGCAAGGCACACGC |
| | | GCGTTATTACAAACGTTAGGTAACTTAGGATAT |
| | | CGTGCGTCCGCGAAAAAGGCGCAAATTTGTCAA |
| | | AAACAGGTAAAGTACCTTGGGTATTTGCTGAAA |
| | | GAAGGTCAACGTTGGATTACTGATGCGCGTAAG |
| | | GAGACCGTAATGGGGCAGCCTACGCCTAAGACG |
| | | CCACGCGAATTGCGTGAATTTTTGGGCACAGCG |
| | | GGATTCTGTCGTTTATGGATTCCTGGGTTCGCT |
| | | GAAATGGCTGCACCCCTGTACCCCTTAACAAAA |
| | | ACAGGGACGCTTTTCAACTGGGGGCCAGACCAG |
| | | CAAAAGGCGTATCAGGAGATCAAACAAGCTTTG |
| | | TTGACCGCACCCGCGTTGGGTCTTCCGGATTTA |
| | | ACCAAGCCCTTTGAGCTGTTCGTTGATGAAAAA |
| | | CAGGGATATGCAAAAGGAGTATTAACCCAAAAG |
| | | TTAGGCCCGTGGCGTCGCCCTGTTGCTTACTTG |
| | | AGTAAAAAATTGGATCCTGTCGCAGCAGGATGG |
| | | CCACCGTGCTTGCGTATGGTCGCGGCAATTGCC |
| | | GTTTTGACAAAGGATGCAGGTAAGTTGACGATG |
| | | GGTCAACCCTTACGTATCTTGGCTCCACATGCT |
| | | GTAGAAGCGTTAGTAAAGCAGCCCCCAGACCGC |
| | | TGGCTTTCTAATGCGCGCATGACCCACTATCAG |
| | | GCGCTTCTGCTTGATACGGATCGTGTACAATTT |
| | | GGACCAGTTGTAGCTTTGAATCCAGCTACTTTG |
| | | CTTCCCCTTCCAGAAGAAGGACTTCAGCACAAT |
| | | TGTTTAGATATTCTGGCCGAGGCACATGGGACG |
| | | CGCCCTGATTTGACGGATCAGCCACTGCCTGAT |
| | | GCCGACCATACATGGTATACTGGCGGCAGTAGT |
| | | CTTCTTCAAGAGGGGCAACGCAAGGCGGGAGCA |
| | | GCCGTCACTACGGAGACCGAAGTTATCTGGGCC |
| | | AAAGCGTTACCCGCGGGAACATCCGCGCAACGT |
| | | GCACAGTTAATCGCTCTGACACAGGCCCTGAAG |
| | | ATGGCAGAGGGCAAAAAGTTGAATGTCTACACC |
| | | AACTCACGTTATGCTTTTGCAACAGCGCATTGG |
| | | CATGGCGAAATTTACCGCCGCCGTGGTCTGCTG |
| | | ACTAGTGAGGGTAAGGAAATTAAAAATAAAGAT |
| | | GAGATTCTTGCGTTGTTAAAAGCTTTATTCTTA |
| | | CCAAAACGCCTTTCGATCATTCATTGCCCGGGG |
| | | CATCAAAAGGGTCACTCAGCGGAGGCTCGTGGA |
| | | AACCGTATGGCGGACCAAGCTGCCCGTAAGGCG |
| | | GCGATCACAGAGACCCCGGATACATCAACGCTG |
| | | TTGATCGAAAACAGCTCTCCCTACACTAGCGAG |
| | | CATTTT |
| 721 | MMLV-II | MTLNIEDEHRLHETSKEPDVSLGSTWLSDFPQA |
| | Q68R/Q79R/L99R/ | WAETGGMGLAVRQAPLIIPLKATSTPVSIKQYP |
| | L280I/E282D/Q299E/ | MSREARLGIKPHIRRLLDQGILVPCQSPWNTPL |
| | V433N/I593W | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWITDARKETVMGQPTPKT |
| | | PRELREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLRILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHW |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 722 | MMLV-II | ATGACTTTAAATATTGAGGATGAGCATCGTTTA |
| | Q68R/Q79R/L82Y/ | CATGAGACATCAAAAGAACCCGACGTGAGCTTA |
| | L99R/L280I/E282D/ | GGGTCAACGTGGCTTTCTGACTTCCCCCAGGCG |
| | Q299E/V433N/I593W | TGGGCGGAGACTGGCGGAATGGGGTTAGCTGTC |
| | | CGCCAAGCACCGTTGATCATCCCGTTAAAGGCA |
| | | ACGTCTACACCTGTCTCTATCAAACAGTACCCC |
| | | ATGAGTCGTGAGGCCCGCCTGGGGATTAAGCCA |
| | | CATATTCGTCGCTTGTATGACCAGGGGATCTTG |
| | | GTCCCATGTCAATCTCCGTGGAACACCCCCCTT |
| | | CGTCCCGTGAAAAAGCCAGGTACAAACGATTAT |
| | | CGTCCAGTTCAAGATCTTCGCGAGGTCAACAAA |
| | | CGCGTAGAAGACATCCATCCGACTGTACCTAAT |
| | | CCTTATAATCTGTTATCAGGCCTGCCCCCATCG |
| | | CACCAATGGTATACAGTATTAGACTTGAAAGAC |
| | | GCGTTCTTTTGCCTGCGTCTGCACCCAACGTCT |
| | | CAGCCGCTGTTTGCGTTCGAATGGCGTGATCCT |
| | | GAAATGGGAATTTCGGGTCAGTTAACCTGGACT |
| | | CGTCTGCCCCAGGGCTTTAAAAACAGCCCCACA |
| | | TTGTTCGATGAAGCACTTCACCGTGACTTAGCA |
| | | GACTTCCGTATCCAACACCCAGACTTAATTCTG |
| | | TTACAGTATGTTGACGACCTTTTGTTGGCGGCA |
| | | ACGTCTGAACTTGACTGTCAGCAAGGCACACGC |
| | | GCGTTATTACAAACGTTAGGTAACTTAGGATAT |
| | | CGTGCGTCCGCGAAAAAGGCGCAAATTTGTCAA |
| | | AAACAGGTAAAGTACCTTGGGTATTTGCTGAAA |
| | | GAAGGTCAACGTTGGATTACTGATGCGCGTAAG |
| | | GAGACCGTAATGGGGCAGCCTACGCCTAAGACG |
| | | CCACGCGAATTGCGTGAATTTTTGGGCACAGCG |
| | | GGATTCTGTCGTTTATGGATTCCTGGGTTCGCT |
| | | GAAATGGCTGCACCCCTGTACCCCTTAACAAAA |
| | | ACAGGGACGCTTTTCAACTGGGGGCCAGACCAG |
| | | CAAAAGGCGTATCAGGAGATCAAACAAGCTTTG |
| | | TTGACCGCACCCGCGTTGGGTCTTCCGGATTTA |
| | | ACCAAGCCCTTTGAGCTGTTCGTTGATGAAAAA |
| | | CAGGGATATGCAAAAGGAGTATTAACCCAAAAG |
| | | TTAGGCCCGTGGCGTCGCCCTGTTGCTTACTTG |
| | | AGTAAAAAATTGGATCCTGTCGCAGCAGGATGG |
| | | CCACCGTGCTTGCGTATGGTCGCGGCAATTGCC |
| | | GTTTTGACAAAGGATGCAGGTAAGTTGACGATG |
| | | GGTCAACCCTTACGTATCTTGGCTCCACATGCT |
| | | GTAGAAGCGTTAGTAAAGCAGCCCCCAGACCGC |
| | | TGGCTTTCTAATGCGCGCATGACCCACTATCAG |
| | | GCGCTTCTGCTTGATACGGATCGTGTACAATTT |
| | | GGACCAGTTGTAGCTTTGAATCCAGCTACTTTG |
| | | CTTCCCCTTCCAGAAGAAGGACTTCAGCACAAT |
| | | TGTTTAGATATTCTGGCCGAGGCACATGGGACG |
| | | CGCCCTGATTTGACGGATCAGCCACTGCCTGAT |
| | | GCCGACCATACATGGTATACTGGCGGCAGTAGT |
| | | CTTCTTCAAGAGGGGCAACGCAAGGCGGGAGCA |
| | | GCCGTCACTACGGAGACCGAAGTTATCTGGGCC |
| | | AAAGCGTTACCCGCGGGAACATCCGCGCAACGT |
| | | GCACAGTTAATCGCTCTGACACAGGCCCTGAAG |
| | | ATGGCAGAGGGCAAAAAGTTGAATGTCTACACC |
| | | AACTCACGTTATGCTTTTGCAACAGCGCATTGG |
| | | CATGGCGAAATTTACCGCCGCCGTGGTCTGCTG |
| | | ACTAGTGAGGGTAAGGAAATTAAAAATAAAGAT |
| | | GAGATTCTTGCGTTGTTAAAAGCTTTATTCTTA |
| | | CCAAAACGCCTTTCGATCATTCATTGCCCGGGG |
| | | CATCAAAAGGGTCACTCAGCGGAGGCTCGTGGA |
| | | AACCGTATGGCGGACCAAGCTGCCCGTAAGGCG |
| | | GCGATCACAGAGACCCCGGATACATCAACGCTG |
| | | TTGATCGAAAACAGCTCTCCCTACACTAGCGAG |
| | | CATTTT |
| 723 | MMLV-II | MTLNIEDEHRLHETSKEPDVSLGSTWLSDFPQA |
| | Q68R/Q79R/L82Y/ | WAETGGMGLAVRQAPLIIPLKATSTPVSIKQYP |
| | L99R/L280I/E282D/ | MSREARLGIKPHIRRLYDQGILVPCQSPWNTPL |
| | Q299E/V433N/I593W | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWITDARKETVMGQPTPKT |
| | | PRELREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLRILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHW |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 724 | MMLV-II | ATGACTTTAAATATTGAGGATGAGCATCGTTTA |
| | Q68R/Q79R/L82Y/ | CATGAGACATCAAAAGAACCCGACGTGAGCTTA |
| | L99R/L280I/E282D/ | GGGTCAACGTGGCTTTCTGACTTCCCCCAGGCG |
| | Q299E/T306K/V433N/ | TGGGCGGAGACTGGCGGAATGGGGTTAGCTGTC |
| | I593W | CGCCAAGCACCGTTGATCATCCCGTTAAAGGCA |
| | | ACGTCTACACCTGTCTCTATCAAACAGTACCCC |
| | | ATGAGTCGTGAGGCCCGCCTGGGGATTAAGCCA |
| | | CATATTCGTCGCTTGTATGACCAGGGGATCTTG |
| | | GTCCCATGTCAATCTCCGTGGAACACCCCCCTT |
| | | CGTCCCGTGAAAAAGCCAGGTACAAACGATTAT |
| | | CGTCCAGTTCAAGATCTTCGCGAGGTCAACAAA |
| | | CGCGTAGAAGACATCCATCCGACTGTACCTAAT |
| | | CCTTATAATCTGTTATCAGGCCTGCCCCCATCG |
| | | CACCAATGGTATACAGTATTAGACTTGAAAGAC |
| | | GCGTTCTTTTGCCTGCGTCTGCACCCAACGTCT |
| | | CAGCCGCTGTTTGCGTTCGAATGGCGTGATCCT |
| | | GAAATGGGAATTTCGGGTCAGTTAACCTGGACT |
| | | CGTCTGCCCCAGGGCTTTAAAAACAGCCCCACA |
| | | TTGTTCGATGAAGCACTTCACCGTGACTTAGCA |
| | | GACTTCCGTATCCAACACCCAGACTTAATTCTG |
| | | TTACAGTATGTTGACGACCTTTTGTTGGCGGCA |
| | | ACGTCTGAACTTGACTGTCAGCAAGGCACACGC |
| | | GCGTTATTACAAACGTTAGGTAACTTAGGATAT |
| | | CGTGCGTCCGCGAAAAAGGCGCAAATTTGTCAA |
| | | AAACAGGTAAAGTACCTTGGGTATTTGCTGAAA |
| | | GAAGGTCAACGTTGGATTACTGATGCGCGTAAG |
| | | GAGACCGTAATGGGGCAGCCTACGCCTAAGACG |
| | | CCACGCGAATTGCGTGAATTTTTGGGCAAAGCG |
| | | GGATTCTGTCGTTTATGGATTCCTGGGTTCGCT |
| | | GAAATGGCTGCACCCCTGTACCCCTTAACAAAA |
| | | ACAGGGACGCTTTTCAACTGGGGGCCAGACCAG |
| | | CAAAAGGCGTATCAGGAGATCAAACAAGCTTTG |
| | | TTGACCGCACCCGCGTTGGGTCTTCCGGATTTA |
| | | ACCAAGCCCTTTGAGCTGTTCGTTGATGAAAAA |
| | | CAGGGATATGCAAAAGGAGTATTAACCCAAAAG |
| | | TTAGGCCCGTGGCGTCGCCCTGTTGCTTACTTG |
| | | AGTAAAAAATTGGATCCTGTCGCAGCAGGATGG |
| | | CCACCGTGCTTGCGTATGGTCGCGGCAATTGCC |
| | | GTTTTGACAAAGGATGCAGGTAAGTTGACGATG |
| | | GGTCAACCCTTAAACATCTTGGCTCCACATGCT |
| | | GTAGAAGCGTTAGTAAAGCAGCCCCCAGACCGC |
| | | TGGCTTTCTAATGCGCGCATGACCCACTATCAG |
| | | GCGCTTCTGCTTGATACGGATCGTGTACAATTT |
| | | GGACCAGTTGTAGCTTTGAATCCAGCTACTTTG |
| | | CTTCCCCTTCCAGAAGAAGGACTTCAGCACAAT |
| | | TGTTTAGATATTCTGGCCGAGGCACATGGGACG |
| | | CGCCCTGATTTGACGGATCAGCCACTGCCTGAT |
| | | GCCGACCATACATGGTATACTGGCGGCAGTAGT |
| | | CTTCTTCAAGAGGGGCAACGCAAGGCGGGAGCA |
| | | GCCGTCACTACGGAGACCGAAGTTATCTGGGCC |
| | | AAAGCGTTACCCGCGGGAACATCCGCGCAACGT |
| | | GCACAGTTAATCGCTCTGACACAGGCCCTGAAG |
| | | ATGGCAGAGGGCAAAAAGTTGAATGTCTACACC |
| | | AACTCACGTTATGCTTTTGCAACAGCGCATTGG |
| | | CATGGCGAAATTTACCGCCGCCGTGGTCTGCTG |
| | | ACTAGTGAGGGTAAGGAAATTAAAAATAAAGAT |
| | | GAGATTCTTGCGTTGTTAAAAGCTTTATTCTTA |
| | | CCAAAACGCCTTTCGATCATTCATTGCCCGGGG |
| | | CATCAAAAGGGTCACTCAGCGGAGGCTCGTGGA |
| | | AACCGTATGGCGGACCAAGCTGCCCGTAAGGCG |
| | | GCGATCACAGAGACCCCGGATACATCAACGCTG |
| | | TTGATCGAAAACAGCTCTCCCTACACTAGCGAG |
| | | CATTTT |
| 725 | MMLV-II | MTLNIEDEHRLHETSKEPDVSLGSTWLSDFPQA |
| | Q68R/Q79R/L82Y/ | WAETGGMGLAVRQAPLIIPLKATSTPVSIKQYP |
| | L99R/L280I/E282D/ | MSREARLGIKPHIRRLYDQGILVPCQSPWNTPL |
| | Q299E/T306K/V433N/ | RPVKKPGTNDYRPVQDLREVNKRVEDIHPTVPN |
| | I593W | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWITDARKETVMGQPTPKT |
| | | PRELREFLGKAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLNILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHW |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 726 | MMLV-II | ATGACTTTAAATATTGAGGATGAGCATCGTTTA |
| | Q68R/Q79R/L99R/ | CATGAGACATCAAAAGAACCCGACGTGAGCTTA |
| | E282D/Q299E/T306K/ | GGGTCAACGTGGCTTTCTGACTTCCCCCAGGCG |
| | V433R/I593E | TGGGCGGAGACTGGCGGAATGGGGTTAGCTGTC |
| | | CGCCAAGCACCGTTGATCATCCCGTTAAAGGCA |
| | | ACGTCTACACCTGTCTCTATCAAACAGTACCCC |
| | | ATGAGTCGTGAGGCCCGCCTGGGGATTAAGCCA |
| | | CATATTCGTCGCTTGCTGGACCAGGGGATCTTG |
| | | GTCCCATGTCAATCTCCGTGGAACACCCCCCTT |
| | | CGTCCCGTGAAAAAGCCAGGTACAAACGATTAT |
| | | CGTCCAGTTCAAGATCTTCGCGAGGTCAACAAA |
| | | CGCGTAGAAGACATCCATCCGACTGTACCTAAT |
| | | CCTTATAATCTGTTATCAGGCCTGCCCCCATCG |
| | | CACCAATGGTATACAGTATTAGACTTGAAAGAC |
| | | GCGTTCTTTTGCCTGCGTCTGCACCCAACGTCT |
| | | CAGCCGCTGTTTGCGTTCGAATGGCGTGATCCT |
| | | GAAATGGGAATTTCGGGTCAGTTAACCTGGACT |
| | | CGTCTGCCCCAGGGCTTTAAAAACAGCCCCACA |
| | | TTGTTCGATGAAGCACTTCACCGTGACTTAGCA |
| | | GACTTCCGTATCCAACACCCAGACTTAATTCTG |
| | | TTACAGTATGTTGACGACCTTTTGTTGGCGGCA |
| | | ACGTCTGAACTTGACTGTCAGCAAGGCACACGC |
| | | GCGTTATTACAAACGTTAGGTAACTTAGGATAT |
| | | CGTGCGTCCGCGAAAAAGGCGCAAATTTGTCAA |
| | | AAACAGGTAAAGTACCTTGGGTATTTGCTGAAA |
| | | GAAGGTCAACGTTGGCTGACTGATGCGCGTAAG |
| | | GAGACCGTAATGGGGCAGCCTACGCCTAAGACG |
| | | CCACGCGAATTGCGTGAATTTTTGGGCAAAGCG |
| | | GGATTCTGTCGTTTATGGATTCCTGGGTTCGCT |
| | | GAAATGGCTGCACCCCTGTACCCCTTAACAAAA |
| | | ACAGGGACGCTTTTCAACTGGGGGCCAGACCAG |
| | | CAAAAGGCGTATCAGGAGATCAAACAAGCTTTG |
| | | TTGACCGCACCCGCGTTGGGTCTTCCGGATTTA |
| | | ACCAAGCCCTTTGAGCTGTTCGTTGATGAAAAA |
| | | CAGGGATATGCAAAAGGAGTATTAACCCAAAAG |
| | | TTAGGCCCGTGGCGTCGCCCTGTTGCTTACTTG |
| | | AGTAAAAAATTGGATCCTGTCGCAGCAGGATGG |
| | | CCACCGTGCTTGCGTATGGTCGCGGCAATTGCC |
| | | GTTTTGACAAAGGATGCAGGTAAGTTGACGATG |
| | | GGTCAACCCTTACGTATCTTGGCTCCACATGCT |
| | | GTAGAAGCGTTAGTAAAGCAGCCCCCAGACCGC |
| | | TGGCTTTCTAATGCGCGCATGACCCACTATCAG |
| | | GCGCTTCTGCTTGATACGGATCGTGTACAATTT |
| | | GGACCAGTTGTAGCTTTGAATCCAGCTACTTTG |
| | | CTTCCCCTTCCAGAAGAAGGACTTCAGCACAAT |
| | | TGTTTAGATATTCTGGCCGAGGCACATGGGACG |
| | | CGCCCTGATTTGACGGATCAGCCACTGCCTGAT |
| | | GCCGACCATACATGGTATACTGGCGGCAGTAGT |
| | | CTTCTTCAAGAGGGGCAACGCAAGGCGGGAGCA |
| | | GCCGTCACTACGGAGACCGAAGTTATCTGGGCC |
| | | AAAGCGTTACCCGCGGGAACATCCGCGCAACGT |
| | | GCACAGTTAATCGCTCTGACACAGGCCCTGAAG |
| | | ATGGCAGAGGGCAAAAAGTTGAATGTCTACACC |
| | | AACTCACGTTATGCTTTTGCAACAGCGCATGAA |
| | | CATGGCGAAATTTACCGCCGCCGTGGTCTGCTG |
| | | ACTAGTGAGGGTAAGGAAATTAAAAATAAAGAT |
| | | GAGATTCTTGCGTTGTTAAAAGCTTTATTCTTA |
| | | CCAAAACGCCTTTCGATCATTCATTGCCCGGGG |
| | | CATCAAAAGGGTCACTCAGCGGAGGCTCGTGGA |
| | | AACCGTATGGCGGACCAAGCTGCCCGTAAGGCG |
| | | GCGATCACAGAGACCCCGGATACATCAACGCTG |
| | | TTGATCGAAAACAGCTCTCCCTACACTAGCGAG |
| | | CATTTT |
| 727 | MMLV-II | MTLNIEDEHRLHETSKEPDVSLGSTWLSDFPQA |
| | Q68R/Q79R/L99R/ | WAETGGMGLAVRQAPLIIPLKATSTPVSIKQYP |
| | E282D/Q299E/T306K/ | MSREARLGIKPHIRRLLDQGILVPCQSPWNTPL |
| | V433R/I593E | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWLTDARKETVMGQPTPKT |
| | | PRELREFLGKAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLRILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHE |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 728 | MMLV-II | ATGACTTTAAATATTGAGGATGAGCATCGTTTA |
| | Q68R/Q79R/L82Y/ | CATGAGACATCAAAAGAACCCGACGTGAGCTTA |
| | L99R/L280I/E282D/ | GGGTCAACGTGGCTTTCTGACTTCCCCCAGGCG |
| | Q299E/V433R/I593E | TGGGCGGAGACTGGCGGAATGGGGTTAGCTGTC |
| | | CGCCAAGCACCGTTGATCATCCCGTTAAAGGCA |
| | | ACGTCTACACCTGTCTCTATCAAACAGTACCCC |
| | | ATGAGTCGTGAGGCCCGCCTGGGGATTAAGCCA |
| | | CATATTCGTCGCTTGTATGACCAGGGGATCTTG |
| | | GTCCCATGTCAATCTCCGTGGAACACCCCCCTT |
| | | CGTCCCGTGAAAAAGCCAGGTACAAACGATTAT |
| | | CGTCCAGTTCAAGATCTTCGCGAGGTCAACAAA |
| | | CGCGTAGAAGACATCCATCCGACTGTACCTAAT |
| | | CCTTATAATCTGTTATCAGGCCTGCCCCCATCG |
| | | CACCAATGGTATACAGTATTAGACTTGAAAGAC |
| | | GCGTTCTTTTGCCTGCGTCTGCACCCAACGTCT |
| | | CAGCCGCTGTTTGCGTTCGAATGGCGTGATCCT |
| | | GAAATGGGAATTTCGGGTCAGTTAACCTGGACT |
| | | CGTCTGCCCCAGGGCTTTAAAAACAGCCCCACA |
| | | TTGTTCGATGAAGCACTTCACCGTGACTTAGCA |
| | | GACTTCCGTATCCAACACCCAGACTTAATTCTG |
| | | TTACAGTATGTTGACGACCTTTTGTTGGCGGCA |
| | | ACGTCTGAACTTGACTGTCAGCAAGGCACACGC |
| | | GCGTTATTACAAACGTTAGGTAACTTAGGATAT |
| | | CGTGCGTCCGCGAAAAAGGCGCAAATTTGTCAA |
| | | AAACAGGTAAAGTACCTTGGGTATTTGCTGAAA |
| | | GAAGGTCAACGTTGGATTACTGATGCGCGTAAG |
| | | GAGACCGTAATGGGGCAGCCTACGCCTAAGACG |
| | | CCACGCGAATTGCGTGAATTTTTGGGCACAGCG |
| | | GGATTCTGTCGTTTATGGATTCCTGGGTTCGCT |
| | | GAAATGGCTGCACCCCTGTACCCCTTAACAAAA |
| | | ACAGGGACGCTTTTCAACTGGGGGCCAGACCAG |
| | | CAAAAGGCGTATCAGGAGATCAAACAAGCTTTG |
| | | TTGACCGCACCCGCGTTGGGTCTTCCGGATTTA |
| | | ACCAAGCCCTTTGAGCTGTTCGTTGATGAAAAA |
| | | CAGGGATATGCAAAAGGAGTATTAACCCAAAAG |
| | | TTAGGCCCGTGGCGTCGCCCTGTTGCTTACTTG |
| | | AGTAAAAAATTGGATCCTGTCGCAGCAGGATGG |
| | | CCACCGTGCTTGCGTATGGTCGCGGCAATTGCC |
| | | GTTTTGACAAAGGATGCAGGTAAGTTGACGATG |
| | | GGTCAACCCTTACGTATCTTGGCTCCACATGCT |
| | | GTAGAAGCGTTAGTAAAGCAGCCCCCAGACCGC |
| | | TGGCTTTCTAATGCGCGCATGACCCACTATCAG |
| | | GCGCTTCTGCTTGATACGGATCGTGTACAATTT |
| | | GGACCAGTTGTAGCTTTGAATCCAGCTACTTTG |
| | | CTTCCCCTTCCAGAAGAAGGACTTCAGCACAAT |
| | | TGTTTAGATATTCTGGCCGAGGCACATGGGACG |
| | | CGCCCTGATTTGACGGATCAGCCACTGCCTGAT |
| | | GCCGACCATACATGGTATACTGGCGGCAGTAGT |
| | | CTTCTTCAAGAGGGGCAACGCAAGGCGGGAGCA |
| | | GCCGTCACTACGGAGACCGAAGTTATCTGGGCC |
| | | AAAGCGTTACCCGCGGGAACATCCGCGCAACGT |
| | | GCACAGTTAATCGCTCTGACACAGGCCCTGAAG |
| | | ATGGCAGAGGGCAAAAAGTTGAATGTCTACACC |
| | | AACTCACGTTATGCTTTTGCAACAGCGCATGAA |
| | | CATGGCGAAATTTACCGCCGCCGTGGTCTGCTG |
| | | ACTAGTGAGGGTAAGGAAATTAAAAATAAAGAT |
| | | GAGATTCTTGCGTTGTTAAAAGCTTTATTCTTA |
| | | CCAAAACGCCTTTCGATCATTCATTGCCCGGGG |
| | | CATCAAAAGGGTCACTCAGCGGAGGCTCGTGGA |
| | | AACCGTATGGCGGACCAAGCTGCCCGTAAGGCG |
| | | GCGATCACAGAGACCCCGGATACATCAACGCTG |
| | | TTGATCGAAAACAGCTCTCCCTACACTAGCGAG |
| | | CATTTT |
| 729 | MMLV-II | MTLNIEDEHRLHETSKEPDVSLGSTWLSDFPQA |
| | Q68R/Q79R/L82Y/ | WAETGGMGLAVRQAPLIIPLKATSTPVSIKQYP |
| | L99R/L280I/E282D/ | MSREARLGIKPHIRRLYDQGILVPCQSPWNTPL |
| | Q299E/V433R/I593E | RPVKKPGTNDYRPVQDLREVNKRVEDIHPTVPN |
| | | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWITDARKETVMGQPTPKT |
| | | PRELREFLGTAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLRILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHE |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |
| 730 | MMLV-II | ATGACTTTAAATATTGAGGATGAGCATCGTTTA |
| | Q68R/Q79R/L82Y/ | CATGAGACATCAAAAGAACCCGACGTGAGCTTA |
| | L99R/L280I/E282D/ | GGGTCAACGTGGCTTTCTGACTTCCCCCAGGCG |
| | Q299E/T306K/V433R/ | TGGGCGGAGACTGGCGGAATGGGGTTAGCTGTC |
| | I593E | CGCCAAGCACCGTTGATCATCCCGTTAAAGGCA |
| | | ACGTCTACACCTGTCTCTATCAAACAGTACCCC |
| | | ATGAGTCGTGAGGCCCGCCTGGGGATTAAGCCA |
| | | CATATTCGTCGCTTGTATGACCAGGGGATCTTG |
| | | GTCCCATGTCAATCTCCGTGGAACACCCCCCTT |
| | | CGTCCCGTGAAAAAGCCAGGTACAAACGATTAT |
| | | CGTCCAGTTCAAGATCTTCGCGAGGTCAACAAA |
| | | CGCGTAGAAGACATCCATCCGACTGTACCTAAT |
| | | CCTTATAATCTGTTATCAGGCCTGCCCCCATCG |
| | | CACCAATGGTATACAGTATTAGACTTGAAAGAC |
| | | GCGTTCTTTTGCCTGCGTCTGCACCCAACGTCT |
| | | CAGCCGCTGTTTGCGTTCGAATGGCGTGATCCT |
| | | GAAATGGGAATTTCGGGTCAGTTAACCTGGACT |
| | | CGTCTGCCCCAGGGCTTTAAAAACAGCCCCACA |
| | | TTGTTCGATGAAGCACTTCACCGTGACTTAGCA |
| | | GACTTCCGTATCCAACACCCAGACTTAATTCTG |
| | | TTACAGTATGTTGACGACCTTTTGTTGGCGGCA |
| | | ACGTCTGAACTTGACTGTCAGCAAGGCACACGC |
| | | GCGTTATTACAAACGTTAGGTAACTTAGGATAT |
| | | CGTGCGTCCGCGAAAAAGGCGCAAATTTGTCAA |
| | | AAACAGGTAAAGTACCTTGGGTATTTGCTGAAA |
| | | GAAGGTCAACGTTGGATTACTGATGCGCGTAAG |
| | | GAGACCGTAATGGGGCAGCCTACGCCTAAGACG |
| | | CCACGCGAATTGCGTGAATTTTTGGGCAAAGCG |
| | | GGATTCTGTCGTTTATGGATTCCTGGGTTCGCT |
| | | GAAATGGCTGCACCCCTGTACCCCTTAACAAAA |
| | | ACAGGGACGCTTTTCAACTGGGGGCCAGACCAG |
| | | CAAAAGGCGTATCAGGAGATCAAACAAGCTTTG |
| | | TTGACCGCACCCGCGTTGGGTCTTCCGGATTTA |
| | | ACCAAGCCCTTTGAGCTGTTCGTTGATGAAAAA |
| | | CAGGGATATGCAAAAGGAGTATTAACCCAAAAG |
| | | TTAGGCCCGTGGCGTCGCCCTGTTGCTTACTTG |
| | | AGTAAAAAATTGGATCCTGTCGCAGCAGGATGG |
| | | CCACCGTGCTTGCGTATGGTCGCGGCAATTGCC |
| | | GTTTTGACAAAGGATGCAGGTAAGTTGACGATG |
| | | GGTCAACCCTTACGTATCTTGGCTCCACATGCT |
| | | GTAGAAGCGTTAGTAAAGCAGCCCCCAGACCGC |
| | | TGGCTTTCTAATGCGCGCATGACCCACTATCAG |
| | | GCGCTTCTGCTTGATACGGATCGTGTACAATTT |
| | | GGACCAGTTGTAGCTTTGAATCCAGCTACTTTG |
| | | CTTCCCCTTCCAGAAGAAGGACTTCAGCACAAT |
| | | TGTTTAGATATTCTGGCCGAGGCACATGGGACG |
| | | CGCCCTGATTTGACGGATCAGCCACTGCCTGAT |
| | | GCCGACCATACATGGTATACTGGCGGCAGTAGT |
| | | CTTCTTCAAGAGGGGCAACGCAAGGCGGGAGCA |
| | | GCCGTCACTACGGAGACCGAAGTTATCTGGGCC |
| | | AAAGCGTTACCCGCGGGAACATCCGCGCAACGT |
| | | GCACAGTTAATCGCTCTGACACAGGCCCTGAAG |
| | | ATGGCAGAGGGCAAAAAGTTGAATGTCTACACC |
| | | AACTCACGTTATGCTTTTGCAACAGCGCATGAA |
| | | CATGGCGAAATTTACCGCCGCCGTGGTCTGCTG |
| | | ACTAGTGAGGGTAAGGAAATTAAAAATAAAGAT |
| | | GAGATTCTTGCGTTGTTAAAAGCTTTATTCTTA |
| | | CCAAAACGCCTTTCGATCATTCATTGCCCGGGG |
| | | CATCAAAAGGGTCACTCAGCGGAGGCTCGTGGA |
| | | AACCGTATGGCGGACCAAGCTGCCCGTAAGGCG |
| | | GCGATCACAGAGACCCCGGATACATCAACGCTG |
| | | TTGATCGAAAACAGCTCTCCCTACACTAGCGAG |
| | | CATTTT |
| 731 | MMLV-II | MTLNIEDEHRLHETSKEPDVSLGSTWLSDFPQA |
| | Q68R/Q79R/L82Y/ | WAETGGMGLAVRQAPLIIPLKATSTPVSIKQYP |
| | L99R/L280I/E282D/ | MSREARLGIKPHIRRLYDQGILVPCQSPWNTPL |
| | Q299E/T306K/V433R/ | RPVKKPGTNDYRPVQDLREVNKRVEDIHPTVPN |
| | I593E | PYNLLSGLPPSHQWYTVLDLKDAFFCLRLHPTS |
| | | QPLFAFEWRDPEMGISGQLTWTRLPQGFKNSPT |
| | | LFDEALHRDLADFRIQHPDLILLQYVDDLLLAA |
| | | TSELDCQQGTRALLQTLGNLGYRASAKKAQICQ |
| | | KQVKYLGYLLKEGQRWITDARKETVMGQPTPKT |
| | | PRELREFLGKAGFCRLWIPGFAEMAAPLYPLTK |
| | | TGTLFNWGPDQQKAYQEIKQALLTAPALGLPDL |
| | | TKPFELFVDEKQGYAKGVLTQKLGPWRRPVAYL |
| | | SKKLDPVAAGWPPCLRMVAAIAVLTKDAGKLTM |
| | | GQPLRILAPHAVEALVKQPPDRWLSNARMTHYQ |
| | | ALLLDTDRVQFGPVVALNPATLLPLPEEGLQHN |
| | | CLDILAEAHGTRPDLTDQPLPDADHTWYTGGSS |
| | | LLQEGQRKAGAAVTTETEVIWAKALPAGTSAQR |
| | | AQLIALTQALKMAEGKKLNVYTNSRYAFATAHE |
| | | HGEIYRRRGLLTSEGKEIKNKDEILALLKALFL |
| | | PKRLSIIHCPGHQKGHSAEARGNRMADQAARKA |
| | | AITETPDTSTLLIENSSPYTSEHF |

For the standard two-step procedure, RTases (1 μL, 620 nM) were added to a reaction mixture containing RNA (20 ng), dNTPs (100 μM), oligo dT primer (5 ng/uL) or both random hexamers and oligo dT primers (5 ng/uL each), first strand synthesis buffer (1×, 50 mM potassium acetate, 20 mM tris-acetate, pH 7.9, 10 mM magnesium acetate, 0.6 M trehalose 100 μg/ml BSA, and 10 mM DTT), and SuperaseIN (0.17 U/4) in a 20 μL volume. The reaction proceeded at 50 or 65° C. for 15 minutes, followed by 80° C. for 10 minutes.

The subsequent cDNA synthesized by the RTase mutants in this disclosure were quantified by qPCR amplification using an assay that identified the SFRS9 gene in human cells. The assay master mix was a composition of Integrated DNA Technologies PrimeTime® Gene Expression Master Mix (GEM, 1×), SFRS9 primer set (500 nM, Table 3) and SFRS9 probe (250 nM, Table 3). The assay master mix and synthesized cDNA were mixed at a 10:1 ratio for a final volume of 20 μL. The reaction proceeded on a qPCR (QuantStudio7 Flex) using the following method: 95° C. hold for 3 minutes, followed by 95° C. for 15 seconds and 60° C. for one minute for 40 cycles. The reactions were analyzed and reported by Ct value (Tables 23-25). All mutant variants of MMLV RTase showed an increase in the overall activity compared to the base construct and three mutant variants of MMLV RTase showed noteworthy activity compared to the others, Q68R/Q79R/L82Y/L99R/L280I/E282D/Q299E/T306K/V433N/I593W; Q68R/Q79R/L99R/E282D/Q299E/T306K/V433R/I593E; and Q68R/Q79R/L83Y/L99R/L280I/E282D/Q299E/T306K/V433R/I593E.

TABLE 23
Two-Step cDNA Synthesis by MMLV-RT mutants using
oligo dT priming. The data was generated via qPCR
human normalizer assay and data is reported by Ct value.

| | RT | | Ct |
|---|---|---|---|
| | Temperature | Ct | Standard |
| MMLV-RT Variant | (° C.) | Mean | Deviation |
| MMLV-II | 50 | 24.873 | 0.043 |
| | 65 | 35.817 | 0.630 |
| MMLV-II Q68R/Q79R/ | 50 | 24.932 | 0.058 |
| L99R/E282D | 65 | 36.668 | 0.614 |
| MMLV-II Q68R/Q79R/ | 50 | 24.750 | 0.036 |
| L99R/E282D/Q299E/ | 65 | 35.782 | 1.366 |
| V433R/I593E | | | |
| MMLV-II Q68R/Q79R/ | 50 | 24.586 | 0.035 |
| L99R/E282D/Q299E/ | 65 | 35.819 | 0.284 |
| V433N/I593W | | | |
| MMLV-II | 50 | 24.638 | 0.028 |
| Q68R/Q79R/L99R/ | 65 | 34.319 | 0.343 |
| E282D/L280I/Q299E/ | | | |
| V433N/I593W | | | |
| MMLV-II | 50 | 24.681 | 0.019 |
| Q68R/Q79R/L82Y/ | 65 | 33.184 | 0.021 |
| L99R/E282D/L280I/ | | | |
| Q299E/V433N/I593W | | | |

TABLE 24
Two-Step cDNA Synthesis by MMLV-RT mutants using
oligo dT priming. The data was generated via qPCR
human normalizer assay and data is reported by Ct value.

| | RT | | Ct |
|---|---|---|---|
| | Temperature | Ct | Standard |
| MMLV-RT Variant | (° C.) | Mean | Deviation |
| MMLV-II | 50 | 24.887 | 0.041 |
| | 65 | 32.730 | 0.053 |
| MMLV-II Q68R/Q79R/ | 50 | 25.061 | 0.126 |
| L99R/E282D/Q299E/ | 65 | 27.898 | 0.070 |
| V433R/I593E | | | |
| MMLV-II | 50 | 24.849 | 0.101 |
| Q68R/Q79R/L82Y/ | 65 | 26.607 | 0.077 |
| L99R/L280I/E282D/ | | | |
| Q299E/V433N/ | | | |
| I593W | | | |
| MMLV-II | 50 | 25.110 | 0.154 |
| Q68R/Q79R/L82Y/ | 65 | 25.701 | 0.062 |
| L99R/L280I/E282D/ | | | |
| Q299E/T306K/ | | | |
| V433N/I593W | | | |
| MMLV-II | 50 | 24.990 | 0.088 |
| Q68R/Q79R/L99R/ | 65 | 25.929 | 0.114 |
| E282D/Q299E/T306K/ | | | |
| V433R/I593E | | | |
| MMLV-II | 50 | 25.133 | 0.114 |
| Q68R/Q79R/L82Y/ | 65 | 27.032 | 0.141 |
| L99R/L280I/E282D/ | | | |
| Q299E/V433R/I593E | | | |
| MMLV-II | 50 | 24.817 | 0.122 |
| Q68R/Q79R/L82Y/ | 65 | 25.721 | 0.187 |
| L99R/L280I/E282D/ | | | |
| Q299E/T306K/V433R/ | | | |
| I593E | | | |

TABLE 25
Two-Step cDNA Synthesis by MMLV-RT mutants using
random priming. The data was generated via qPCR human
normalizer assay and data is reported by Ct value.

| | RT | | Ct |
|---|---|---|---|
| | Temperature | Ct | Standard |
| MMLV-RT Variant | (° C.) | Mean | Deviation |
| MMLV-II | 50 | 25.048 | 0.075 |
| | 65 | 32.563 | 0.156 |
| MMLV-II | 50 | 25.002 | 0.027 |
| Q68R/Q79R/L99R/ | 65 | 28.062 | 0.106 |
| E282D/Q299E/V433R/ | | | |
| I593E | | | |
| MMLV-II | 50 | 25.016 | 0.179 |
| Q68R/Q79R/L82Y/ | 65 | 26.724 | 0.040 |
| L99R/L280I/E282D/ | | | |
| Q299E/V433N/I593W | | | |
| MMLV-II | 50 | 24.973 | 0.021 |
| Q68R/Q79R/L82Y/ | 65 | 25.732 | 0.061 |
| L99R/L280I/E282D/ | | | |
| Q299E/T306K/V433N/ | | | |
| I593W | | | |
| MMLV-II | 50 | 24.982 | 0.030 |
| Q68R/Q79R/L99R/ | 65 | 26.006 | 0.020 |
| E282D/Q299E/T306K/ | | | |
| V433R/I593E | | | |
| MMLV-II | 50 | 25.078 | 0.065 |
| Q68R/Q79R/L82Y/ | 65 | 27.080 | 0.122 |
| L99R/L280I/E282D/ | | | |
| Q299E/V433R/I593E | | | |
| MMLV-II | 50 | 25.074 | 0.094 |
| Q68R/Q79R/L82Y/ | 65 | 25.784 | 0.100 |
| L99R/L280I/E282D/ | | | |
| Q299E/T306K/V433R/ | | | |
| I593E | | | |

### Example 7. Reverse Transcriptase Mutant Evaluation by Gene Specific Priming

This example demonstrates the procedure used to evaluate each mutant RTase's ability to synthesize cDNA from purified RNA ultramers (Integrated DNA Technologies) compared to the base construct of MMLV RTase. The mutant MMLV RTases were tested by a one-step addition of the RTase in GEM as described in Example 5. The reactions were analyzed and reported by Ct value (Table 26). Twelve mutant variants of MMLV RTase showed an increase in the overall activity compared to the base construct, H77A, D83E, D83R, Y271E, Q299E, G308E, F396A, V433R, I593E, I597A, and I597R.

TABLE 26
One-Step cDNA Synthesis by MMLV-RT single mutants
by gene specific priming. The data was generated via
qPCR human normalizer assay and data is reported by Ct value.

| MMLV-RT Variant | Ct Mean | Ct Standard Deviation |
|---|---|---|
| MMLV-II | 29.065 | 0.277 |
| MMLV-II D209A | 29.583 | 0.166 |
| MMLV-II D209E | 28.900 | 0.088 |
| MMLV-II D209R | 29.266 | 0.068 |
| MMLV-II D83A | 29.588 | 0.082 |
| MMLV-II D83E | 28.499 | 0.087 |
| MMLV-II D83R | 28.724 | 0.087 |
| MMLV-II E201A | 30.692 | 0.173 |
| MMLV-II E201D | 29.130 | 0.157 |
| MMLV-II E201R | 29.333 | 0.141 |
| MMLV-II E367A | 31.153 | 0.021 |
| MMLV-II E367D | 31.070 | 0.187 |
| MMLV-II E367R | 34.221 | 0.475 |
| MMLV-II E596A | 29.150 | 0.121 |
| MMLV-II E596D | 30.494 | 0.081 |
| MMLV-II E596R | 31.787 | 0.227 |
| MMLV-II F210A | 33.639 | 0.196 |
| MMLV-II F210E | 34.982 | 0.065 |
| MMLV-II F210R | 37.201 | 1.986 |
| MMLV-II F369A | 29.055 | 0.063 |
| MMLV-II F369E | 36.856 | 0.508 |
| MMLV-II F369R | 36.149 | 0.308 |
| MMLV-II G308A | 30.226 | 0.170 |
| MMLV-II G308E | 28.772 | 0.121 |
| MMLV-II G308R | 40.000 | 0.000 |
| MMLV-II G331A | 30.412 | 0.137 |
| MMLV-II G331E | 31.321 | 0.160 |
| MMLV-II G331R | 31.340 | 0.020 |
| MMLV-II G73A | 30.741 | 0.125 |
| MMLV-II G73E | 34.319 | 0.369 |
| MMLV-II G73R | 29.721 | 0.061 |
| MMLV-II H77A | 28.581 | 0.070 |
| MMLV-II H77E | 29.475 | 0.107 |
| MMLV-II H77R | 29.726 | 0.120 |
| MMLV-II I125A | 29.812 | 0.043 |
| MMLV-II I125E | 30.712 | 0.147 |
| MMLV-II I125R | 30.324 | 0.012 |
| MMLV-II I212A | 29.586 | 0.086 |
| MMLV-II I212E | 29.459 | 0.073 |
| MMLV-II I212R | 29.037 | 0.092 |
| MMLV-II I593A | 30.560 | 0.101 |
| MMLV-II I593E | 27.779 | 0.056 |
| MMLV-II I593R | 29.268 | 0.012 |
| MMLV-II I597A | 28.983 | 0.024 |
| MMLV-II I597E | 29.583 | 0.143 |
| MMLV-II I597R | 28.671 | 0.103 |
| MMLV-II K285A | 32.375 | 0.158 |
| MMLV-II K285E | 37.065 | 0.044 |
| MMLV-II K285R | 30.564 | 0.075 |
| MMLV-II K348A | 34.241 | 0.516 |
| MMLV-II K348E | 34.533 | 0.432 |
| MMLV-II K348R | 29.703 | 0.225 |
| MMLV-II L198A | 31.900 | 0.054 |
| MMLV-II L198E | 34.193 | 0.167 |
| MMLV-II L198R | 30.819 | 0.077 |
| MMLV-II L280A | 35.724 | 0.175 |
| MMLV-II L280E | 40.000 | 0.000 |
| MMLV-II L280R | 40.000 | 0.000 |
| MMLV-II L352A | 28.936 | 0.043 |
| MMLV-II L352E | 30.177 | 0.059 |
| MMLV-II L352R | 29.371 | 0.063 |
| MMLV-II L357A | 38.802 | 1.694 |
| MMLV-II L357E | 40.000 | 0.000 |
| MMLV-II L357R | 40.000 | 0.000 |
| MMLV-II L82A | 31.245 | 0.035 |
| MMLV-II L82E | 31.384 | 0.122 |
| MMLV-II L82R | 29.682 | 0.116 |
| MMLV-II N335A | 29.668 | 0.086 |
| MMLV-II N335E | 29.113 | 0.058 |
| MMLV-II N335R | 32.323 | 5.429 |
| MMLV-II P76A | 29.463 | 0.123 |
| MMLV-II P76E | 30.030 | 0.163 |
| MMLV-II P76R | 29.443 | 0.028 |
| MMLV-II Q213A | 29.833 | 0.223 |
| MMLV-II Q213E | 29.677 | 0.196 |
| MMLV-II Q213R | 29.704 | 0.053 |
| MMLV-II Q299A | 31.314 | 0.200 |
| MMLV-II Q299E | 28.652 | 0.149 |
| MMLV-II Q299R | 31.711 | 0.062 |
| MMLV-II Q654A | 29.415 | 0.117 |
| MMLV-II Q654E | 30.523 | 0.057 |
| MMLV-II Q654R | 29.523 | 0.052 |
| MMLV-II R205A | 29.140 | 0.138 |
| MMLV-II R205E | 29.356 | 0.179 |
| MMLV-II R205K | 29.162 | 0.206 |
| MMLV-II R211A | 29.491 | 0.025 |
| MMLV-II R211E | 30.049 | 0.205 |
| MMLV-II R211K | 30.196 | 0.147 |
| MMLV-II R311A | 31.237 | 0.425 |
| MMLV-II R311E | 40.000 | 0.000 |
| MMLV-II R311K | 29.857 | 0.091 |
| MMLV-II R389A | 32.173 | 0.151 |
| MMLV-II R389E | 32.717 | 0.105 |
| MMLV-II R389K | 31.944 | 0.166 |
| MMLV-II R650A | 29.734 | 0.060 |
| MMLV-II R650E | 31.012 | 0.074 |
| MMLV-II R650K | 29.404 | 0.094 |
| MMLV-II R657A | 31.470 | 0.133 |
| MMLV-II R657E | 32.785 | 0.145 |
| MMLV-II R657K | 29.468 | 0.274 |
| MMLV-II S67A | 29.268 | 0.090 |
| MMLV-II S67E | 30.157 | 0.254 |
| MMLV-II S67R | 27.274 | 0.054 |
| MMLV-II T328A | 40.000 | 0.000 |
| MMLV-II T328E | 37.699 | 1.627 |
| MMLV-II T328R | 37.169 | 0.848 |
| MMLV-II T332A | 29.219 | 0.075 |
| MMLV-II T332E | 29.714 | 0.057 |
| MMLV-II T332R | 30.462 | 0.130 |
| MMLV-II V129A | 29.305 | 0.077 |
| MMLV-II V129E | 31.188 | 0.181 |
| MMLV-II V129R | 30.383 | 0.081 |
| MMLV-II V433A | 30.483 | 0.059 |
| MMLV-II V433E | 30.106 | 0.144 |
| MMLV-II V433R | 29.297 | 0.457 |
| MMLV-II V476A | 31.295 | 0.244 |
| MMLV-II V476E | 34.664 | 0.364 |
| MMLV-II V476R | 31.223 | 0.166 |
| MMLV-II Y271A | 30.854 | 0.086 |
| MMLV-II Y271E | 28.620 | 0.068 |
| MMLV-II Y271R | 33.280 | 0.258 |
| MMLV-IV | 26.368 | 0.057 |

### Example 8. Further Stacking of Reverse Transcriptase Mutants with Enhanced Activity

This example demonstrates the procedure used to stack the enhanced mutants found in Examples 6 and 7 to further improve the MMLV RTase's ability to synthesize cDNA from purified total RNA (DNased, isolated from HeLa cells) compared to the the base construct and previously found mutant MMLV RTase containing the following mutations: Q68R/Q79R/L99R/E282D. The stacked mutant MMLV RTases were cloned, overexpressed and purified as described in Examples 1 and 2 and tested as described in Examples 6 and 7. Both the two- and one-step reactions were analyzed and reported by Ct value (Tables 27-29). Six of the eight stacked mutant variants of MMLV RTase increased the overall activity and thermostability compared to the base construct, Q68R/Q79R/L99R/E282D/V433R, Q68R/Q79R/L99R/E282D/I593E, Q68R/Q79R/L99R/E282D/Q299E, Q68R/Q79R/L99R/E282D/T332E, Q68R/L82R/L99R/E282D and Q68R/Q79R/L82R/L99R/E282D. Subsequentially, four of those six stacked mutant variants of MMLV RTase increased the overall activity and thermostability compared to the previously identified mutant RTase (Q68R/Q79R/L99R/E282D), Q68R/Q79R/L99R/E282D/I593E, Q68R/Q79R/L99R/E282D/Q299E, Q68R/L82R/L99R/E282D and Q68R/Q79R/L82R/L99R/E282D.

Following these stacked mutant variants, MMLV RTase mutations were stacked further to improve the ability of MMLV RTase to synthesize cDNA from purified total RNA (DNased, isolated from HeLa cells) as compared to the MMLV RTase base construct (RNase H minus construct). Eight MMLV RTase sextuple or more mutant variants were cloned as described in Example 1 and overexpressed and purified as in Example 5.

MMLV RTase base construct and MMLV RTase mutant variants evaluated as described in Example 3. Temperatures were adjusted for both two-step and one-step reactions to 42/55 and 50/60° C., respectively. The two-step first strand synthesis buffer was modified from 50 mM Tris-hydrochloride, pH 8.3, 75 mM potassium chloride, 3 mM magnesium chloride and 10 mM DTT to 50 mM potassium acetate, 20 mM Tris-acetate, pH 7.0, 10 mM magnesium acetate, 100 μg/ml bovine serum albumin and 10 mM DTT. The two-step and one-step reactions for MMLV RTase base construct and MMLV RTase mutant variants were analyzed and reported by Ct output from the qPCR (Tables 27-29).

Four of the eleven MMLV RTase sextuple or more mutant variants were found to exhibit increased overall activity and thermostability as compared to the other MMLV RTase stacked mutant variants, and almost all of the MMLV RTase stacked mutant variants exhibited increased overall activity and thermostability as compared to the MMLV RTase base construct. The four MMLV RTase mutant variants that were found to exhibit the highest overall activity were Q68R/Q79R/L99R/E282D/Q299E/V433R/I593E, Q68R/Q79R/L82R/L99R/E282D/Q299E/V433R/I593E, Q68R/Q79R/L82R/L99R/E282D/Q299E/T332E/I593E, and Q68R/Q79R/L82R/L99R/E282D/Q299E/T332E/V433R/I593E.

TABLE 27
Two-Step cDNA Synthesis by MMLV-RT stacked mutants using
oligo dT priming. The data was generated via qPCR human
normalizer assay and data is reported by Ct value.

| | | Ct Standard |
|---|---|---|
| MMLV-RT Variant | Ct Mean | Deviation |
| MMLV-II | 37.388 | 0.396 |
| MMLV-II Q68R/Q79R/L99R/E282D/V433R | 29.215 | 0.113 |
| MMLV-II Q68R/Q79R/L99R/E282D/I593E | 33.563 | 0.118 |
| MMLV-II Q68R/Q79R/L99R/E282D/Q299E | 31.902 | 0.169 |
| MMLV-II Q68R/Q79R/L99R/E282D/T332E | 33.988 | 0.108 |
| MMLV-II Q68R/Q79R/L99R/L280R | 40.000 | 0.000 |
| MMLV-II Q68R/Q79R/L99R/L280R/E282D | 40.000 | 0.000 |
| MMLV-II Q68R/L82R/L99R/E282D | 39.259 | 1.047 |
| MMLV-II Q68R/Q79R/L82R/L99R/E282D | 30.623 | 0.076 |
| MMLV-IV | 25.880 | 0.023 |

TABLE 28
Two-Step cDNA Synthesis by MMLV-RT stacked mutants
using random priming. The data was generated via qPCR
human normalizer assay and data is reported by Ct value.

| | Ct | Ct Standard |
|---|---|---|
| MMLV-RT Variant | Mean | Deviation |
| MMLV-II | 36.638 | 1.014 |
| MMLV-II Q68R/Q79R/L99R/E282D/V433R | 40.000 | 0.000 |
| MMLV-II Q68R/Q79R/L99R/E282D/I593E | 32.331 | 0.111 |
| MMLV-II Q68R/Q79R/L99R/E282D/Q299E | 30.430 | 0.154 |
| MMLV-II Q68R/Q79R/L99R/E282D/T332E | 33.720 | 0.266 |
| MMLV-II Q68R/Q79R/L99R/L280R | 40.000 | 0.000 |
| MMLV-II Q68R/Q79R/L99R/L280R/E282D | 40.000 | 0.000 |
| MMLV-II Q68R/L82R/L99R/E282D | 35.325 | 0.422 |
| MMLV-II Q68R/Q79R/L82R/L99R/E282D | 31.928 | 0.177 |
| MMLV-IV | 25.840 | 0.049 |

TABLE 29
One-Step cDNA Synthesis by MMLV-RT stacked mutants
by gene specific priming. The data was generated via qPCR
human normalizer assay and data is reported by Ct value.

| | Ct | Ct Standard |
|---|---|---|
| MMLV-RT Variant | Mean | Deviation |
| MMLV-II | 33.027 | 0.048 |
| MMLV-II Q68R/Q79R/L99R/E282D/V433R | 29.937 | 0.040 |
| MMLV-II Q68R/Q79R/L99R/E282D/I593E | 28.724 | 0.081 |
| MMLV-II Q68R/Q79R/L99R/E282D/Q299E | 29.341 | 0.022 |
| MMLV-II Q68R/Q79R/L99R/E282D/T332E | 30.330 | 0.036 |
| MMLV-II Q68R/Q79R/L99R/L280R | 40.000 | 0.000 |
| MMLV-II Q68R/Q79R/L99R/L280R/E282D | 40.000 | 0.000 |
| MMLV-II Q68R/L82R/L99R/E282D | 30.559 | 0.045 |
| MMLV-II Q68R/Q79R/L82R/L99R/E282D | 30.097 | 0.033 |
| MMLV-IV | 28.975 | 0.012 |

a. Evaluation of Ability of Purified MMLV RTase Mutant Variants to Synthesize DNA Over a Wide Range of Temperatures

MMLV RTase base construct MMLV RTase mutant variants evaluated as described in Example 5. Oligo-dT or random hexamer priming conditions and reaction temperatures were adjusted for the two-step reactions and RTase concentration was normalized to 31 nM. The two-step reactions for MMLV RTase base construct and MMLV RTase mutant variants were analyzed and reported by Ct output from the qPCR (see Tables 25 and 26)

Five MMLV RTase mutants were found to exhibit high overall activity as compared to the MMLV RTase base construct over a wide range of temperatures, spanning from 37.0 to 51° C., regardless of which priming method used. All of the MMLV RTase stacked mutant variants exhibited increased overall activity and thermostability as compared to the MMLV RTase base construct. The five MMLV RTas mutant variants that were found to exhibit the highest overall activity at a wide range of temperatures were Q68R/Q79R/L99R/E282D, Q68R/Q79R/L99R/E282D/Q299E/V433R/I593E, Q68R/Q79R/L82R/L99R/E282D/Q299E/V433R/I593E, Q68R/Q79R/L82R/L99R/E282D/Q299E/T332E/I593E and Q68R/Q79R/L82R/L99R/E282D/Q299E/T332E/V433R/I593E

TABLE 30
Two-Step cDNA synthesis by MMLV RT quadruple and
more mutants by Oligo-dT priming. Data was generated via qPCR
human normalizer assay and data is reported by Ct value.

| | Temperature | | |
|---|---|---|---|
| | of Reaction | Ct | Ct |
| MMLV RT Mutant | (° C.) | Mean | SD |
| MMLV-II | 37.0 | 26.340 | 0.033 |
| MMLV-II | 37.8 | 26.130 | 0.061 |
| MMLV-II | 39.5 | 25.830 | 0.014 |
| MMLV-II | 42.0 | 25.753 | 0.041 |
| MMLV-II | 45.2 | 25.632 | 0.077 |
| MMLV-II | 47.8 | 25.935 | 0.026 |
| MMLV-II | 49.2 | 26.478 | 0.042 |
| MMLV-II | 50.0 | 29.461 | 0.120 |
| MMLV-II | 51.0 | 29.430 | 0.098 |
| MMLV-II | 51.9 | 31.123 | 0.066 |
| MMLV-II | 53.8 | 33.632 | 0.073 |
| MMLV-II | 56.5 | 36.499 | 0.385 |
| MMLV-II | 59.9 | 37.158 | 0.427 |
| MMLV-II | 62.6 | 37.464 | 0.440 |
| MMLV-II | 64.2 | 37.082 | 0.022 |
| MMLV-II | 65.0 | 37.518 | 0.370 |
| MMLV-II Q68R/Q79R/L99R/E282D | 37.0 | 25.688 | 0.031 |
| MMLV-II Q68R/Q79R/L99R/E282D | 37.8 | 25.734 | 0.032 |
| MMLV-II Q68R/Q79R/L99R/E282D | 39.5 | 25.613 | 0.040 |
| MMLV-II Q68R/Q79R/L99R/E282D | 42.0 | 25.528 | 0.032 |
| MMLV-II Q68R/Q79R/L99R/E282D | 45.2 | 25.525 | 0.029 |
| MMLV-II Q68R/Q79R/L99R/E282D | 47.8 | 25.471 | 0.105 |
| MMLV-II Q68R/Q79R/L99R/E282D | 49.2 | 25.491 | 0.047 |
| MMLV-II Q68R/Q79R/L99R/E282D | 50.0 | 25.608 | 0.061 |
| MMLV-II Q68R/Q79R/L99R/E282D | 51.0 | 25.679 | 0.006 |
| MMLV-II Q68R/Q79R/L99R/E282D | 51.9 | 25.969 | 0.032 |
| MMLV-II Q68R/Q79R/L99R/E282D | 53.8 | 27.251 | 0.053 |
| MMLV-II Q68R/Q79R/L99R/E282D | 56.5 | 33.619 | 0.195 |
| MMLV-II Q68R/Q79R/L99R/E282D | 59.9 | 36.635 | 0.059 |
| MMLV-II Q68R/Q79R/L99R/E282D | 62.6 | 36.929 | 0.500 |
| MMLV-II Q68R/Q79R/L99R/E282D | 64.2 | 37.515 | 0.478 |
| MMLV-II Q68R/Q79R/L99R/E282D | 65.0 | 37.107 | 0.285 |
| MMLV-II Q68R/Q79R/L99R/E282D/I593E | 37.0 | 26.133 | 0.054 |
| MMLV-II Q68R/Q79R/L99R/E282D/I593E | 37.8 | 26.029 | 0.012 |
| MMLV-II Q68R/Q79R/L99R/E282D/I593E | 39.5 | 25.850 | 0.047 |
| MMLV-II Q68R/Q79R/L99R/E282D/I593E | 42.0 | 25.793 | 0.012 |
| MMLV-II Q68R/Q79R/L99R/E282D/I593E | 45.2 | 25.614 | 0.018 |
| MMLV-II Q68R/Q79R/L99R/E282D/I593E | 47.8 | 25.658 | 0.005 |
| MMLV-II Q68R/Q79R/L99R/E282D/I593E | 49.2 | 25.663 | 0.024 |
| MMLV-II Q68R/Q79R/L99R/E282D/I593E | 50.0 | 25.791 | 0.041 |
| MMLV-II Q68R/Q79R/L99R/E282D/I593E | 51.0 | 25.877 | 0.067 |
| MMLV-II Q68R/Q79R/L99R/E282D/I593E | 51.9 | 26.602 | 0.038 |
| MMLV-II Q68R/Q79R/L99R/E282D/I593E | 53.8 | 29.535 | 0.086 |
| MMLV-II Q68R/Q79R/L99R/E282D/I593E | 56.5 | 35.912 | 0.439 |
| MMLV-II Q68R/Q79R/L99R/E282D/I593E | 59.9 | 37.158 | 0.566 |
| MMLV-II Q68R/Q79R/L99R/E282D/I593E | 62.6 | 37.187 | 0.158 |
| MMLV-II Q68R/Q79R/L99R/E282D/I593E | 64.2 | 37.958 | 0.236 |
| MMLV-II Q68R/Q79R/L99R/E282D/I593E | 65.0 | 36.861 | 0.416 |
| MMLV-II Q68R/Q79R/L99R/E282D/Q299E | 37.0 | 26.106 | 0.070 |
| MMLV-II Q68R/Q79R/L99R/E282D/Q299E | 37.8 | 26.024 | 0.092 |
| MMLV-II Q68R/Q79R/L99R/E282D/Q299E | 39.5 | 25.830 | 0.122 |
| MMLV-II Q68R/Q79R/L99R/E282D/Q299E | 42.0 | 25.788 | 0.025 |
| MMLV-II Q68R/Q79R/L99R/E282D/Q299E | 45.2 | 25.634 | 0.022 |
| MMLV-II Q68R/Q79R/L99R/E282D/Q299E | 47.8 | 25.681 | 0.016 |
| MMLV-II Q68R/Q79R/L99R/E282D/Q299E | 49.2 | 25.684 | 0.029 |
| MMLV-II Q68R/Q79R/L99R/E282D/Q299E | 50.0 | 25.743 | 0.096 |
| MMLV-II Q68R/Q79R/L99R/E282D/Q299E | 51.0 | 25.870 | 0.003 |
| MMLV-II Q68R/Q79R/L99R/E282D/Q299E | 51.9 | 26.301 | 0.033 |
| MMLV-II Q68R/Q79R/L99R/E282D/Q299E | 53.8 | 28.283 | 0.036 |
| MMLV-II Q68R/Q79R/L99R/E282D/Q299E | 56.5 | 34.732 | 0.445 |
| MMLV-II Q68R/Q79R/L99R/E282D/Q299E | 59.9 | 36.947 | 0.407 |
| MMLV-II Q68R/Q79R/L99R/E282D/Q299E | 62.6 | 37.140 | 0.280 |
| MMLV-II Q68R/Q79R/L99R/E282D/Q299E | 64.2 | 37.403 | 0.205 |
| MMLV-II Q68R/Q79R/L99R/E282D/Q299E | 65.0 | 37.347 | 0.438 |
| MMLV-II Q68R/Q79R/L82R/L99R/E282D | 37.0 | 25.961 | 0.170 |
| MMLV-II Q68R/Q79R/L82R/L99R/E282D | 37.8 | 26.065 | 0.085 |
| MMLV-II Q68R/Q79R/L82R/L99R/E282D | 39.5 | 25.909 | 0.028 |
| MMLV-II Q68R/Q79R/L82R/L99R/E282D | 42.0 | 25.802 | 0.055 |
| MMLV-II Q68R/Q79R/L82R/L99R/E282D | 45.2 | 25.632 | 0.087 |
| MMLV-II Q68R/Q79R/L82R/L99R/E282D | 47.8 | 25.728 | 0.065 |
| MMLV-II Q68R/Q79R/L82R/L99R/E282D | 49.2 | 25.612 | 0.165 |
| MMLV-II Q68R/Q79R/L82R/L99R/E282D | 50.0 | 25.795 | 0.038 |
| MMLV-II Q68R/Q79R/L82R/L99R/E282D | 51.0 | 25.830 | 0.009 |
| MMLV-II Q68R/Q79R/L82R/L99R/E282D | 51.9 | 26.477 | 0.037 |
| MMLV-II Q68R/Q79R/L82R/L99R/E282D | 53.8 | 28.496 | 0.040 |
| MMLV-II Q68R/Q79R/L82R/L99R/E282D | 56.5 | 34.329 | 0.177 |
| MMLV-II Q68R/Q79R/L82R/L99R/E282D | 59.9 | 36.564 | 0.315 |
| MMLV-II Q68R/Q79R/L82R/L99R/E282D | 62.6 | 37.152 | 0.322 |
| MMLV-II Q68R/Q79R/L82R/L99R/E282D | 64.2 | 37.340 | 0.585 |
| MMLV-II Q68R/Q79R/L82R/L99R/E282D | 65.0 | 38.351 | 1.016 |
| MMLV-II | 37.0 | 25.853 | 0.057 |
| Q68R/Q79R/L99R/E282D/Q299E/V433R/ | | | |
| I593E | | | |
| MMLV-II | 37.8 | 25.898 | 0.016 |
| Q68R/Q79R/L99R/E282D/Q299E/V433R/ | | | |
| I593E | | | |
| MMLV-II | 39.5 | 25.716 | 0.093 |
| Q68R/Q79R/L99R/E282D/Q299E/V433R/ | | | |
| I593E | | | |
| MMLV-II | 42.0 | 25.669 | 0.064 |
| Q68R/Q79R/L99R/E282D/Q299E/V433R/ | | | |
| I593E | | | |
| MMLV-II | 45.2 | 25.643 | 0.056 |
| Q68R/Q79R/L99R/E282D/Q299E/V433R/ | | | |
| I593E | | | |
| MMLV-II | 47.8 | 25.680 | 0.016 |
| Q68R/Q79R/L99R/E282D/Q299E/V433R/ | | | |
| I593E | | | |
| MMLV-II | 49.2 | 25.663 | 0.057 |
| Q68R/Q79R/L99R/E282D/Q299E/V433R/ | | | |
| I593E | | | |
| MMLV-II | 50.0 | 25.708 | 0.045 |
| Q68R/Q79R/L99R/E282D/Q299E/V433R/ | | | |
| I593E | | | |
| MMLV-II | 51.0 | 25.557 | 0.025 |
| Q68R/Q79R/L99R/E282D/Q299E/V433R/ | | | |
| I593E | | | |
| MMLV-II | 51.9 | 26.015 | 0.125 |
| Q68R/Q79R/L99R/E282D/Q299E/V433R/ | | | |
| I593E | | | |
| MMLV-II | 53.8 | 27.812 | 0.048 |
| Q68R/Q79R/L99R/E282D/Q299E/V433R/ | | | |
| I593E | | | |
| MMLV-II | 56.5 | 34.073 | 0.217 |
| Q68R/Q79R/L99R/E282D/Q299E/V433R/ | | | |
| I593E | | | |
| MMLV-II | 59.9 | 36.512 | 0.168 |
| Q68R/Q79R/L99R/E282D/Q299E/V433R/ | | | |
| I593E | | | |
| MMLV-II | 62.6 | 37.182 | 0.167 |
| Q68R/Q79R/L99R/E282D/Q299E/V433R/ | | | |
| I593E | | | |
| MMLV-II | 64.2 | 37.239 | 0.291 |
| Q68R/Q79R/L99R/E282D/Q299E/V433R/ | | | |
| I593E | | | |
| MMLV-II | 65.0 | 36.573 | 0.232 |
| Q68R/Q79R/L99R/E282D/Q299E/V433R/ | | | |
| I593E | | | |
| MMLV-II | 37.0 | 25.789 | 0.075 |
| Q68R/Q79R/L82R/L99R/E282D/Q299E/ | | | |
| V433R/I593E | | | |
| MMLV-II | 37.8 | 25.784 | 0.103 |
| Q68R/Q79R/L82R/L99R/E282D/Q299E/ | | | |
| V433R/I593E | | | |
| MMLV-II | 39.5 | 25.714 | 0.025 |
| Q68R/Q79R/L82R/L99R/E282D/Q299E/ | | | |
| V433R/I593E | | | |
| MMLV-II | 42.0 | 25.713 | 0.027 |
| Q68R/Q79R/L82R/L99R/E282D/Q299E/ | | | |
| V433R/I593E | | | |
| MMLV-II | 45.2 | 25.690 | 0.030 |
| Q68R/Q79R/L82R/L99R/E282D/Q299E/ | | | |
| V433R/I593E | | | |
| MMLV-II | 47.8 | 25.662 | 0.026 |
| Q68R/Q79R/L82R/L99R/E282D/Q299E/ | | | |
| V433R/I593E | | | |
| MMLV-II | 49.2 | 25.713 | 0.021 |
| Q68R/Q79R/L82R/L99R/E282D/Q299E/ | | | |
| V433R/I593E | | | |
| MMLV-II | 50.0 | 25.551 | 0.092 |
| Q68R/Q79R/L82R/L99R/E282D/Q299E/ | | | |
| V433R/I593E | | | |
| MMLV-II | 51.0 | 25.561 | 0.107 |
| Q68R/Q79R/L82R/L99R/E282D/Q299E/ | | | |
| V433R/I593E | | | |
| MMLV-II | 51.9 | 25.975 | 0.125 |
| Q68R/Q79R/L82R/L99R/E282D/Q299E/ | | | |
| V433R/I593E | | | |
| MMLV-II | 53.8 | 27.556 | 0.023 |
| Q68R/Q79R/L82R/L99R/E282D/Q299E/ | | | |
| V433R/I593E | | | |
| MMLV-II | 56.5 | 33.934 | 0.249 |
| Q68R/Q79R/L82R/L99R/E282D/Q299E/ | | | |
| V433R/I593E | | | |
| MMLV-II | 59.9 | 36.473 | 0.285 |
| Q68R/Q79R/L82R/L99R/E282D/Q299E/ | | | |
| V433R/I593E | | | |
| MMLV-II | 62.6 | 37.411 | 0.377 |
| Q68R/Q79R/L82R/L99R/E282D/Q299E/ | | | |
| V433R/I593E | | | |
| MMLV-II | 64.2 | 37.656 | 0.478 |
| Q68R/Q79R/L82R/L99R/E282D/Q299E/ | | | |
| V433R/I593E | | | |
| MMLV-II | 65.0 | 37.950 | 1.451 |
| Q68R/Q79R/L82R/L99R/E282D/Q299E/ | | | |
| V433R/I593E | | | |
| MMLV-II | 37.0 | 25.788 | 0.028 |
| Q68R/Q79R/L82R/L99R/E282D/Q299E/ | | | |
| T332E/I593E | | | |
| MMLV-II | 37.8 | 25.680 | 0.229 |
| Q68R/Q79R/L82R/L99R/E282D/Q299E/ | | | |
| T332E/I593E | | | |
| MMLV-II | 39.5 | 25.794 | 0.051 |
| Q68R/Q79R/L82R/L99R/E282D/Q299E/ | | | |
| T332E/I593E | | | |
| MMLV-II | 42.0 | 25.415 | 0.270 |
| Q68R/Q79R/L82R/L99R/E282D/Q299E/ | | | |
| T332E/I593E | | | |
| MMLV-II | 45.2 | 25.631 | 0.047 |
| Q68R/Q79R/L82R/L99R/E282D/Q299E/ | | | |
| T332E/I593E | | | |
| MMLV-II | 47.8 | 25.672 | 0.027 |
| Q68R/Q79R/L82R/L99R/E282D/Q299E/ | | | |
| T332E/I593E | | | |
| MMLV-II | 49.2 | 25.792 | 0.045 |
| Q68R/Q79R/L82R/L99R/E282D/Q299E/ | | | |
| T332E/I593E | | | |
| MMLV-II | 50.0 | 25.759 | 0.022 |
| Q68R/Q79R/L82R/L99R/E282D/Q299E/ | | | |
| T332E/I593E | | | |
| MMLV-II | 51.0 | 25.852 | 0.015 |
| Q68R/Q79R/L82R/L99R/E282D/Q299E/ | | | |
| T332E/I593E | | | |
| MMLV-II | 51.9 | 26.425 | 0.033 |
| Q68R/Q79R/L82R/L99R/E282D/Q299E/ | | | |
| T332E/I593E | | | |
| MMLV-II | 53.8 | 29.964 | 0.023 |
| Q68R/Q79R/L82R/L99R/E282D/Q299E/ | | | |
| T332E/I593E | | | |
| MMLV-II | 56.5 | 36.532 | 0.113 |
| Q68R/Q79R/L82R/L99R/E282D/Q299E/ | | | |
| T332E/I593E | | | |
| MMLV-II | 59.9 | 38.246 | 0.608 |
| Q68R/Q79R/L82R/L99R/E282D/Q299E/ | | | |
| T332E/I593E | | | |
| MMLV-II | 62.6 | 37.333 | 0.446 |
| Q68R/Q79R/L82R/L99R/E282D/Q299E/ | | | |
| T332E/I593E | | | |
| MMLV-II | 64.2 | 37.223 | 0.212 |
| Q68R/Q79R/L82R/L99R/E282D/Q299E/ | | | |
| T332E/I593E | | | |
| MMLV-II | 65.0 | 36.930 | 0.527 |
| Q68R/Q79R/L82R/L99R/E282D/Q299E/ | | | |
| T332E/I593E | | | |
| MMLV-II | 37.0 | 25.863 | 0.014 |
| Q68R/Q79R/L82R/L99R/E282D/Q299E/ | | | |
| T332E/V433R/I593E | | | |
| MMLV-II | 37.8 | 25.649 | 0.036 |
| Q68R/Q79R/L82R/L99R/E282D/Q299E/ | | | |
| T332E/V433R/I593E | | | |
| MMLV-II | 39.5 | 25.573 | 0.057 |
| Q68R/Q79R/L82R/L99R/E282D/Q299E/ | | | |
| T332E/V433R/I593E | | | |
| MMLV-II | 42.0 | 25.453 | 0.023 |
| Q68R/Q79R/L82R/L99R/E282D/Q299E/ | | | |
| T332E/V433R/I593E | | | |
| MMLV-II | 45.2 | 25.447 | 0.083 |
| Q68R/Q79R/L82R/L99R/E282D/Q299E/ | | | |
| T332E/V433R/I593E | | | |
| MMLV-II | 47.8 | 25.413 | 0.061 |
| Q68R/Q79R/L82R/L99R/E282D/Q299E/ | | | |
| T332E/V433R/I593E | | | |
| MMLV-II | 49.2 | 25.542 | 0.035 |
| Q68R/Q79R/L82R/L99R/E282D/Q299E/ | | | |
| T332E/V433R/I593E | | | |
| MMLV-II | 50.0 | 25.567 | 0.060 |
| Q68R/Q79R/L82R/L99R/E282D/Q299E/ | | | |
| T332E/V433R/I593E | | | |
| MMLV-II | 51.0 | 25.741 | 0.093 |
| Q68R/Q79R/L82R/L99R/E282D/Q299E/ | | | |
| T332E/V433R/I593E | | | |
| MMLV-II | 51.9 | 26.231 | 0.225 |
| Q68R/Q79R/L82R/L99R/E282D/Q299E/ | | | |
| T332E/V433R/I593E | | | |
| MMLV-II | 53.8 | 28.556 | 0.142 |
| Q68R/Q79R/L82R/L99R/E282D/Q299E/ | | | |
| T332E/V433R/I593E | | | |
| MMLV-II | 56.5 | 35.202 | 0.208 |
| Q68R/Q79R/L82R/L99R/E282D/Q299E/ | | | |
| T332E/V433R/I593E | | | |
| MMLV-II | 59.9 | 36.991 | 0.419 |
| Q68R/Q79R/L82R/L99R/E282D/Q299E/ | | | |
| T332E/V433R/I593E | | | |
| MMLV-II | 62.6 | 37.168 | 0.463 |
| Q68R/Q79R/L82R/L99R/E282D/Q299E/ | | | |
| T332E/V433R/I593E | | | |
| MMLV-II | 64.2 | 37.670 | 0.410 |
| Q68R/Q79R/L82R/L99R/E282D/Q299E/ | | | |
| T332E/V433R/I593E | | | |
| MMLV-II | 65.0 | 37.680 | 0.273 |
| Q68R/Q79R/L82R/L99R/E282D/Q299E/ | | | |
| T332E/V433R/I593E | | | |

TABLE 31
Two-Step cDNA synthesis by MMLV RT quadruple and
more mutants by Random priming. Data was generated via qPCR
human normalizer assay and data is reported by Ct value.

| | Temperature | | |
|---|---|---|---|
| | of Reaction | Ct | Ct |
| MMLV RT Mutant | (° C.) | Mean | SD |
| MMLV-II | 37.0 | 26.365 | 0.066 |
| MMLV-II | 37.8 | 26.390 | 0.006 |
| MMLV-II | 39.5 | 25.939 | 0.016 |
| MMLV-II | 42.0 | 25.798 | 0.029 |
| MMLV-II | 45.2 | 25.849 | 0.064 |
| MMLV-II | 47.8 | 26.647 | 0.050 |
| MMLV-II | 49.2 | 28.326 | 0.028 |
| MMLV-II | 50.0 | 29.340 | 0.010 |
| MMLV-II | 51.0 | 30.684 | 0.099 |
| MMLV-II | 51.9 | 32.462 | 0.163 |
| MMLV-II | 53.8 | 33.855 | 0.307 |
| MMLV-II | 56.5 | 35.376 | 0.461 |
| MMLV-II | 59.9 | 36.098 | 0.481 |
| MMLV-II | 62.6 | 36.391 | 0.367 |
| MMLV-II | 64.2 | 36.442 | 0.547 |
| MMLV-II | 65.0 | 35.871 | 0.301 |
| MMLV-II Q68R/Q79R/L99R/E282D | 37.0 | 25.699 | 0.009 |
| MMLV-II Q68R/Q79R/L99R/E282D | 37.8 | 25.674 | 0.038 |
| MMLV-II Q68R/Q79R/L99R/E282D | 39.5 | 25.594 | 0.029 |
| MMLV-II Q68R/Q79R/L99R/E282D | 42.0 | 25.496 | 0.016 |
| MMLV-II Q68R/Q79R/L99R/E282D | 45.2 | 25.431 | 0.011 |
| MMLV-II Q68R/Q79R/L99R/E282D | 47.8 | 25.420 | 0.036 |
| MMLV-II Q68R/Q79R/L99R/E282D | 49.2 | 25.481 | 0.023 |
| MMLV-II Q68R/Q79R/L99R/E282D | 50.0 | 25.646 | 0.035 |
| MMLV-II Q68R/Q79R/L99R/E282D | 51.0 | 25.979 | 0.012 |
| MMLV-II Q68R/Q79R/L99R/E282D | 51.9 | 26.591 | 0.053 |
| MMLV-II Q68R/Q79R/L99R/E282D | 53.8 | 28.345 | 0.091 |
| MMLV-II Q68R/Q79R/L99R/E282D | 56.5 | 32.976 | 0.109 |
| MMLV-II Q68R/Q79R/L99R/E282D | 59.9 | 34.407 | 0.158 |
| MMLV-II Q68R/Q79R/L99R/E282D | 62.6 | 35.130 | 0.014 |
| MMLV-II Q68R/Q79R/L99R/E282D | 64.2 | 34.866 | 0.258 |
| MMLV-II Q68R/Q79R/L99R/E282D | 65.0 | 35.317 | 0.299 |
| MMLV-II Q68R/Q79R/L99R/E282D/I593E | 37.0 | 26.079 | 0.036 |
| MMLV-II Q68R/Q79R/L99R/E282D/I593E | 37.8 | 25.951 | 0.015 |
| MMLV-II Q68R/Q79R/L99R/E282D/I593E | 39.5 | 25.801 | 0.055 |
| MMLV-II Q68R/Q79R/L99R/E282D/I593E | 42.0 | 25.602 | 0.087 |
| MMLV-II Q68R/Q79R/L99R/E282D/I593E | 45.2 | 25.424 | 0.038 |
| MMLV-II Q68R/Q79R/L99R/E282D/I593E | 47.8 | 25.520 | 0.011 |
| MMLV-II Q68R/Q79R/L99R/E282D/I593E | 49.2 | 25.674 | 0.046 |
| MMLV-II Q68R/Q79R/L99R/E282D/I593E | 50.0 | 25.922 | 0.015 |
| MMLV-II Q68R/Q79R/L99R/E282D/I593E | 51.0 | 26.351 | 0.014 |
| MMLV-II Q68R/Q79R/L99R/E282D/I593E | 51.9 | 27.411 | 0.092 |
| MMLV-II Q68R/Q79R/L99R/E282D/I593E | 53.8 | 30.482 | 0.048 |
| MMLV-II Q68R/Q79R/L99R/E282D/I593E | 56.5 | 33.914 | 0.075 |
| MMLV-II Q68R/Q79R/L99R/E282D/I593E | 59.9 | 35.443 | 0.191 |
| MMLV-II Q68R/Q79R/L99R/E282D/I593E | 62.6 | 35.872 | 0.445 |
| MMLV-II Q68R/Q79R/L99R/E282D/I593E | 64.2 | 36.107 | 0.011 |
| MMLV-II Q68R/Q79R/L99R/E282D/I593E | 65.0 | 35.715 | 0.299 |
| MMLV-II Q68R/Q79R/L99R/E282D/Q299E | 37.0 | 25.955 | 0.040 |
| MMLV-II Q68R/Q79R/L99R/E282D/Q299E | 37.8 | 25.934 | 0.023 |
| MMLV-II Q68R/Q79R/L99R/E282D/Q299E | 39.5 | 25.669 | 0.035 |
| MMLV-II Q68R/Q79R/L99R/E282D/Q299E | 42.0 | 25.523 | 0.016 |
| MMLV-II Q68R/Q79R/L99R/E282D/Q299E | 45.2 | 25.532 | 0.054 |
| MMLV-II Q68R/Q79R/L99R/E282D/Q299E | 47.8 | 25.550 | 0.021 |
| MMLV-II Q68R/Q79R/L99R/E282D/Q299E | 49.2 | 25.620 | 0.030 |
| MMLV-II Q68R/Q79R/L99R/E282D/Q299E | 50.0 | 25.711 | 0.035 |
| MMLV-II Q68R/Q79R/L99R/E282D/Q299E | 51.0 | 26.215 | 0.056 |
| MMLV-II Q68R/Q79R/L99R/E282D/Q299E | 51.9 | 26.969 | 0.013 |
| MMLV-II Q68R/Q79R/L99R/E282D/Q299E | 53.8 | 29.622 | 0.060 |
| MMLV-II Q68R/Q79R/L99R/E282D/Q299E | 56.5 | 33.679 | 0.234 |
| MMLV-II Q68R/Q79R/L99R/E282D/Q299E | 59.9 | 35.253 | 0.144 |
| MMLV-II Q68R/Q79R/L99R/E282D/Q299E | 62.6 | 35.408 | 0.441 |
| MMLV-II Q68R/Q79R/L99R/E282D/Q299E | 64.2 | 35.586 | 0.139 |
| MMLV-II Q68R/Q79R/L99R/E282D/Q299E | 65.0 | 36.076 | 0.700 |
| MMLV-II Q68R/Q79R/L82R/L99R/E282D | 37.0 | 25.884 | 0.012 |
| MMLV-II Q68R/Q79R/L82R/L99R/E282D | 37.8 | 25.833 | 0.009 |
| MMLV-II Q68R/Q79R/L82R/L99R/E282D | 39.5 | 25.684 | 0.077 |
| MMLV-II Q68R/Q79R/L82R/L99R/E282D | 42.0 | 25.553 | 0.026 |
| MMLV-II Q68R/Q79R/L82R/L99R/E282D | 45.2 | 25.471 | 0.043 |
| MMLV-II Q68R/Q79R/L82R/L99R/E282D | 47.8 | 25.491 | 0.085 |
| MMLV-II Q68R/Q79R/L82R/L99R/E282D | 49.2 | 25.646 | 0.014 |
| MMLV-II Q68R/Q79R/L82R/L99R/E282D | 50.0 | 25.765 | 0.039 |
| MMLV-II Q68R/Q79R/L82R/L99R/E282D | 51.0 | 26.365 | 0.044 |
| MMLV-II Q68R/Q79R/L82R/L99R/E282D | 51.9 | 27.170 | 0.071 |
| MMLV-II Q68R/Q79R/L82R/L99R/E282D | 53.8 | 29.662 | 0.048 |
| MMLV-II Q68R/Q79R/L82R/L99R/E282D | 56.5 | 33.853 | 0.162 |
| MMLV-II Q68R/Q79R/L82R/L99R/E282D | 59.9 | 34.899 | 0.325 |
| MMLV-II Q68R/Q79R/L82R/L99R/E282D | 62.6 | 35.557 | 0.145 |
| MMLV-II Q68R/Q79R/L82R/L99R/E282D | 64.2 | 35.360 | 0.222 |
| MMLV-II Q68R/Q79R/L82R/L99R/E282D | 65.0 | 35.614 | 0.403 |
| MMLV-II | 37.0 | 25.706 | 0.031 |
| Q68R/Q79R/L99R/E282D/Q299E/V433R/I593E | | | |
| MMLV-II | 37.8 | 25.757 | 0.101 |
| Q68R/Q79R/L99R/E282D/Q299E/V433R/I593E | | | |
| MMLV-II | 39.5 | 25.435 | 0.036 |
| Q68R/Q79R/L99R/E282D/Q299E/V433R/I593E | | | |
| MMLV-II | 42.0 | 25.417 | 0.025 |
| Q68R/Q79R/L99R/E282D/Q299E/V433R/I593E | | | |
| MMLV-II | 45.2 | 25.425 | 0.023 |
| Q68R/Q79R/L99R/E282D/Q299E/V433R/I593E | | | |
| MMLV-II | 47.8 | 25.401 | 0.049 |
| Q68R/Q79R/L99R/E282D/Q299E/V433R/I593E | | | |
| MMLV-II | 49.2 | 25.467 | 0.009 |
| Q68R/Q79R/L99R/E282D/Q299E/V433R/I593E | | | |
| MMLV-II | 50.0 | 25.516 | 0.056 |
| Q68R/Q79R/L99R/E282D/Q299E/V433R/I593E | | | |
| MMLV-II | 51.0 | 25.880 | 0.039 |
| Q68R/Q79R/L99R/E282D/Q299E/V433R/I593E | | | |
| MMLV-II | 51.9 | 26.348 | 0.064 |
| Q68R/Q79R/L99R/E282D/Q299E/V433R/I593E | | | |
| MMLV-II | 53.8 | 28.506 | 0.018 |
| Q68R/Q79R/L99R/E282D/Q299E/V433R/I593E | | | |
| MMLV-II | 56.5 | 32.812 | 0.242 |
| Q68R/Q79R/L99R/E282D/Q299E/V433R/I593E | | | |
| MMLV-II | 59.9 | 34.123 | 0.163 |
| Q68R/Q79R/L99R/E282D/Q299E/V433R/I593E | | | |
| MMLV-II | 62.6 | 35.108 | 0.027 |
| Q68R/Q79R/L99R/E282D/Q299E/V433R/I593E | | | |
| MMLV-II | 64.2 | 34.796 | 0.171 |
| Q68R/Q79R/L99R/E282D/Q299E/V433R/I593E | | | |
| MMLV-II | 65.0 | 34.999 | 0.064 |
| Q68R/Q79R/L99R/E282D/Q299E/V433R/I593E | | | |
| MMLV-II | 37.0 | 25.711 | 0.080 |
| Q68R/Q79R/L82R/L99R/E282D/Q299E/V433R/I593E | | | |
| MMLV-II | 37.8 | 25.916 | 0.224 |
| Q68R/Q79R/L82R/L99R/E282D/Q299E/V433R/I593E | | | |
| MMLV-II | 39.5 | 25.665 | 0.052 |
| Q68R/Q79R/L82R/L99R/E282D/Q299E/V433R/I593E | | | |
| MMLV-II | 42.0 | 25.527 | 0.016 |
| Q68R/Q79R/L82R/L99R/E282D/Q299E/V433R/I593E | | | |
| MMLV-II | 45.2 | 25.504 | 0.065 |
| Q68R/Q79R/L82R/L99R/E282D/Q299E/V433R/I593E | | | |
| MMLV-II | 47.8 | 25.437 | 0.070 |
| Q68R/Q79R/L82R/L99R/E282D/Q299E/V433R/I593E | | | |
| MMLV-II | 49.2 | 25.555 | 0.065 |
| Q68R/Q79R/L82R/L99R/E282D/Q299E/V433R/I593E | | | |
| MMLV-II | 50.0 | 25.571 | 0.028 |
| Q68R/Q79R/L82R/L99R/E282D/Q299E/V433R/I593E | | | |
| MMLV-II | 51.0 | 25.854 | 0.029 |
| Q68R/Q79R/L82R/L99R/E282D/Q299E/V433R/I593E | | | |
| MMLV-II | 51.9 | 26.259 | 0.057 |
| Q68R/Q79R/L82R/L99R/E282D/Q299E/V433R/I593E | | | |
| MMLV-II | 53.8 | 28.329 | 0.053 |
| Q68R/Q79R/L82R/L99R/E282D/Q299E/V433R/I593E | | | |
| MMLV-II | 56.5 | 32.962 | 0.212 |
| Q68R/Q79R/L82R/L99R/E282D/Q299E/V433R/I593E | | | |
| MMLV-II | 59.9 | 34.072 | 0.446 |
| Q68R/Q79R/L82R/L99R/E282D/Q299E/V433R/I593E | | | |
| MMLV-II | 62.6 | 34.931 | 0.205 |
| Q68R/Q79R/L82R/L99R/E282D/Q299E/V433R/I593E | | | |
| MMLV-II | 64.2 | 34.626 | 0.169 |
| Q68R/Q79R/L82R/L99R/E282D/Q299E/V433R/I593E | | | |
| MMLV-II | 65.0 | 35.085 | 0.230 |
| Q68R/Q79R/L82R/L99R/E282D/Q299E/V433R/I593E | | | |
| MMLV-II | 37.0 | 25.940 | 0.130 |
| Q68R/Q79R/L82R/L99R/E282D/Q299E/T332E/I593E | | | |
| MMLV-II | 37.8 | 25.793 | 0.129 |
| Q68R/Q79R/L82R/L99R/E282D/Q299E/T332E/I593E | | | |
| MMLV-II | 39.5 | 25.599 | 0.015 |
| Q68R/Q79R/L82R/L99R/E282D/Q299E/T332E/I593E | | | |
| MMLV-II | 42.0 | 25.504 | 0.016 |
| Q68R/Q79R/L82R/L99R/E282D/Q299E/T332E/I593E | | | |
| MMLV-II | 45.2 | 25.602 | 0.041 |
| Q68R/Q79R/L82R/L99R/E282D/Q299E/T332E/I593E | | | |
| MMLV-II | 47.8 | 25.604 | 0.058 |
| Q68R/Q79R/L82R/L99R/E282D/Q299E/T332E/I593E | | | |
| MMLV-II | 49.2 | 25.665 | 0.007 |
| Q68R/Q79R/L82R/L99R/E282D/Q299E/T332E/I593E | | | |
| MMLV-II | 50.0 | 25.821 | 0.068 |
| Q68R/Q79R/L82R/L99R/E282D/Q299E/T332E/I593E | | | |
| MMLV-II | 51.0 | 26.315 | 0.047 |
| Q68R/Q79R/L82R/L99R/E282D/Q299E/T332E/I593E | | | |
| MMLV-II | 51.9 | 27.036 | 0.059 |
| Q68R/Q79R/L82R/L99R/E282D/Q299E/T332E/I593E | | | |
| MMLV-II | 53.8 | 31.004 | 0.089 |
| Q68R/Q79R/L82R/L99R/E282D/Q299E/T332E/I593E | | | |
| MMLV-II | 56.5 | 33.765 | 0.274 |
| Q68R/Q79R/L82R/L99R/E282D/Q299E/T332E/I593E | | | |
| MMLV-II | 59.9 | 34.656 | 0.209 |
| Q68R/Q79R/L82R/L99R/E282D/Q299E/T332E/I593E | | | |
| MMLV-II | 62.6 | 35.561 | 0.468 |
| Q68R/Q79R/L82R/L99R/E282D/Q299E/T332E/I593E | | | |
| MMLV-II | 64.2 | 35.877 | 0.154 |
| Q68R/Q79R/L82R/L99R/E282D/Q299E/T332E/I593E | | | |
| MMLV-II | 65.0 | 35.659 | 0.477 |
| Q68R/Q79R/L82R/L99R/E282D/Q299E/T332E/I593E | | | |
| MMLV-II | 37.0 | 25.780 | 0.046 |
| Q68R/Q79R/L82R/L99R/E282D/Q299E/T332E/V433R/ | | | |
| I593E | | | |
| MMLV-II | 37.8 | 25.652 | 0.026 |
| Q68R/Q79R/L82R/L99R/E282D/Q299E/T332E/V433R/ | | | |
| I593E | | | |
| MMLV-II | 39.5 | 25.641 | 0.037 |
| Q68R/Q79R/L82R/L99R/E282D/Q299E/T332E/V433R/ | | | |
| I593E | | | |
| MMLV-II | 42.0 | 25.507 | 0.005 |
| Q68R/Q79R/L82R/L99R/E282D/Q299E/T332E/V433R/ | | | |
| I593E | | | |
| MMLV-II | 45.2 | 25.484 | 0.067 |
| Q68R/Q79R/L82R/L99R/E282D/Q299E/T332E/V433R/ | | | |
| I593E | | | |
| MMLV-II | 47.8 | 25.438 | 0.027 |
| Q68R/Q79R/L82R/L99R/E282D/Q299E/T332E/V433R/ | | | |
| I593E | | | |
| MMLV-II | 49.2 | 25.534 | 0.022 |
| Q68R/Q79R/L82R/L99R/E282D/Q299E/T332E/V433R/ | | | |
| I593E | | | |
| MMLV-II | 50.0 | 25.755 | 0.085 |
| Q68R/Q79R/L82R/L99R/E282D/Q299E/T332E/V433R/ | | | |
| I593E | | | |
| MMLV-II | 51.0 | 25.981 | 0.027 |
| Q68R/Q79R/L82R/L99R/E282D/Q299E/T332E/V433R/ | | | |
| I593E | | | |
| MMLV-II | 51.9 | 26.242 | 0.052 |
| Q68R/Q79R/L82R/L99R/E282D/Q299E/T332E/V433R/ | | | |
| I593E | | | |
| MMLV-II | 53.8 | 29.146 | 0.069 |
| Q68R/Q79R/L82R/L99R/E282D/Q299E/T332E/V433R/ | | | |
| I593E | | | |
| MMLV-II | 56.5 | 33.138 | 0.159 |
| Q68R/Q79R/L82R/L99R/E282D/Q299E/T332E/V433R/ | | | |
| I593E | | | |
| MMLV-II | 59.9 | 34.551 | 0.152 |
| Q68R/Q79R/L82R/L99R/E282D/Q299E/T332E/V433R/ | | | |
| I593E | | | |
| MMLV-II | 62.6 | 35.186 | 0.322 |
| Q68R/Q79R/L82R/L99R/E282D/Q299E/T332E/V433R/ | | | |
| I593E | | | |
| MMLV-II | 64.2 | 35.550 | 0.368 |
| Q68R/Q79R/L82R/L99R/E282D/Q299E/T332E/V433R/ | | | |
| I593E | | | |
| MMLV-II | 65.0 | 35.459 | 0.295 |
| Q68R/Q79R/L82R/L99R/E282D/Q299E/T332E/V433R/ | | | |
| I593E | | | |

### Example 9: Extension of Reverse Transcriptase Single Mutants

The amino acid positions that enclosed the MMLV RTase single mutants identified in Examples 6 and 7 were further evaluated to include all possible amino acid substitutions at that position. The single mutants were cloned, overexpressed, and purified as described in Examples 1 and 2, and evaluated as described in Examples 6 and 7. The two-step and one-step reactions for MMLV RTase base construct and MMLV RTase double mutant variants were analyzed and reported by Ct output from the qPCR (Tables 32-34). Numerous single mutant MMLV RTase variants were found to exhibit an increase in the overall activity and thermostability as compared to the MMLV RTase base construct. The most prevalent among these were: L82F, L82K, L82T, L82Y, L280I, T332V, V433K, V433N, and I593W.

TABLE 32
Two-Step cDNA Synthesis by MMLV-RT single mutants using
Oligo-dT priming. The data was generated via qPCR human
normalizer assay and data is reported by Ct value.

| MMLV-RT Variant | Ct Mean | Ct Standard Deviation |
|---|---|---|
| MMLV-II | 40.000 | 0.000 |
| MMLV-II I593A | 40.000 | 0.000 |
| MMLV-II I593C | 37.874 | 0.991 |
| MMLV-II I593D | 40.000 | 0.000 |
| MMLV-II I593E | 40.000 | 0.000 |
| MMLV-II I593F | 40.000 | 0.000 |
| MMLV-II 1593G | 39.748 | 0.356 |
| MMLV-II I593H | 39.502 | 0.704 |
| MMLV-II I593K | 40.000 | 0.000 |
| MMLV-II I593L | 38.994 | 1.423 |
| MMLV-II I593M | 39.383 | 0.873 |
| MMLV-II I593N | 40.000 | 0.000 |
| MMLV-II I593P | 40.000 | 0.000 |
| MMLV-II I593Q | 40.000 | 0.000 |
| MMLV-II I593R | 40.000 | 0.000 |
| MMLV-II I593S | 39.614 | 0.545 |
| MMLV-II I593T | 37.709 | 0.520 |
| MMLV-II I593V | 40.000 | 0.000 |
| MMLV-II I593W | 30.504 | 0.073 |
| MMLV-II I593Y | 40.000 | 0.000 |
| MMLV-II L280A | 40.000 | 0.000 |
| MMLV-II L280C | 40.000 | 0.000 |
| MMLV-II L280D | 40.000 | 0.000 |
| MMLV-II L280E | 40.000 | 0.000 |
| MMLV-II L280F | 40.000 | 0.000 |
| MMLV-II L280G | 40.000 | 0.000 |
| MMLV-II L280H | 40.000 | 0.000 |
| MMLV-II L280I | 30.951 | 0.076 |
| MMLV-II L280K | 40.000 | 0.000 |
| MMLV-II L280M | 40.000 | 0.000 |
| MMLV-II L280N | 39.727 | 0.386 |
| MMLV-II L280P | 40.000 | 0.000 |
| MMLV-II L280Q | 40.000 | 0.000 |
| MMLV-II L280R | 39.994 | 0.009 |
| MMLV-II L280S | 40.000 | 0.000 |
| MMLV-II L280T | 40.000 | 0.000 |
| MMLV-II L280V | 37.749 | 0.142 |
| MMLV-II L280W | 40.000 | 0.000 |
| MMLV-II L280Y | 40.000 | 0.000 |
| MMLV-II L82A | 40.000 | 0.000 |
| MMLV-II L82C | 39.565 | 0.615 |
| MMLV-II L82D | 40.000 | 0.000 |
| MMLV-II L82E | 40.000 | 0.000 |
| MMLV-II L82F | 39.347 | 0.924 |
| MMLV-II L82G | 40.000 | 0.000 |
| MMLV-II L82H | 40.000 | 0.000 |
| MMLV-II L82I | 40.000 | 0.000 |
| MMLV-II L82K | 37.136 | 0.593 |
| MMLV-II L82M | 38.649 | 1.260 |
| MMLV-II L82N | 40.000 | 0.000 |
| MMLV-II L82P | 40.000 | 0.000 |
| MMLV-II L82Q | 39.098 | 1.275 |
| MMLV-II L82R | 40.000 | 0.000 |
| MMLV-II L82S | 39.346 | 0.925 |
| MMLV-II L82T | 38.695 | 1.845 |
| MMLV-II L82V | 38.047 | 1.381 |
| MMLV-II L82W | 37.151 | 0.308 |
| MMLV-II L82Y | 35.014 | 0.421 |
| MMLV-II Q299A | 40.000 | 0.000 |
| MMLV-II Q299C | 40.000 | 0.000 |
| MMLV-II Q299D | 40.000 | 0.000 |
| MMLV-II Q299E | 39.061 | 1.328 |
| MMLV-II Q299F | 40.000 | 0.000 |
| MMLV-II Q299G | 40.000 | 0.000 |
| MMLV-II Q299H | 39.398 | 0.852 |
| MMLV-II Q299I | 39.183 | 1.155 |
| MMLV-II Q299K | 40.000 | 0.000 |
| MMLV-II Q299L | 39.474 | 0.743 |
| MMLV-II Q299M | 40.000 | 0.000 |
| MMLV-II Q299N | 40.000 | 0.000 |
| MMLV-II Q299P | 40.000 | 0.000 |
| MMLV-II Q299R | 40.000 | 0.000 |
| MMLV-II Q299S | 40.000 | 0.000 |
| MMLV-II Q299T | 40.000 | 0.000 |
| MMLV-II Q299V | 40.000 | 0.000 |
| MMLV-II Q299W | 40.000 | 0.000 |
| MMLV-II Q299Y | 40.000 | 0.000 |
| MMLV-II T332A | 39.087 | 1.291 |
| MMLV-II T332C | 38.956 | 1.476 |
| MMLV-II T332D | 40.000 | 0.000 |
| MMLV-II T332E | 39.554 | 0.631 |
| MMLV-II T332F | 40.000 | 0.000 |
| MMLV-II T332G | 37.321 | 2.009 |
| MMLV-II T332H | 39.215 | 1.110 |
| MMLV-II T332I | 39.344 | 0.927 |
| MMLV-II T332K | 40.000 | 0.000 |
| MMLV-II T332L | 40.000 | 0.000 |
| MMLV-II T332M | 37.775 | 1.632 |
| MMLV-II T332N | 37.326 | 0.834 |
| MMLV-II T332P | 40.000 | 0.000 |
| MMLV-II T332Q | 39.509 | 0.694 |
| MMLV-II T332R | 39.588 | 0.582 |
| MMLV-II T332S | 39.765 | 0.332 |
| MMLV-II T332V | 36.977 | 0.384 |
| MMLV-II T332W | 40.000 | 0.000 |
| MMLV-II T332Y | 40.000 | 0.000 |
| MMLV-II V433A | 40.000 | 0.000 |
| MMLV-II V433C | 37.504 | 0.682 |
| MMLV-II V433D | 40.000 | 0.000 |
| MMLV-II V433E | 35.189 | 0.336 |
| MMLV-II V433F | 39.379 | 0.878 |
| MMLV-II V433G | 39.482 | 0.732 |
| MMLV-II V433H | 40.000 | 0.000 |
| MMLV-II V433I | 39.781 | 0.310 |
| MMLV-II V433K | 35.770 | 0.623 |
| MMLV-II V433L | 39.015 | 0.744 |
| MMLV-II V433M | 39.119 | 1.247 |
| MMLV-II V433N | 33.981 | 0.185 |
| MMLV-II V433P | 40.000 | 0.000 |
| MMLV-II V433Q | 40.000 | 0.000 |
| MMLV-II V433R | 37.230 | 1.247 |
| MMLV-II V433S | 37.850 | 0.846 |
| MMLV-II V433T | 37.564 | 1.895 |
| MMLV-II V433W | 37.770 | 1.622 |
| MMLV-II V433Y | 40.000 | 0.000 |
| MMLV-IV | 26.102 | 0.033 |

TABLE 33
Two-Step cDNA Synthesis by MMLV-RT single mutants using
random priming. The data was generated via qPCR human
normalizer assay and data is reported by Ct value.

| MMLV-RT Variant | Ct Mean | Ct Standard Deviation |
|---|---|---|
| MMLV-II | 40.000 | 0.000 |
| MMLV-II I593A | 40.000 | 0.000 |
| MMLV-II I593C | 40.000 | 0.000 |
| MMLV-II I593D | 39.992 | 0.012 |
| MMLV-II I593E | 40.000 | 0.000 |
| MMLV-II I593F | 39.189 | 1.147 |
| MMLV-II 1593G | 40.000 | 0.000 |
| MMLV-II I593H | 40.000 | 0.000 |
| MMLV-II I593K | 40.000 | 0.000 |
| MMLV-II I593L | 40.000 | 0.000 |
| MMLV-II I593M | 40.000 | 0.000 |
| MMLV-II I593N | 40.000 | 0.000 |
| MMLV-II I593P | 40.000 | 0.000 |
| MMLV-II I593Q | 39.201 | 0.853 |
| MMLV-II I593R | 38.928 | 1.516 |
| MMLV-II I593S | 39.025 | 1.379 |
| MMLV-II I593T | 38.385 | 1.227 |
| MMLV-II I593V | 39.574 | 0.603 |
| MMLV-II I593W | 32.572 | 0.054 |
| MMLV-II I593Y | 40.000 | 0.000 |
| MMLV-II L280A | 40.000 | 0.000 |
| MMLV-II L280C | 40.000 | 0.000 |
| MMLV-II L280D | 40.000 | 0.000 |
| MMLV-II L280E | 40.000 | 0.000 |
| MMLV-II L280F | 40.000 | 0.000 |
| MMLV-II L280G | 40.000 | 0.000 |
| MMLV-II L280H | 40.000 | 0.000 |
| MMLV-II L280I | 34.152 | 0.276 |
| MMLV-II L280K | 40.000 | 0.000 |
| MMLV-II L280M | 39.973 | 0.038 |
| MMLV-II L280N | 40.000 | 0.000 |
| MMLV-II L280P | 40.000 | 0.000 |
| MMLV-II L280Q | 40.000 | 0.000 |
| MMLV-II L280R | 40.000 | 0.000 |
| MMLV-II L280S | 40.000 | 0.000 |
| MMLV-II L280T | 40.000 | 0.000 |
| MMLV-II L280V | 39.260 | 1.046 |
| MMLV-II L280W | 40.000 | 0.000 |
| MMLV-II L280Y | 40.000 | 0.000 |
| MMLV-II L82A | 40.000 | 0.000 |
| MMLV-II L82C | 40.000 | 0.000 |
| MMLV-II L82D | 40.000 | 0.000 |
| MMLV-II L82E | 39.672 | 0.463 |
| MMLV-II L82F | 36.854 | 0.708 |
| MMLV-II L82G | 40.000 | 0.000 |
| MMLV-II L82H | 37.705 | 0.557 |
| MMLV-II L82I | 39.231 | 1.087 |
| MMLV-II L82K | 39.437 | 0.443 |
| MMLV-II L82M | 40.000 | 0.000 |
| MMLV-II L82N | 40.000 | 0.000 |
| MMLV-II L82P | 40.000 | 0.000 |
| MMLV-II L82Q | 40.000 | 0.000 |
| MMLV-II L82R | 38.595 | 1.191 |
| MMLV-II L82S | 40.000 | 0.000 |
| MMLV-II L82T | 38.449 | 1.192 |
| MMLV-II L82V | 39.438 | 0.795 |
| MMLV-II L82W | 39.178 | 1.163 |
| MMLV-II L82Y | 36.758 | 0.962 |
| MMLV-II Q299A | 40.000 | 0.000 |
| MMLV-II Q299C | 40.000 | 0.000 |
| MMLV-II Q299D | 38.003 | 1.414 |
| MMLV-II Q299E | 39.338 | 0.936 |
| MMLV-II Q299F | 40.000 | 0.000 |
| MMLV-II Q299G | 40.000 | 0.000 |
| MMLV-II Q299H | 40.000 | 0.000 |
| MMLV-II Q299I | 39.850 | 0.212 |
| MMLV-II Q299K | 40.000 | 0.000 |
| MMLV-II Q299L | 40.000 | 0.000 |
| MMLV-II Q299M | 40.000 | 0.000 |
| MMLV-II Q299N | 40.000 | 0.000 |
| MMLV-II Q299P | 40.000 | 0.000 |
| MMLV-II Q299R | 40.000 | 0.000 |
| MMLV-II Q299S | 40.000 | 0.000 |
| MMLV-II Q299T | 40.000 | 0.000 |
| MMLV-II Q299V | 40.000 | 0.000 |
| MMLV-II Q299W | 40.000 | 0.000 |
| MMLV-II Q299Y | 40.000 | 0.000 |
| MMLV-II T332A | 39.814 | 0.264 |
| MMLV-II T332C | 40.000 | 0.000 |
| MMLV-II T332D | 40.000 | 0.000 |
| MMLV-II T332E | 40.000 | 0.000 |
| MMLV-II T332F | 40.000 | 0.000 |
| MMLV-II T332G | 38.897 | 1.560 |
| MMLV-II T332H | 40.000 | 0.000 |
| MMLV-II T332I | 40.000 | 0.000 |
| MMLV-II T332K | 40.000 | 0.000 |
| MMLV-II T332L | 38.169 | 2.589 |
| MMLV-II T332M | 37.410 | 1.906 |
| MMLV-II T332N | 38.983 | 1.362 |
| MMLV-II T332P | 39.046 | 1.350 |
| MMLV-II T332Q | 40.000 | 0.000 |
| MMLV-II T332R | 40.000 | 0.000 |
| MMLV-II T332S | 40.000 | 0.000 |
| MMLV-II T332V | 38.650 | 1.326 |
| MMLV-II T332W | 40.000 | 0.000 |
| MMLV-II T332Y | 40.000 | 0.000 |
| MMLV-II V433A | 40.000 | 0.000 |
| MMLV-II V433C | 37.605 | 0.184 |
| MMLV-II V433D | 40.000 | 0.000 |
| MMLV-II V433E | 34.693 | 0.193 |
| MMLV-II V433F | 40.000 | 0.000 |
| MMLV-II V433G | 40.000 | 0.000 |
| MMLV-II V433H | 40.000 | 0.000 |
| MMLV-II V433I | 39.792 | 0.294 |
| MMLV-II V433K | 35.725 | 0.464 |
| MMLV-II V433L | 40.000 | 0.000 |
| MMLV-II V433M | 40.000 | 0.000 |
| MMLV-II V433N | 34.604 | 0.554 |
| MMLV-II V433P | 40.000 | 0.000 |
| MMLV-II V433Q | 38.844 | 1.001 |
| MMLV-II V433R | 38.817 | 0.839 |
| MMLV-II V433S | 38.202 | 1.372 |
| MMLV-II V433T | 37.573 | 0.623 |
| MMLV-II V433W | 37.611 | 1.690 |
| MMLV-II V433Y | 40.000 | 0.000 |
| MMLV-IV | 26.053 | 0.098 |

TABLE 34
One-Step cDNA Synthesis by MMLV-RT single mutants by
gene specific priming. The data was generated via qPCR
human normalizer assay and data is reported by Ct value.

| MMLV-RT Variant | Ct Mean | Ct Standard Deviation |
|---|---|---|
| MMLV-II | 32.775 | 0.189 |
| MMLV-II I593A | 32.438 | 0.209 |
| MMLV-II I593C | 32.680 | 0.053 |
| MMLV-II I593D | 31.775 | 0.237 |
| MMLV-II I593E | 30.635 | 0.048 |
| MMLV-II I593F | 30.411 | 0.008 |
| MMLV-II I593G | 30.904 | 0.098 |
| MMLV-II I593H | 29.686 | 0.131 |
| MMLV-II I593K | 31.832 | 0.259 |
| MMLV-II I593L | 32.289 | 0.273 |
| MMLV-II I593M | 32.162 | 0.078 |
| MMLV-II I593N | 31.410 | 0.251 |
| MMLV-II I593P | 34.728 | 0.201 |
| MMLV-II I593Q | 31.609 | 0.032 |
| MMLV-II I593R | 31.144 | 0.133 |
| MMLV-II I593S | 30.548 | 0.247 |
| MMLV-II I593T | 29.572 | 0.236 |
| MMLV-II I593V | 30.673 | 0.142 |
| MMLV-II I593W | 28.179 | 0.092 |
| MMLV-II I593Y | 30.858 | 0.067 |
| MMLV-II L280A | 36.160 | 0.729 |
| MMLV-II L280C | 32.097 | 0.261 |
| MMLV-II L280D | 40.000 | 0.000 |
| MMLV-II L280E | 39.115 | 1.251 |
| MMLV-II L280F | 34.573 | 0.371 |
| MMLV-II L280G | 40.000 | 0.000 |
| MMLV-II L280H | 37.255 | 0.322 |
| MMLV-II L280I | 29.267 | 1.032 |
| MMLV-II L280K | 34.274 | 0.095 |
| MMLV-II L280M | 32.746 | 0.223 |
| MMLV-II L280N | 39.677 | 0.457 |
| MMLV-II L280P | 33.045 | 0.095 |
| MMLV-II L280Q | 39.190 | 1.145 |
| MMLV-II L280R | 40.000 | 0.000 |
| MMLV-II L280S | 40.000 | 0.000 |
| MMLV-II L280T | 37.074 | 0.325 |
| MMLV-II L280V | 30.461 | 0.052 |
| MMLV-II L280W | 40.000 | 0.000 |
| MMLV-II L280Y | 40.000 | 0.000 |
| MMLV-II L82A | 31.729 | 0.308 |
| MMLV-II L82C | 31.131 | 0.192 |
| MMLV-II L82D | 34.280 | 0.227 |
| MMLV-II L82E | 32.973 | 0.430 |
| MMLV-II L82F | 29.760 | 0.030 |
| MMLV-II L82G | 33.066 | 0.217 |
| MMLV-II L82H | 30.098 | 0.078 |
| MMLV-II L82I | 31.605 | 0.083 |
| MMLV-II L82K | 29.258 | 0.015 |
| MMLV-II L82M | 30.280 | 0.027 |
| MMLV-II L82N | 33.074 | 0.323 |
| MMLV-II L82P | 38.754 | 1.762 |
| MMLV-II L82Q | 32.001 | 0.164 |
| MMLV-II L82R | 30.208 | 0.128 |
| MMLV-II L82S | 31.841 | 0.231 |
| MMLV-II L82T | 28.908 | 0.044 |
| MMLV-II L82V | 29.533 | 0.057 |
| MMLV-II L82W | 29.580 | 0.056 |
| MMLV-II L82Y | 28.934 | 0.073 |
| MMLV-II Q299A | 31.113 | 0.138 |
| MMLV-II Q299C | 35.953 | 0.542 |
| MMLV-II Q299D | 32.292 | 0.080 |
| MMLV-II Q299E | 31.663 | 0.027 |
| MMLV-II Q299F | 36.143 | 0.317 |
| MMLV-II Q299G | 31.929 | 0.131 |
| MMLV-II Q299H | 32.387 | 0.133 |
| MMLV-II Q299I | 37.763 | 1.582 |
| MMLV-II Q299K | 32.326 | 0.096 |
| MMLV-II Q299L | 34.807 | 0.180 |
| MMLV-II Q299M | 32.514 | 0.375 |
| MMLV-II Q299N | 34.040 | 0.186 |
| MMLV-II Q299P | 39.460 | 0.764 |
| MMLV-II Q299R | 33.044 | 0.354 |
| MMLV-II Q299S | 33.438 | 0.256 |
| MMLV-II Q299T | 35.093 | 0.926 |
| MMLV-II Q299V | 35.114 | 1.045 |
| MMLV-II Q299W | 38.998 | 1.417 |
| MMLV-II Q299Y | 39.055 | 1.336 |
| MMLV-II T332A | 30.528 | 0.084 |
| MMLV-II T332C | 30.785 | 0.135 |
| MMLV-II T332D | 33.310 | 0.348 |
| MMLV-II T332E | 32.711 | 0.106 |
| MMLV-II T332F | 33.201 | 0.179 |
| MMLV-II T332G | 30.424 | 0.054 |
| MMLV-II T332H | 31.913 | 0.306 |
| MMLV-II T332I | 32.072 | 0.115 |
| MMLV-II T332K | 31.591 | 0.082 |
| MMLV-II T332L | 34.011 | 0.133 |
| MMLV-II T332M | 29.039 | 0.164 |
| MMLV-II T332N | 29.500 | 0.135 |
| MMLV-II T332P | 33.976 | 0.272 |
| MMLV-II T332Q | 31.599 | 0.041 |
| MMLV-II T332R | 32.950 | 0.130 |
| MMLV-II T332S | 31.003 | 0.341 |
| MMLV-II T332V | 29.835 | 0.061 |
| MMLV-II T332W | 35.431 | 0.099 |
| MMLV-II T332Y | 33.384 | 0.164 |
| MMLV-II V433A | 30.757 | 0.105 |
| MMLV-II V433C | 29.901 | 0.305 |
| MMLV-II V433D | 34.152 | 0.170 |
| MMLV-II V433E | 28.868 | 0.011 |
| MMLV-II V433F | 31.529 | 0.009 |
| MMLV-II V433G | 33.663 | 0.412 |
| MMLV-II V433H | 31.811 | 0.069 |
| MMLV-II V433I | 30.460 | 0.071 |
| MMLV-II V433K | 30.040 | 0.109 |
| MMLV-II V433L | 31.758 | 0.063 |
| MMLV-II V433M | 30.791 | 0.095 |
| MMLV-II V433N | 28.566 | 0.074 |
| MMLV-II V433P | 37.436 | 1.824 |
| MMLV-II V433Q | 30.586 | 0.104 |
| MMLV-II V433R | 30.773 | 0.080 |
| MMLV-II V433S | 29.768 | 0.074 |
| MMLV-II V433T | 29.096 | 0.107 |
| MMLV-II V433W | 29.130 | 0.064 |
| MMLV-II V433Y | 32.676 | 0.279 |
| MMLV-IV | 25.979 | 0.043 |

TABLE 35
Two-Step cDNA Synthesis by MMLV-RT stacked mutants using
oligo dT priming. The data was generated via qPCR human
normalizer assay and data is reported by Ct value.

| | Temperature | Ct | Ct Standard |
|---|---|---|---|
| MMLV-RT Variant | (° C.) | Mean | Deviation |
| MMLV-II | 42 | 25.207 | 0.025 |
| MMLV-II | 55 | 28.180 | 0.022 |
| MMLV-II Q68R/Q79R/L99R/E282D | 42 | 25.287 | 0.068 |
| | 55 | 26.442 | 0.044 |
| MMLV-II | 42 | 25.344 | 0.065 |
| Q68R/Q79R/L99R/E282D/V433R | 55 | 26.586 | 0.077 |
| MMLV-II | 42 | 25.266 | 0.112 |
| Q68R/Q79R/L99R/E282D/I593E | 55 | 27.389 | 0.069 |
| MMLV-II | 42 | 25.357 | 0.087 |
| Q68R/Q79R/L99R/E282D/Q299E | 55 | 26.953 | 0.034 |
| MMLV-II | 42 | 25.394 | 0.011 |
| Q68R/Q79R/L82R/L99R/E282D | 55 | 27.171 | 0.028 |
| MMLV-II | 42 | 25.371 | 0.061 |
| Q68R/Q79R/L99R/E282D/Q299E/ | 55 | 26.689 | 0.068 |
| I593E | | | |
| MMLV-II | 42 | 25.258 | 0.035 |
| Q68R/Q79R/L82R/L99R/E282D/ | 55 | 26.979 | 0.034 |
| Q299E/I593E | | | |
| MMLV-II | 42 | 25.171 | 0.006 |
| Q68R/Q79R/L99R/E282D/Q299E/ | 55 | 26.299 | 0.025 |
| V433R/I593E | | | |
| MMLV-II | 42 | 25.146 | 0.052 |
| Q68R/Q79R/L82R/L99R/E282D/ | 55 | 26.320 | 0.036 |
| Q299E/V433R/I593E | | | |
| MMLV-II | 42 | 25.176 | 0.044 |
| Q68R/Q79R/L82R/L99R/E282D/ | 55 | 26.750 | 0.040 |
| Q299E/T332E/I593E | | | |
| MMLV-II | 42 | 25.110 | 0.046 |
| Q68R/Q79R/L82R/L99R/E282D/ | 55 | 26.587 | 0.049 |
| Q299E/T332E/V433R/I593E | | | |
| MMLV-IV | 42 | 25.184 | 0.025 |
| MMLV-IV | 55 | 25.153 | 0.037 |
| SuperScript-IV | 42 | 25.082 | 0.073 |
| SuperScript-IV | 55 | 25.080 | 0.047 |

TABLE 36
Two-Step cDNA Synthesis by MMLV-RT stacked mutants using random
priming. The data was generated via qPCR human
normalizer assay and data is reported by Ct value.

| | Temper- | | Ct |
|---|---|---|---|
| | ature | Ct | Standard |
| MMLV-RT Variant | (° C.) | Mean | Deviation |
| MMLV-II | 42 | 25.264 | 0.019 |
| MMLV-II | 55 | 28.443 | 0.014 |
| MMLV-II Q68R/Q79R/L99R/E282D | 42 | 25.399 | 0.040 |
| | 55 | 26.484 | 0.072 |
| MMLV-II Q68R/Q79R/L99R/E282D/V433R | 42 | 25.324 | 0.063 |
| | 55 | 26.794 | 0.065 |
| MMLV-II Q68R/Q79R/L99R/E282D/I593E | 42 | 25.278 | 0.025 |
| | 55 | 27.616 | 0.058 |
| MMLV-II Q68R/Q79R/L99R/E282D/Q299E | 42 | 25.281 | 0.079 |
| | 55 | 27.148 | 0.025 |
| MMLV-II Q68R/Q79R/L82R/L99R/E282D | 42 | 25.279 | 0.053 |
| | 55 | 27.243 | 0.008 |
| MMLV-II Q68R/Q79R/L99R/ | 42 | 25.409 | 0.065 |
| E282D/Q299E/I593E | 55 | 26.704 | 0.066 |
| MMLV-II | 42 | 25.581 | 0.062 |
| Q68R/Q79R/L82R/L99R/ | 55 | 26.605 | 0.028 |
| E282D/Q299E/I593E | | | |
| MMLV-II | 42 | 25.355 | 0.158 |
| Q68R/Q79R/L99R/E282D/ | 55 | 26.305 | 0.066 |
| Q299E/V433R/I593E | | | |
| MMLV-II | 42 | 25.418 | 0.120 |
| Q68R/Q79R/L82R/L99R/E282D/ | 55 | 26.403 | 0.055 |
| Q299E/V433R/I593E | | | |
| MMLV-II | 42 | 25.374 | 0.115 |
| Q68R/Q79R/L82R/L99R/E282D/ | 55 | 26.747 | 0.065 |
| Q299E/T332E/I593E | | | |
| MMLV-II | 42 | 25.426 | 0.082 |
| Q68R/Q79R/L82R/L99R/E282D/ | 55 | 26.481 | 0.017 |
| Q299E/T332E/V433R/I593E | | | |
| MMLV-IV | 42 | 25.394 | 0.162 |
| MMLV-IV | 55 | 25.185 | 0.022 |
| SuperScript-IV | 42 | 25.299 | 0.132 |
| SuperScript-IV | 55 | 25.214 | 0.021 |

TABLE 37
One-Step cDNA Synthesis by MMLV-RT stacked mutants by
gene specific priming. The data was generated via qPCR human
normalizer assay and data is reported by Ct value.

| | Temper- | Con- | | Ct |
|---|---|---|---|---|
| | ature | centration | Ct | Standard |
| MMLV-RT Variant | (° C.) | of RT (nM) | Mean | Deviation |
| MMLV-II | 50 | 0.28 | 26.401 | 0.022 |
| | | 1.4 | 24.701 | 0.061 |
| | | 7.0 | 24.664 | 0.007 |
| | 60 | 0.28 | 31.134 | 0.205 |
| | | 1.4 | 28.109 | 0.042 |
| | | 7.0 | 27.644 | 0.061 |
| MMLV-II | 50 | 0.28 | 25.171 | 0.046 |
| Q68R/Q79R/L99R/ | | 1.4 | 24.440 | 0.037 |
| E282D | | 7.0 | 24.406 | 0.010 |
| | 60 | 0.28 | 28.848 | 0.114 |
| | | 1.4 | 25.905 | 0.066 |
| | | 7.0 | 25.618 | 0.057 |
| MMLV-II | 50 | 0.28 | 24.967 | 0.068 |
| Q68R/Q79R/L99R/ | | 1.4 | 24.386 | 0.015 |
| E282D/V433R | | 7.0 | 24.433 | 0.079 |
| | 60 | 0.28 | 28.516 | 0.051 |
| | | 1.4 | 25.803 | 0.063 |
| | | 7.0 | 25.620 | 0.035 |
| MMLV-II | 50 | 0.28 | 24.660 | 0.053 |
| Q68R/Q79R/L99R/ | | 1.4 | 24.377 | 0.028 |
| E282D/I593E | | 7.0 | 24.355 | 0.021 |
| | 60 | 0.28 | 27.488 | 0.074 |
| | | 1.4 | 25.413 | 0.049 |
| | | 7.0 | 25.209 | 0.136 |
| MMLV-II | 50 | 0.28 | 25.044 | 0.094 |
| Q68R/Q79R/L99R/ | | 1.4 | 24.422 | 0.023 |
| E282D/Q299E | | 7.0 | 24.528 | 0.055 |
| | 60 | 0.28 | 28.818 | 0.137 |
| | | 1.4 | 25.953 | 0.082 |
| | | 7.0 | 25.754 | 0.098 |
| MMLV-II | 50 | 0.28 | 25.014 | 0.152 |
| Q68R/Q79R/L82R/ | | 1.4 | 24.467 | 0.020 |
| L99R/E282D | | 7.0 | 24.507 | 0.046 |
| | 60 | 0.28 | 28.743 | 0.076 |
| | | 1.4 | 26.662 | 0.012 |
| | | 7.0 | 25.883 | 0.022 |
| MMLV-II | 50 | 0.28 | 24.771 | 0.027 |
| Q68R/Q79R/L99R/ | | 1.4 | 24.501 | 0.008 |
| E282D/Q299E/I593E | | 7.0 | 24.485 | 0.087 |
| | 60 | 0.28 | 27.721 | 0.057 |
| | | 1.4 | 25.836 | 0.030 |
| | | 7.0 | 25.199 | 0.016 |
| MMLV-II | 50 | 0.28 | 24.777 | 0.029 |
| Q68R/Q79R/L82R/ | | 1.4 | 24.432 | 0.033 |
| L99R/E282D/Q299E/ | | 7.0 | 24.435 | 0.024 |
| I593E | 60 | 0.28 | 27.854 | 0.035 |
| | | 1.4 | 25.613 | 0.028 |
| | | 7.0 | 25.072 | 0.030 |
| MMLV-II | 50 | 0.28 | 24.550 | 0.003 |
| Q68R/Q79R/L99R/ | | 1.4 | 24.333 | 0.033 |
| E282D/Q299E/V433R/ | | 7.0 | 24.345 | 0.030 |
| I593E | 60 | 0.28 | 26.399 | 0.051 |
| | | 1.4 | 25.236 | 0.040 |
| | | 7.0 | 25.105 | 0.050 |
| MMLV-II | 50 | 0.28 | 24.562 | 0.047 |
| Q68R/Q79R/L82R/ | | 1.4 | 24.350 | 0.039 |
| L99R/E282D/Q299E/ | | 7.0 | 24.302 | 0.015 |
| V433R/I593E | 60 | 0.28 | 26.459 | 0.022 |
| | | 1.4 | 25.247 | 0.069 |
| | | 7.0 | 25.001 | 0.050 |
| MMLV-II | 50 | 0.28 | 24.614 | 0.047 |
| Q68R/Q79R/L82R/ | | 1.4 | 24.420 | 0.051 |
| L99R/E282D/Q299E/ | | 7.0 | 24.361 | 0.021 |
| T332E/I593E | 60 | 0.28 | 26.769 | 0.089 |
| | | 1.4 | 25.609 | 0.041 |
| | | 7.0 | 25.348 | 0.043 |
| MMLV-II | 50 | 0.28 | 24.594 | 0.075 |
| Q68R/Q79R/L82R/ | | 1.4 | 24.402 | 0.045 |
| L99R/E282D/Q299E/ | | 7.0 | 24.291 | 0.057 |
| T332E/V433R/I593E | 60 | 0.28 | 26.591 | 0.018 |
| | | 1.4 | 25.517 | 0.048 |
| | | 7.0 | 25.193 | 0.027 |
| MMLV-IV | 50 | 0.28 | 24.397 | 0.091 |
| | | 1.4 | 24.303 | 0.062 |
| | | 7.0 | 24.189 | 0.039 |
| | 60 | 0.28 | 25.807 | 0.045 |
| | | 1.4 | 25.180 | 0.037 |
| | | 7.0 | 24.625 | 0.011 |
| SuperScript-IV | 50 | 0.28 | 24.743 | 0.049 |
| | | 1.4 | 24.213 | 0.017 |
| | | 7.0 | 24.008 | 0.036 |
| | 60 | 0.28 | 26.124 | 0.103 |
| | | 1.4 | 24.681 | 0.070 |
| | | 7.0 | 24.180 | 0.082 |

TABLE 38
Sequence of quadruple or more mutant MMLV RTase variants.

| SEQ ID NO: | Construct | Construct Sequence (AA) |
|---|---|---|
| 686 | MMLV-II | TLNIEDEHRLHETSKEPDVSLGSTWLSDFPQAWAE |
| | Q68R/Q79R/ | TGGMGLAVRQAPLIIPLKATSTPVSIKQYPMSREA |
| | L99R/E282D/ | RLGIKPHIRRLLDQGILVPCQSPWNTPLRPVKKPG |
| | V433R | TNDYRPVQDLREVNKRVEDIHPTVPNPYNLLSGLP |
| | | PSHQWYTVLDLKDAFFCLRLHPTSQPLFAFEWRDP |
| | | EMGISGOLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRQLREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLRILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTOALKMAEGKKLNVYTNSRYAFATAHIHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 687 | MMLV-II | TLNIEDEHRLHETSKEPDVSLGSTWLSDFPQAWAE |
| | Q68R/Q79R/ | TGGMGLAVRQAPLIIPLKATSTPVSIKQYPMSREA |
| | L99R/E282D/ | RLGIKPHIRRLLDQGILVPCQSPWNTPLRPVKKPG |
| | I593E | TNDYRPVQDLREVNKRVEDIHPTVPNPYNLLSGLP |
| | | PSHQWYTVLDLKDAFFCLRLHPTSQPLFAFEWRDP |
| | | EMGISGQLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRQLREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLVILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHEHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 688 | MMLV-II | TLNIEDEHRLHETSKEPDVSLGSTWLSDFPQAWAE |
| | Q68R/Q79R/ | TGGMGLAVRQAPLIIPLKATSTPVSIKQYPMSREA |
| | L99R/E282D/ | RLGIKPHIRRLLDQGILVPCQSPWNTPLRPVKKPG |
| | Q299E | TNDYRPVQDLREVNKRVEDIHPTVPNPYNLLSGLP |
| | | PSHQWYTVLDLKDAFFCLRLHPTSQPLFAFEWRDP |
| | | EMGISGQLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRELREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLVILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHIHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 689 | MMLV-II | TLNIEDEHRLHETSKEPDVSLGSTWLSDFPQAWAE |
| | Q68R/Q79R/ | TGGMGLAVRQAPLIIPLKATSTPVSIKQYPMSREA |
| | L99R/E282D/ | RLGIKPHIRRLLDQGILVPCQSPWNTPLRPVKKPG |
| | T332E | TNDYRPVQDLREVNKRVEDIHPTVPNPYNLLSGLP |
| | | PSHQWYTVLDLKDAFFCLRLHPTSQPLFAFEWRDP |
| | | EMGISGQLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRQLREFLGTAGFCRLWIP |
| | | GFAEMAAPLYPLTKTGELFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLVILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHIHG |
| | | ETYRRRGLLTSEGKEIKNKDEILALLKALFLPKRL |
| | | SIIHCPGHQKGHSAEARGNRMADQAARKAAITETP |
| | | DTSTLLIENSSPYTSEHE |
| 690 | MMLV-II | TLNIEDEHRLHETSKEPDVSLGSTWLSDFPQAWAE |
| | Q68R/Q79R/ | TGGMGLAVRQAPLIIPLKATSTPVSIKQYPMSREA |
| | L99R/L280R | RLGIKPHIRRLLDQGILVPCQSPWNTPLRPVKKPG |
| | | TNDYRPVQDLREVNKRVEDIHPTVPNPYNLLSGLP |
| | | PSHQWYTVLDLKDAFFCLRLHPTSQPLFAFEWRDP |
| | | EMGISGOLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWR |
| | | TEARKETVMGQPTPKTPRQLREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLVILAPHAVEALVKQPPDRWLSNA |
| | | RMTHYQALLLDTDRVQFGPVVALNPATLLPLPEEG |
| | | LQHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHIHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 691 | MMLV-II | TLNIEDEHRLHETSKEPDVSLGSTWLSDFPQAWAE |
| | Q68R/Q79R/ | TGGMGLAVRQAPLIIPLKATSTPVSIKQYPMSREA |
| | L99R/L280R/ | RLGIKPHIRRLLDQGILVPCQSPWNTPLRPVKKPG |
| | E282D | TNDYRPVQDLREVNKRVEDIHPTVPNPYNLLSGLP |
| | | PSHQWYTVLDLKDAFFCLRLHPTSQPLFAFEWRDP |
| | | EMGISGOLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWR |
| | | TDARKETVMGQPTPKTPRQLREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLVILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGORKAGAAVTTETEVIWAKALPAGTSAQR |
| | | AQLIALTQALKMAEGKKLNVYTNSRYAFATAHIHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 692 | MMLV-II | TLNIEDEHRLHETSKEPDVSLGSTWLSDFPQAWAE |
| | Q68R/L82R/ | TGGMGLAVROAPLIIPLKATSTPVSIKQYPMSREA |
| | L99R/E282D | RLGIKPHIQRLRDQGILVPCQSPWNTPLRPVKKPG |
| | | TNDYRPVQDLREVNKRVEDIHPTVPNPYNLLSGLP |
| | | PSHQWYTVLDLKDAFFCLRLHPTSQPLFAFEWRDP |
| | | EMGISGOLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRQLREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLVILAPHAVEALVKQPPDRWLSNA |
| | | RMT HYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHIHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHE |
| 693 | MMLV-II | TLNIEDEHRLHETSKEPDVSLGSTWLSDFPQAWAE |
| | Q68R/Q79R/ | TGGMGLAVRQAPLIIPLKATSTPVSIKQYPMSREA |
| | L82R/L99R/ | RLGIKPHIRRLRDQGILVPCQSPWNTPLRPVKKPG |
| | E282D | TNDYRPVQDLREVNKRVEDIHPTVPNPYNLLSGLP |
| | | PSHQWYTVLDLKDAFFCLRLHPTSQPLFAFEWRDP |
| | | EMGISGOLTWTRLPQGFKNSPTLFDEALHRDLADF |
| | | RIQHPDLILLQYVDDLLLAATSELDCQQGTRALLQ |
| | | TLGNLGYRASAKKAQICQKOVKYLGYLLKEGQRWL |
| | | TDARKETVMGQPTPKTPRQLREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLVILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHIHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 694 | MMLV-II | TLNIEDEHRLHETSKEPDVSLGSTWLSDFPQAWAE |
| | Q68R/Q79R/ | TGGMGLAVROAPLIIPLKATSTPVSIKQYPMSREA |
| | L99R/E282D/ | RLGIKPHIRRLLDQGILVPCQSPWNTPLRPVKKPG |
| | Q299E/I593E | TNDYRPVQDLREVNKRVEDIHPTVPNPYNLLSGLP |
| | | PSHQWYTVLDLKDAFFCLRLHPTSQPLFAFEWRDP |
| | | EMGISGOLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRELREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLVILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHEHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 695 | MMLV-II | TLNIEDEHRLHETSKEPDVSLGSTWLSDFPQAWAE |
| | Q68R/Q79R/ | TGGMGLAVRQAPLIIPLKATSTPVSIKQYPMSREA |
| | L82R/L99R/ | RLGIKPHIRRLRDQGILVPCQSPWNTPLRPVKKPG |
| | E282D/Q299E/ | TNDYRPVQDLREVNKRVEDIHPTVPNPYNLLSGLP |
| | I593E | PSHQWYTVLDLKDAFFCLRLHPTSQPLFAFEWRDP |
| | | EMGISGQLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRELREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLVILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHEHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 696 | MMLV-II | TLNIEDEHRLHETSKEPDVSLGSTWLSDFPQAWAE |
| | Q68R/Q79R/ | TGGMGLAVRQAPLIIPLKATSTPVSIKQYPMSREA |
| | L99R/E282D/ | RLGIKPHIRRLLDQGILVPCQSPWNTPLRPVKKPG |
| | Q299E/V433R/ | TNDYRPVQDLREVNKRVEDIHPTVPNPYNLLSGLP |
| | I593E | PSHQWYTVLDLKDAFFCLRLHPTSQPLFAFEWRDP |
| | | EMGISGQLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRELREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLRILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTOALKMAEGKKLNVYTNSRYAFATAHEHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 697 | MMLV-II | TLNIEDEHRLHETSKEPDVSLGSTWLSDFPQAWAE |
| | Q68R/Q79R/ | TGGMGLAVROAPLIIPLKATSTPVSIKQYPMSREA |
| | L82R/L99R/ | RLGIKPHIRRLRDQGILVPCQSPWNTPLRPVKKPG |
| | E282D/Q299E/ | TNDYRPVQDLREVNKRVEDIHPTVPNPYNLLSGLP |
| | V433R/I593E | PSHQWYTVLDLKDAFFCLRLHPTSQPLFAFEWRDP |
| | | EMGISGOLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRELREFLGTAGFCRLWIP |
| | | GFAEMAAPLYPLTKTGTLFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLRILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHEHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 698 | MMLV-II | TLNIEDEHRLHETSKEPDVSLGSTWLSDFPQAWAE |
| | Q68R/Q79R/ | TGGMGLAVRQAPLIIPLKATSTPVSIKQYPMSREA |
| | L82R/L99R/ | RLGIKPHIRRLRDQGILVPCQSPWNTPLRPVKKPG |
| | E282D/Q299E/ | TNDYRPVQDLREVNKRVEDIHPTVPNPYNLLSGLP |
| | T332E/I593E | PSHQWYTVLDLKDAFFCLRLHPTSQPLFAFEWRDP |
| | | EMGISGOLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRELREFLGTAGFCRLWIP |
| | | GFAEMAAPLYPLTKTGELFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLVILAPHAVEALVKQPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHEHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |
| 699 | MMLV-II | TLNIEDEHRLHETSKEPDVSLGSTWLSDFPQAWAE |
| | Q68R/Q79R/ | TGGMGLAVROAPLIIPLKATSTPVSIKQYPMSREA |
| | L82R/L99R/ | RLGIKPHIRRLRDQGILVPCQSPWNTPLRPVKKPG |
| | E282D/Q299E/ | TNDYRPVQDLREVNKRVEDIHPTVPNPYNLLSGLP |
| | T332E/V433R/ | PSHQWYTVLDLKDAFFCLRLHPTSQPLFAFEWRDP |
| | I593E | EMGISGQLTWTRLPQGFKNSPTLFDEALHRDLADF |
| | | RIQHPDLILLQYVDDLLLAATSELDCQQGTRALLQ |
| | | TLGNLGYRASAKKAQICQKQVKYLGYLLKEGQRWL |
| | | TDARKETVMGQPTPKTPRELREFLGTAGFCRLWIP |
| | | GFAEMAAPLYPLTKTGELFNWGPDQQKAYQEIKQA |
| | | LLTAPALGLPDLTKPFELFVDEKQGYAKGVLTQKL |
| | | GPWRRPVAYLSKKLDPVAAGWPPCLRMVAAIAVLT |
| | | KDAGKLTMGQPLRILAPHAVEALVKOPPDRWLSNA |
| | | RMTHYQALLLDTDRVQFGPVVALNPATLLPLPEEG |
| | | LOHNCLDILAEAHGTRPDLTDQPLPDADHTWYTGG |
| | | SSLLQEGQRKAGAAVTTETEVIWAKALPAGTSAQR |
| | | AQLIALTQALKMAEGKKLNVYTNSRYAFATAHEHG |
| | | EIYRRRGLLTSEGKEIKNKDEILALLKALFLPKRL |
| | | SIIHCPGHQKGHSAEARGNRMADQAARKAAITETP |
| | | DTSTLLIENSSPYTSEHF |

### Example 10: Selection of C-Terminal Peptide Extensions of MMLV RTase for Increased Activity and Thermostability

C-terminal peptide extensions were selected from use in previous studies demonstrating an increase in thermostability of a non-RTase related protein attached to the N-terminal or C-terminal end of the desired protein. The origin, amino acid sequence, and reference of the C-terminal extensions are summarized in Table 39.

TABLE 39
C-terminal peptide studies.

| | Size | | | |
|---|---|---|---|---|
| C-terminal | (amino acid | | | |
| peptide | residues) | Origin | Amino Acid Sequence | Reference |
| Control Tag 1 | 16 | Random generation of peptide | RDRNKNNDRRKAKENE (SEQ | Hogrefe et al. |
| | | tag | ID NO: 732) | |
| ATS | 21 | C-end tail of human α- | DPDNEAYEMPSEEGYQDYEP | Lee et al. (2005) |
| | | synuclein (NCBI accession | EA (SEQ ID NO: 733) | |
| | | no. NP_001362216.1) | | |
| ATS | 42 | C-end tail of human α- | QLGKNEEGAPQEGILEDMP | Zhang et al. (2015); |
| | | synuclein (NCBI accession | VDPDNEAYEMPSEEGYQDY | Park et al. (2002) |
| | | no. NP_001362216.1) | EPEA (SEQ ID NO: 734) | |
| ATTa Peptide | 40 | C-end tail of Arabidopsis | EGMEEGEFSEAREDLAALE | Zhang et al. (2015) |
| | | tubulins, TUA2 (NCBI | KDYEEVGAEGGDDEDDEGE | |
| | | accession no. NP_175423.1) | EY (SEQ ID NO: 735) | |
| ATTb Peptide | 50 | C-end tail of Arabidopsis | EGMDEMEFTEAESNMNDLV | Zhang et al. (2015) |
| | | tubulins, TUA3 (NCBI | SEYQQYQDATADEEGDYED | |
| | | accession no. NP_568960.1) | EEEGEYQQEEEY (SEQ ID NO: | |
| | | | 736) | |
| Msb | 114 | E. coli msyB (NCBI | IDAAREEFLADNPGIDAEDA | Zhang et al. (2015); |
| | | accession no. | NVQQFNAQKYVLQDGDIM | Zou et al. (2008) |
| | | CAD6011033.1) | WQVEFFADEGEEGECLPML | |
| | | | SGEAAQSVFDGDYDEIEIRQ | |
| | | | EWQEENTLHEWDEGEFQLE | |
| | | | PPLDTEEGRAAADEWDER | |
| | | | (SEQ ID NO: 737) | |
| Yd | 137 | E. coli hypothetical E. coli | ANPEQLEEQREETRLIIEELL | Zou et al. (2008) |
| | | ORF, yjgD (NCBI accession | EDGSDPDALYTIEHHLSADD | |
| | | no. AAG59454.1) | LETLEKAAVEAFKLGYEVTD | |
| | | | PEELEVEDGDIVICCDILSEC | |
| | | | ALNADLIDAQVEQLMTLAE | |
| | | | KFDVEYDGWGTYFEDPNGE | |
| | | | DGDDEDFVDEDDDGVRH | |
| | | | (SEQ ID NO: 738) | |
| Od | 182 | N-terminal domain of E. coli | DIVDSDQIEDIIQMINDMGIQ | Zou et al. (2008) |
| | | rpoD (NCBI accession no. | VMEEAPDADDLMLAENTAD | |
| | | CAD6003062.1) | EDAAEAAAQVLSSVESEIGR | |
| | | | TTDPVRMYMREMGTVELLT | |
| | | | REGEIDIAKRIEDGINQVQCS | |
| | | | VAEYPEAITYLLEQYDRVEA | |
| | | | EEARLSDLITGFVDPNAEED | |
| | | | LAPTATHVGSELSQEDLDDDE | |
| | | | DEDEEDGDDDSADDD | |
| | | | NSIDPE (SEQ ID NO: 739) | |
| ATYd | | E. coli yjgD (NCBI accession | PNGEDGDDEDFVDEDDDGV | Zhang et al. (2015) |
| | | ho. AAP43518.1) | (SEQ ID NO: 740) | |
| Trx | 102 | E. coli thioredoxin (NCBI | MTTATFSRHVERSDLPLLVD | Zou et al. (2008) |
| | | accession no. | FWAPCGPCKMMAPQFQQAA | |
| | | WP_187194155.1) | HQLEPTIRLAKVNIEAEPHLAA | |
| | | | QFGIRSIPTLALFQGGREIARQ | |
| | | | AGVMGAQDIVRWTSTOVGR | |
| | | | (SEQ ID NO: 741) | |
| Syn96-140 | 45 | C-end tail of human α- | KKDQLGKNEEGAPQEGILE | Park et al. (2004) |
| | | synuclein (NCBI accession | DMPVDPDNEAYEMPSEEGY | |
| | | no. NP_001362216.1) | QDYEPEA (SEQ ID NO: 742) | |
| Syn103-115 | | C-end tail of human α- | NEEGAPQEGILED (SEQ ID | Park et al. (2004) |
| | | synuclein (NCBI accessionno. | NO: 743) | |
| | | NP_001362216.1) | | |
| Syn114-126 | 13 | C-end tail of human α- | NDMPVDPDNEAYE (SEQ ID | Park et al. (2004) |
| | | synuclein (NCBI accession | NO: 744) | |
| | | no. NP_001362216.1) | | |
| Syn119-140 | 22 | C-end tail of human α- | DPDNEAYEMPSEEGYQDYEP | Park et al. (2004) |
| | | synuclein (NCBI accessionno. | EA (SEQ ID NO: 745) | |
| | | NP_001362216.1) | | |
| Syn130-140 | 11 | C-end tail of human α- | EEGYQDYEPEA (SEQ ID NO: | Park et al. (2004) |
| | | synuclein (NCBI accessionno. | 746) | |
| | | NP_001362216.1) | | |
| LipB | 26 | C-end tail of Fusarium | DMSDEELEKKLTQYSEMDQ | Nagao et al. (1998) |
| | | heterosporum Lipase B | EFVKQMI (SEQ ID NO: 747) | |
| Xyn | 22 | Linker region of XynAS9 (PDB | SGSGTTTTTTTSTTTGGTDPT | Li et al. (2019) |
| | | ID of 3WUB) from | (SEQ ID NO: 748) | |
| | | Streptomycessp. S9 | | |
| HP-76 | 76 | chicken villin headpiece | VFTATTTLVPTKLETFPLDV | McKnight et al. |
| | | | LVNTAAEDLPRGVDPSRKEN | (1996) |
| | | | HLSDEDFKAVFGMTRSAFAN | |
| | | | LPLWKQQNLKKEKGLF (SEQ | |
| | | | ID NO: 749) | |
| HP-35 | 35 | C-terminus of chicken villin | LSDEDFKAVFGMTRSAFANL | McKnight et al. |
| | | headpiece | PLWKQQNLKKEKGLF (SEQ | (1996) |
| | | | ID NO: 750) | |
| Foldon | 27 | derived from the native T4 | GYIPEAPRDGQAYVRKDGE | Du et al. (2008) |
| | | phage fibritin | WVLLSTFL (SEQ ID NO: 751) | |
| PPC1 | 184 | Full pre-peptidase C-terminal | TNVTFTMSGGTGDADLYVR | Yan et al. (2009) |
| | | domain of deep-sea | AGSKPTSTTYDCRPYKGGNS | |
| | | psychroolerant bacterium | EECSIDSPTAGTYHVMLRGY | |
| | | Pseudoalteromonas sp. SM9913 | SAYSGVSLVGNITGGSTGGG | |
| | | | SGTPQAGGGTVSDITANAGQ | |
| | | | WKHYTLDVPAGMANFTVTT | |
| | | | SGGTGDADLFVKFGSQPTSS | |
| | | | SYDCRPYKNGNAETCTFSNP | |
| | | | QAGTWHLSVNAYQTFSGLT | |
| | | | LSGQYQP (SEQ ID NO: 752) | |
| PPC2 | 67 | Half of pre-peptidase C- | TNVTFTMSGGTGDADLYVR | Yan et al. (2009) |
| | | terminaldomain of deep- | AGSKPTSTTYDCRPYKGGNS | |
| | | sea psychrotolerant bacterium | EECSIDSPTAGTYHVMLRGY | |
| | | Pseudoalteromonas sp. | SAYSGVSL (SEQ ID NO: 753) | |
| | | SM9913 | | |
| PPC3 | 85 | Half of pre-peptidase C- | AGQWKHYTLDVPAGMANF | Yan et al. (2009) |
| | | terminaldomain of deep- | TVTTSGGTGDADLFVKFGSQ | |
| | | sea psychrotolerant bacterium | PTSSSYDCRPYKNGNAETCT | |
| | | Pseudoalteromonas sp. | FSNPQAGTWHLSVNAYQTFS | |
| | | SM9913 | GLTLSGQ (SEQ ID NO: 754) | |
| KerSMF | 105 | pre-peptidase C-terminal | NPGGNVLQNNVPVTGLGAA | Fang et al. (2016); |
| | | domainof keratinase from | TGAELNYTVAVPAGSSQLRV | Fang et al. (2017) |
| | | Stenotrophomonas maltophilia | TISGGSGDADLYVRQGSAPT | |
| | | (KerSMF, NCBI accession no. | DTSYTCRPYLSGNSETCTINS | |
| | | AGK29593.1) | PAAGTWYVRVKAYSTFSGV | |
| | | | TLNAQY (SEQ ID NO: 755) | |
| KerSMD | 106 | pre-peptidase C-terminal | SCGPVATPLTNKAAVGGLN | Fang et al. (2016); |
| | | domain of keratinase from | GTAGSSRLYSFEAAAGKQLS | Fang et al. (2017) |
| | | Stenotrophomonas maltophilia | VITYGGTGNVSVYIAQGREP | |
| | | (KerSMD, NCBI accession no. | SASDNDGKSTRPGTSETVRV | |
| | | AGK12420.1) | NKPVAGTYYIKVVGEAAYN | |
| | | | GVSILATQ (SEQ ID NO: 756) | |
| DDFD1 | 217 | Fusion of two pre-peptidase C- | NPGGNVLQNNVPVTGLGAA | Fang et al. (2017) |
| | | terminal domain of keratinase | TGAELNYTVAVPAGSSQLRV | |
| | | from Stenotrophomonas | TISGGSGDADLYVRQGSAPT | |
| | | maltophilia (KerSMF, | DTSYTCRPYLSGNSETCTINS | |
| | | followed by KerSMD) | PAAGTWYVRVKAYSTFSGV | |
| | | | TLNAQYEEPCTESCGPVATP | |
| | | | LINKAAVGGLNGTAGSSRL | |
| | | | YSFEAAAGKQLSVITYGGTG | |
| | | | NVSVYIAQGREPSASDNDGK | |
| | | | STRPGTSETVRVNKPVAGTY | |
| | | | YIKVVGEAAYNGVSILATQ | |
| | | | (SEQ ID NO: 757) | |
| DDFD2 | 217 | Fusion of two pre-peptidase C- | SCGPVATPLTNKAAVGGLN | Fang et al. (2017) |
| | | terminal domain of keratinase | GTAGSSRLYSFEAAAGKQLS | |
| | | from Stenotrophomonas | VITYGGTGNVSVYIAQGREP | |
| | | maltophilia (KerSMF, | SASDNDGKSTRPGTSETVRV | |
| | | followed by KerSMD | NKPVAGTYYIKVVGEAAYN | |
| | | | GVSILATQEEPCTENPGGNV | |
| | | | LQNNVPVTGLGAATGAELN | |
| | | | YTVAVPAGSSQLRVTISGGS | |
| | | | GDADLYVRQGSAPTDTSYTC | |
| | | | RPYLSGNSETCTINSPAAGT | |
| | | | WYVRVKAYSTFSGVTLNAQY | |
| | | | (SEQ ID NO: 758) | |
| GD-95 | 20 | C-terminal region of Lipasefrom | SFDIRAFYLRLAEQLASLRP | Gudiukaite et al. |
| | | Geobacillus sp. 95 | (SEQ ID NO: 759) | (2014) |
| BACa | 12 | C-terminal region of the A | REEKPSSAPSS (SEQ ID NO: | Carver et al. (1998) |
| | | subunit of bovine a-crystallin | 760) | |
| BACb | 14 | C-terminal region of the B | REEKPAVTAAPKK (SEQ ID | Carver et al. (1998); |
| | | subunit of bovine a-crystallin | NO: 761) | Treweek et al. (2007) |

### Example 11: Evaluation of cDNA Synthesis Facilitated by MMLV RTase Mutant Fusions with C-Terminal Peptide Extensions

The ability of an MMLV RTase with a C-terminal peptide extension to synthesize cDNA from purified total RNA was evaluated compared to the base construct of MMLV RTase without a C-terminal peptide extension. MMLV RTases with C-terminal peptide extensions were tested by random hexamer priming using standard two-step cDNA synthesis.

A colony of BL21(DE3) cells with the appropriate strain (Table 39) was inoculated in TB media (5 mL) with kanamycin (0.05 mg/mL) and grown at 37° C. until an OD of approximately 0.9 was achieved, followed by cooling of cultures on ice for 5 minutes. Protein expression was induced by the addition of 1M IPTG (2.5 uL), followed by growth at 18° C. for 21 hours. Cells were harvested via centrifugation at 4,700×g for 10 minutes and cell pellets re-suspended in lysis buffer containing 50 mM NaPO₄, pH 7.8, 5% glycerol, 300 mM NaCl, and 10 mM imidazole. Cells were lysed by the addition of 1×BugBuster (Millipore Sigma) and incubated on an end-over-end mixer for 15 minutes at room temperature. Cellular debris was removed from the lysate by centrifugation at 4,700×g for for 10 minutes at 4° C.

Cleared lysates were applied to a HisPur™ Ni-NTA spin plate (ThermoFisher) after equilibrating the resin with Screening His-Bind buffer (50 mM NaPO₄, pH 7.8, 5% glycerol, 300 mM NaCl, and 10 mM imidazole). Samples were washed three times with Screening His-Wash buffer (50 mM NaPO₄, pH 7.8, 5% glycerol, 300 mM NaCl, and 25 mM imidazole) and eluted using Screening His-Elution buffer (50 mM NaPO₄, pH 7.8, 5% glycerol, 300 mM NaCl, and 250 mM imidazole). Purified proteins were normalized to a set concentration (375 nM) and standard two-step cDNA synthesis carried out. More specifically, RTases (4 μL, 375 nM) were added to a reaction mixture containing: RNA (90 ng), dNTPs (100 μM), random hexamers and oligo dT primers (5 ng/uL each), first strand synthesis buffer (1×, 50 mM Potassium Acetate, 20 mM Tris-acetate, 10 mM Magnesium Acetate, 100 m/ml BSA, 0.6 M trehalose, 10 mM DTT, pH 7.9), SuperaseIN (0.17 U/μL) in a 50 μL volume. The reaction was run at 25° C. for 2 minutes, 50° C. for 15 minutes, and 80° C. for 10 minutes.

Resultant cDNA, synthesized by the RTase mutants, was quantified by qPCR amplification for identification of the human SFRS9 gene. Reactions were performed in qPCR assay master mix comprised of Integrated DNA Technologies PrimeTime Gene Expression Master Mix (GEM, 1×), SFRS9 primer set (500 nM, Table 2), and SFRS9 probe (250 nM, Table 2) in a 10:1 ratio for a final volume of 20 μL. Reactions were run on a qPCR instrument (QuantStudio7 Flex) on a 95° C. hold for 3 minutes, followed by 40 cycles of 95° C. for 15 seconds, and 60° C. for one minute for 40 cycles. Reactions were analyzed and Ct value reported (Table 40).

TABLE 40
Two-Step cDNA Synthesis by MMLV-RTase with
C-terminal peptide extension using random priming.

| C-terminal Peptide | Ct Mean | Ct Standard Deviation |
|---|---|---|
| No tag | 29.565 | 0.130 |
| Control Tag 1 | 29.260 | 0.020 |
| ATS-21 | 26.996 | 0.019 |
| ATS-42 | 28.942 | 0.044 |
| ATTa Peptide | 26.679 | 0.138 |
| ATTb Peptide | 25.907 | 0.077 |
| ATYd | 29.697 | 0.105 |
| BACa | 34.043 | 0.126 |
| DDFD1 | 27.716 | 0.053 |
| DDFD2 | 33.042 | 0.195 |
| Foldon | 30.500 | 0.031 |
| GD-95 | 29.925 | 0.043 |
| HP-35 | 29.328 | 0.110 |
| HP-76 | 30.324 | 0.034 |
| KerSMD | 29.362 | 0.054 |
| KerSMF | 33.338 | 0.167 |
| LipB | 26.097 | 0.109 |
| Msb | 26.998 | 0.041 |
| Od | 28.048 | 0.125 |
| PPC1 | 27.410 | 0.047 |
| PPC2 | 26.595 | 0.099 |
| PPC3 | 28.040 | 0.094 |
| Syn103-115 | 27.055 | 0.011 |
| Syn114-126 | 26.288 | 0.062 |
| Syn119-140 | 34.974 | 0.975 |
| Syn130-140 | 26.678 | 0.068 |
| Syn96-140 | 28.049 | 0.099 |

Eighteen of the thirty C-terminal peptides tested demonstrated an increase in the overall activity using random priming compared to the base construct. Ten of the eighteen C-terminal peptides (i.e., ATTb Peptide, LipB, Syn114-126, PPC2, Syn130-140, ATTa Peptide, ATS-21, Msb, Syn103-115 and PPC1) demonstrated a 6-fold or higher increase in overall activity as compared to the base construct.

### Example 12: C-Terminal Peptide Extension of Reverse Transcriptase Evaluation at High Temperatures

The ability of RTase with a C-terminal peptide extension versus a base construct without a C-terminal peptide to synthesize cDNA from purified total RNA was compared. MMLV RTases with C-terminal peptides were tested at higher temperatures to determine robust reverse transcription activity. The standard two-step procedure was used in which RTases (4 μL, 375 nM) were added to a reaction mixture containing RNA (90 ng), dNTPs (100 μM), random hexamers and oligo dT primers (5 ng/uL each), first strand synthesis buffer (1×, 50 mM Potassium Acetate, 20 mM Tris-acetate, 10 mM Magnesium Acetate, 100 μg/ml BSA, 0.6M trehalose, 10 mM DTT, pH 7.9), and SuperaseIN (0.17 U/μL) in a 50 μL volume. The reaction was run at 25° C. for 2 minutes, followed by 55° C. or 60° C. for 15 minutes, and 80° C. for 10 minutes.

cDNA synthesized by RTase mutants was quantified by qPCR amplification using a SFRS9 human cell gene assay that included a master mix comprised of Integrated DNA Technologies PrimeTime Gene Expression Master Mix (GEM, 1×), SFRS9 primer set (500 nM, Table 2), and SFRS9 probe (250 nM, Table 2) in a 10:1 ratio (assay master mix:synthesized cDNA) in a final volume of 20 μL and reaction run on a qPCR (QuantStudio7 Flex) at a 95° C. hold for 3 minutes, followed by 40 cycles of 95° C. for 15 seconds, and 60° C. for one minute and Ct value reported in Table 41.

TABLE 41
Ct value from Two-Step cDNA Synthesis reactions by
MMLV-RTase with C-terminal peptide extensions
using random primimg at higher temperatures

| C-terminal | Temperature | Ct | Ct Standard |
|---|---|---|---|
| Peptide | (° C.) | Mean | Deviation |
| No tag | 55 | 33.627 | 0.072 |
| | 60 | 35.028 | 0.332 |
| Control Tag 1 | 55 | 34.544 | 0.147 |
| | 60 | 35.175 | 0.241 |
| ATS-21 | 55 | 35.176 | 0.720 |
| | 60 | 37.374 | 0.370 |
| ATS-42 | 55 | 34.450 | 0.113 |
| | 60 | 36.448 | 0.451 |
| ATTa | 55 | 30.802 | 0.063 |
| | 60 | 34.967 | 1.278 |
| ATTb | 55 | 30.796 | 0.166 |
| | 60 | 33.003 | 0.082 |
| ATYd | 55 | 35.835 | 0.632 |
| | 60 | 36.123 | 0.096 |
| BACa | 55 | 36.154 | 0.816 |
| | 60 | 36.950 | 0.733 |
| DDFD1 | 55 | 32.733 | 0.081 |
| | 60 | 34.499 | 0.395 |
| DDFD2 | 55 | 36.891 | 0.972 |
| | 60 | 36.537 | 0.525 |
| Foldon | 55 | 34.633 | 0.657 |
| | 60 | 36.545 | 1.237 |
| GD-95 | 55 | 34.310 | 0.772 |
| | 60 | 36.007 | 0.793 |
| HP-35 | 55 | 35.310 | 0.055 |
| | 60 | 35.917 | 0.347 |
| HP-76 | 55 | 36.183 | 0.344 |
| | 60 | 36.006 | 0.267 |
| KerSMD | 55 | 34.195 | 0.392 |
| | 60 | 34.830 | 0.144 |
| KerSMF | 55 | 35.961 | 0.901 |
| | 60 | 36.713 | 0.309 |
| LipB | 55 | 31.123 | 0.108 |
| | 60 | 33.129 | 0.207 |
| Msb | 55 | 32.471 | 0.116 |
| | 60 | 35.981 | 0.526 |
| Od | 55 | 31.560 | 0.122 |
| | 60 | 33.713 | 0.255 |
| PPC1 | 55 | 32.073 | 0.169 |
| | 60 | 33.963 | 0.404 |
| PPC2 | 55 | 33.545 | 0.092 |
| | 60 | 35.072 | 0.235 |
| PPC3 | 55 | 33.125 | 0.623 |
| | 60 | 33.794 | 0.134 |
| Syn103-115 | 55 | 32.716 | 0.081 |
| | 60 | 34.455 | 0.564 |
| Syn114-126 | 55 | 30.674 | 0.136 |
| | 60 | 32.459 | 0.143 |
| Syn119-140 | 55 | 36.978 | 0.420 |
| | 60 | 36.920 | 0.752 |
| Syn130-140 | 55 | 32.242 | 0.234 |
| | 60 | 34.022 | 0.388 |
| Syn96-140 | 55 | 34.978 | 0.604 |
| | 60 | 35.918 | 1.100 |
| Trx | 55 | 34.821 | 0.236 |
| | 60 | 36.102 | 0.649 |
| Xyn | 55 | 35.125 | 0.268 |
| | 60 | 36.063 | 0.585 |
| Yd | 55 | 35.424 | 0.126 |
| | 60 | 36.527 | 0.585 |

Among the 30 C-terminal peptides tested, 11 demonstrated increased overall activity when using random priming as compared to the base construct. A 6-fold or higher increase in overall activity was demonstrated in 5 of the 11 C-terminal peptides (i.e., Syn114-126, ATTb Peptide, ATTa, Peptide, LipB and Od) at 55° C. as compared to the base construct. Two of the 11 C-terminal peptides (i.e., Syn114-126 and ATTb peptide) demonstrated a 6-fold or higher increase in overall activity at 60° C. as compared to the base construct.

### Example 13: C- or N-Terminal Peptide Extension of Reverse Transcriptase Evaluation by Random Priming

The ability of an MMLV RTase with a C-terminal peptide extension to synthesize cDNA from purified total RNA was evaluated as compared to the base construct of MMLV RTase without a C-terminal peptide extension. MMLV RTases with C-terminal or N-terminal peptide extensions (Table 42) were expressed and crudely extracted from BL21(DE3) E. coli cells and purified via HisPur™ Ni-NTA spin plate (ThermoFisher).

TABLE 42
C-terminal or N-terminal peptide studies.

| C-terminal | Size (AA | | | |
|---|---|---|---|---|
| peptide | residues) | Origin | Amino Acid Sequence | Reference |
| ATTa | 40 | C-end tail of Arabidopsis | EGMEEGEFSEAREDLAALE | Zhang et al. |
| Peptide | | tubulins, TUA2 (NCBI accession | KDYEEVGAEGGDDEDDEGE | (2015) |
| | | no. NP_175423.1) | EY (SEQ ID NO: 735) | |
| ATTb | 50 | C-end tail of Arabidopsis | EGMDEMEFTEAESNMNDLV | Zhang et al. |
| Peptide | | tubulins, TUA3 (NCBI accession | SEYQQYQDATADEEGDYED | (2015) |
| | | no. NP_568960.1) | EEEGEYQQEEEY(SEQ ID NO: | |
| | | | 736 | |
| Od | 182 | N-terminal domain of E. coli | DIVDSDQIEDIIQMINDMGIQ | Zou et al. |
| | | rpoD (NCBI accessionno. | VMEEAPDADDLMLAENTAD | (2008) |
| | | CAD6003062.1) | EDAAEAAAQVLSSVESEIGR | |
| | | | TTDPVRMYMREMGTVELLT | |
| | | | REGEIDIAKRIEDGINQVQCS | |
| | | | VAEYPEAITYLLEQYDRVEA | |
| | | | EEARLSDLITGFVDPNAEED | |
| | | | LAPTATHVGSELSQEDLDDD | |
| | | | EDEDEEDGDDDSADDD | |
| | | | NSIDPE (SEQ ID NO: 739) | |
| Syn114-126 | 3 | C-end tail of human a- | NDMPVDPDNEAYE (SEQ ID | Park et al. |
| | | synuclein (NCBI accessionno. | NO: 744) | (2004) |
| | | NP_001362216.1) | | |
| LipB | 26 | C-end tail of Fusarium | DMSDEELEKKLTQYSEMDQ | Nagao et al. |
| | | heterosporum Lipase B | EFVKQMI (SEQ ID NO: 747) | (1998) |
| PPC1 D | 184 | Full pre-peptidase C-terminal | TNVTFTMSGGTGDADLYVR | Yan et al. |
| | | domain of deep- | AGSKPTSTTYDCRPYKGGNS | (2009) |
| | | sea psychrotolerant bacterium | EECSIDSPTAGTYHVMLRGY | |
| | | Pseudoalteromonas sp. SM9913 | SAYSGVSLVGNITGGSTGGG | |
| | | | SGTPQAGGGTVSDITANAGQ | |
| | | | WKHYTLDVPAGMANFTVT | |
| | | | SGGTGDADLFVKFGSQPTSS | |
| | | | SYDCRPYKNGNAETCTFSNP | |
| | | | QAGTWHLSVNAYQTFSGLT | |
| | | | LSGQYQP (SEQ ID NO: 752) | |
| Sto7d+K12L | 64 | “7 kDa DNA-binding” proteinfrom | MVTVKFKYKGEELEVDISKI | Kalichuk et |
| | | Sulfolobus solfataricus | KKVWRVGKMISFTYDDNGK | al. (2016) |
| | | | TGRGAVSEKDAPKELLQML | |
| | | | EKSGKK (SEQ ID NO: 762) | |

Resultant RTases were tested by random hexamer priming using a standard two-step cDNA synthesis. More specifically, RTases (4 μL, 375 nM) were added to a reaction mixture containing RNA (90 ng), dNTPs (100 μM), random hexamers and oligo dT primers (5 ng/uL each), first strand synthesis buffer (1×, 50 mM Potassium Acetate, 20 mM Tris-acetate, 10 mM Magnesium Acetate, 100 m/ml BSA, 0.6 M trehalose, 10 mM DTT, pH 7.9), SuperaseIN (0.17 U/μL) in a 50 μL volume. The reaction was run at 25° C. for 2 minutes, 55 or 60° C. for 15 minutes, and 80° C. for 10 minutes.

Resultant cDNA, synthesized by the RTase mutants, was quantified by qPCR amplification for identification of the human SFRS9 gene. Reactions were performed in qPCR assay master mix comprised of Integrated DNA Technologies PrimeTime Gene Expression Master Mix (GEM, 1×), SFRS9 primer set (500 nM, Table 2), and SFRS9 probe (250 nM, Table 2) in a 10:1 ratio for a final volume of 20 μL. Reactions were run on a qPCR instrument (QuantStudio7 Flex) on a 95° C. hold for 3 minutes, followed by 40 cycles of 95° C. for 15 seconds, and 60° C. for one minute for 40 cycles. Reactions were analyzed and Ct value reported (Table 42).

Seven of the 43 C-terminal or N-terminal peptide extensions (i.e., C-terminal ATTa Peptide, C-terminal ATTb Peptide, C-terminal LipB, C- and N-terminal Syn114-126, C-terminal Od and C-terminal LipB+Od) demonstrated either an increase or negligible affect in the overall activity using random priming as compared to the base construct (Table 43).

TABLE 43
Two-Step cDNA Synthesis by MMLV-RTase with C- or N-terminal
peptide extension using random priming.

| | Temper- | Ct | Ct Standard |
|---|---|---|---|
| RTase | ature (C) | Mean | Deviation |
| MMLV-II | 55 | 29.352 | 0.568 |
| | 60 | 31.560 | 0.13 |
| MMLV-II with CTD Od + Od | 55 | 33.932 | 0.808 |
| | 60 | 34.854 | 2.151 |
| MMLV-II with CTD ATTa | 55 | 26.589 | 0.075 |
| | 60 | 29.887 | 0.179 |
| MMLV-II with CTD | 55 | 32.573 | 0.962 |
| LipB + ATTb | 60 | 32.253 | 0.589 |
| MMLV-II with CTD | 55 | 32.451 | 0.106 |
| ATTa + LipB | 60 | 33.326 | 0.526 |
| MMLV-II with CTD | 55 | 33.277 | 1.124 |
| ATTb + ATTa | 60 | 33.094 | 0.868 |
| MMLV-II with CTD | 55 | 34.883 | 0.606 |
| ATTb + LipB | 60 | 33.887 | 1.635 |
| MMLV-II with CTD Syn114- | 55 | 33.284 | 0.368 |
| 126 + ATTb | 60 | 34.582 | 2.122 |
| MMLV-II with CTD ATTb | 55 | 27.949 | 0.303 |
| | 60 | 31.919 | 1.536 |
| MMLV-II with NTD ATTb | 55 | 32.659 | 0.525 |
| | 60 | 33.757 | 0.268 |
| MMLV-II with CTD Syn114- | 55 | 32.876 | 0.857 |
| 126 + LipB | 60 | 33.598 | 0.227 |
| MMLV-II with CTD | 55 | 33.510 | 0.871 |
| Od + Syn114-126 | 60 | 33.011 | 0.435 |
| MMLV-II with CTD | 55 | 32.355 | 0.535 |
| LipB + Syn114-126 | 60 | 33.490 | 0.931 |
| MMLV-II with CTD | 55 | 32.604 | 0.446 |
| ATTa + PPC1 | 60 | 34.108 | 1.18 |

### Example 14: C-Terminal Peptide Extension of Reverse Transcriptase Evaluation by Random Priming

The ability of an MMLV RTase with a C-terminal peptide extension to synthesize cDNA from purified total RNA was evaluated as compared to the base construct of MMLV RTase without a C-terminal peptide extension. MMLV RTases with C-terminal peptides were tested by random hexamer priming using standard two-step cDNA synthesis.

More specifically, RTases (1 μL, 620 nM) were added to a reaction mixture containing RNA (20 ng), dNTPs (100 μM), random hexamers, and oligo dT primers (5 ng/uL each), first strand synthesis buffer (1×, 50 mM, Potassium Acetate, 20 mM Tris-acetate, 10 mM Magnesium Acetate, 100 μg/ml BSA, 0.6, M trehalose, 10 mM DTT, pH 7.9), SuperaseIN (0.17 U/μL)) in a 20 μL volume and run at 25° C. for 2 minutes, followed by 42-65° C. for 15 minutes, and 80° C. for 10 minutes.

Resultant cDNA, synthesized by the RTase mutants, was quantified by qPCR amplification for identification of the human SFRS9 gene. Reactions were performed in qPCR assay master mix comprising Integrated DNA Technologies PrimeTime Gene Expression Master Mix (GEM, 1×), SFRS9 primer set (500 nM, Table 2), and SFRS9 probe (250 nM, Table 2) in a 10:1 ratio for a final volume of 20 μL. Reactions were run on a qPCR instrument (QuantStudio7 Flex) on a 95° C. hold for 3 minutes, followed by 40 cycles of 95° C. for 15 seconds, and 60° C. for one minute for 40 cycles. Reactions were analyzed and Ct value reported (Table 44).

All five C-terminal peptide extensions tested demonstrated an increase in the overall activity using random priming compared to the base construct. Two of the five C-terminal peptide extensions (i.e., LipB and Syn114-126) retained or showed an increase in overall activity as compared to the mutant variant without the C-terminal peptide.

TABLE 44
Two-Step cDNA Synthesis by MMLV-RTase with C-terminal peptide
extensions using random priming.

| | Temper- | | Ct |
|---|---|---|---|
| | ature | Ct | Standard |
| RTase | (° C.) | Mean | Deviation |
| MMLV-II | 42 | 24.643 | 0.039 |
| | 43.4 | 24.780 | 0.066 |
| | 46.4 | 24.753 | 0.079 |
| | 50.8 | 25.282 | 0.040 |
| | 56.4 | 30.126 | 0.135 |
| | 61 | 31.817 | 0.036 |
| | 63.6 | 32.628 | 0.220 |
| | 65 | 33.110 | 0.201 |
| SuperScript-IV | 42 | 24.501 | 0.066 |
| | 43.4 | 24.731 | 0.085 |
| | 46.4 | 24.689 | 0.072 |
| | 50.8 | 24.637 | 0.021 |
| | 56.4 | 25.041 | 0.070 |
| | 61 | 25.808 | 0.034 |
| | 63.6 | 25.972 | 0.118 |
| | 65 | 26.097 | 0.160 |
| MMLV-II Q68R/Q79R/L82Y/L99R/L280I/ | 42 | 24.809 | 0.089 |
| E282D/Q299E/T306K/V433N/I593W | 43.4 | 24.817 | 0.068 |
| | 46.4 | 24.820 | 0.095 |
| | 50.8 | 24.745 | 0.032 |
| | 56.4 | 25.400 | 0.072 |
| | 61 | 25.898 | 0.083 |
| | 63.6 | 26.123 | 0.116 |
| | 65 | 26.079 | 0.035 |
| MMLV-II Q68R/Q79R/L82Y/L99R/L280I/ | 42 | 24.672 | 0.075 |
| | 43.4 | 24.800 | 0.056 |
| | 46.4 | 24.631 | 0.069 |
| E282D/Q299E/T306K/V433N/I593W | 50.8 | 24.591 | 0.018 |
| with ATTa | 56.4 | 24.858 | 0.058 |
| | 61 | 26.147 | 0.083 |
| | 63.6 | 26.682 | 0.144 |
| | 65 | 26.880 | 0.103 |
| MMLV-II Q68R/Q79R/L82Y/L99R/L280I/ | 42 | 24.906 | 0.076 |
| E282D/Q299E/T306K/V433N/I593W | 43.4 | 24.759 | 0.074 |
| with ATTb | 46.4 | 24.618 | 0.007 |
| | 50.8 | 24.879 | 0.185 |
| | 56.4 | 25.388 | 0.065 |
| | 61 | 29.436 | 0.154 |
| | 63.6 | 30.592 | 0.128 |
| | 65 | 30.882 | 0.109 |
| MMLV-II Q68R/Q79R/L82Y/L99R/L280I/ | 42 | 24.677 | 0.044 |
| E282D/Q299E/T306K/V433N/I593W | 43.4 | 24.685 | 0.009 |
| with LipB | 46.4 | 24.785 | 0.147 |
| | 50.8 | 24.751 | 0.063 |
| | 56.4 | 24.885 | 0.133 |
| | 6 | 25.815 | 0.151 |
| | 63.6 | 25.919 | 0.116 |
| | 65 | 26.136 | 0.087 |
| MMLV-II Q68R/Q79R/L82Y/L99R/L280I/ | 42 | 24.823 | 0.260 |
| E282D/Q299E/T306K/V433N/I593W | 43.4 | 24.869 | 0.140 |
| with Od | 46.4 | 24.613 | 0.043 |
| | 50.8 | 24.722 | 0.199 |
| | 56.4 | 25.933 | 0.137 |
| | 61 | 28.688 | 0.190 |
| | 63.6 | 28.985 | 0.167 |
| | 65 | 29.440 | 0.043 |
| MMLV-II Q68R/Q79R/L82Y/L99R/L280I/ | 42 | 24.624 | 0.071 |
| E282D/Q299E/T306K/V433N/I593W | 43.4 | 24.648 | 0.065 |
| with Syn114-126 | 46.4 | 24.694 | 0.010 |
| | 50.8 | 24.614 | 0.091 |
| | 56.4 | 25.016 | 0.064 |
| | 61 | 25.667 | 0.030 |
| | 63.6 | 25.913 | 0.053 |
| | 65 | 25.723 | 0.055 |

### BIBLIOGRAPHY

1. Carver et al., “NMR spectroscopy of a-crystallin. Insights into the structure, interactions and chaperone action of small heat-shock proteins,” Int. J. Biol. Macromol. 22(3-4): 197-209 (1998).
2. Coffin et al., “The discovery of reverse transcriptase,” Ann. Rev. Virol. 3(1): 29-51 (2016).
3. Du et al., “Improvement of thermostability of recombinant collagen-like protein by incorporating a foldon sequence,” Appl. Microbiol. Biotechnol. 79(2): 195-202 (2008).
4. Fang et al., “Enhancement of the catalytic efficiency and thermostability of Stenotrophomonas sp. keratinase KerSMD by domain exchange with KerSMF,” Microb. Biotechnol. 9(1): 35-46 (2016).
5. Fang et al., “Rational protein engineering approaches to further improve the keratinolytic activity and thermostability of engineered keratinase KerSMD,” Biochem. Eng. J. 127: 147-53 (2017).
6. Gudiukaite et al., “Influence of N- and/or C-terminal regions on activity, expression, characteristics and structure of lipase from Geobacillus sp. 95,” Extremophiles 18(1): 131-145 (2014).
7. Hogrefe et al., “Mutant reverse transcriptase and methods of use,” U.S. Pat. No. 9,783,791.
8. Kalichuk et al., “The archaeal ‘7 kDa DNA-binding’ proteins: extended characterization of an old gifted family,” Sci. Rep. 6: 37274 (2016).
8. Kotewicz et al., “Cloned genes encoding reverse transcriptase lacking RNase H activity,” U.S. Pat. No. 5,405,776.
9. Kotewicz et al., “Isolation of cloned Moloney murine leukemia virus reverse transcriptase lacking ribonuclease H activity,” Nucleic Acids Res. 16(1): 265-77 (1988).
10. Lee et al., “Stabilizing peptide fusion for solving the stability and solubility problems of therapeutic proteins,” Pharm. Res. 22(10): 1735-46 (2005).
11. Li et al., “Improvement of GH10 family xylanase thermostability by introducing of an extra α-helix at the C-terminal,” Biochem. Biophys. Res. Commun. 515(3): 417-22 (2019).
12. McKnight et al., “A Thermostable 35-Residue Subdomain within Villin Headpiece,” J. Mol. Biol. 260: 126-34 (1996).
13. Nagao et al., “C-terminal peptide of Fusarium heterosporum lipase is necessary for its increasing thermostability,” J. Biochem. 124(6): 1124-29 (1998).
14. Park et al., “Stress-Induced Aggregation Profiles of GST-R-Synuclein Fusion Proteins: Role of the C-Terminal Acidic Tail of R-Synuclein in Protein Thermosolubility and Stability,” Biochemistry 41(12): 4137-46 (2002).
15. Park et al., “Effects of novel peptides derived from the acidic tail of synuclein (ATS) on the aggregation and stability of fusion proteins,” Protein Eng. Des. Sel. 17(3): 251-60 (2004).
16. Rogers et al., “Novel reverse transcriptases for use in high temperature nucleic acid synthesis,” U.S. Patent Application Publication No. US 2015/0210989 A1.
17. Treweek et al., “Site-directed mutations in the C-terminal extension of human abcrystallin affect chaperone function and block amyloid fibril formation,” PLoS One 2(10): e1046 (2007).
18. Yan et al., “Molecular analysis of the gene encoding a cold-adapted halophilic subtilase from deep-sea psychrotolerant bacterium Pseudoalteromonas sp. SM9913: cloning, expression, characterization and function analysis of the C-terminal PPC domains,” Extremophiles 13(4): 725-33 (2009).
19. Zhang et al., “Improvement on the thermal stability and activity of plant cytosolic ascorbate peroxidase 1 by tailing hyper-acidic fusion partners,” Biotechnol. Lett. 37(4): 891-98 (2015).
20. Zou et al., “Hyper-acidic protein fusion partners improve solubility and assist correct folding of recombinant proteins expressed in Escherichia coli,” J. Biotechnol. 135(4): 333-39 (2008).

What is claimed is:

1. An isolated Moloney murine leukemia virus (MMLV) reverse transcriptase (RTase) mutant comprising a C-terminal peptide extension, an N-terminal peptide extension, or both a C-terminal and an N-terminal peptide extension.

2. The isolated MMLV RTase mutant of claim 1, wherein the C-terminal peptide extension comprises an amino acid sequence of SEQ ID NO: 732-761.

3. The isolated MMLV RTase mutant of claim 1, wherein the N-terminal peptide extension comprises an amino acid sequence of SEQ ID NO: 732-761.

4. The isolated MMLV RTase mutant of claim 1, wherein the C-terminal peptide extension and N-terminal peptide extension comprise an amino acid sequence of SEQ ID NO: 732-761.

5. The isolated MMLV RTase mutant of claim 1, wherein the C-terminal peptide extension and/or N-terminal peptide extension are unnatural peptide tags.

6. The isolated MMLV RTase mutant of claim 1, wherein the C-terminal peptide extension or N-terminal peptide extension is the amino acid sequence of SEQ ID NO: 735, SEQ ID NO: 736, SEQ ID NO: 739, SEQ ID NO: 744, or SEQ ID NO: 747.

7. The isolated MMLV RTase mutant of claim 6, wherein the C-terminal peptide extension or N-extension peptide extension is the amino acid sequence of SEQ ID NO: 736 or SEQ ID NO: 744.

8. The isolated MMLV RTase mutant of claim 6, wherein the C-terminal peptide extension or N-terminal peptide extension is the amino acid sequence of SEQ ID NO: 744 or SEQ ID NO: 747.

9. An isolated Moloney murine leukemia virus (MMLV) reverse transcriptase (RTase) mutant comprising the amino acid sequence of SEQ ID NO: 674 (MMLV-II), wherein the amino acid sequence of the MMLV RTase mutant further comprises a C-terminal peptide extension or N-terminal peptide extension and at least two amino acid substitutions that are:
(a) a glutamine to arginine substitution at position 68 (Q68R);
(b) a glutamine to arginine substitution at position 79 (Q79R);
(c) a leucine to tyrosine at position 82 (L82Y);
(d) a leucine to arginine substitution at position 99 (L99R);
(e) a leucine to isoluecine at position 280 (L280I);
(f) a glutamic acid to aspartic acid substitution at position 282 (E282D);
(g) a glutamine to glutamic acid substitution at position 299 (Q299E);
(h) threonine to lysine at position 306 (T306K);
(i) a valine to asparagine at position 433 (V433N); or
(j) an isoleucine to tryptophan at position 593 (I593W).

10. The isolated MMLV Rtase mutant of claim 9, wherein the C-terminal peptide extension or N-terminal peptide extension comprises an amino acid sequence of SEQ ID NO: 735, SEQ ID NO: 736, SEQ ID NO: 739, SEQ ID NO: 744, or SEQ ID NO: 747.

11. The isolated MMLV RTase mutant of any one of claims 1 to 10, wherein the MMLV RTase mutant lacks RNase H activity.

12. The isolated MMLV RTase mutant of any one of claims 1 to 10, wherein the MMLV RTase mutant possesses at least one of the following characteristics: enhanced DNA synthesis, increased fidelity, or enhanced thermostability.

13. An isolated nucleic acid molecule comprising a nucleotide sequence encoding the MMLV Rtase mutant of any one of claims 1 to 10.

14. A composition comprising the isolated MMLV RTase mutant of of any one of claims 1 to 10.

15. The composition of claim 14, wherein the isolated MMLV RTase mutant possesses at least one of the following characteristics: enhanced DNA synthesis, increased fidelity, or enhanced thermostability.

16. A kit comprising the isolated MMLV RTase mutant of mutant of any one of claims 1 to 10.

17. The kit of claim 16, wherein the isolated MMLV RTase mutant lacks RNAse H activity.

18. The kit of claim 16, wherein the isolated MMLV RTase mutant possesses at least one of the following characteristics: enhanced DNA synthesis, increased fidelity, or enhanced thermostability.

19. A method for synthesizing complementary deoxyribonucleic acid (cDNA) comprising:
(a) providing the isolated MMLV RTase mutant of any one of claims 1 to 10; and
(b) contacting the isolated MMLV RTase mutant with a nucleic acid template to permit synthesis of cDNA.

20. A method for performing reverse transcription-polymerase chain reaction (RT-PCR) comprising:
(a) providing the isolated MMLV RTase mutant of any one of claims 1 to 10; and
(b) contacting the isolated MMLV RTase mutant with a nucleic acid template to replicate and amplify the nucleic acid template.


SEQUENCE LISTING (762 sequences)

SEQ ID NO: 1 - DNA, 69 bp, synthetic construct
  misc_feature 1..69: note = Synthetic oligonucleotide
  source 1..69: mol_type = other DNA
ggtatatctccttcttaaagttaaacaaaattatttctagaggggaatgatccggctgctaacaaagcc

SEQ ID NO: 2 - DNA, 21 bp, synthetic construct
  misc_feature 1..21: note = Synthetic oligonucleotide
  source 1..21: mol_type = other DNA
gatccggctgctaacaaagcc

SEQ ID NO: 3 - DNA, 52 bp, synthetic construct
  misc_feature 1..52: note = Synthetic oligonucleotide
  source 1..52: mol_type = other DNA
ttttgtttaactttaagaaggagatataccatgggcagcagccatcatcatc

SEQ ID NO: 4 - DNA, 59 bp, synthetic construct
  misc_feature 1..59: note = Synthetic oligonucleotide
  source 1..59: mol_type = other DNA
gcagccaactcagcttcctttcgggctttgttaaaaatgctcgctagtgtagggagagc

SEQ ID NO: 5 - DNA, 54 bp, synthetic construct
  misc_feature 1..54: note = Synthetic oligonucleotide
  source 1..54: mol_type = other DNA
aagcaccgttgatcatcccgttagcggcaacgtctacacctgtctctatcaaac

SEQ ID NO: 6 - DNA, 54 bp, synthetic construct
  misc_feature 1..54: note = Synthetic oligonucleotide
  source 1..54: mol_type = other DNA
aagcaccgttgatcatcccgttacgtgcaacgtctacacctgtctctatcaaac

SEQ ID NO: 7 - DNA, 54 bp, synthetic construct
  misc_feature 1..54: note = Synthetic oligonucleotide
  source 1..54: mol_type = other DNA
aagcaccgttgatcatcccgttagaagcaacgtctacacctgtctctatcaaac

SEQ ID NO: 8 - DNA, 57 bp, synthetic construct
  misc_feature 1..57: note = Synthetic oligonucleotide
  source 1..57: mol_type = other DNA
ccgttgatcatcccgttaaaggcagcgtctacacctgtctctatcaaacagtacccc

SEQ ID NO: 9 - DNA, 57 bp, synthetic construct
  misc_feature 1..57: note = Synthetic oligonucleotide
  source 1..57: mol_type = other DNA
ccgttgatcatcccgttaaaggcacgttctacacctgtctctatcaaacagtacccc

SEQ ID NO: 10 - DNA, 57 bp, synthetic construct
  misc_feature 1..57: note = Synthetic oligonucleotide
  source 1..57: mol_type = other DNA
ccgttgatcatcccgttaaaggcagaatctacacctgtctctatcaaacagtacccc

SEQ ID NO: 11 - DNA, 56 bp, synthetic construct
  misc_feature 1..56: note = Synthetic oligonucleotide
  source 1..56: mol_type = other DNA
atcatcccgttaaaggcaacgtctgcgcctgtctctatcaaacagtaccccatgag

SEQ ID NO: 12 - DNA, 56 bp, synthetic construct
  misc_feature 1..56: note = Synthetic oligonucleotide
  source 1..56: mol_type = other DNA
atcatcccgttaaaggcaacgtctcgtcctgtctctatcaaacagtaccccatgag

SEQ ID NO: 13 - DNA, 56 bp, synthetic construct
  misc_feature 1..56: note = Synthetic oligonucleotide
  source 1..56: mol_type = other DNA
atcatcccgttaaaggcaacgtctgaacctgtctctatcaaacagtaccccatgag

SEQ ID NO: 14 - DNA, 58 bp, synthetic construct
  misc_feature 1..58: note = Synthetic oligonucleotide
  source 1..58: mol_type = other DNA
ccgttaaaggcaacgtctacacctgcgtctatcaaacagtaccccatgagtcaagagg

SEQ ID NO: 15 - DNA, 58 bp, synthetic construct
  misc_feature 1..58: note = Synthetic oligonucleotide
  source 1..58: mol_type = other DNA
ccgttaaaggcaacgtctacacctcgttctatcaaacagtaccccatgagtcaagagg

SEQ ID NO: 16 - DNA, 58 bp, synthetic construct
  misc_feature 1..58: note = Synthetic oligonucleotide
  source 1..58: mol_type = other DNA
ccgttaaaggcaacgtctacacctgaatctatcaaacagtaccccatgagtcaagagg

SEQ ID NO: 17 - DNA, 54 bp, synthetic construct
  misc_feature 1..54: note = Synthetic oligonucleotide
  source 1..54: mol_type = other DNA
taaaggcaacgtctacacctgtctctgcgaaacagtaccccatgagtcaagagg

SEQ ID NO: 18 - DNA, 54 bp, synthetic construct
  misc_feature 1..54: note = Synthetic oligonucleotide
  source 1..54: mol_type = other DNA
taaaggcaacgtctacacctgtctctcgtaaacagtaccccatgagtcaagagg

SEQ ID NO: 19 - DNA, 54 bp, synthetic construct
  misc_feature 1..54: note = Synthetic oligonucleotide
  source 1..54: mol_type = other DNA
taaaggcaacgtctacacctgtctctgaaaaacagtaccccatgagtcaagagg

SEQ ID NO: 20 - DNA, 51 bp, synthetic construct
  misc_feature 1..51: note = Synthetic oligonucleotide
  source 1..51: mol_type = other DNA
ggcaacgtctacacctgtctctatcgcgcagtaccccatgagtcaagaggc

SEQ ID NO: 21 - DNA, 51 bp, synthetic construct
  misc_feature 1..51: note = Synthetic oligonucleotide
  source 1..51: mol_type = other DNA
ggcaacgtctacacctgtctctatccgtcagtaccccatgagtcaagaggc

SEQ ID NO: 22 - DNA, 51 bp, synthetic construct
  misc_feature 1..51: note = Synthetic oligonucleotide
  source 1..51: mol_type = other DNA
ggcaacgtctacacctgtctctatcgaacagtaccccatgagtcaagaggc

SEQ ID NO: 23 - DNA, 46 bp, synthetic construct
  misc_feature 1..46: note = Synthetic oligonucleotide
  source 1..46: mol_type = other DNA
ctgtctctatcaaacagtaccccatgagtgcggaggcccgcctggg

SEQ ID NO: 24 - DNA, 46 bp, synthetic construct
  misc_feature 1..46: note = Synthetic oligonucleotide
  source 1..46: mol_type = other DNA
ctgtctctatcaaacagtaccccatgagtcgtgaggcccgcctggg

SEQ ID NO: 25 - DNA, 46 bp, synthetic construct
  misc_feature 1..46: note = Synthetic oligonucleotide
  source 1..46: mol_type = other DNA
ctgtctctatcaaacagtaccccatgagtgaagaggcccgcctggg

SEQ ID NO: 26 - DNA, 45 bp, synthetic construct
  misc_feature 1..45: note = Synthetic oligonucleotide
  source 1..45: mol_type = other DNA
ggcccgcctggggattgcgccacatattcagcgcttgctggacca

SEQ ID NO: 27 - DNA, 45 bp, synthetic construct
  misc_feature 1..45: note = Synthetic oligonucleotide
  source 1..45: mol_type = other DNA
ggcccgcctggggattcgtccacatattcagcgcttgctggacca

SEQ ID NO: 28 - DNA, 45 bp, synthetic construct
  misc_feature 1..45: note = Synthetic oligonucleotide
  source 1..45: mol_type = other DNA
ggcccgcctggggattgaaccacatattcagcgcttgctggacca

SEQ ID NO: 29 - DNA, 45 bp, synthetic construct
  misc_feature 1..45: note = Synthetic oligonucleotide
  source 1..45: mol_type = other DNA
cgcctggggattaagccacatattgcgcgcttgctggaccagggg

SEQ ID NO: 30 - DNA, 45 bp, synthetic construct
  misc_feature 1..45: note = Synthetic oligonucleotide
  source 1..45: mol_type = other DNA
cgcctggggattaagccacatattcgtcgcttgctggaccagggg

SEQ ID NO: 31 - DNA, 45 bp, synthetic construct
  misc_feature 1..45: note = Synthetic oligonucleotide
  source 1..45: mol_type = other DNA
cgcctggggattaagccacatattgaacgcttgctggaccagggg

SEQ ID NO: 32 - DNA, 45 bp, synthetic construct
  misc_feature 1..45: note = Synthetic oligonucleotide
  source 1..45: mol_type = other DNA
ccgtggaacaccccccttgcgcccgtgaaaaagccaggtacaaac

SEQ ID NO: 33 - DNA, 45 bp, synthetic construct
  misc_feature 1..45: note = Synthetic oligonucleotide
  source 1..45: mol_type = other DNA
ccgtggaacaccccccttcgtcccgtgaaaaagccaggtacaaac

SEQ ID NO: 34 - DNA, 45 bp, synthetic construct
  misc_feature 1..45: note = Synthetic oligonucleotide
  source 1..45: mol_type = other DNA
ccgtggaacaccccccttgaacccgtgaaaaagccaggtacaaac

SEQ ID NO: 35 - DNA, 48 bp, synthetic construct
  misc_feature 1..48: note = Synthetic oligonucleotide
  source 1..48: mol_type = other DNA
caccccccttctgcccgcgaaaaagccaggtacaaacgattatcgtcc

SEQ ID NO: 36 - DNA, 48 bp, synthetic construct
  misc_feature 1..48: note = Synthetic oligonucleotide
  source 1..48: mol_type = other DNA
caccccccttctgccccgtaaaaagccaggtacaaacgattatcgtcc

SEQ ID NO: 37 - DNA, 48 bp, synthetic construct
  misc_feature 1..48: note = Synthetic oligonucleotide
  source 1..48: mol_type = other DNA
caccccccttctgcccgaaaaaaagccaggtacaaacgattatcgtcc

SEQ ID NO: 38 - DNA, 45 bp, synthetic construct
  misc_feature 1..45: note = Synthetic oligonucleotide
  source 1..45: mol_type = other DNA
cccccttctgcccgtggcgaagccaggtacaaacgattatcgtcc

SEQ ID NO: 39 - DNA, 45 bp, synthetic construct
  misc_feature 1..45: note = Synthetic oligonucleotide
  source 1..45: mol_type = other DNA
cccccttctgcccgtgcgtaagccaggtacaaacgattatcgtcc

SEQ ID NO: 40 - DNA, 45 bp, synthetic construct
  misc_feature 1..45: note = Synthetic oligonucleotide
  source 1..45: mol_type = other DNA
cccccttctgcccgtggaaaagccaggtacaaacgattatcgtcc

SEQ ID NO: 41 - DNA, 49 bp, synthetic construct
  misc_feature 1..49: note = Synthetic oligonucleotide
  source 1..49: mol_type = other DNA
cccccttctgcccgtgaaagcgccaggtacaaacgattatcgtccagtt

SEQ ID NO: 42 - DNA, 49 bp, synthetic construct
  misc_feature 1..49: note = Synthetic oligonucleotide
  source 1..49: mol_type = other DNA
cccccttctgcccgtgaaacgtccaggtacaaacgattatcgtccagtt

SEQ ID NO: 43 - DNA, 49 bp, synthetic construct
  misc_feature 1..49: note = Synthetic oligonucleotide
  source 1..49: mol_type = other DNA
cccccttctgcccgtgaaagaaccaggtacaaacgattatcgtccagtt

SEQ ID NO: 44 - DNA, 51 bp, synthetic construct
  misc_feature 1..51: note = Synthetic oligonucleotide
  source 1..51: mol_type = other DNA
gcccgtgaaaaagccaggtgcgaacgattatcgtccagttcaagatcttcg

SEQ ID NO: 45 - DNA, 51 bp, synthetic construct
  misc_feature 1..51: note = Synthetic oligonucleotide
  source 1..51: mol_type = other DNA
gcccgtgaaaaagccaggtcgtaacgattatcgtccagttcaagatcttcg

SEQ ID NO: 46 - DNA, 51 bp, synthetic construct
  misc_feature 1..51: note = Synthetic oligonucleotide
  source 1..51: mol_type = other DNA
gcccgtgaaaaagccaggtgaaaacgattatcgtccagttcaagatcttcg

SEQ ID NO: 47 - DNA, 52 bp, synthetic construct
  misc_feature 1..52: note = Synthetic oligonucleotide
  source 1..52: mol_type = other DNA
cccgtgaaaaagccaggtacagcggattatcgtccagttcaagatcttcgcg

SEQ ID NO: 48 - DNA, 52 bp, synthetic construct
  misc_feature 1..52: note = Synthetic oligonucleotide
  source 1..52: mol_type = other DNA
cccgtgaaaaagccaggtacacgtgattatcgtccagttcaagatcttcgcg

SEQ ID NO: 49 - DNA, 52 bp, synthetic construct
  misc_feature 1..52: note = Synthetic oligonucleotide
  source 1..52: mol_type = other DNA
cccgtgaaaaagccaggtacagaagattatcgtccagttcaagatcttcgcg

SEQ ID NO: 50 - DNA, 50 bp, synthetic construct
  misc_feature 1..50: note = Synthetic oligonucleotide
  source 1..50: mol_type = other DNA
cgtgaaaaagccaggtacaaacgatgcgcgtccagttcaagatcttcgcg

SEQ ID NO: 51 - DNA, 50 bp, synthetic construct
  misc_feature 1..50: note = Synthetic oligonucleotide
  source 1..50: mol_type = other DNA
cgtgaaaaagccaggtacaaacgatcgtcgtccagttcaagatcttcgcg

SEQ ID NO: 52 - DNA, 50 bp, synthetic construct
  misc_feature 1..50: note = Synthetic oligonucleotide
  source 1..50: mol_type = other DNA
cgtgaaaaagccaggtacaaacgatgaacgtccagttcaagatcttcgcg

SEQ ID NO: 53 - DNA, 53 bp, synthetic construct
  misc_feature 1..53: note = Synthetic oligonucleotide
  source 1..53: mol_type = other DNA
cgtgaaaaagccaggtacaaacgattatgcgccagttcaagatcttcgcgagg

SEQ ID NO: 54 - DNA, 53 bp, synthetic construct
  misc_feature 1..53: note = Synthetic oligonucleotide
  source 1..53: mol_type = other DNA
cgtgaaaaagccaggtacaaacgattataaaccagttcaagatcttcgcgagg

SEQ ID NO: 55 - DNA, 53 bp, synthetic construct
  misc_feature 1..53: note = Synthetic oligonucleotide
  source 1..53: mol_type = other DNA
cgtgaaaaagccaggtacaaacgattatgaaccagttcaagatcttcgcgagg

SEQ ID NO: 56 - DNA, 53 bp, synthetic construct
  misc_feature 1..53: note = Synthetic oligonucleotide
  source 1..53: mol_type = other DNA
gccaggtacaaacgattatcgtccagcgcaagatcttcgcgaggtcaacaaac

SEQ ID NO: 57 - DNA, 53 bp, synthetic construct
  misc_feature 1..53: note = Synthetic oligonucleotide
  source 1..53: mol_type = other DNA
gccaggtacaaacgattatcgtccacgtcaagatcttcgcgaggtcaacaaac

SEQ ID NO: 58 - DNA, 53 bp, synthetic construct
  misc_feature 1..53: note = Synthetic oligonucleotide
  source 1..53: mol_type = other DNA
gccaggtacaaacgattatcgtccagaacaagatcttcgcgaggtcaacaaac

SEQ ID NO: 59 - DNA, 51 bp, synthetic construct
  misc_feature 1..51: note = Synthetic oligonucleotide
  source 1..51: mol_type = other DNA
agttcaagatcttcgcgaggtcaacgcgcgcgtagaagacatccatccgac

SEQ ID NO: 60 - DNA, 51 bp, synthetic construct
  misc_feature 1..51: note = Synthetic oligonucleotide
  source 1..51: mol_type = other DNA
agttcaagatcttcgcgaggtcaaccgtcgcgtagaagacatccatccgac

SEQ ID NO: 61 - DNA, 51 bp, synthetic construct
  misc_feature 1..51: note = Synthetic oligonucleotide
  source 1..51: mol_type = other DNA
agttcaagatcttcgcgaggtcaacgaacgcgtagaagacatccatccgac

SEQ ID NO: 62 - DNA, 49 bp, synthetic construct
  misc_feature 1..49: note = Synthetic oligonucleotide
  source 1..49: mol_type = other DNA
gcgaggtcaacaaacgcgtagcggacatccatccgactgtacctaatcc

SEQ ID NO: 63 - DNA, 49 bp, synthetic construct
  misc_feature 1..49: note = Synthetic oligonucleotide
  source 1..49: mol_type = other DNA
gcgaggtcaacaaacgcgtacgtgacatccatccgactgtacctaatcc

SEQ ID NO: 64 - DNA, 49 bp, synthetic construct
  misc_feature 1..49: note = Synthetic oligonucleotide
  source 1..49: mol_type = other DNA
gcgaggtcaacaaacgcgtagatgacatccatccgactgtacctaatcc

SEQ ID NO: 65 - DNA, 59 bp, synthetic construct
  misc_feature 1..59: note = Synthetic oligonucleotide
  source 1..59: mol_type = other DNA
acgcgtagaagacatccatccggtggtacctaatccttataatctgttatcaggcctgc

SEQ ID NO: 66 - DNA, 59 bp, synthetic construct
  misc_feature 1..59: note = Synthetic oligonucleotide
  source 1..59: mol_type = other DNA
acgcgtagaagacatccatccgcgtgtacctaatccttataatctgttatcaggcctgc

SEQ ID NO: 67 - DNA, 59 bp, synthetic construct
  misc_feature 1..59: note = Synthetic oligonucleotide
  source 1..59: mol_type = other DNA
acgcgtagaagacatccatccggaagtacctaatccttataatctgttatcaggcctgc

SEQ ID NO: 68 - DNA, 45 bp, synthetic construct
  misc_feature 1..45: note = Synthetic oligonucleotide
  source 1..45: mol_type = other DNA
cgtctgccccagggctttgcgaacagccccacattgttcgatgaa

SEQ ID NO: 69 - DNA, 45 bp, synthetic construct
  misc_feature 1..45: note = Synthetic oligonucleotide
  source 1..45: mol_type = other DNA
cgtctgccccagggctttcgtaacagccccacattgttcgatgaa

SEQ ID NO: 70 - DNA, 45 bp, synthetic construct
  misc_feature 1..45: note = Synthetic oligonucleotide
  source 1..45: mol_type = other DNA
cgtctgccccagggctttgaaaacagccccacattgttcgatgaa

SEQ ID NO: 71 - DNA, 46 bp, synthetic construct
  misc_feature 1..46: note = Synthetic oligonucleotide
  source 1..46: mol_type = other DNA
agaaggtcaacgttggctgactgcggcgcgtaaggagaccgtaatg

SEQ ID NO: 72 - DNA, 46 bp, synthetic construct
  misc_feature 1..46: note = Synthetic oligonucleotide
  source 1..46: mol_type = other DNA
agaaggtcaacgttggctgactcgtgcgcgtaaggagaccgtaatg

SEQ ID NO: 73 - DNA, 46 bp, synthetic construct
  misc_feature 1..46: note = Synthetic oligonucleotide
  source 1..46: mol_type = other DNA
agaaggtcaacgttggctgactgatgcgcgtaaggagaccgtaatg

SEQ ID NO: 74 - DNA, 49 bp, synthetic construct
  misc_feature 1..49: note = Synthetic oligonucleotide
  source 1..49: mol_type = other DNA
gaaggtcaacgttggctgactgaagtgcgtaaggagaccgtaatggggc

SEQ ID NO: 75 - DNA, 49 bp, synthetic construct
  misc_feature 1..49: note = Synthetic oligonucleotide
  source 1..49: mol_type = other DNA
gaaggtcaacgttggctgactgaacgtcgtaaggagaccgtaatggggc

SEQ ID NO: 76 - DNA, 49 bp, synthetic construct
  misc_feature 1..49: note = Synthetic oligonucleotide
  source 1..49: mol_type = other DNA
gaaggtcaacgttggctgactgaagaacgtaaggagaccgtaatggggc

SEQ ID NO: 77 - DNA, 45 bp, synthetic construct
  misc_feature 1..45: note = Synthetic oligonucleotide
  source 1..45: mol_type = other DNA
gcgtaaggagaccgtaatgggggcgcctacgcctaagacgccacg

SEQ ID NO: 78 - DNA, 45 bp, synthetic construct
  misc_feature 1..45: note = Synthetic oligonucleotide
  source 1..45: mol_type = other DNA
gcgtaaggagaccgtaatggggcgtcctacgcctaagacgccacg

SEQ ID NO: 79 - DNA, 45 bp, synthetic construct
  misc_feature 1..45: note = Synthetic oligonucleotide
  source 1..45: mol_type = other DNA
gcgtaaggagaccgtaatgggggaacctacgcctaagacgccacg

SEQ ID NO: 80 - DNA, 45 bp, synthetic construct
  misc_feature 1..45: note = Synthetic oligonucleotide
  source 1..45: mol_type = other DNA
gagaccgtaatggggcagcctgcgcctaagacgccacgccagttg

SEQ ID NO: 81 - DNA, 45 bp, synthetic construct
  misc_feature 1..45: note = Synthetic oligonucleotide
  source 1..45: mol_type = other DNA
gagaccgtaatggggcagcctcgtcctaagacgccacgccagttg

SEQ ID NO: 82 - DNA, 45 bp, synthetic construct
  misc_feature 1..45: note = Synthetic oligonucleotide
  source 1..45: mol_type = other DNA
gagaccgtaatggggcagcctgaacctaagacgccacgccagttg

SEQ ID NO: 83 - DNA, 45 bp, synthetic construct
  misc_feature 1..45: note = Synthetic oligonucleotide
  source 1..45: mol_type = other DNA
gtaatggggcagcctacgcctgcgacgccacgccagttgcgtgaa

SEQ ID NO: 84 - DNA, 45 bp, synthetic construct
  misc_feature 1..45: note = Synthetic oligonucleotide
  source 1..45: mol_type = other DNA
gtaatggggcagcctacgcctcgtacgccacgccagttgcgtgaa

SEQ ID NO: 85 - DNA, 45 bp, synthetic construct
  misc_feature 1..45: note = Synthetic oligonucleotide
  source 1..45: mol_type = other DNA
gtaatggggcagcctacgcctgaaacgccacgccagttgcgtgaa

SEQ ID NO: 86 - DNA, 45 bp, synthetic construct
  misc_feature 1..45: note = Synthetic oligonucleotide
  source 1..45: mol_type = other DNA
tggggcagcctacgcctaaggcgccacgccagttgcgtgaatttt

SEQ ID NO: 87 - DNA, 45 bp, synthetic construct
  misc_feature 1..45: note = Synthetic oligonucleotide
  source 1..45: mol_type = other DNA
tggggcagcctacgcctaagcgtccacgccagttgcgtgaatttt

SEQ ID NO: 88 - DNA, 45 bp, synthetic construct
  misc_feature 1..45: note = Synthetic oligonucleotide
  source 1..45: mol_type = other DNA
tggggcagcctacgcctaaggaaccacgccagttgcgtgaatttt

SEQ ID NO: 89 - DNA, 47 bp, synthetic construct
  misc_feature 1..47: note = Synthetic oligonucleotide
  source 1..47: mol_type = other DNA
gcctacgcctaagacgccagcgcagttgcgtgaatttttgggcacag

SEQ ID NO: 90 - DNA, 47 bp, synthetic construct
  misc_feature 1..47: note = Synthetic oligonucleotide
  source 1..47: mol_type = other DNA
gcctacgcctaagacgccaaaacagttgcgtgaatttttgggcacag

SEQ ID NO: 91 - DNA, 47 bp, synthetic construct
  misc_feature 1..47: note = Synthetic oligonucleotide
  source 1..47: mol_type = other DNA
gcctacgcctaagacgccagaacagttgcgtgaatttttgggcacag

SEQ ID NO: 92 - DNA, 45 bp, synthetic construct
  misc_feature 1..45: note = Synthetic oligonucleotide
  source 1..45: mol_type = other DNA
cctaagacgccacgccagttggcggaatttttgggcacagcggga

SEQ ID NO: 93 - DNA, 45 bp, synthetic construct
  misc_feature 1..45: note = Synthetic oligonucleotide
  source 1..45: mol_type = other DNA
cctaagacgccacgccagttgaaagaatttttgggcacagcggga

SEQ ID NO: 94 - DNA, 45 bp, synthetic construct
  misc_feature 1..45: note = Synthetic oligonucleotide
  source 1..45: mol_type = other DNA
cctaagacgccacgccagttggaagaatttttgggcacagcggga

SEQ ID NO: 95 - DNA, 45 bp, synthetic construct
  misc_feature 1..45: note = Synthetic oligonucleotide
  source 1..45: mol_type = other DNA
gcacccctgtaccccttaacagcgacagggacgcttttcaactgg

SEQ ID NO: 96 - DNA, 45 bp, synthetic construct
  misc_feature 1..45: note = Synthetic oligonucleotide
  source 1..45: mol_type = other DNA
gcacccctgtaccccttaacacgtacagggacgcttttcaactgg

SEQ ID NO: 97 - DNA, 45 bp, synthetic construct
  misc_feature 1..45: note = Synthetic oligonucleotide
  source 1..45: mol_type = other DNA
gcacccctgtaccccttaacagaaacagggacgcttttcaactgg

SEQ ID NO: 98 - DNA, 54 bp, synthetic construct
  misc_feature 1..54: note = Synthetic oligonucleotide
  source 1..54: mol_type = other DNA
gtttgatagagacaggtgtagacgttgccgctaacgggatgatcaacggtgctt

SEQ ID NO: 99 - DNA, 54 bp, synthetic construct
  misc_feature 1..54: note = Synthetic oligonucleotide
  source 1..54: mol_type = other DNA
gtttgatagagacaggtgtagacgttgcacgtaacgggatgatcaacggtgctt

SEQ ID NO: 100 - DNA, 54 bp, synthetic construct
  misc_feature 1..54: note = Synthetic oligonucleotide
  source 1..54: mol_type = other DNA
gtttgatagagacaggtgtagacgttgcttctaacgggatgatcaacggtgctt

SEQ ID NO: 101 - DNA, 57 bp, synthetic construct
  misc_feature 1..57: note = Synthetic oligonucleotide
  source 1..57: mol_type = other DNA
ggggtactgtttgatagagacaggtgtagacgctgcctttaacgggatgatcaacgg

SEQ ID NO: 102 - DNA, 57 bp, synthetic construct
  misc_feature 1..57: note = Synthetic oligonucleotide
  source 1..57: mol_type = other DNA
ggggtactgtttgatagagacaggtgtagaacgtgcctttaacgggatgatcaacgg

SEQ ID NO: 103 - DNA, 57 bp, synthetic construct
  misc_feature 1..57: note = Synthetic oligonucleotide
  source 1..57: mol_type = other DNA
ggggtactgtttgatagagacaggtgtagattctgcctttaacgggatgatcaacgg

SEQ ID NO: 104 - DNA, 56 bp, synthetic construct
  misc_feature 1..56: note = Synthetic oligonucleotide
  source 1..56: mol_type = other DNA
ctcatggggtactgtttgatagagacaggcgcagacgttgcctttaacgggatgat

SEQ ID NO: 105 - DNA, 56 bp, synthetic construct
  misc_feature 1..56: note = Synthetic oligonucleotide
  source 1..56: mol_type = other DNA
ctcatggggtactgtttgatagagacaggacgagacgttgcctttaacgggatgat

SEQ ID NO: 106 - DNA, 56 bp, synthetic construct
  misc_feature 1..56: note = Synthetic oligonucleotide
  source 1..56: mol_type = other DNA
ctcatggggtactgtttgatagagacaggttcagacgttgcctttaacgggatgat

SEQ ID NO: 107 - DNA, 58 bp, synthetic construct
  misc_feature 1..58: note = Synthetic oligonucleotide
  source 1..58: mol_type = other DNA
cctcttgactcatggggtactgtttgatagacgcaggtgtagacgttgcctttaacgg

SEQ ID NO: 108 - DNA, 58 bp, synthetic construct
  misc_feature 1..58: note = Synthetic oligonucleotide
  source 1..58: mol_type = other DNA
cctcttgactcatggggtactgtttgatagaacgaggtgtagacgttgcctttaacgg

SEQ ID NO: 109 - DNA, 58 bp, synthetic construct
  misc_feature 1..58: note = Synthetic oligonucleotide
  source 1..58: mol_type = other DNA
cctcttgactcatggggtactgtttgatagattcaggtgtagacgttgcctttaacgg

SEQ ID NO: 110 - DNA, 54 bp, synthetic construct
  misc_feature 1..54: note = Synthetic oligonucleotide
  source 1..54: mol_type = other DNA
cctcttgactcatggggtactgtttcgcagagacaggtgtagacgttgccttta

SEQ ID NO: 111 - DNA, 54 bp, synthetic construct
  misc_feature 1..54: note = Synthetic oligonucleotide
  source 1..54: mol_type = other DNA
cctcttgactcatggggtactgtttacgagagacaggtgtagacgttgccttta

SEQ ID NO: 112 - DNA, 54 bp, synthetic construct
  misc_feature 1..54: note = Synthetic oligonucleotide
  source 1..54: mol_type = other DNA
cctcttgactcatggggtactgtttttcagagacaggtgtagacgttgccttta

SEQ ID NO: 113 - DNA, 51 bp, synthetic construct
  misc_feature 1..51: note = Synthetic oligonucleotide
  source 1..51: mol_type = other DNA
gcctcttgactcatggggtactgcgcgatagagacaggtgtagacgttgcc

SEQ ID NO: 114 - DNA, 51 bp, synthetic construct
  misc_feature 1..51: note = Synthetic oligonucleotide
  source 1..51: mol_type = other DNA
gcctcttgactcatggggtactgacggatagagacaggtgtagacgttgcc

SEQ ID NO: 115 - DNA, 51 bp, synthetic construct
  misc_feature 1..51: note = Synthetic oligonucleotide
  source 1..51: mol_type = other DNA
gcctcttgactcatggggtactgttcgatagagacaggtgtagacgttgcc

SEQ ID NO: 116 - DNA, 46 bp, synthetic construct; identical to SEQ ID NO: 23
  misc_feature 1..46: note = Synthetic oligonucleotide
  source 1..46: mol_type = other DNA

SEQ ID NO: 117 - DNA, 46 bp, synthetic construct; identical to SEQ ID NO: 24
  misc_feature 1..46: note = Synthetic oligonucleotide
  source 1..46: mol_type = other DNA

SEQ ID NO: 118 - DNA, 46 bp, synthetic construct; identical to SEQ ID NO: 25
  misc_feature 1..46: note = Synthetic oligonucleotide
  source 1..46: mol_type = other DNA

SEQ ID NO: 119 - DNA, 45 bp, synthetic construct
  misc_feature 1..45: note = Synthetic oligonucleotide
  source 1..45: mol_type = other DNA
tggtccagcaagcgctgaatatgtggcgcaatccccaggcgggcc

SEQ ID NO: 120 - DNA, 45 bp, synthetic construct
  misc_feature 1..45: note = Synthetic oligonucleotide
  source 1..45: mol_type = other DNA
tggtccagcaagcgctgaatatgtggacgaatccccaggcgggcc

SEQ ID NO: 121 - DNA, 45 bp, synthetic construct
  misc_feature 1..45: note = Synthetic oligonucleotide
  source 1..45: mol_type = other DNA
tggtccagcaagcgctgaatatgtggttcaatccccaggcgggcc

SEQ ID NO: 122 - DNA, 45 bp, synthetic construct
  misc_feature 1..45: note = Synthetic oligonucleotide
  source 1..45: mol_type = other DNA
cccctggtccagcaagcgcgcaatatgtggcttaatccccaggcg

SEQ ID NO: 123 - DNA, 45 bp, synthetic construct
  misc_feature 1..45: note = Synthetic oligonucleotide
  source 1..45: mol_type = other DNA
cccctggtccagcaagcgacgaatatgtggcttaatccccaggcg

SEQ ID NO: 124 - DNA, 45 bp, synthetic construct
  misc_feature 1..45: note = Synthetic oligonucleotide
  source 1..45: mol_type = other DNA
cccctggtccagcaagcgttcaatatgtggcttaatccccaggcg

SEQ ID NO: 125 - DNA, 45 bp, synthetic construct
  misc_feature 1..45: note = Synthetic oligonucleotide
  source 1..45: mol_type = other DNA
gtttgtacctggctttttcacgggcgcaaggggggtgttccacgg

SEQ ID NO: 126 - DNA, 45 bp, synthetic construct
  misc_feature 1..45: note = Synthetic oligonucleotide
  source 1..45: mol_type = other DNA
gtttgtacctggctttttcacgggacgaaggggggtgttccacgg

SEQ ID NO: 127 - DNA, 45 bp, synthetic construct
  misc_feature 1..45: note = Synthetic oligonucleotide
  source 1..45: mol_type = other DNA
gtttgtacctggctttttcacgggttcaaggggggtgttccacgg

SEQ ID NO: 128 - DNA, 48 bp, synthetic construct
  misc_feature 1..48: note = Synthetic oligonucleotide
  source 1..48: mol_type = other DNA
ggacgataatcgtttgtacctggctttttcgcgggcagaaggggggtg

SEQ ID NO: 129 - DNA, 48 bp, synthetic construct
  misc_feature 1..48: note = Synthetic oligonucleotide
  source 1..48: mol_type = other DNA
ggacgataatcgtttgtacctggctttttacggggcagaaggggggtg

SEQ ID NO: 130 - DNA, 48 bp, synthetic construct
  misc_feature 1..48: note = Synthetic oligonucleotide
  source 1..48: mol_type = other DNA
ggacgataatcgtttgtacctggctttttttcgggcagaaggggggtg

SEQ ID NO: 131 - DNA, 45 bp, synthetic construct
  misc_feature 1..45: note = Synthetic oligonucleotide
  source 1..45: mol_type = other DNA
ggacgataatcgtttgtacctggcttcgccacgggcagaaggggg

SEQ ID NO: 132 - DNA, 45 bp, synthetic construct
  misc_feature 1..45: note = Synthetic oligonucleotide
  source 1..45: mol_type = other DNA
ggacgataatcgtttgtacctggcttacgcacgggcagaaggggg

SEQ ID NO: 133 - DNA, 45 bp, synthetic construct
  misc_feature 1..45: note = Synthetic oligonucleotide
  source 1..45: mol_type = other DNA
ggacgataatcgtttgtacctggcttttccacgggcagaaggggg

SEQ ID NO: 134 - DNA, 49 bp, synthetic construct
  misc_feature 1..49: note = Synthetic oligonucleotide
  source 1..49: mol_type = other DNA
aactggacgataatcgtttgtacctggcgctttcacgggcagaaggggg

SEQ ID NO: 135 - DNA, 49 bp, synthetic construct
  misc_feature 1..49: note = Synthetic oligonucleotide
  source 1..49: mol_type = other DNA
aactggacgataatcgtttgtacctggacgtttcacgggcagaaggggg

SEQ ID NO: 136 - DNA, 49 bp, synthetic construct
  misc_feature 1..49: note = Synthetic oligonucleotide
  source 1..49: mol_type = other DNA
aactggacgataatcgtttgtacctggttctttcacgggcagaaggggg

SEQ ID NO: 137 - DNA, 51 bp, synthetic construct
  misc_feature 1..51: note = Synthetic oligonucleotide
  source 1..51: mol_type = other DNA
cgaagatcttgaactggacgataatcgttcgcacctggctttttcacgggc

SEQ ID NO: 138 - DNA, 51 bp, synthetic construct
  misc_feature 1..51: note = Synthetic oligonucleotide
  source 1..51: mol_type = other DNA
cgaagatcttgaactggacgataatcgttacgacctggctttttcacgggc

SEQ ID NO: 139 - DNA, 51 bp, synthetic construct
  misc_feature 1..51: note = Synthetic oligonucleotide
  source 1..51: mol_type = other DNA
cgaagatcttgaactggacgataatcgttttcacctggctttttcacgggc

SEQ ID NO: 140 - DNA, 52 bp, synthetic construct
  misc_feature 1..52: note = Synthetic oligonucleotide
  source 1..52: mol_type = other DNA
cgcgaagatcttgaactggacgataatccgctgtacctggctttttcacggg

SEQ ID NO: 141 - DNA, 52 bp, synthetic construct
  misc_feature 1..52: note = Synthetic oligonucleotide
  source 1..52: mol_type = other DNA
cgcgaagatcttgaactggacgataatcacgtgtacctggctttttcacggg

SEQ ID NO: 142 - DNA, 52 bp, synthetic construct
  misc_feature 1..52: note = Synthetic oligonucleotide
  source 1..52: mol_type = other DNA
cgcgaagatcttgaactggacgataatcttctgtacctggctttttcacggg

SEQ ID NO: 143 - DNA, 50 bp, synthetic construct
  misc_feature 1..50: note = Synthetic oligonucleotide
  source 1..50: mol_type = other DNA
cgcgaagatcttgaactggacgcgcatcgtttgtacctggctttttcacg

SEQ ID NO: 144 - DNA, 50 bp, synthetic construct
  misc_feature 1..50: note = Synthetic oligonucleotide
  source 1..50: mol_type = other DNA
cgcgaagatcttgaactggacgacgatcgtttgtacctggctttttcacg

SEQ ID NO: 145 - DNA, 50 bp, synthetic construct
  misc_feature 1..50: note = Synthetic oligonucleotide
  source 1..50: mol_type = other DNA
cgcgaagatcttgaactggacgttcatcgtttgtacctggctttttcacg

SEQ ID NO: 146 - DNA, 53 bp, synthetic construct
  misc_feature 1..53: note = Synthetic oligonucleotide
  source 1..53: mol_type = other DNA
cctcgcgaagatcttgaactggcgcataatcgtttgtacctggctttttcacg

SEQ ID NO: 147 - DNA, 53 bp, synthetic construct
  misc_feature 1..53: note = Synthetic oligonucleotide
  source 1..53: mol_type = other DNA
cctcgcgaagatcttgaactggtttataatcgtttgtacctggctttttcacg

SEQ ID NO: 148 - DNA, 53 bp, synthetic construct
  misc_feature 1..53: note = Synthetic oligonucleotide
  source 1..53: mol_type = other DNA
cctcgcgaagatcttgaactggttcataatcgtttgtacctggctttttcacg

SEQ ID NO: 149 - DNA, 53 bp, synthetic construct
  misc_feature 1..53: note = Synthetic oligonucleotide
  source 1..53: mol_type = other DNA
gtttgttgacctcgcgaagatcttgcgctggacgataatcgtttgtacctggc

SEQ ID NO: 150 - DNA, 53 bp, synthetic construct
  misc_feature 1..53: note = Synthetic oligonucleotide
  source 1..53: mol_type = other DNA
gtttgttgacctcgcgaagatcttgacgtggacgataatcgtttgtacctggc

SEQ ID NO: 151 - DNA, 53 bp, synthetic construct
  misc_feature 1..53: note = Synthetic oligonucleotide
  source 1..53: mol_type = other DNA
gtttgttgacctcgcgaagatcttgttctggacgataatcgtttgtacctggc

SEQ ID NO: 152 - DNA, 51 bp, synthetic construct
  misc_feature 1..51: note = Synthetic oligonucleotide
  source 1..51: mol_type = other DNA
gtcggatggatgtcttctacgcgcgcgttgacctcgcgaagatcttgaact

SEQ ID NO: 153 - DNA, 51 bp, synthetic construct
  misc_feature 1..51: note = Synthetic oligonucleotide
  source 1..51: mol_type = other DNA
gtcggatggatgtcttctacgcgacggttgacctcgcgaagatcttgaact

SEQ ID NO: 154 - DNA, 51 bp, synthetic construct
  misc_feature 1..51: note = Synthetic oligonucleotide
  source 1..51: mol_type = other DNA
gtcggatggatgtcttctacgcgttcgttgacctcgcgaagatcttgaact

SEQ ID NO: 155 - DNA, 49 bp, synthetic construct
  misc_feature 1..49: note = Synthetic oligonucleotide
  source 1..49: mol_type = other DNA
ggattaggtacagtcggatggatgtccgctacgcgtttgttgacctcgc

SEQ ID NO: 156 - DNA, 49 bp, synthetic construct
  misc_feature 1..49: note = Synthetic oligonucleotide
  source 1..49: mol_type = other DNA
ggattaggtacagtcggatggatgtcacgtacgcgtttgttgacctcgc

SEQ ID NO: 157 - DNA, 49 bp, synthetic construct
  misc_feature 1..49: note = Synthetic oligonucleotide
  source 1..49: mol_type = other DNA
ggattaggtacagtcggatggatgtcatctacgcgtttgttgacctcgc

SEQ ID NO: 158 - DNA, 59 bp, synthetic construct
  misc_feature 1..59: note = Synthetic oligonucleotide
  source 1..59: mol_type = other DNA
gcaggcctgataacagattataaggattaggtaccaccggatggatgtcttctacgcgt

SEQ ID NO: 159 - DNA, 59 bp, synthetic construct
  misc_feature 1..59: note = Synthetic oligonucleotide
  source 1..59: mol_type = other DNA
gcaggcctgataacagattataaggattaggtacacgcggatggatgtcttctacgcgt

SEQ ID NO: 160 - DNA, 59 bp, synthetic construct
  misc_feature 1..59: note = Synthetic oligonucleotide
  source 1..59: mol_type = other DNA
gcaggcctgataacagattataaggattaggtacttccggatggatgtcttctacgcgt

SEQ ID NO: 161 - DNA, 45 bp, synthetic construct
  misc_feature 1..45: note = Synthetic oligonucleotide
  source 1..45: mol_type = other DNA
ttcatcgaacaatgtggggctgttcgcaaagccctggggcagacg

SEQ ID NO: 162 - DNA, 45 bp, synthetic construct
  misc_feature 1..45: note = Synthetic oligonucleotide
  source 1..45: mol_type = other DNA
ttcatcgaacaatgtggggctgttacgaaagccctggggcagacg

SEQ ID NO: 163 - DNA, 45 bp, synthetic construct
  misc_feature 1..45: note = Synthetic oligonucleotide
  source 1..45: mol_type = other DNA
ttcatcgaacaatgtggggctgttttcaaagccctggggcagacg

SEQ ID NO: 164 - DNA, 46 bp, synthetic construct
  misc_feature 1..46: note = Synthetic oligonucleotide
  source 1..46: mol_type = other DNA
cattacggtctccttacgcgccgcagtcagccaacgttgaccttct

SEQ ID NO: 165 - DNA, 46 bp, synthetic construct
  misc_feature 1..46: note = Synthetic oligonucleotide
  source 1..46: mol_type = other DNA
cattacggtctccttacgcgcacgagtcagccaacgttgaccttct

SEQ ID NO: 166 - DNA, 46 bp, synthetic construct
  misc_feature 1..46: note = Synthetic oligonucleotide
  source 1..46: mol_type = other DNA
cattacggtctccttacgcgcatcagtcagccaacgttgaccttct

SEQ ID NO: 167 - DNA, 49 bp, synthetic construct
  misc_feature 1..49: note = Synthetic oligonucleotide
  source 1..49: mol_type = other DNA
gccccattacggtctccttacgcacttcagtcagccaacgttgaccttc

SEQ ID NO: 168 - DNA, 49 bp, synthetic construct
  misc_feature 1..49: note = Synthetic oligonucleotide
  source 1..49: mol_type = other DNA
gccccattacggtctccttacgacgttcagtcagccaacgttgaccttc

SEQ ID NO: 169 - DNA, 49 bp, synthetic construct
  misc_feature 1..49: note = Synthetic oligonucleotide
  source 1..49: mol_type = other DNA
gccccattacggtctccttacgttcttcagtcagccaacgttgaccttc

SEQ ID NO: 170 - DNA, 45 bp, synthetic construct
  misc_feature 1..45: note = Synthetic oligonucleotide
  source 1..45: mol_type = other DNA
cgtggcgtcttaggcgtaggcgcccccattacggtctccttacgc

SEQ ID NO: 171 - DNA, 45 bp, synthetic construct
  misc_feature 1..45: note = Synthetic oligonucleotide
  source 1..45: mol_type = other DNA
cgtggcgtcttaggcgtaggacgccccattacggtctccttacgc

SEQ ID NO: 172 - DNA, 45 bp, synthetic construct
  misc_feature 1..45: note = Synthetic oligonucleotide
  source 1..45: mol_type = other DNA
cgtggcgtcttaggcgtaggttcccccattacggtctccttacgc

SEQ ID NO: 173 - DNA, 45 bp, synthetic construct
  misc_feature 1..45: note = Synthetic oligonucleotide
  source 1..45: mol_type = other DNA
caactggcgtggcgtcttaggcgcaggctgccccattacggtctc

SEQ ID NO: 174 - DNA, 45 bp, synthetic construct
  misc_feature 1..45: note = Synthetic oligonucleotide
  source 1..45: mol_type = other DNA
caactggcgtggcgtcttaggacgaggctgccccattacggtctc

SEQ ID NO: 175 - DNA, 45 bp, synthetic construct
  misc_feature 1..45: note = Synthetic oligonucleotide
  source 1..45: mol_type = other DNA
caactggcgtggcgtcttaggttcaggctgccccattacggtctc

SEQ ID NO: 176 - DNA, 45 bp, synthetic construct
  misc_feature 1..45: note = Synthetic oligonucleotide
  source 1..45: mol_type = other DNA
ttcacgcaactggcgtggcgtcgcaggcgtaggctgccccattac

SEQ ID NO: 177 - DNA, 45 bp, synthetic construct
  misc_feature 1..45: note = Synthetic oligonucleotide
  source 1..45: mol_type = other DNA
ttcacgcaactggcgtggcgtacgaggcgtaggctgccccattac

SEQ ID NO: 178 - DNA, 45 bp, synthetic construct
  misc_feature 1..45: note = Synthetic oligonucleotide
  source 1..45: mol_type = other DNA
ttcacgcaactggcgtggcgtttcaggcgtaggctgccccattac

SEQ ID NO: 179 - DNA, 45 bp, synthetic construct
  misc_feature 1..45: note = Synthetic oligonucleotide
  source 1..45: mol_type = other DNA
aaaattcacgcaactggcgtggcgccttaggcgtaggctgcccca

SEQ ID NO: 180 - DNA, 45 bp, synthetic construct
  misc_feature 1..45: note = Synthetic oligonucleotide
  source 1..45: mol_type = other DNA
aaaattcacgcaactggcgtggacgcttaggcgtaggctgcccca

SEQ ID NO: 181 - DNA, 45 bp, synthetic construct
  misc_feature 1..45: note = Synthetic oligonucleotide
  source 1..45: mol_type = other DNA
aaaattcacgcaactggcgtggttccttaggcgtaggctgcccca

SEQ ID NO: 182 - DNA, 47 bp, synthetic construct
  misc_feature 1..47: note = Synthetic oligonucleotide
  source 1..47: mol_type = other DNA
ctgtgcccaaaaattcacgcaactgcgctggcgtcttaggcgtaggc

SEQ ID NO: 183 - DNA, 47 bp, synthetic construct
  misc_feature 1..47: note = Synthetic oligonucleotide
  source 1..47: mol_type = other DNA
ctgtgcccaaaaattcacgcaactgttttggcgtcttaggcgtaggc

SEQ ID NO: 184 - DNA, 47 bp, synthetic construct
  misc_feature 1..47: note = Synthetic oligonucleotide
  source 1..47: mol_type = other DNA
ctgtgcccaaaaattcacgcaactgttctggcgtcttaggcgtaggc

SEQ ID NO: 185 - DNA, 45 bp, synthetic construct
  misc_feature 1..45: note = Synthetic oligonucleotide
  source 1..45: mol_type = other DNA
tcccgctgtgcccaaaaattccgccaactggcgtggcgtcttagg

SEQ ID NO: 186 - DNA, 45 bp, synthetic construct
  misc_feature 1..45: note = Synthetic oligonucleotide
  source 1..45: mol_type = other DNA
tcccgctgtgcccaaaaattctttcaactggcgtggcgtcttagg

SEQ ID NO: 187 - DNA, 45 bp, synthetic construct
  misc_feature 1..45: note = Synthetic oligonucleotide
  source 1..45: mol_type = other DNA
tcccgctgtgcccaaaaattcttccaactggcgtggcgtcttagg

SEQ ID NO: 188 - DNA, 45 bp, synthetic construct
  misc_feature 1..45: note = Synthetic oligonucleotide
  source 1..45: mol_type = other DNA
ccagttgaaaagcgtccctgtcgctgttaaggggtacaggggtgc

SEQ ID NO: 189 - DNA, 45 bp, synthetic construct
  misc_feature 1..45: note = Synthetic oligonucleotide
  source 1..45: mol_type = other DNA
ccagttgaaaagcgtccctgtacgtgttaaggggtacaggggtgc

SEQ ID NO: 190 - DNA, 45 bp, synthetic construct
  misc_feature 1..45: note = Synthetic oligonucleotide
  source 1..45: mol_type = other DNA
ccagttgaaaagcgtccctgtttctgttaaggggtacaggggtgc

SEQ ID NO: 191 - DNA, 54 bp, synthetic construct
  misc_feature 1..54: note = Synthetic oligonucleotide
  source 1..54: mol_type = other DNA
taaaggcaacgtctacacctgtctctggcaaacagtaccccatgagtcaagagg

SEQ ID NO: 192 - DNA, 54 bp, synthetic construct
  misc_feature 1..54: note = Synthetic oligonucleotide
  source 1..54: mol_type = other DNA
cctcttgactcatggggtactgtttgccagagacaggtgtagacgttgccttta

SEQ ID NO: 193 - DNA, 54 bp, synthetic construct
  misc_feature 1..54: note = Synthetic oligonucleotide
  source 1..54: mol_type = other DNA
taaaggcaacgtctacacctgtctctctgaaacagtaccccatgagtcaagagg

SEQ ID NO: 194 - DNA, 54 bp, synthetic construct
  misc_feature 1..54: note = Synthetic oligonucleotide
  source 1..54: mol_type = other DNA
cctcttgactcatggggtactgtttcagagagacaggtgtagacgttgccttta

SEQ ID NO: 195 - DNA, 54 bp, synthetic construct
  misc_feature 1..54: note = Synthetic oligonucleotide
  source 1..54: mol_type = other DNA
taaaggcaacgtctacacctgtctctgtgaaacagtaccccatgagtcaagagg

SEQ ID NO: 196 - DNA, 54 bp, synthetic construct
  misc_feature 1..54: note = Synthetic oligonucleotide
  source 1..54: mol_type = other DNA
cctcttgactcatggggtactgtttcacagagacaggtgtagacgttgccttta

SEQ ID NO: 197 - DNA, 54 bp, synthetic construct
  misc_feature 1..54: note = Synthetic oligonucleotide
  source 1..54: mol_type = other DNA
taaaggcaacgtctacacctgtctctccgaaacagtaccccatgagtcaagagg

SEQ ID NO: 198 - DNA, 54 bp, synthetic construct
  misc_feature 1..54: note = Synthetic oligonucleotide
  source 1..54: mol_type = other DNA
cctcttgactcatggggtactgtttcggagagacaggtgtagacgttgccttta

SEQ ID NO: 199 - DNA, 54 bp, synthetic construct
  misc_feature 1..54: note = Synthetic oligonucleotide
  source 1..54: mol_type = other DNA
taaaggcaacgtctacacctgtctctatgaaacagtaccccatgagtcaagagg

SEQ ID NO: 200 - DNA, 54 bp, synthetic construct
  misc_feature 1..54: note = Synthetic oligonucleotide
  source 1..54: mol_type = other DNA
cctcttgactcatggggtactgtttcatagagacaggtgtagacgttgccttta

SEQ ID NO: 201 - DNA, 54 bp, synthetic construct
  misc_feature 1..54: note = Synthetic oligonucleotide
  source 1..54: mol_type = other DNA
taaaggcaacgtctacacctgtctctagcaaacagtaccccatgagtcaagagg

SEQ ID NO: 202 - DNA, 54 bp, synthetic construct
  misc_feature 1..54: note = Synthetic oligonucleotide
  source 1..54: mol_type = other DNA
cctcttgactcatggggtactgtttgctagagacaggtgtagacgttgccttta

SEQ ID NO: 203 - DNA, 54 bp, synthetic construct
  misc_feature 1..54: note = Synthetic oligonucleotide
  source 1..54: mol_type = other DNA
taaaggcaacgtctacacctgtctctaccaaacagtaccccatgagtcaagagg

SEQ ID NO: 204 - DNA, 54 bp, synthetic construct
  misc_feature 1..54: note = Synthetic oligonucleotide
  source 1..54: mol_type = other DNA
cctcttgactcatggggtactgtttggtagagacaggtgtagacgttgccttta

SEQ ID NO: 205 - DNA, 54 bp, synthetic construct
  misc_feature 1..54: note = Synthetic oligonucleotide
  source 1..54: mol_type = other DNA
taaaggcaacgtctacacctgtctcttgcaaacagtaccccatgagtcaagagg

SEQ ID NO: 206 - DNA, 54 bp, synthetic construct
  misc_feature 1..54: note = Synthetic oligonucleotide
  source 1..54: mol_type = other DNA
cctcttgactcatggggtactgtttgcaagagacaggtgtagacgttgccttta

SEQ ID NO: 207 - DNA, 54 bp, synthetic construct
  misc_feature 1..54: note = Synthetic oligonucleotide
  source 1..54: mol_type = other DNA
taaaggcaacgtctacacctgtctcttttaaacagtaccccatgagtcaagagg

SEQ ID NO: 208 - DNA, 54 bp, synthetic construct
  misc_feature 1..54: note = Synthetic oligonucleotide
  source 1..54: mol_type = other DNA
cctcttgactcatggggtactgtttaaaagagacaggtgtagacgttgccttta

SEQ ID NO: 209 - DNA, 54 bp, synthetic construct
  misc_feature 1..54: note = Synthetic oligonucleotide
  source 1..54: mol_type = other DNA
taaaggcaacgtctacacctgtctcttataaacagtaccccatgagtcaagagg

SEQ ID NO: 210 - DNA, 54 bp, synthetic construct
  misc_feature 1..54: note = Synthetic oligonucleotide
  source 1..54: mol_type = other DNA
cctcttgactcatggggtactgtttataagagacaggtgtagacgttgccttta

SEQ ID NO: 211 - DNA, 54 bp, synthetic construct
  misc_feature 1..54: note = Synthetic oligonucleotide
  source 1..54: mol_type = other DNA
taaaggcaacgtctacacctgtctctcataaacagtaccccatgagtcaagagg

SEQ ID NO: 212 - DNA, 54 bp, synthetic construct
  misc_feature 1..54: note = Synthetic oligonucleotide
  source 1..54: mol_type = other DNA
cctcttgactcatggggtactgtttatgagagacaggtgtagacgttgccttta

SEQ ID NO: 213 - DNA, 54 bp, synthetic construct
  misc_feature 1..54: note = Synthetic oligonucleotide
  source 1..54: mol_type = other DNA
taaaggcaacgtctacacctgtctcttggaaacagtaccccatgagtcaagagg

SEQ ID NO: 214 - DNA, 54 bp, synthetic construct
  misc_feature 1..54: note = Synthetic oligonucleotide
  source 1..54: mol_type = other DNA
cctcttgactcatggggtactgtttccaagagacaggtgtagacgttgccttta

SEQ ID NO: 215 - DNA, 54 bp, synthetic construct
  misc_feature 1..54: note = Synthetic oligonucleotide
  source 1..54: mol_type = other DNA
taaaggcaacgtctacacctgtctctgataaacagtaccccatgagtcaagagg

SEQ ID NO: 216 - DNA, 54 bp, synthetic construct
  misc_feature 1..54: note = Synthetic oligonucleotide
  source 1..54: mol_type = other DNA
cctcttgactcatggggtactgtttatcagagacaggtgtagacgttgccttta

SEQ ID NO: 217 - DNA, 54 bp, synthetic construct
  misc_feature 1..54: note = Synthetic oligonucleotide
  source 1..54: mol_type = other DNA
taaaggcaacgtctacacctgtctctaacaaacagtaccccatgagtcaagagg

SEQ ID NO: 218 - DNA, 54 bp, synthetic construct
  misc_feature 1..54: note = Synthetic oligonucleotide
  source 1..54: mol_type = other DNA
cctcttgactcatggggtactgtttgttagagacaggtgtagacgttgccttta

SEQ ID NO: 219 - DNA, 54 bp, synthetic construct
  misc_feature 1..54: note = Synthetic oligonucleotide
  source 1..54: mol_type = other DNA
taaaggcaacgtctacacctgtctctcagaaacagtaccccatgagtcaagagg

SEQ ID NO: 220 - DNA, 54 bp, synthetic construct
  misc_feature 1..54: note = Synthetic oligonucleotide
  source 1..54: mol_type = other DNA
cctcttgactcatggggtactgtttctgagagacaggtgtagacgttgccttta

SEQ ID NO: 221 - DNA, 54 bp, synthetic construct
  misc_feature 1..54: note = Synthetic oligonucleotide
  source 1..54: mol_type = other DNA
taaaggcaacgtctacacctgtctctaaaaaacagtaccccatgagtcaagagg

SEQ ID NO: 222 - DNA, 54 bp, synthetic construct
  misc_feature 1..54: note = Synthetic oligonucleotide
  source 1..54: mol_type = other DNA
cctcttgactcatggggtactgttttttagagacaggtgtagacgttgccttta

SEQ ID NO: 223 - DNA, 46 bp, synthetic construct
  misc_feature 1..46: note = Synthetic oligonucleotide
  source 1..46: mol_type = other DNA
ctgtctctatcaaacagtaccccatgagtggcgaggcccgcctggg

SEQ ID NO: 224 - DNA, 46 bp, synthetic construct
  misc_feature 1..46: note = Synthetic oligonucleotide
  source 1..46: mol_type = other DNA
cccaggcgggcctcgccactcatggggtactgtttgatagagacag

SEQ ID NO: 225 - DNA, 46 bp, synthetic construct
  misc_feature 1..46: note = Synthetic oligonucleotide
  source 1..46: mol_type = other DNA
ctgtctctatcaaacagtaccccatgagtctggaggcccgcctggg

SEQ ID NO: 226 - DNA, 46 bp, synthetic construct
  misc_feature 1..46: note = Synthetic oligonucleotide
  source 1..46: mol_type = other DNA
cccaggcgggcctccagactcatggggtactgtttgatagagacag

SEQ ID NO: 227 - DNA, 46 bp, synthetic construct
  misc_feature 1..46: note = Synthetic oligonucleotide
  source 1..46: mol_type = other DNA
ctgtctctatcaaacagtaccccatgagtattgaggcccgcctggg

SEQ ID NO: 228 - DNA, 46 bp, synthetic construct
  misc_feature 1..46: note = Synthetic oligonucleotide
  source 1..46: mol_type = other DNA
cccaggcgggcctcaatactcatggggtactgtttgatagagacag

SEQ ID NO: 229 - DNA, 46 bp, synthetic construct
  misc_feature 1..46: note = Synthetic oligonucleotide
  source 1..46: mol_type = other DNA
ctgtctctatcaaacagtaccccatgagtgtggaggcccgcctggg

SEQ ID NO: 230 - DNA, 46 bp, synthetic construct
  misc_feature 1..46: note = Synthetic oligonucleotide
  source 1..46: mol_type = other DNA
cccaggcgggcctccacactcatggggtactgtttgatagagacag

SEQ ID NO: 231 - DNA, 46 bp, synthetic construct
  misc_feature 1..46: note = Synthetic oligonucleotide
  source 1..46: mol_type = other DNA
ctgtctctatcaaacagtaccccatgagtccggaggcccgcctggg

SEQ ID NO: 232 - DNA, 46 bp, synthetic construct
  misc_feature 1..46: note = Synthetic oligonucleotide
  source 1..46: mol_type = other DNA
cccaggcgggcctccggactcatggggtactgtttgatagagacag

SEQ ID NO: 233 - DNA, 46 bp, synthetic construct
  misc_feature 1..46: note = Synthetic oligonucleotide
  source 1..46: mol_type = other DNA
ctgtctctatcaaacagtaccccatgagtatggaggcccgcctggg

SEQ ID NO: 234 - DNA, 46 bp, synthetic construct
  misc_feature 1..46: note = Synthetic oligonucleotide
  source 1..46: mol_type = other DNA
cccaggcgggcctccatactcatggggtactgtttgatagagacag

SEQ ID NO: 235 - DNA, 46 bp, synthetic construct
  misc_feature 1..46: note = Synthetic oligonucleotide
  source 1..46: mol_type = other DNA
ctgtctctatcaaacagtaccccatgagtagcgaggcccgcctggg

SEQ ID NO: 236 - DNA, 46 bp, synthetic construct
  misc_feature 1..46: note = Synthetic oligonucleotide
  source 1..46: mol_type = other DNA
cccaggcgggcctcgctactcatggggtactgtttgatagagacag

SEQ ID NO: 237 - DNA, 46 bp, synthetic construct
  misc_feature 1..46: note = Synthetic oligonucleotide
  source 1..46: mol_type = other DNA
ctgtctctatcaaacagtaccccatgagtaccgaggcccgcctggg

SEQ ID NO: 238 - DNA, 46 bp, synthetic construct
  misc_feature 1..46: note = Synthetic oligonucleotide
  source 1..46: mol_type = other DNA
cccaggcgggcctcggtactcatggggtactgtttgatagagacag

SEQ ID NO: 239 - DNA, 46 bp, synthetic construct
  misc_feature 1..46: note = Synthetic oligonucleotide
  source 1..46: mol_type = other DNA
ctgtctctatcaaacagtaccccatgagttgcgaggcccgcctggg

SEQ ID NO: 240 - DNA, 46 bp, synthetic construct
  misc_feature 1..46: note = Synthetic oligonucleotide
  source 1..46: mol_type = other DNA
cccaggcgggcctcgcaactcatggggtactgtttgatagagacag

SEQ ID NO: 241 - DNA, 46 bp, synthetic construct
  misc_feature 1..46: note = Synthetic oligonucleotide
  source 1..46: mol_type = other DNA
ctgtctctatcaaacagtaccccatgagttttgaggcccgcctggg

SEQ ID NO: 242 - DNA, 46 bp, synthetic construct
  misc_feature 1..46: note = Synthetic oligonucleotide
  source 1..46: mol_type = other DNA
cccaggcgggcctcaaaactcatggggtactgtttgatagagacag

SEQ ID NO: 243 - DNA, 46 bp, synthetic construct
  misc_feature 1..46: note = Synthetic oligonucleotide
  source 1..46: mol_type = other DNA
ctgtctctatcaaacagtaccccatgagttatgaggcccgcctggg

SEQ ID NO: 244 - DNA, 46 bp, synthetic construct
  misc_feature 1..46: note = Synthetic oligonucleotide
  source 1..46: mol_type = other DNA
cccaggcgggcctcataactcatggggtactgtttgatagagacag

SEQ ID NO: 245 - DNA, 46 bp, synthetic construct
  misc_feature 1..46: note = Synthetic oligonucleotide
  source 1..46: mol_type = other DNA
ctgtctctatcaaacagtaccccatgagtcatgaggcccgcctggg

SEQ ID NO: 246 - DNA, 46 bp, synthetic construct
  misc_feature 1..46: note = Synthetic oligonucleotide
  source 1..46: mol_type = other DNA
cccaggcgggcctcatgactcatggggtactgtttgatagagacag

SEQ ID NO: 247 - DNA, 46 bp, synthetic construct
  misc_feature 1..46: note = Synthetic oligonucleotide
  source 1..46: mol_type = other DNA
ctgtctctatcaaacagtaccccatgagttgggaggcccgcctggg

SEQ ID NO: 248 - DNA, 46 bp, synthetic construct
  misc_feature 1..46: note = Synthetic oligonucleotide
  source 1..46: mol_type = other DNA
cccaggcgggcctcccaactcatggggtactgtttgatagagacag

SEQ ID NO: 249 - DNA, 46 bp, synthetic construct
  misc_feature 1..46: note = Synthetic oligonucleotide
  source 1..46: mol_type = other DNA
ctgtctctatcaaacagtaccccatgagtgatgaggcccgcctggg

SEQ ID NO: 250 - DNA, 46 bp, synthetic construct
  misc_feature 1..46: note = Synthetic oligonucleotide
  source 1..46: mol_type = other DNA
cccaggcgggcctcatcactcatggggtactgtttgatagagacag

SEQ ID NO: 251 - DNA, 46 bp, synthetic construct
  misc_feature 1..46: note = Synthetic oligonucleotide
  source 1..46: mol_type = other DNA
ctgtctctatcaaacagtaccccatgagtaacgaggcccgcctggg

SEQ ID NO: 252 - DNA, 46 bp, synthetic construct
  misc_feature 1..46: note = Synthetic oligonucleotide
  source 1..46: mol_type = other DNA
cccaggcgggcctcgttactcatggggtactgtttgatagagacag

SEQ ID NO: 253 - DNA, 46 bp, synthetic construct
  misc_feature 1..46: note = Synthetic oligonucleotide
  source 1..46: mol_type = other DNA
ctgtctctatcaaacagtaccccatgagtaaagaggcccgcctggg

SEQ ID NO: 254 - DNA, 46 bp, synthetic construct
  misc_feature 1..46: note = Synthetic oligonucleotide
  source 1..46: mol_type = other DNA
cccaggcgggcctctttactcatggggtactgtttgatagagacag

SEQ ID NO: 255 - DNA, 45 bp, synthetic construct
  misc_feature 1..45: note = Synthetic oligonucleotide
  source 1..45: mol_type = other DNA
cgcctggggattaagccacatattggccgcttgctggaccagggg

SEQ ID NO: 256 - DNA, 45 bp, synthetic construct
  misc_feature 1..45: note = Synthetic oligonucleotide
  source 1..45: mol_type = other DNA
cccctggtccagcaagcggccaatatgtggcttaatccccaggcg

SEQ ID NO: 257 - DNA, 45 bp, synthetic construct
  misc_feature 1..45: note = Synthetic oligonucleotide
  source 1..45: mol_type = other DNA
cgcctggggattaagccacatattctgcgcttgctggaccagggg

SEQ ID NO: 258 - DNA, 45 bp, synthetic construct
  misc_feature 1..45: note = Synthetic oligonucleotide
  source 1..45: mol_type = other DNA
cccctggtccagcaagcgcagaatatgtggcttaatccccaggcg

SEQ ID NO: 259 - DNA, 45 bp, synthetic construct
  misc_feature 1..45: note = Synthetic oligonucleotide
  source 1..45: mol_type = other DNA
cgcctggggattaagccacatattattcgcttgctggaccagggg

SEQ ID NO: 260 - DNA, 45 bp, synthetic construct
  misc_feature 1..45: note = Synthetic oligonucleotide
  source 1..45: mol_type = other DNA
cccctggtccagcaagcgaataatatgtggcttaatccccaggcg

SEQ ID NO: 261 - DNA, 45 bp, synthetic construct
  misc_feature 1..45: note = Synthetic oligonucleotide
  source 1..45: mol_type = other DNA
cgcctggggattaagccacatattgtgcgcttgctggaccagggg

SEQ ID NO: 262 - DNA, 45 bp, synthetic construct
  misc_feature 1..45: note = Synthetic oligonucleotide
  source 1..45: mol_type = other DNA
cccctggtccagcaagcgcacaatatgtggcttaatccccaggcg

SEQ ID NO: 263 - DNA, 45 bp, synthetic construct
  misc_feature 1..45: note = Synthetic oligonucleotide
  source 1..45: mol_type = other DNA
cgcctggggattaagccacatattccgcgcttgctggaccagggg

SEQ ID NO: 264 - DNA, 45 bp, synthetic construct
  misc_feature 1..45: note = Synthetic oligonucleotide
  source 1..45: mol_type = other DNA
cccctggtccagcaagcgcggaatatgtggcttaatccccaggcg

SEQ ID NO: 265 - DNA, 45 bp, synthetic construct
  misc_feature 1..45: note = Synthetic oligonucleotide
  source 1..45: mol_type = other DNA
cgcctggggattaagccacatattatgcgcttgctggaccagggg

SEQ ID NO: 266 - DNA, 45 bp, synthetic construct
  misc_feature 1..45: note = Synthetic oligonucleotide
  source 1..45: mol_type = other DNA
cccctggtccagcaagcgcataatatgtggcttaatccccaggcg

SEQ ID NO: 267 - DNA, 45 bp, synthetic construct
  misc_feature 1..45: note = Synthetic oligonucleotide
  source 1..45: mol_type = other DNA
cgcctggggattaagccacatattagccgcttgctggaccagggg

SEQ ID NO: 268 - DNA, 45 bp, synthetic construct
  misc_feature 1..45: note = Synthetic oligonucleotide
  source 1..45: mol_type = other DNA
cccctggtccagcaagcggctaatatgtggcttaatccccaggcg

SEQ ID NO: 269 - DNA, 45 bp, synthetic construct
  misc_feature 1..45: note = Synthetic oligonucleotide
  source 1..45: mol_type = other DNA
cgcctggggattaagccacatattacccgcttgctggaccagggg

SEQ ID NO: 270 - DNA, 45 bp, synthetic construct
  misc_feature 1..45: note = Synthetic oligonucleotide
  source 1..45: mol_type = other DNA
cccctggtccagcaagcgggtaatatgtggcttaatccccaggcg

SEQ ID NO: 271 - DNA, 45 bp, synthetic construct
  misc_feature 1..45: note = Synthetic oligonucleotide
  source 1..45: mol_type = other DNA
cgcctggggattaagccacatatttgccgcttgctggaccagggg

SEQ ID NO: 272 - DNA, 45 bp, synthetic construct
  misc_feature 1..45: note = Synthetic oligonucleotide
  source 1..45: mol_type = other DNA
cccctggtccagcaagcggcaaatatgtggcttaatccccaggcg

SEQ ID NO: 273 - DNA, 45 bp, synthetic construct
  misc_feature 1..45: note = Synthetic oligonucleotide
  source 1..45: mol_type = other DNA
cgcctggggattaagccacatatttttcgcttgctggaccagggg

SEQ ID NO: 274 - DNA, 45 bp, synthetic construct
  misc_feature 1..45: note = Synthetic oligonucleotide
  source 1..45: mol_type = other DNA
cccctggtccagcaagcgaaaaatatgtggcttaatccccaggcg

SEQ ID NO: 275 - DNA, 45 bp, synthetic construct
  misc_feature 1..45: note = Synthetic oligonucleotide
  source 1..45: mol_type = other DNA
cgcctggggattaagccacatatttatcgcttgctggaccagggg

SEQ ID NO: 276 - DNA, 45 bp, synthetic construct
  misc_feature 1..45: note = Synthetic oligonucleotide
  source 1..45: mol_type = other DNA
cccctggtccagcaagcgataaatatgtggcttaatccccaggcg

SEQ ID NO: 277 - DNA, 45 bp, synthetic construct
  misc_feature 1..45: note = Synthetic oligonucleotide
  source 1..45: mol_type = other DNA
cgcctggggattaagccacatattcatcgcttgctggaccagggg

SEQ ID NO: 278 - DNA, 45 bp, synthetic construct
  misc_feature 1..45: note = Synthetic oligonucleotide
  source 1..45: mol_type = other DNA
cccctggtccagcaagcgatgaatatgtggcttaatccccaggcg

SEQ ID NO: 279 - DNA, 45 bp, synthetic construct
  misc_feature 1..45: note = Synthetic oligonucleotide
  source 1..45: mol_type = other DNA
cgcctggggattaagccacatatttggcgcttgctggaccagggg

SEQ ID NO: 280 - DNA, 45 bp, synthetic construct
  misc_feature 1..45: note = Synthetic oligonucleotide
  source 1..45: mol_type = other DNA
cccctggtccagcaagcgccaaatatgtggcttaatccccaggcg

SEQ ID NO: 281 - DNA, 45 bp, synthetic construct
  misc_feature 1..45: note = Synthetic oligonucleotide
  source 1..45: mol_type = other DNA
cgcctggggattaagccacatattgatcgcttgctggaccagggg

SEQ ID NO: 282 - DNA, 45 bp, synthetic construct
  misc_feature 1..45: note = Synthetic oligonucleotide
  source 1..45: mol_type = other DNA
cccctggtccagcaagcgatcaatatgtggcttaatccccaggcg

SEQ ID NO: 283 - DNA, 45 bp, synthetic construct
  misc_feature 1..45: note = Synthetic oligonucleotide
  source 1..45: mol_type = other DNA
cgcctggggattaagccacatattaaccgcttgctggaccagggg

SEQ ID NO: 284 - DNA, 45 bp, synthetic construct
  misc_feature 1..45: note = Synthetic oligonucleotide
  source 1..45: mol_type = other DNA
cccctggtccagcaagcggttaatatgtggcttaatccccaggcg

SEQ ID NO: 285 - DNA, 45 bp, synthetic construct
  misc_feature 1..45: note = Synthetic oligonucleotide
  source 1..45: mol_type = other DNA
cgcctggggattaagccacatattaaacgcttgctggaccagggg

SEQ ID NO: 286 - DNA, 45 bp, synthetic construct
  misc_feature 1..45: note = Synthetic oligonucleotide
  source 1..45: mol_type = other DNA
cccctggtccagcaagcgtttaatatgtggcttaatccccaggcg

SEQ ID NO: 287 - DNA, 45 bp, synthetic construct
  misc_feature 1..45: note = Synthetic oligonucleotide
  source 1..45: mol_type = other DNA
ccgtggaacaccccccttggccccgtgaaaaagccaggtacaaac

SEQ ID NO: 288 - DNA, 45 bp, synthetic construct
  misc_feature 1..45: note = Synthetic oligonucleotide
  source 1..45: mol_type = other DNA
gtttgtacctggctttttcacggggccaaggggggtgttccacgg

SEQ ID NO: 289 - DNA, 45 bp, synthetic construct
  misc_feature 1..45: note = Synthetic oligonucleotide
  source 1..45: mol_type = other DNA
ccgtggaacaccccccttattcccgtgaaaaagccaggtacaaac

SEQ ID NO: 290 - DNA, 45 bp, synthetic construct
  misc_feature 1..45: note = Synthetic oligonucleotide
  source 1..45: mol_type = other DNA
gtttgtacctggctttttcacgggaataaggggggtgttccacgg

SEQ ID NO: 291 - DNA, 45 bp, synthetic construct
  misc_feature 1..45: note = Synthetic oligonucleotide
  source 1..45: mol_type = other DNA
ccgtggaacaccccccttgtgcccgtgaaaaagccaggtacaaac

SEQ ID NO: 292 - DNA, 45 bp, synthetic construct
  misc_feature 1..45: note = Synthetic oligonucleotide
  source 1..45: mol_type = other DNA
gtttgtacctggctttttcacgggcacaaggggggtgttccacgg

SEQ ID NO: 293 - DNA, 45 bp, synthetic construct
  misc_feature 1..45: note = Synthetic oligonucleotide
  source 1..45: mol_type = other DNA
ccgtggaacaccccccttccgcccgtgaaaaagccaggtacaaac

SEQ ID NO: 294 - DNA, 45 bp, synthetic construct
  misc_feature 1..45: note = Synthetic oligonucleotide
  source 1..45: mol_type = other DNA
gtttgtacctggctttttcacgggcggaaggggggtgttccacgg

SEQ ID NO: 295 - DNA, 45 bp, synthetic construct
  misc_feature 1..45: note = Synthetic oligonucleotide
  source 1..45: mol_type = other DNA
ccgtggaacaccccccttatgcccgtgaaaaagccaggtacaaac

SEQ ID NO: 296 - DNA, 45 bp, synthetic construct
  misc_feature 1..45: note = Synthetic oligonucleotide
  source 1..45: mol_type = other DNA
gtttgtacctggctttttcacgggcataaggggggtgttccacgg

SEQ ID NO: 297 - DNA, 45 bp, synthetic construct
  misc_feature 1..45: note = Synthetic oligonucleotide
  source 1..45: mol_type = other DNA
ccgtggaacaccccccttagccccgtgaaaaagccaggtacaaac

SEQ ID NO: 298 - DNA, 45 bp, synthetic construct
  misc_feature 1..45: note = Synthetic oligonucleotide
  source 1..45: mol_type = other DNA
gtttgtacctggctttttcacggggctaaggggggtgttccacgg

SEQ ID NO: 299 - DNA, 45 bp, synthetic construct
  misc_feature 1..45: note = Synthetic oligonucleotide
  source 1..45: mol_type = other DNA
ccgtggaacaccccccttacccccgtgaaaaagccaggtacaaac

SEQ ID NO: 300 - DNA, 45 bp, synthetic construct
  misc_feature 1..45: note = Synthetic oligonucleotide
  source 1..45: mol_type = other DNA
gtttgtacctggctttttcacgggggtaaggggggtgttccacgg

SEQ ID NO: 301 - DNA, 45 bp, synthetic construct
  misc_feature 1..45: note = Synthetic oligonucleotide
  source 1..45: mol_type = other DNA
ccgtggaacacccccctttgccccgtgaaaaagccaggtacaaac

SEQ ID NO: 302 - DNA, 45 bp, synthetic construct
  misc_feature 1..45: note = Synthetic oligonucleotide
  source 1..45: mol_type = other DNA
gtttgtacctggctttttcacggggcaaaggggggtgttccacgg

SEQ ID NO: 303 - DNA, 45 bp, synthetic construct
  misc_feature 1..45: note = Synthetic oligonucleotide
  source 1..45: mol_type = other DNA
ccgtggaacacccccctttttcccgtgaaaaagccaggtacaaac

SEQ ID NO: 304 - DNA, 45 bp, synthetic construct
  misc_feature 1..45: note = Synthetic oligonucleotide
  source 1..45: mol_type = other DNA
gtttgtacctggctttttcacgggaaaaaggggggtgttccacgg

SEQ ID NO: 305 - DNA, 45 bp, synthetic construct
  misc_feature 1..45: note = Synthetic oligonucleotide
  source 1..45: mol_type = other DNA
ccgtggaacacccccctttatcccgtgaaaaagccaggtacaaac

SEQ ID NO: 306 - DNA, 45 bp, synthetic construct
  misc_feature 1..45: note = Synthetic oligonucleotide
  source 1..45: mol_type = other DNA
gtttgtacctggctttttcacgggataaaggggggtgttccacgg

SEQ ID NO: 307 - DNA, 45 bp, synthetic construct
  misc_feature 1..45: note = Synthetic oligonucleotide
  source 1..45: mol_type = other DNA
ccgtggaacaccccccttcatcccgtgaaaaagccaggtacaaac

SEQ ID NO: 308 - DNA, 45 bp, synthetic construct
  misc_feature 1..45: note = Synthetic oligonucleotide
  source 1..45: mol_type = other DNA
gtttgtacctggctttttcacgggatgaaggggggtgttccacgg

SEQ ID NO: 309 - DNA, 45 bp, synthetic construct
  misc_feature 1..45: note = Synthetic oligonucleotide
  source 1..45: mol_type = other DNA
ccgtggaacacccccctttggcccgtgaaaaagccaggtacaaac

SEQ ID NO: 310 - DNA, 45 bp, synthetic construct
  misc_feature 1..45: note = Synthetic oligonucleotide
  source 1..45: mol_type = other DNA
gtttgtacctggctttttcacgggccaaaggggggtgttccacgg

SEQ ID NO: 311 - DNA, 45 bp, synthetic construct
  misc_feature 1..45: note = Synthetic oligonucleotide
  source 1..45: mol_type = other DNA
ccgtggaacaccccccttgatcccgtgaaaaagccaggtacaaac

SEQ ID NO: 312 - DNA, 45 bp, synthetic construct
  misc_feature 1..45: note = Synthetic oligonucleotide
  source 1..45: mol_type = other DNA
gtttgtacctggctttttcacgggatcaaggggggtgttccacgg

SEQ ID NO: 313 - DNA, 45 bp, synthetic construct
  misc_feature 1..45: note = Synthetic oligonucleotide
  source 1..45: mol_type = other DNA
ccgtggaacaccccccttaaccccgtgaaaaagccaggtacaaac

SEQ ID NO: 314 - DNA, 45 bp, synthetic construct
  misc_feature 1..45: note = Synthetic oligonucleotide
  source 1..45: mol_type = other DNA
gtttgtacctggctttttcacggggttaaggggggtgttccacgg

SEQ ID NO: 315 - DNA, 45 bp, synthetic construct
  misc_feature 1..45: note = Synthetic oligonucleotide
  source 1..45: mol_type = other DNA
ccgtggaacaccccccttcagcccgtgaaaaagccaggtacaaac

SEQ ID NO: 316 - DNA, 45 bp, synthetic construct
  misc_feature 1..45: note = Synthetic oligonucleotide
  source 1..45: mol_type = other DNA
gtttgtacctggctttttcacgggctgaaggggggtgttccacgg

SEQ ID NO: 317 - DNA, 45 bp, synthetic construct
  misc_feature 1..45: note = Synthetic oligonucleotide
  source 1..45: mol_type = other DNA
ccgtggaacaccccccttaaacccgtgaaaaagccaggtacaaac

SEQ ID NO: 318 - DNA, 45 bp, synthetic construct
  misc_feature 1..45: note = Synthetic oligonucleotide
  source 1..45: mol_type = other DNA
gtttgtacctggctttttcacgggtttaaggggggtgttccacgg

SEQ ID NO: 319 - DNA, 46 bp, synthetic construct
  misc_feature 1..46: note = Synthetic oligonucleotide
  source 1..46: mol_type = other DNA
agaaggtcaacgttggctgactggcgcgcgtaaggagaccgtaatg

SEQ ID NO: 320 - DNA, 46 bp, synthetic construct
  misc_feature 1..46: note = Synthetic oligonucleotide
  source 1..46: mol_type = other DNA
cattacggtctccttacgcgcgccagtcagccaacgttgaccttct

SEQ ID NO: 321 - DNA, 46 bp, synthetic construct
  misc_feature 1..46: note = Synthetic oligonucleotide
  source 1..46: mol_type = other DNA
agaaggtcaacgttggctgactctggcgcgtaaggagaccgtaatg

SEQ ID NO: 322 - DNA, 46 bp, synthetic construct
  misc_feature 1..46: note = Synthetic oligonucleotide
  source 1..46: mol_type = other DNA
cattacggtctccttacgcgccagagtcagccaacgttgaccttct

SEQ ID NO: 323 - DNA, 46 bp, synthetic construct
  misc_feature 1..46: note = Synthetic oligonucleotide
  source 1..46: mol_type = other DNA
agaaggtcaacgttggctgactattgcgcgtaaggagaccgtaatg

SEQ ID NO: 324 - DNA, 46 bp, synthetic construct
  misc_feature 1..46: note = Synthetic oligonucleotide
  source 1..46: mol_type = other DNA
cattacggtctccttacgcgcaatagtcagccaacgttgaccttct

SEQ ID NO: 325 - DNA, 46 bp, synthetic construct
  misc_feature 1..46: note = Synthetic oligonucleotide
  source 1..46: mol_type = other DNA
agaaggtcaacgttggctgactgtggcgcgtaaggagaccgtaatg

SEQ ID NO: 326 - DNA, 46 bp, synthetic construct
  misc_feature 1..46: note = Synthetic oligonucleotide
  source 1..46: mol_type = other DNA
cattacggtctccttacgcgccacagtcagccaacgttgaccttct

SEQ ID NO: 327 - DNA, 46 bp, synthetic construct
  misc_feature 1..46: note = Synthetic oligonucleotide
  source 1..46: mol_type = other DNA
agaaggtcaacgttggctgactccggcgcgtaaggagaccgtaatg

SEQ ID NO: 328 - DNA, 46 bp, synthetic construct
  misc_feature 1..46: note = Synthetic oligonucleotide
  source 1..46: mol_type = other DNA
cattacggtctccttacgcgccggagtcagccaacgttgaccttct

SEQ ID NO: 329 - DNA, 46 bp, synthetic construct
  misc_feature 1..46: note = Synthetic oligonucleotide
  source 1..46: mol_type = other DNA
agaaggtcaacgttggctgactatggcgcgtaaggagaccgtaatg

SEQ ID NO: 330 - DNA, 46 bp, synthetic construct
  misc_feature 1..46: note = Synthetic oligonucleotide
  source 1..46: mol_type = other DNA
cattacggtctccttacgcgccatagtcagccaacgttgaccttct

SEQ ID NO: 331 - DNA, 46 bp, synthetic construct
  misc_feature 1..46: note = Synthetic oligonucleotide
  source 1..46: mol_type = other DNA
agaaggtcaacgttggctgactagcgcgcgtaaggagaccgtaatg

SEQ ID NO: 332 - DNA, 46 bp, synthetic construct
  misc_feature 1..46: note = Synthetic oligonucleotide
  source 1..46: mol_type = other DNA
cattacggtctccttacgcgcgctagtcagccaacgttgaccttct

SEQ ID NO: 333 - DNA, 46 bp, synthetic construct
  misc_feature 1..46: note = Synthetic oligonucleotide
  source 1..46: mol_type = other DNA
agaaggtcaacgttggctgactaccgcgcgtaaggagaccgtaatg

SEQ ID NO: 334 - DNA, 46 bp, synthetic construct
  misc_feature 1..46: note = Synthetic oligonucleotide
  source 1..46: mol_type = other DNA
cattacggtctccttacgcgcggtagtcagccaacgttgaccttct

SEQ ID NO: 335 - DNA, 46 bp, synthetic construct
  misc_feature 1..46: note = Synthetic oligonucleotide
  source 1..46: mol_type = other DNA
agaaggtcaacgttggctgacttgcgcgcgtaaggagaccgtaatg

SEQ ID NO: 336 - DNA, 46 bp, synthetic construct
  misc_feature 1..46: note = Synthetic oligonucleotide
  source 1..46: mol_type = other DNA
cattacggtctccttacgcgcgcaagtcagccaacgttgaccttct

SEQ ID NO: 337 - DNA, 46 bp, synthetic construct
  misc_feature 1..46: note = Synthetic oligonucleotide
  source 1..46: mol_type = other DNA
agaaggtcaacgttggctgacttttgcgcgtaaggagaccgtaatg

SEQ ID NO: 338 - DNA, 46 bp, synthetic construct
  misc_feature 1..46: note = Synthetic oligonucleotide
  source 1..46: mol_type = other DNA
cattacggtctccttacgcgcaaaagtcagccaacgttgaccttct

SEQ ID NO: 339 - DNA, 46 bp, synthetic construct
  misc_feature 1..46: note = Synthetic oligonucleotide
  source 1..46: mol_type = other DNA
agaaggtcaacgttggctgacttatgcgcgtaaggagaccgtaatg

SEQ ID NO: 340 - DNA, 46 bp, synthetic construct
  misc_feature 1..46: note = Synthetic oligonucleotide
  source 1..46: mol_type = other DNA
cattacggtctccttacgcgcataagtcagccaacgttgaccttct

SEQ ID NO: 341 - DNA, 46 bp, synthetic construct
  misc_feature 1..46: note = Synthetic oligonucleotide
  source 1..46: mol_type = other DNA
agaaggtcaacgttggctgactcatgcgcgtaaggagaccgtaatg

SEQ ID NO: 342 - DNA, 46 bp, synthetic construct
  misc_feature 1..46: note = Synthetic oligonucleotide
  source 1..46: mol_type = other DNA
cattacggtctccttacgcgcatgagtcagccaacgttgaccttct

SEQ ID NO: 343 - DNA, 46 bp, synthetic construct
  misc_feature 1..46: note = Synthetic oligonucleotide
  source 1..46: mol_type = other DNA
agaaggtcaacgttggctgacttgggcgcgtaaggagaccgtaatg

SEQ ID NO: 344 - DNA, 46 bp, synthetic construct
  misc_feature 1..46: note = Synthetic oligonucleotide
  source 1..46: mol_type = other DNA
cattacggtctccttacgcgcccaagtcagccaacgttgaccttct

SEQ ID NO: 345 - DNA, 46 bp, synthetic construct
  misc_feature 1..46: note = Synthetic oligonucleotide
  source 1..46: mol_type = other DNA
agaaggtcaacgttggctgactaacgcgcgtaaggagaccgtaatg

SEQ ID NO: 346 - DNA, 46 bp, synthetic construct
  misc_feature 1..46: note = Synthetic oligonucleotide
  source 1..46: mol_type = other DNA
cattacggtctccttacgcgcgttagtcagccaacgttgaccttct

SEQ ID NO: 347 - DNA, 46 bp, synthetic construct
  misc_feature 1..46: note = Synthetic oligonucleotide
  source 1..46: mol_type = other DNA
agaaggtcaacgttggctgactcaggcgcgtaaggagaccgtaatg

SEQ ID NO: 348 - DNA, 46 bp, synthetic construct
  misc_feature 1..46: note = Synthetic oligonucleotide
  source 1..46: mol_type = other DNA
cattacggtctccttacgcgcctgagtcagccaacgttgaccttct

SEQ ID NO: 349 - DNA, 46 bp, synthetic construct
  misc_feature 1..46: note = Synthetic oligonucleotide
  source 1..46: mol_type = other DNA
agaaggtcaacgttggctgactaaagcgcgtaaggagaccgtaatg

SEQ ID NO: 350 - DNA, 46 bp, synthetic construct
  misc_feature 1..46: note = Synthetic oligonucleotide
  source 1..46: mol_type = other DNA
cattacggtctccttacgcgctttagtcagccaacgttgaccttct

SEQ ID NO: 351 - DNA, 47 bp, synthetic construct
  misc_feature 1..47: note = Synthetic oligonucleotide
  source 1..47: mol_type = other DNA
gcctacgcctaagacgccaggccagttgcgtgaatttttgggcacag

SEQ ID NO: 352 - DNA, 47 bp, synthetic construct
  misc_feature 1..47: note = Synthetic oligonucleotide
  source 1..47: mol_type = other DNA
ctgtgcccaaaaattcacgcaactggcctggcgtcttaggcgtaggc

SEQ ID NO: 353 - DNA, 47 bp, synthetic construct
  misc_feature 1..47: note = Synthetic oligonucleotide
  source 1..47: mol_type = other DNA
gcctacgcctaagacgccactgcagttgcgtgaatttttgggcacag

SEQ ID NO: 354 - DNA, 47 bp, synthetic construct
  misc_feature 1..47: note = Synthetic oligonucleotide
  source 1..47: mol_type = other DNA
ctgtgcccaaaaattcacgcaactgcagtggcgtcttaggcgtaggc

SEQ ID NO: 355 - DNA, 47 bp, synthetic construct
  misc_feature 1..47: note = Synthetic oligonucleotide
  source 1..47: mol_type = other DNA
gcctacgcctaagacgccaattcagttgcgtgaatttttgggcacag

SEQ ID NO: 356 - DNA, 47 bp, synthetic construct
  misc_feature 1..47: note = Synthetic oligonucleotide
  source 1..47: mol_type = other DNA
ctgtgcccaaaaattcacgcaactgaattggcgtcttaggcgtaggc

SEQ ID NO: 357 - DNA, 47 bp, synthetic construct
  misc_feature 1..47: note = Synthetic oligonucleotide
  source 1..47: mol_type = other DNA
gcctacgcctaagacgccagtgcagttgcgtgaatttttgggcacag

SEQ ID NO: 358 - DNA, 47 bp, synthetic construct
  misc_feature 1..47: note = Synthetic oligonucleotide
  source 1..47: mol_type = other DNA
ctgtgcccaaaaattcacgcaactgcactggcgtcttaggcgtaggc

SEQ ID NO: 359 - DNA, 47 bp, synthetic construct
  misc_feature 1..47: note = Synthetic oligonucleotide
  source 1..47: mol_type = other DNA
gcctacgcctaagacgccaccgcagttgcgtgaatttttgggcacag

SEQ ID NO: 360 - DNA, 47 bp, synthetic construct
  misc_feature 1..47: note = Synthetic oligonucleotide
  source 1..47: mol_type = other DNA
ctgtgcccaaaaattcacgcaactgcggtggcgtcttaggcgtaggc

SEQ ID NO: 361 - DNA, 47 bp, synthetic construct
  misc_feature 1..47: note = Synthetic oligonucleotide
  source 1..47: mol_type = other DNA
gcctacgcctaagacgccaatgcagttgcgtgaatttttgggcacag

SEQ ID NO: 362 - DNA, 47 bp, synthetic construct
  misc_feature 1..47: note = Synthetic oligonucleotide
  source 1..47: mol_type = other DNA
ctgtgcccaaaaattcacgcaactgcattggcgtcttaggcgtaggc

SEQ ID NO: 363 - DNA, 47 bp, synthetic construct
  misc_feature 1..47: note = Synthetic oligonucleotide
  source 1..47: mol_type = other DNA
gcctacgcctaagacgccaagccagttgcgtgaatttttgggcacag

SEQ ID NO: 364 - DNA, 47 bp, synthetic construct
  misc_feature 1..47: note = Synthetic oligonucleotide
  source 1..47: mol_type = other DNA
ctgtgcccaaaaattcacgcaactggcttggcgtcttaggcgtaggc

SEQ ID NO: 365 - DNA, 47 bp, synthetic construct
  misc_feature 1..47: note = Synthetic oligonucleotide
  source 1..47: mol_type = other DNA
gcctacgcctaagacgccaacccagttgcgtgaatttttgggcacag

SEQ ID NO: 366 - DNA, 47 bp, synthetic construct
  misc_feature 1..47: note = Synthetic oligonucleotide
  source 1..47: mol_type = other DNA
ctgtgcccaaaaattcacgcaactgggttggcgtcttaggcgtaggc

SEQ ID NO: 367 - DNA, 47 bp, synthetic construct
  misc_feature 1..47: note = Synthetic oligonucleotide
  source 1..47: mol_type = other DNA
gcctacgcctaagacgccatgccagttgcgtgaatttttgggcacag

SEQ ID NO: 368 - DNA, 47 bp, synthetic construct
  misc_feature 1..47: note = Synthetic oligonucleotide
  source 1..47: mol_type = other DNA
ctgtgcccaaaaattcacgcaactggcatggcgtcttaggcgtaggc

SEQ ID NO: 369 - DNA, 47 bp, synthetic construct
  misc_feature 1..47: note = Synthetic oligonucleotide
  source 1..47: mol_type = other DNA
gcctacgcctaagacgccatttcagttgcgtgaatttttgggcacag

SEQ ID NO: 370 - DNA, 47 bp, synthetic construct
  misc_feature 1..47: note = Synthetic oligonucleotide
  source 1..47: mol_type = other DNA
ctgtgcccaaaaattcacgcaactgaaatggcgtcttaggcgtaggc

SEQ ID NO: 371 - DNA, 47 bp, synthetic construct
  misc_feature 1..47: note = Synthetic oligonucleotide
  source 1..47: mol_type = other DNA
gcctacgcctaagacgccatatcagttgcgtgaatttttgggcacag

SEQ ID NO: 372 - DNA, 47 bp, synthetic construct
  misc_feature 1..47: note = Synthetic oligonucleotide
  source 1..47: mol_type = other DNA
ctgtgcccaaaaattcacgcaactgatatggcgtcttaggcgtaggc

SEQ ID NO: 373 - DNA, 47 bp, synthetic construct
  misc_feature 1..47: note = Synthetic oligonucleotide
  source 1..47: mol_type = other DNA
gcctacgcctaagacgccacatcagttgcgtgaatttttgggcacag

SEQ ID NO: 374 - DNA, 47 bp, synthetic construct
  misc_feature 1..47: note = Synthetic oligonucleotide
  source 1..47: mol_type = other DNA
ctgtgcccaaaaattcacgcaactgatgtggcgtcttaggcgtaggc

SEQ ID NO: 375 - DNA, 47 bp, synthetic construct
  misc_feature 1..47: note = Synthetic oligonucleotide
  source 1..47: mol_type = other DNA
gcctacgcctaagacgccatggcagttgcgtgaatttttgggcacag

SEQ ID NO: 376 - DNA, 47 bp, synthetic construct
  misc_feature 1..47: note = Synthetic oligonucleotide
  source 1..47: mol_type = other DNA
ctgtgcccaaaaattcacgcaactgccatggcgtcttaggcgtaggc

SEQ ID NO: 377 - DNA, 47 bp, synthetic construct
  misc_feature 1..47: note = Synthetic oligonucleotide
  source 1..47: mol_type = other DNA
gcctacgcctaagacgccagatcagttgcgtgaatttttgggcacag

SEQ ID NO: 378 - DNA, 47 bp, synthetic construct
  misc_feature 1..47: note = Synthetic oligonucleotide
  source 1..47: mol_type = other DNA
ctgtgcccaaaaattcacgcaactgatctggcgtcttaggcgtaggc

SEQ ID NO: 379 - DNA, 47 bp, synthetic construct
  misc_feature 1..47: note = Synthetic oligonucleotide
  source 1..47: mol_type = other DNA
gcctacgcctaagacgccaaaccagttgcgtgaatttttgggcacag

SEQ ID NO: 380 - DNA, 47 bp, synthetic construct
  misc_feature 1..47: note = Synthetic oligonucleotide
  source 1..47: mol_type = other DNA
ctgtgcccaaaaattcacgcaactggtttggcgtcttaggcgtaggc

SEQ ID NO: 381 - DNA, 47 bp, synthetic construct
  misc_feature 1..47: note = Synthetic oligonucleotide
  source 1..47: mol_type = other DNA
gcctacgcctaagacgccacagcagttgcgtgaatttttgggcacag

SEQ ID NO: 382 - DNA, 47 bp, synthetic construct
  misc_feature 1..47: note = Synthetic oligonucleotide
  source 1..47: mol_type = other DNA
ctgtgcccaaaaattcacgcaactgctgtggcgtcttaggcgtaggc

SEQ ID NO: 383 - DNA, 62 bp, synthetic construct
  misc_feature 1..62: note = Synthetic oligonucleotide
  source 1..62: mol_type = other DNA
aggcaacgtctacacctgtctctcgtaaacagtaccccatgagtcgtgaggcccgcctgggg

SEQ ID NO: 384 - DNA, 62 bp, synthetic construct
  misc_feature 1..62: note = Synthetic oligonucleotide
  source 1..62: mol_type = other DNA
ccccaggcgggcctcacgactcatggggtactgtttacgagagacaggtgtagacgttgcct

SEQ ID NO: 385 - DNA, 51 bp, synthetic construct
  misc_feature 1..51: note = Synthetic oligonucleotide
  source 1..51: mol_type = other DNA
aggcaacgtctacacctgtctctaaaaaacagtaccccatgagtcgtgagg

SEQ ID NO: 386 - DNA, 51 bp, synthetic construct
  misc_feature 1..51: note = Synthetic oligonucleotide
  source 1..51: mol_type = other DNA
cctcacgactcatggggtactgttttttagagacaggtgtagacgttgcct

SEQ ID NO: 387 - DNA, 51 bp, synthetic construct
  misc_feature 1..51: note = Synthetic oligonucleotide
  source 1..51: mol_type = other DNA
aggcaacgtctacacctgtctctatgaaacagtaccccatgagtcgtgagg

SEQ ID NO: 388 - DNA, 51 bp, synthetic construct
  misc_feature 1..51: note = Synthetic oligonucleotide
  source 1..51: mol_type = other DNA
cctcacgactcatggggtactgtttcatagagacaggtgtagacgttgcct

SEQ ID NO: 389 - DNA, 53 bp, synthetic construct
  misc_feature 1..53: note = Synthetic oligonucleotide
  source 1..53: mol_type = other DNA
aggcaacgtctacacctgtctctatgaaacagtaccccatgagtattgaggcc

SEQ ID NO: 390-392 - skipped (3 entries)

SEQ ID NO: 393 - DNA, 44 bp, synthetic construct
  misc_feature 1..44: note = Synthetic oligonucleotide
  source 1..44: mol_type = other DNA
gtctctatcaaacagtaccccatggcgcaagaggcccgcctggg

SEQ ID NO: 394 - DNA, 44 bp, synthetic construct
  misc_feature 1..44: note = Synthetic oligonucleotide
  source 1..44: mol_type = other DNA
gtctctatcaaacagtaccccatgcgtcaagaggcccgcctggg

SEQ ID NO: 395 - DNA, 45 bp, synthetic construct
  misc_feature 1..45: note = Synthetic oligonucleotide
  source 1..45: mol_type = other DNA
catgagtcaagaggcccgcgaggggattaagccacatattcagcg

SEQ ID NO: 396 - DNA, 47 bp, synthetic construct
  misc_feature 1..47: note = Synthetic oligonucleotide
  source 1..47: mol_type = other DNA
gagtcaagaggcccgcctggcgattaagccacatattcagcgcttgc

SEQ ID NO: 397 - DNA, 47 bp, synthetic construct
  misc_feature 1..47: note = Synthetic oligonucleotide
  source 1..47: mol_type = other DNA
gagtcaagaggcccgcctgcgtattaagccacatattcagcgcttgc

SEQ ID NO: 398 - DNA, 47 bp, synthetic construct
  misc_feature 1..47: note = Synthetic oligonucleotide
  source 1..47: mol_type = other DNA
gagtcaagaggcccgcctggagattaagccacatattcagcgcttgc

SEQ ID NO: 399 - DNA, 44 bp, synthetic construct
  misc_feature 1..44: note = Synthetic oligonucleotide
  source 1..44: mol_type = other DNA
ggcccgcctggggattaaggcgcatattcagcgcttgctggacc

SEQ ID NO: 400 - DNA, 44 bp, synthetic construct
  misc_feature 1..44: note = Synthetic oligonucleotide
  source 1..44: mol_type = other DNA
ggcccgcctggggattaagcgtcatattcagcgcttgctggacc

SEQ ID NO: 401 - DNA, 44 bp, synthetic construct
  misc_feature 1..44: note = Synthetic oligonucleotide
  source 1..44: mol_type = other DNA
ggcccgcctggggattaaggagcatattcagcgcttgctggacc

SEQ ID NO: 402 - DNA, 43 bp, synthetic construct
  misc_feature 1..43: note = Synthetic oligonucleotide
  source 1..43: mol_type = other DNA
ccgcctggggattaagccagcgattcagcgcttgctggaccag

SEQ ID NO: 403 - DNA, 43 bp, synthetic construct
  misc_feature 1..43: note = Synthetic oligonucleotide
  source 1..43: mol_type = other DNA
ccgcctggggattaagccacgtattcagcgcttgctggaccag

SEQ ID NO: 404 - DNA, 43 bp, synthetic construct
  misc_feature 1..43: note = Synthetic oligonucleotide
  source 1..43: mol_type = other DNA
ccgcctggggattaagccagagattcagcgcttgctggaccag

SEQ ID NO: 405 - DNA, 47 bp, synthetic construct
  misc_feature 1..47: note = Synthetic oligonucleotide
  source 1..47: mol_type = other DNA
gattaagccacatattcagcgcttggcggaccaggggatcttggtcc

SEQ ID NO: 406 - DNA, 47 bp, synthetic construct
  misc_feature 1..47: note = Synthetic oligonucleotide
  source 1..47: mol_type = other DNA
gattaagccacatattcagcgcttgcgtgaccaggggatcttggtcc

SEQ ID NO: 407 - DNA, 47 bp, synthetic construct
  misc_feature 1..47: note = Synthetic oligonucleotide
  source 1..47: mol_type = other DNA
gattaagccacatattcagcgcttggaggaccaggggatcttggtcc

SEQ ID NO: 408 - DNA, 45 bp, synthetic construct
  misc_feature 1..45: note = Synthetic oligonucleotide
  source 1..45: mol_type = other DNA
gccacatattcagcgcttgctggcgcaggggatcttggtcccatg

SEQ ID NO: 409 - DNA, 45 bp, synthetic construct
  misc_feature 1..45: note = Synthetic oligonucleotide
  source 1..45: mol_type = other DNA
gccacatattcagcgcttgctgcgtcaggggatcttggtcccatg

SEQ ID NO: 410 - DNA, 45 bp, synthetic construct
  misc_feature 1..45: note = Synthetic oligonucleotide
  source 1..45: mol_type = other DNA
gccacatattcagcgcttgctggagcaggggatcttggtcccatg

SEQ ID NO: 411 - DNA, 53 bp, synthetic construct
  misc_feature 1..53: note = Synthetic oligonucleotide
  source 1..53: mol_type = other DNA
aggtcaacaaacgcgtagaagacgcgcatccgactgtacctaatccttataat

SEQ ID NO: 412 - DNA, 53 bp, synthetic construct
  misc_feature 1..53: note = Synthetic oligonucleotide
  source 1..53: mol_type = other DNA
aggtcaacaaacgcgtagaagaccgtcatccgactgtacctaatccttataat

SEQ ID NO: 413 - DNA, 53 bp, synthetic construct
  misc_feature 1..53: note = Synthetic oligonucleotide
  source 1..53: mol_type = other DNA
aggtcaacaaacgcgtagaagacgagcatccgactgtacctaatccttataat

SEQ ID NO: 414 - DNA, 53 bp, synthetic construct
  misc_feature 1..53: note = Synthetic oligonucleotide
  source 1..53: mol_type = other DNA
gcgtagaagacatccatccgactgcgcctaatccttataatctgttatcaggc

SEQ ID NO: 415 - DNA, 53 bp, synthetic construct
  misc_feature 1..53: note = Synthetic oligonucleotide
  source 1..53: mol_type = other DNA
gcgtagaagacatccatccgactcgtcctaatccttataatctgttatcaggc

SEQ ID NO: 416 - DNA, 53 bp, synthetic construct
  misc_feature 1..53: note = Synthetic oligonucleotide
  source 1..53: mol_type = other DNA
gcgtagaagacatccatccgactgagcctaatccttataatctgttatcaggc

SEQ ID NO: 417 - DNA, 49 bp, synthetic construct
  misc_feature 1..49: note = Synthetic oligonucleotide
  source 1..49: mol_type = other DNA
agggctttaaaaacagccccacagcgttcgatgaagcacttcaccgtga

SEQ ID NO: 418 - DNA, 49 bp, synthetic construct
  misc_feature 1..49: note = Synthetic oligonucleotide
  source 1..49: mol_type = other DNA
agggctttaaaaacagccccacacgtttcgatgaagcacttcaccgtga

SEQ ID NO: 419 - DNA, 49 bp, synthetic construct
  misc_feature 1..49: note = Synthetic oligonucleotide
  source 1..49: mol_type = other DNA
agggctttaaaaacagccccacagagttcgatgaagcacttcaccgtga

SEQ ID NO: 420 - DNA, 52 bp, synthetic construct
  misc_feature 1..52: note = Synthetic oligonucleotide
  source 1..52: mol_type = other DNA
tttaaaaacagccccacattgttcgatgcggcacttcaccgtgacttagcag

SEQ ID NO: 421 - DNA, 52 bp, synthetic construct
  misc_feature 1..52: note = Synthetic oligonucleotide
  source 1..52: mol_type = other DNA
tttaaaaacagccccacattgttcgatcgtgcacttcaccgtgacttagcag

SEQ ID NO: 422 - DNA, 52 bp, synthetic construct
  misc_feature 1..52: note = Synthetic oligonucleotide
  source 1..52: mol_type = other DNA
tttaaaaacagccccacattgttcgatgatgcacttcaccgtgacttagcag

SEQ ID NO: 423 - DNA, 51 bp, synthetic construct
  misc_feature 1..51: note = Synthetic oligonucleotide
  source 1..51: mol_type = other DNA
cacattgttcgatgaagcacttcacgcggacttagcagacttccgtatcca

SEQ ID NO: 424 - DNA, 51 bp, synthetic construct
  misc_feature 1..51: note = Synthetic oligonucleotide
  source 1..51: mol_type = other DNA
cacattgttcgatgaagcacttcacaaagacttagcagacttccgtatcca

SEQ ID NO: 425 - DNA, 49 bp, synthetic construct
  misc_feature 1..49: note = Synthetic oligonucleotide
  source 1..49: mol_type = other DNA
gatgaagcacttcaccgtgacttagaggacttccgtatccaacacccag

SEQ ID NO: 426 - DNA, 49 bp, synthetic construct
  misc_feature 1..49: note = Synthetic oligonucleotide
  source 1..49: mol_type = other DNA
aagcacttcaccgtgacttagcagcgttccgtatccaacacccagactt

SEQ ID NO: 427 - DNA, 49 bp, synthetic construct
  misc_feature 1..49: note = Synthetic oligonucleotide
  source 1..49: mol_type = other DNA
aagcacttcaccgtgacttagcacgtttccgtatccaacacccagactt

SEQ ID NO: 428 - DNA, 49 bp, synthetic construct
  misc_feature 1..49: note = Synthetic oligonucleotide
  source 1..49: mol_type = other DNA
aagcacttcaccgtgacttagcagagttccgtatccaacacccagactt

SEQ ID NO: 429 - DNA, 51 bp, synthetic construct
  misc_feature 1..51: note = Synthetic oligonucleotide
  source 1..51: mol_type = other DNA
cacttcaccgtgacttagcagacgcgcgtatccaacacccagacttaattc

SEQ ID NO: 430 - DNA, 51 bp, synthetic construct
  misc_feature 1..51: note = Synthetic oligonucleotide
  source 1..51: mol_type = other DNA
cacttcaccgtgacttagcagaccgtcgtatccaacacccagacttaattc

SEQ ID NO: 431 - DNA, 51 bp, synthetic construct
  misc_feature 1..51: note = Synthetic oligonucleotide
  source 1..51: mol_type = other DNA
cacttcaccgtgacttagcagacgagcgtatccaacacccagacttaattc

SEQ ID NO: 432 - DNA, 53 bp, synthetic construct
  misc_feature 1..53: note = Synthetic oligonucleotide
  source 1..53: mol_type = other DNA
ttcaccgtgacttagcagacttcgcgatccaacacccagacttaattctgtta

SEQ ID NO: 433 - DNA, 53 bp, synthetic construct
  misc_feature 1..53: note = Synthetic oligonucleotide
  source 1..53: mol_type = other DNA
ttcaccgtgacttagcagacttcaaaatccaacacccagacttaattctgtta

SEQ ID NO: 434 - DNA, 53 bp, synthetic construct
  misc_feature 1..53: note = Synthetic oligonucleotide
  source 1..53: mol_type = other DNA
ttcaccgtgacttagcagacttcgagatccaacacccagacttaattctgtta

SEQ ID NO: 435 - DNA, 52 bp, synthetic construct
  misc_feature 1..52: note = Synthetic oligonucleotide
  source 1..52: mol_type = other DNA
ccgtgacttagcagacttccgtgcgcaacacccagacttaattctgttacag

SEQ ID NO: 436 - DNA, 52 bp, synthetic construct
  misc_feature 1..52: note = Synthetic oligonucleotide
  source 1..52: mol_type = other DNA
ccgtgacttagcagacttccgtcgtcaacacccagacttaattctgttacag

SEQ ID NO: 437 - DNA, 52 bp, synthetic construct
  misc_feature 1..52: note = Synthetic oligonucleotide
  source 1..52: mol_type = other DNA
ccgtgacttagcagacttccgtgagcaacacccagacttaattctgttacag

SEQ ID NO: 438 - DNA, 53 bp, synthetic construct
  misc_feature 1..53: note = Synthetic oligonucleotide
  source 1..53: mol_type = other DNA
gtgacttagcagacttccgtatcgcgcacccagacttaattctgttacagtat

SEQ ID NO: 439 - DNA, 53 bp, synthetic construct
  misc_feature 1..53: note = Synthetic oligonucleotide
  source 1..53: mol_type = other DNA
gtgacttagcagacttccgtatccgtcacccagacttaattctgttacagtat

SEQ ID NO: 440 - DNA, 53 bp, synthetic construct
  misc_feature 1..53: note = Synthetic oligonucleotide
  source 1..53: mol_type = other DNA
gtgacttagcagacttccgtatcgagcacccagacttaattctgttacagtat

SEQ ID NO: 441 - DNA, 47 bp, synthetic construct
  misc_feature 1..47: note = Synthetic oligonucleotide
  source 1..47: mol_type = other DNA
agcaaaaggcgtatcaggagatcgcgcaagctttgttgaccgcaccc

SEQ ID NO: 442 - DNA, 47 bp, synthetic construct
  misc_feature 1..47: note = Synthetic oligonucleotide
  source 1..47: mol_type = other DNA
agcaaaaggcgtatcaggagatccgtcaagctttgttgaccgcaccc

SEQ ID NO: 443 - DNA, 47 bp, synthetic construct
  misc_feature 1..47: note = Synthetic oligonucleotide
  source 1..47: mol_type = other DNA
agcaaaaggcgtatcaggagatcgagcaagctttgttgaccgcaccc

SEQ ID NO: 444 - DNA, 46 bp, synthetic construct
  misc_feature 1..46: note = Synthetic oligonucleotide
  source 1..46: mol_type = other DNA
cgtatcaggagatcaaacaagctttggcgaccgcacccgcgttggg

SEQ ID NO: 445 - DNA, 46 bp, synthetic construct
  misc_feature 1..46: note = Synthetic oligonucleotide
  source 1..46: mol_type = other DNA
cgtatcaggagatcaaacaagctttgcgtaccgcacccgcgttggg

SEQ ID NO: 446 - DNA, 46 bp, synthetic construct
  misc_feature 1..46: note = Synthetic oligonucleotide
  source 1..46: mol_type = other DNA
cgtatcaggagatcaaacaagctttggagaccgcacccgcgttggg

SEQ ID NO: 447 - DNA, 42 bp, synthetic construct
  misc_feature 1..42: note = Synthetic oligonucleotide
  source 1..42: mol_type = other DNA
gttggctgactgaagcgcgtgcggagaccgtaatggggcagc

SEQ ID NO: 448 - DNA, 42 bp, synthetic construct
  misc_feature 1..42: note = Synthetic oligonucleotide
  source 1..42: mol_type = other DNA
gttggctgactgaagcgcgtcgtgagaccgtaatggggcagc

SEQ ID NO: 449 - DNA, 42 bp, synthetic construct
  misc_feature 1..42: note = Synthetic oligonucleotide
  source 1..42: mol_type = other DNA
gttggctgactgaagcgcgtgaggagaccgtaatggggcagc

SEQ ID NO: 450 - DNA, 45 bp, synthetic construct
  misc_feature 1..45: note = Synthetic oligonucleotide
  source 1..45: mol_type = other DNA
tacgcctaagacgccacgcgcgttgcgtgaatttttgggcacagc

SEQ ID NO: 451 - DNA, 45 bp, synthetic construct
  misc_feature 1..45: note = Synthetic oligonucleotide
  source 1..45: mol_type = other DNA
tacgcctaagacgccacgccgtttgcgtgaatttttgggcacagc

SEQ ID NO: 452 - DNA, 45 bp, synthetic construct
  misc_feature 1..45: note = Synthetic oligonucleotide
  source 1..45: mol_type = other DNA
tacgcctaagacgccacgcgagttgcgtgaatttttgggcacagc

SEQ ID NO: 453 - DNA, 49 bp, synthetic construct
  misc_feature 1..49: note = Synthetic oligonucleotide
  source 1..49: mol_type = other DNA
gcgtgaatttttgggcacagcggcgttctgtcgtttatggattcctggg

SEQ ID NO: 454 - DNA, 49 bp, synthetic construct
  misc_feature 1..49: note = Synthetic oligonucleotide
  source 1..49: mol_type = other DNA
gcgtgaatttttgggcacagcgcgtttctgtcgtttatggattcctggg

SEQ ID NO: 455 - DNA, 49 bp, synthetic construct
  misc_feature 1..49: note = Synthetic oligonucleotide
  source 1..49: mol_type = other DNA
gcgtgaatttttgggcacagcggagttctgtcgtttatggattcctggg

SEQ ID NO: 456 - DNA, 45 bp, synthetic construct
  misc_feature 1..45: note = Synthetic oligonucleotide
  source 1..45: mol_type = other DNA
gggcacagcgggattctgtgcgttatggattcctgggttcgctga

SEQ ID NO: 457 - DNA, 45 bp, synthetic construct
  misc_feature 1..45: note = Synthetic oligonucleotide
  source 1..45: mol_type = other DNA
gggcacagcgggattctgtaaattatggattcctgggttcgctga

SEQ ID NO: 458 - DNA, 45 bp, synthetic construct
  misc_feature 1..45: note = Synthetic oligonucleotide
  source 1..45: mol_type = other DNA
gggcacagcgggattctgtgagttatggattcctgggttcgctga

SEQ ID NO: 459 - DNA, 53 bp, synthetic construct
  misc_feature 1..53: note = Synthetic oligonucleotide
  source 1..53: mol_type = other DNA
gtcaaaaacaggtaaagtaccttggggcgttgctgaaagaaggtcaacgttgg

SEQ ID NO: 460 - DNA, 53 bp, synthetic construct
  misc_feature 1..53: note = Synthetic oligonucleotide
  source 1..53: mol_type = other DNA
gtcaaaaacaggtaaagtaccttgggcgtttgctgaaagaaggtcaacgttgg

SEQ ID NO: 461 - DNA, 53 bp, synthetic construct
  misc_feature 1..53: note = Synthetic oligonucleotide
  source 1..53: mol_type = other DNA
gtcaaaaacaggtaaagtaccttggggagttgctgaaagaaggtcaacgttgg

SEQ ID NO: 462 - DNA, 47 bp, synthetic construct
  misc_feature 1..47: note = Synthetic oligonucleotide
  source 1..47: mol_type = other DNA
tgctgaaagaaggtcaacgttgggcgactgaagcgcgtaaggagacc

SEQ ID NO: 463 - DNA, 47 bp, synthetic construct
  misc_feature 1..47: note = Synthetic oligonucleotide
  source 1..47: mol_type = other DNA
tgctgaaagaaggtcaacgttggcgtactgaagcgcgtaaggagacc

SEQ ID NO: 464 - DNA, 47 bp, synthetic construct
  misc_feature 1..47: note = Synthetic oligonucleotide
  source 1..47: mol_type = other DNA
tgctgaaagaaggtcaacgttgggagactgaagcgcgtaaggagacc

SEQ ID NO: 465 - DNA, 45 bp, synthetic construct
  misc_feature 1..45: note = Synthetic oligonucleotide
  source 1..45: mol_type = other DNA
tttgttgaccgcacccgcggcgggtcttccggatttaaccaagcc

SEQ ID NO: 466 - DNA, 45 bp, synthetic construct
  misc_feature 1..45: note = Synthetic oligonucleotide
  source 1..45: mol_type = other DNA
tttgttgaccgcacccgcgcgtggtcttccggatttaaccaagcc

SEQ ID NO: 467 - DNA, 45 bp, synthetic construct
  misc_feature 1..45: note = Synthetic oligonucleotide
  source 1..45: mol_type = other DNA
tttgttgaccgcacccgcggagggtcttccggatttaaccaagcc

SEQ ID NO: 468 - DNA, 47 bp, synthetic construct
  misc_feature 1..47: note = Synthetic oligonucleotide
  source 1..47: mol_type = other DNA
ctgcacccctgtaccccttagcgaaaacagggacgcttttcaactgg

SEQ ID NO: 469 - DNA, 47 bp, synthetic construct
  misc_feature 1..47: note = Synthetic oligonucleotide
  source 1..47: mol_type = other DNA
ctgcacccctgtaccccttacgtaaaacagggacgcttttcaactgg

SEQ ID NO: 470 - DNA, 47 bp, synthetic construct
  misc_feature 1..47: note = Synthetic oligonucleotide
  source 1..47: mol_type = other DNA
ctgcacccctgtaccccttagagaaaacagggacgcttttcaactgg

SEQ ID NO: 471 - DNA, 47 bp, synthetic construct
  misc_feature 1..47: note = Synthetic oligonucleotide
  source 1..47: mol_type = other DNA
cccctgtaccccttaacaaaaacagcgacgcttttcaactgggggcc

SEQ ID NO: 472 - DNA, 47 bp, synthetic construct
  misc_feature 1..47: note = Synthetic oligonucleotide
  source 1..47: mol_type = other DNA
cccctgtaccccttaacaaaaacacgtacgcttttcaactgggggcc

SEQ ID NO: 473 - DNA, 47 bp, synthetic construct
  misc_feature 1..47: note = Synthetic oligonucleotide
  source 1..47: mol_type = other DNA
cccctgtaccccttaacaaaaacagagacgcttttcaactgggggcc

SEQ ID NO: 474 - DNA, 48 bp, synthetic construct
  misc_feature 1..48: note = Synthetic oligonucleotide
  source 1..48: mol_type = other DNA
ctgtaccccttaacaaaaacaggggcgcttttcaactgggggccagac

SEQ ID NO: 475 - DNA, 48 bp, synthetic construct
  misc_feature 1..48: note = Synthetic oligonucleotide
  source 1..48: mol_type = other DNA
ctgtaccccttaacaaaaacagggcgtcttttcaactgggggccagac

SEQ ID NO: 476 - DNA, 48 bp, synthetic construct
  misc_feature 1..48: note = Synthetic oligonucleotide
  source 1..48: mol_type = other DNA
ctgtaccccttaacaaaaacaggggagcttttcaactgggggccagac

SEQ ID NO: 477 - DNA, 48 bp, synthetic construct
  misc_feature 1..48: note = Synthetic oligonucleotide
  source 1..48: mol_type = other DNA
ccttaacaaaaacagggacgcttttcgcgtgggggccagaccagcaaa

SEQ ID NO: 478 - DNA, 48 bp, synthetic construct
  misc_feature 1..48: note = Synthetic oligonucleotide
  source 1..48: mol_type = other DNA
ccttaacaaaaacagggacgcttttccgttgggggccagaccagcaaa

SEQ ID NO: 479 - DNA, 54 bp, synthetic construct
  misc_feature 1..54: note = Synthetic oligonucleotide
  source 1..54: mol_type = other DNA
cttccggatttaaccaagccctttgcgctgttcgttgatgaaaaacagggatat

SEQ ID NO: 480 - DNA, 54 bp, synthetic construct
  misc_feature 1..54: note = Synthetic oligonucleotide
  source 1..54: mol_type = other DNA
cttccggatttaaccaagccctttcgtctgttcgttgatgaaaaacagggatat

SEQ ID NO: 481 - DNA, 54 bp, synthetic construct
  misc_feature 1..54: note = Synthetic oligonucleotide
  source 1..54: mol_type = other DNA
cttccggatttaaccaagccctttgatctgttcgttgatgaaaaacagggatat

SEQ ID NO: 482 - DNA, 55 bp, synthetic construct
  misc_feature 1..55: note = Synthetic oligonucleotide
  source 1..55: mol_type = other DNA
gatttaaccaagccctttgagctggcggttgatgaaaaacagggatatgcaaaag

SEQ ID NO: 483 - DNA, 55 bp, synthetic construct
  misc_feature 1..55: note = Synthetic oligonucleotide
  source 1..55: mol_type = other DNA
gatttaaccaagccctttgagctgcgtgttgatgaaaaacagggatatgcaaaag

SEQ ID NO: 484 - DNA, 55 bp, synthetic construct
  misc_feature 1..55: note = Synthetic oligonucleotide
  source 1..55: mol_type = other DNA
gatttaaccaagccctttgagctggaggttgatgaaaaacagggatatgcaaaag

SEQ ID NO: 485 - DNA, 46 bp, synthetic construct
  misc_feature 1..46: note = Synthetic oligonucleotide
  source 1..46: mol_type = other DNA
cccaaaagttaggcccgtgggcgcgccctgttgcttacttgagtaa

SEQ ID NO: 486 - DNA, 46 bp, synthetic construct
  misc_feature 1..46: note = Synthetic oligonucleotide
  source 1..46: mol_type = other DNA
cccaaaagttaggcccgtggaaacgccctgttgcttacttgagtaa

SEQ ID NO: 487 - DNA, 46 bp, synthetic construct
  misc_feature 1..46: note = Synthetic oligonucleotide
  source 1..46: mol_type = other DNA
cccaaaagttaggcccgtgggagcgccctgttgcttacttgagtaa

SEQ ID NO: 488 - DNA, 49 bp, synthetic construct
  misc_feature 1..49: note = Synthetic oligonucleotide
  source 1..49: mol_type = other DNA
agttgacgatgggtcaacccttagcgatcttggctccacatgctgtaga

SEQ ID NO: 489 - DNA, 49 bp, synthetic construct
  misc_feature 1..49: note = Synthetic oligonucleotide
  source 1..49: mol_type = other DNA
agttgacgatgggtcaacccttacgtatcttggctccacatgctgtaga

SEQ ID NO: 490 - DNA, 49 bp, synthetic construct
  misc_feature 1..49: note = Synthetic oligonucleotide
  source 1..49: mol_type = other DNA
agttgacgatgggtcaacccttagagatcttggctccacatgctgtaga

SEQ ID NO: 491 - DNA, 53 bp, synthetic construct
  misc_feature 1..53: note = Synthetic oligonucleotide
  source 1..53: mol_type = other DNA
ggatcgtgtacaatttggaccagttgcggctttgaatccagctactttgcttc

SEQ ID NO: 492 - DNA, 53 bp, synthetic construct
  misc_feature 1..53: note = Synthetic oligonucleotide
  source 1..53: mol_type = other DNA
ggatcgtgtacaatttggaccagttcgtgctttgaatccagctactttgcttc

SEQ ID NO: 493 - DNA, 53 bp, synthetic construct
  misc_feature 1..53: note = Synthetic oligonucleotide
  source 1..53: mol_type = other DNA
ggatcgtgtacaatttggaccagttgaggctttgaatccagctactttgcttc

SEQ ID NO: 494 - DNA, 48 bp, synthetic construct
  misc_feature 1..48: note = Synthetic oligonucleotide
  source 1..48: mol_type = other DNA
cgttatgcttttgcaacagcgcatgcgcatggcgaaatttaccgccgc

SEQ ID NO: 495 - DNA, 48 bp, synthetic construct
  misc_feature 1..48: note = Synthetic oligonucleotide
  source 1..48: mol_type = other DNA
cgttatgcttttgcaacagcgcatcgtcatggcgaaatttaccgccgc

SEQ ID NO: 496 - DNA, 48 bp, synthetic construct
  misc_feature 1..48: note = Synthetic oligonucleotide
  source 1..48: mol_type = other DNA
cgttatgcttttgcaacagcgcatgagcatggcgaaatttaccgccgc

SEQ ID NO: 497 - DNA, 43 bp, synthetic construct
  misc_feature 1..43: note = Synthetic oligonucleotide
  source 1..43: mol_type = other DNA
gcaacagcgcatatccatggcgcgatttaccgccgccgtggtc

SEQ ID NO: 498 - DNA, 43 bp, synthetic construct
  misc_feature 1..43: note = Synthetic oligonucleotide
  source 1..43: mol_type = other DNA
gcaacagcgcatatccatggccgtatttaccgccgccgtggtc

SEQ ID NO: 499 - DNA, 43 bp, synthetic construct
  misc_feature 1..43: note = Synthetic oligonucleotide
  source 1..43: mol_type = other DNA
gcaacagcgcatatccatggcgatatttaccgccgccgtggtc

SEQ ID NO: 500 - DNA, 44 bp, synthetic construct
  misc_feature 1..44: note = Synthetic oligonucleotide
  source 1..44: mol_type = other DNA
caacagcgcatatccatggcgaagcgtaccgccgccgtggtctg

SEQ ID NO: 501 - DNA, 44 bp, synthetic construct
  misc_feature 1..44: note = Synthetic oligonucleotide
  source 1..44: mol_type = other DNA
caacagcgcatatccatggcgaacgttaccgccgccgtggtctg

SEQ ID NO: 502 - DNA, 44 bp, synthetic construct
  misc_feature 1..44: note = Synthetic oligonucleotide
  source 1..44: mol_type = other DNA
caacagcgcatatccatggcgaagagtaccgccgccgtggtctg

SEQ ID NO: 503 - DNA, 41 bp, synthetic construct
  misc_feature 1..41: note = Synthetic oligonucleotide
  source 1..41: mol_type = other DNA
agcggaggctcgtggaaacgcgatggcggaccaagctgccc

SEQ ID NO: 504 - DNA, 41 bp, synthetic construct
  misc_feature 1..41: note = Synthetic oligonucleotide
  source 1..41: mol_type = other DNA
agcggaggctcgtggaaacaaaatggcggaccaagctgccc

SEQ ID NO: 505 - DNA, 41 bp, synthetic construct
  misc_feature 1..41: note = Synthetic oligonucleotide
  source 1..41: mol_type = other DNA
agcggaggctcgtggaaacgagatggcggaccaagctgccc

SEQ ID NO: 506 - DNA, 40 bp, synthetic construct
  misc_feature 1..40: note = Synthetic oligonucleotide
  source 1..40: mol_type = other DNA
gtggaaaccgtatggcggacgcggctgcccgtaaggcggc

SEQ ID NO: 507 - DNA, 40 bp, synthetic construct
  misc_feature 1..40: note = Synthetic oligonucleotide
  source 1..40: mol_type = other DNA
gtggaaaccgtatggcggaccgtgctgcccgtaaggcggc

SEQ ID NO: 508 - DNA, 40 bp, synthetic construct
  misc_feature 1..40: note = Synthetic oligonucleotide
  source 1..40: mol_type = other DNA
gtggaaaccgtatggcggacgaggctgcccgtaaggcggc

SEQ ID NO: 509 - DNA, 42 bp, synthetic construct
  misc_feature 1..42: note = Synthetic oligonucleotide
  source 1..42: mol_type = other DNA
tatggcggaccaagctgccgcgaaggcggcgatcacagagac

SEQ ID NO: 510 - DNA, 42 bp, synthetic construct
  misc_feature 1..42: note = Synthetic oligonucleotide
  source 1..42: mol_type = other DNA
tatggcggaccaagctgccaaaaaggcggcgatcacagagac

SEQ ID NO: 511 - DNA, 42 bp, synthetic construct
  misc_feature 1..42: note = Synthetic oligonucleotide
  source 1..42: mol_type = other DNA
tatggcggaccaagctgccgagaaggcggcgatcacagagac

SEQ ID NO: 512 - DNA, 47 bp, synthetic construct
  misc_feature 1..47: note = Synthetic oligonucleotide
  source 1..47: mol_type = other DNA
gcaagcgctgaatatgtggcttaatcgccaggcgggcctcttgactc

SEQ ID NO: 513 - DNA, 47 bp, synthetic construct
  misc_feature 1..47: note = Synthetic oligonucleotide
  source 1..47: mol_type = other DNA
gcaagcgctgaatatgtggcttaatacgcaggcgggcctcttgactc

SEQ ID NO: 514 - DNA, 47 bp, synthetic construct
  misc_feature 1..47: note = Synthetic oligonucleotide
  source 1..47: mol_type = other DNA
gcaagcgctgaatatgtggcttaatctccaggcgggcctcttgactc

SEQ ID NO: 515 - DNA, 44 bp, synthetic construct
  misc_feature 1..44: note = Synthetic oligonucleotide
  source 1..44: mol_type = other DNA
ggtccagcaagcgctgaatatgcgccttaatccccaggcgggcc

SEQ ID NO: 516 - DNA, 44 bp, synthetic construct
  misc_feature 1..44: note = Synthetic oligonucleotide
  source 1..44: mol_type = other DNA
ggtccagcaagcgctgaatatgacgcttaatccccaggcgggcc

SEQ ID NO: 517 - DNA, 44 bp, synthetic construct
  misc_feature 1..44: note = Synthetic oligonucleotide
  source 1..44: mol_type = other DNA
ggtccagcaagcgctgaatatgctccttaatccccaggcgggcc

SEQ ID NO: 518 - DNA, 43 bp, synthetic construct
  misc_feature 1..43: note = Synthetic oligonucleotide
  source 1..43: mol_type = other DNA
ctggtccagcaagcgctgaatcgctggcttaatccccaggcgg

SEQ ID NO: 519 - DNA, 43 bp, synthetic construct
  misc_feature 1..43: note = Synthetic oligonucleotide
  source 1..43: mol_type = other DNA
ctggtccagcaagcgctgaatacgtggcttaatccccaggcgg

SEQ ID NO: 520 - DNA, 43 bp, synthetic construct
  misc_feature 1..43: note = Synthetic oligonucleotide
  source 1..43: mol_type = other DNA
ctggtccagcaagcgctgaatctctggcttaatccccaggcgg

SEQ ID NO: 521 - DNA, 47 bp, synthetic construct
  misc_feature 1..47: note = Synthetic oligonucleotide
  source 1..47: mol_type = other DNA
ggaccaagatcccctggtccgccaagcgctgaatatgtggcttaatc

SEQ ID NO: 522 - DNA, 47 bp, synthetic construct
  misc_feature 1..47: note = Synthetic oligonucleotide
  source 1..47: mol_type = other DNA
ggaccaagatcccctggtcacgcaagcgctgaatatgtggcttaatc

SEQ ID NO: 523 - DNA, 47 bp, synthetic construct
  misc_feature 1..47: note = Synthetic oligonucleotide
  source 1..47: mol_type = other DNA
ggaccaagatcccctggtcctccaagcgctgaatatgtggcttaatc

SEQ ID NO: 524 - DNA, 45 bp, synthetic construct
  misc_feature 1..45: note = Synthetic oligonucleotide
  source 1..45: mol_type = other DNA
catgggaccaagatcccctgcgccagcaagcgctgaatatgtggc

SEQ ID NO: 525 - DNA, 45 bp, synthetic construct
  misc_feature 1..45: note = Synthetic oligonucleotide
  source 1..45: mol_type = other DNA
catgggaccaagatcccctgacgcagcaagcgctgaatatgtggc

SEQ ID NO: 526 - DNA, 45 bp, synthetic construct
  misc_feature 1..45: note = Synthetic oligonucleotide
  source 1..45: mol_type = other DNA
catgggaccaagatcccctgctccagcaagcgctgaatatgtggc

SEQ ID NO: 527 - DNA, 53 bp, synthetic construct
  misc_feature 1..53: note = Synthetic oligonucleotide
  source 1..53: mol_type = other DNA
attataaggattaggtacagtcggatgcgcgtcttctacgcgtttgttgacct

SEQ ID NO: 528 - DNA, 53 bp, synthetic construct
  misc_feature 1..53: note = Synthetic oligonucleotide
  source 1..53: mol_type = other DNA
attataaggattaggtacagtcggatgacggtcttctacgcgtttgttgacct

SEQ ID NO: 529 - DNA, 53 bp, synthetic construct
  misc_feature 1..53: note = Synthetic oligonucleotide
  source 1..53: mol_type = other DNA
attataaggattaggtacagtcggatgctcgtcttctacgcgtttgttgacct

SEQ ID NO: 530 - DNA, 53 bp, synthetic construct
  misc_feature 1..53: note = Synthetic oligonucleotide
  source 1..53: mol_type = other DNA
gcctgataacagattataaggattaggcgcagtcggatggatgtcttctacgc

SEQ ID NO: 531 - DNA, 53 bp, synthetic construct
  misc_feature 1..53: note = Synthetic oligonucleotide
  source 1..53: mol_type = other DNA
gcctgataacagattataaggattaggacgagtcggatggatgtcttctacgc

SEQ ID NO: 532 - DNA, 53 bp, synthetic construct
  misc_feature 1..53: note = Synthetic oligonucleotide
  source 1..53: mol_type = other DNA
gcctgataacagattataaggattaggctcagtcggatggatgtcttctacgc

SEQ ID NO: 533 - DNA, 49 bp, synthetic construct
  misc_feature 1..49: note = Synthetic oligonucleotide
  source 1..49: mol_type = other DNA
tcacggtgaagtgcttcatcgaacgctgtggggctgtttttaaagccct

SEQ ID NO: 534 - DNA, 49 bp, synthetic construct
  misc_feature 1..49: note = Synthetic oligonucleotide
  source 1..49: mol_type = other DNA
tcacggtgaagtgcttcatcgaaacgtgtggggctgtttttaaagccct

SEQ ID NO: 535 - DNA, 49 bp, synthetic construct
  misc_feature 1..49: note = Synthetic oligonucleotide
  source 1..49: mol_type = other DNA
tcacggtgaagtgcttcatcgaactctgtggggctgtttttaaagccct

SEQ ID NO: 536 - DNA, 52 bp, synthetic construct
  misc_feature 1..52: note = Synthetic oligonucleotide
  source 1..52: mol_type = other DNA
ctgctaagtcacggtgaagtgccgcatcgaacaatgtggggctgtttttaaa

SEQ ID NO: 537 - DNA, 52 bp, synthetic construct
  misc_feature 1..52: note = Synthetic oligonucleotide
  source 1..52: mol_type = other DNA
ctgctaagtcacggtgaagtgcacgatcgaacaatgtggggctgtttttaaa

SEQ ID NO: 538 - DNA, 52 bp, synthetic construct
  misc_feature 1..52: note = Synthetic oligonucleotide
  source 1..52: mol_type = other DNA
ctgctaagtcacggtgaagtgcatcatcgaacaatgtggggctgtttttaaa

SEQ ID NO: 539 - DNA, 51 bp, synthetic construct
  misc_feature 1..51: note = Synthetic oligonucleotide
  source 1..51: mol_type = other DNA
tggatacggaagtctgctaagtccgcgtgaagtgcttcatcgaacaatgtg

SEQ ID NO: 540 - DNA, 51 bp, synthetic construct
  misc_feature 1..51: note = Synthetic oligonucleotide
  source 1..51: mol_type = other DNA
tggatacggaagtctgctaagtctttgtgaagtgcttcatcgaacaatgtg

SEQ ID NO: 541 - DNA, 51 bp, synthetic construct
  misc_feature 1..51: note = Synthetic oligonucleotide
  source 1..51: mol_type = other DNA
tggatacggaagtctgctaagtcctcgtgaagtgcttcatcgaacaatgtg

SEQ ID NO: 542 - DNA, 49 bp, synthetic construct
  misc_feature 1..49: note = Synthetic oligonucleotide
  source 1..49: mol_type = other DNA
aagtctgggtgttggatacggaacgctgctaagtcacggtgaagtgctt

SEQ ID NO: 543 - DNA, 49 bp, synthetic construct
  misc_feature 1..49: note = Synthetic oligonucleotide
  source 1..49: mol_type = other DNA
aagtctgggtgttggatacggaaacgtgctaagtcacggtgaagtgctt

SEQ ID NO: 544 - DNA, 49 bp, synthetic construct
  misc_feature 1..49: note = Synthetic oligonucleotide
  source 1..49: mol_type = other DNA
aagtctgggtgttggatacggaactctgctaagtcacggtgaagtgctt

SEQ ID NO: 545 - DNA, 51 bp, synthetic construct
  misc_feature 1..51: note = Synthetic oligonucleotide
  source 1..51: mol_type = other DNA
gaattaagtctgggtgttggatacgcgcgtctgctaagtcacggtgaagtg

SEQ ID NO: 546 - DNA, 51 bp, synthetic construct
  misc_feature 1..51: note = Synthetic oligonucleotide
  source 1..51: mol_type = other DNA
gaattaagtctgggtgttggatacgacggtctgctaagtcacggtgaagtg

SEQ ID NO: 547 - DNA, 51 bp, synthetic construct
  misc_feature 1..51: note = Synthetic oligonucleotide
  source 1..51: mol_type = other DNA
gaattaagtctgggtgttggatacgctcgtctgctaagtcacggtgaagtg

SEQ ID NO: 548 - DNA, 53 bp, synthetic construct
  misc_feature 1..53: note = Synthetic oligonucleotide
  source 1..53: mol_type = other DNA
taacagaattaagtctgggtgttggatcgcgaagtctgctaagtcacggtgaa

SEQ ID NO: 549 - DNA, 53 bp, synthetic construct
  misc_feature 1..53: note = Synthetic oligonucleotide
  source 1..53: mol_type = other DNA
taacagaattaagtctgggtgttggattttgaagtctgctaagtcacggtgaa

SEQ ID NO: 550 - DNA, 53 bp, synthetic construct
  misc_feature 1..53: note = Synthetic oligonucleotide
  source 1..53: mol_type = other DNA
taacagaattaagtctgggtgttggatctcgaagtctgctaagtcacggtgaa

SEQ ID NO: 551 - DNA, 52 bp, synthetic construct
  misc_feature 1..52: note = Synthetic oligonucleotide
  source 1..52: mol_type = other DNA
ctgtaacagaattaagtctgggtgttgcgcacggaagtctgctaagtcacgg

SEQ ID NO: 552 - DNA, 52 bp, synthetic construct
  misc_feature 1..52: note = Synthetic oligonucleotide
  source 1..52: mol_type = other DNA
ctgtaacagaattaagtctgggtgttgacgacggaagtctgctaagtcacgg

SEQ ID NO: 553 - DNA, 52 bp, synthetic construct
  misc_feature 1..52: note = Synthetic oligonucleotide
  source 1..52: mol_type = other DNA
ctgtaacagaattaagtctgggtgttgctcacggaagtctgctaagtcacgg

SEQ ID NO: 554 - DNA, 53 bp, synthetic construct
  misc_feature 1..53: note = Synthetic oligonucleotide
  source 1..53: mol_type = other DNA
atactgtaacagaattaagtctgggtgcgcgatacggaagtctgctaagtcac

SEQ ID NO: 555 - DNA, 53 bp, synthetic construct
  misc_feature 1..53: note = Synthetic oligonucleotide
  source 1..53: mol_type = other DNA
atactgtaacagaattaagtctgggtgacggatacggaagtctgctaagtcac

SEQ ID NO: 556 - DNA, 53 bp, synthetic construct
  misc_feature 1..53: note = Synthetic oligonucleotide
  source 1..53: mol_type = other DNA
atactgtaacagaattaagtctgggtgctcgatacggaagtctgctaagtcac

SEQ ID NO: 557 - DNA, 47 bp, synthetic construct
  misc_feature 1..47: note = Synthetic oligonucleotide
  source 1..47: mol_type = other DNA
gggtgcggtcaacaaagcttgcgcgatctcctgatacgccttttgct

SEQ ID NO: 558 - DNA, 47 bp, synthetic construct
  misc_feature 1..47: note = Synthetic oligonucleotide
  source 1..47: mol_type = other DNA
gggtgcggtcaacaaagcttgacggatctcctgatacgccttttgct

SEQ ID NO: 559 - DNA, 47 bp, synthetic construct
  misc_feature 1..47: note = Synthetic oligonucleotide
  source 1..47: mol_type = other DNA
gggtgcggtcaacaaagcttgctcgatctcctgatacgccttttgct

SEQ ID NO: 560 - DNA, 46 bp, synthetic construct
  misc_feature 1..46: note = Synthetic oligonucleotide
  source 1..46: mol_type = other DNA
cccaacgcgggtgcggtcgccaaagcttgtttgatctcctgatacg

SEQ ID NO: 561 - DNA, 46 bp, synthetic construct
  misc_feature 1..46: note = Synthetic oligonucleotide
  source 1..46: mol_type = other DNA
cccaacgcgggtgcggtacgcaaagcttgtttgatctcctgatacg

SEQ ID NO: 562 - DNA, 46 bp, synthetic construct
  misc_feature 1..46: note = Synthetic oligonucleotide
  source 1..46: mol_type = other DNA
cccaacgcgggtgcggtctccaaagcttgtttgatctcctgatacg

SEQ ID NO: 563 - DNA, 42 bp, synthetic construct
  misc_feature 1..42: note = Synthetic oligonucleotide
  source 1..42: mol_type = other DNA
gctgccccattacggtctccgcacgcgcttcagtcagccaac

SEQ ID NO: 564 - DNA, 42 bp, synthetic construct
  misc_feature 1..42: note = Synthetic oligonucleotide
  source 1..42: mol_type = other DNA
gctgccccattacggtctcacgacgcgcttcagtcagccaac

SEQ ID NO: 565 - DNA, 42 bp, synthetic construct
  misc_feature 1..42: note = Synthetic oligonucleotide
  source 1..42: mol_type = other DNA
gctgccccattacggtctcctcacgcgcttcagtcagccaac

SEQ ID NO: 566 - DNA, 45 bp, synthetic construct
  misc_feature 1..45: note = Synthetic oligonucleotide
  source 1..45: mol_type = other DNA
gctgtgcccaaaaattcacgcaacgcgcgtggcgtcttaggcgta

SEQ ID NO: 567 - DNA, 45 bp, synthetic construct
  misc_feature 1..45: note = Synthetic oligonucleotide
  source 1..45: mol_type = other DNA
gctgtgcccaaaaattcacgcaaacggcgtggcgtcttaggcgta

SEQ ID NO: 568 - DNA, 45 bp, synthetic construct
  misc_feature 1..45: note = Synthetic oligonucleotide
  source 1..45: mol_type = other DNA
gctgtgcccaaaaattcacgcaactcgcgtggcgtcttaggcgta

SEQ ID NO: 569 - DNA, 49 bp, synthetic construct
  misc_feature 1..49: note = Synthetic oligonucleotide
  source 1..49: mol_type = other DNA
cccaggaatccataaacgacagaacgccgctgtgcccaaaaattcacgc

SEQ ID NO: 570 - DNA, 49 bp, synthetic construct
  misc_feature 1..49: note = Synthetic oligonucleotide
  source 1..49: mol_type = other DNA
cccaggaatccataaacgacagaaacgcgctgtgcccaaaaattcacgc

SEQ ID NO: 571 - DNA, 49 bp, synthetic construct
  misc_feature 1..49: note = Synthetic oligonucleotide
  source 1..49: mol_type = other DNA
cccaggaatccataaacgacagaactccgctgtgcccaaaaattcacgc

SEQ ID NO: 572 - DNA, 45 bp, synthetic construct
  misc_feature 1..45: note = Synthetic oligonucleotide
  source 1..45: mol_type = other DNA
tcagcgaacccaggaatccataacgcacagaatcccgctgtgccc

SEQ ID NO: 573 - DNA, 45 bp, synthetic construct
  misc_feature 1..45: note = Synthetic oligonucleotide
  source 1..45: mol_type = other DNA
tcagcgaacccaggaatccataatttacagaatcccgctgtgccc

SEQ ID NO: 574 - DNA, 45 bp, synthetic construct
  misc_feature 1..45: note = Synthetic oligonucleotide
  source 1..45: mol_type = other DNA
tcagcgaacccaggaatccataactcacagaatcccgctgtgccc

SEQ ID NO: 575 - DNA, 53 bp, synthetic construct
  misc_feature 1..53: note = Synthetic oligonucleotide
  source 1..53: mol_type = other DNA
ccaacgttgaccttctttcagcaacgccccaaggtactttacctgtttttgac

SEQ ID NO: 576 - DNA, 53 bp, synthetic construct
  misc_feature 1..53: note = Synthetic oligonucleotide
  source 1..53: mol_type = other DNA
ccaacgttgaccttctttcagcaaacgcccaaggtactttacctgtttttgac

SEQ ID NO: 577 - DNA, 53 bp, synthetic construct
  misc_feature 1..53: note = Synthetic oligonucleotide
  source 1..53: mol_type = other DNA
ccaacgttgaccttctttcagcaactccccaaggtactttacctgtttttgac

SEQ ID NO: 578 - DNA, 47 bp, synthetic construct
  misc_feature 1..47: note = Synthetic oligonucleotide
  source 1..47: mol_type = other DNA
ggtctccttacgcgcttcagtcgcccaacgttgaccttctttcagca

SEQ ID NO: 579 - DNA, 47 bp, synthetic construct
  misc_feature 1..47: note = Synthetic oligonucleotide
  source 1..47: mol_type = other DNA
ggtctccttacgcgcttcagtacgccaacgttgaccttctttcagca

SEQ ID NO: 580 - DNA, 47 bp, synthetic construct
  misc_feature 1..47: note = Synthetic oligonucleotide
  source 1..47: mol_type = other DNA
ggtctccttacgcgcttcagtctcccaacgttgaccttctttcagca

SEQ ID NO: 581 - DNA, 45 bp, synthetic construct
  misc_feature 1..45: note = Synthetic oligonucleotide
  source 1..45: mol_type = other DNA
ggcttggttaaatccggaagacccgccgcgggtgcggtcaacaaa

SEQ ID NO: 582 - DNA, 45 bp, synthetic construct
  misc_feature 1..45: note = Synthetic oligonucleotide
  source 1..45: mol_type = other DNA
ggcttggttaaatccggaagaccacgcgcgggtgcggtcaacaaa

SEQ ID NO: 583 - DNA, 45 bp, synthetic construct
  misc_feature 1..45: note = Synthetic oligonucleotide
  source 1..45: mol_type = other DNA
ggcttggttaaatccggaagaccctccgcgggtgcggtcaacaaa

SEQ ID NO: 584 - DNA, 47 bp, synthetic construct
  misc_feature 1..47: note = Synthetic oligonucleotide
  source 1..47: mol_type = other DNA
ccagttgaaaagcgtccctgttttcgctaaggggtacaggggtgcag

SEQ ID NO: 585 - DNA, 47 bp, synthetic construct
  misc_feature 1..47: note = Synthetic oligonucleotide
  source 1..47: mol_type = other DNA
ccagttgaaaagcgtccctgttttacgtaaggggtacaggggtgcag

SEQ ID NO: 586 - DNA, 47 bp, synthetic construct
  misc_feature 1..47: note = Synthetic oligonucleotide
  source 1..47: mol_type = other DNA
ccagttgaaaagcgtccctgttttctctaaggggtacaggggtgcag

SEQ ID NO: 587 - DNA, 47 bp, synthetic construct
  misc_feature 1..47: note = Synthetic oligonucleotide
  source 1..47: mol_type = other DNA
ggcccccagttgaaaagcgtcgctgtttttgttaaggggtacagggg

SEQ ID NO: 588 - DNA, 47 bp, synthetic construct
  misc_feature 1..47: note = Synthetic oligonucleotide
  source 1..47: mol_type = other DNA
ggcccccagttgaaaagcgtacgtgtttttgttaaggggtacagggg

SEQ ID NO: 589 - DNA, 47 bp, synthetic construct
  misc_feature 1..47: note = Synthetic oligonucleotide
  source 1..47: mol_type = other DNA
ggcccccagttgaaaagcgtctctgtttttgttaaggggtacagggg

SEQ ID NO: 590 - DNA, 48 bp, synthetic construct
  misc_feature 1..48: note = Synthetic oligonucleotide
  source 1..48: mol_type = other DNA
gtctggcccccagttgaaaagcgcccctgtttttgttaaggggtacag

SEQ ID NO: 591 - DNA, 48 bp, synthetic construct
  misc_feature 1..48: note = Synthetic oligonucleotide
  source 1..48: mol_type = other DNA
gtctggcccccagttgaaaagacgccctgtttttgttaaggggtacag

SEQ ID NO: 592 - DNA, 48 bp, synthetic construct
  misc_feature 1..48: note = Synthetic oligonucleotide
  source 1..48: mol_type = other DNA
gtctggcccccagttgaaaagctcccctgtttttgttaaggggtacag

SEQ ID NO: 593 - DNA, 48 bp, synthetic construct
  misc_feature 1..48: note = Synthetic oligonucleotide
  source 1..48: mol_type = other DNA
tttgctggtctggcccccacgcgaaaagcgtccctgtttttgttaagg

SEQ ID NO: 594 - DNA, 48 bp, synthetic construct
  misc_feature 1..48: note = Synthetic oligonucleotide
  source 1..48: mol_type = other DNA
tttgctggtctggcccccaacggaaaagcgtccctgtttttgttaagg

SEQ ID NO: 595 - DNA, 48 bp, synthetic construct
  misc_feature 1..48: note = Synthetic oligonucleotide
  source 1..48: mol_type = other DNA
tttgctggtctggcccccactcgaaaagcgtccctgtttttgttaagg

SEQ ID NO: 596 - DNA, 54 bp, synthetic construct
  misc_feature 1..54: note = Synthetic oligonucleotide
  source 1..54: mol_type = other DNA
atatccctgtttttcatcaacgaacagcgcaaagggcttggttaaatccggaag

SEQ ID NO: 597 - DNA, 54 bp, synthetic construct
  misc_feature 1..54: note = Synthetic oligonucleotide
  source 1..54: mol_type = other DNA
atatccctgtttttcatcaacgaacagacgaaagggcttggttaaatccggaag

SEQ ID NO: 598 - DNA, 54 bp, synthetic construct
  misc_feature 1..54: note = Synthetic oligonucleotide
  source 1..54: mol_type = other DNA
atatccctgtttttcatcaacgaacagatcaaagggcttggttaaatccggaag

SEQ ID NO: 599 - DNA, 55 bp, synthetic construct
  misc_feature 1..55: note = Synthetic oligonucleotide
  source 1..55: mol_type = other DNA
cttttgcatatccctgtttttcatcaaccgccagctcaaagggcttggttaaatc

SEQ ID NO: 600 - DNA, 55 bp, synthetic construct
  misc_feature 1..55: note = Synthetic oligonucleotide
  source 1..55: mol_type = other DNA
cttttgcatatccctgtttttcatcaacacgcagctcaaagggcttggttaaatc

SEQ ID NO: 601 - DNA, 55 bp, synthetic construct
  misc_feature 1..55: note = Synthetic oligonucleotide
  source 1..55: mol_type = other DNA
cttttgcatatccctgtttttcatcaacctccagctcaaagggcttggttaaatc

SEQ ID NO: 602 - DNA, 46 bp, synthetic construct
  misc_feature 1..46: note = Synthetic oligonucleotide
  source 1..46: mol_type = other DNA
ttactcaagtaagcaacagggcgcgcccacgggcctaacttttggg

SEQ ID NO: 603 - DNA, 46 bp, synthetic construct
  misc_feature 1..46: note = Synthetic oligonucleotide
  source 1..46: mol_type = other DNA
ttactcaagtaagcaacagggcgtttccacgggcctaacttttggg

SEQ ID NO: 604 - DNA, 46 bp, synthetic construct
  misc_feature 1..46: note = Synthetic oligonucleotide
  source 1..46: mol_type = other DNA
ttactcaagtaagcaacagggcgctcccacgggcctaacttttggg

SEQ ID NO: 605 - DNA, 49 bp, synthetic construct
  misc_feature 1..49: note = Synthetic oligonucleotide
  source 1..49: mol_type = other DNA
tctacagcatgtggagccaagatcgctaagggttgacccatcgtcaact

SEQ ID NO: 606 - DNA, 49 bp, synthetic construct
  misc_feature 1..49: note = Synthetic oligonucleotide
  source 1..49: mol_type = other DNA
tctacagcatgtggagccaagatacgtaagggttgacccatcgtcaact

SEQ ID NO: 607 - DNA, 49 bp, synthetic construct
  misc_feature 1..49: note = Synthetic oligonucleotide
  source 1..49: mol_type = other DNA
tctacagcatgtggagccaagatctctaagggttgacccatcgtcaact

SEQ ID NO: 608 - DNA, 53 bp, synthetic construct
  misc_feature 1..53: note = Synthetic oligonucleotide
  source 1..53: mol_type = other DNA
gaagcaaagtagctggattcaaagccgcaactggtccaaattgtacacgatcc

SEQ ID NO: 609 - DNA, 53 bp, synthetic construct
  misc_feature 1..53: note = Synthetic oligonucleotide
  source 1..53: mol_type = other DNA
gaagcaaagtagctggattcaaagcacgaactggtccaaattgtacacgatcc

SEQ ID NO: 610 - DNA, 53 bp, synthetic construct
  misc_feature 1..53: note = Synthetic oligonucleotide
  source 1..53: mol_type = other DNA
gaagcaaagtagctggattcaaagcctcaactggtccaaattgtacacgatcc

SEQ ID NO: 611 - DNA, 48 bp, synthetic construct
  misc_feature 1..48: note = Synthetic oligonucleotide
  source 1..48: mol_type = other DNA
gcggcggtaaatttcgccatgcgcatgcgctgttgcaaaagcataacg

SEQ ID NO: 612 - DNA, 48 bp, synthetic construct
  misc_feature 1..48: note = Synthetic oligonucleotide
  source 1..48: mol_type = other DNA
gcggcggtaaatttcgccatgacgatgcgctgttgcaaaagcataacg

SEQ ID NO: 613 - DNA, 48 bp, synthetic construct
  misc_feature 1..48: note = Synthetic oligonucleotide
  source 1..48: mol_type = other DNA
gcggcggtaaatttcgccatgctcatgcgctgttgcaaaagcataacg

SEQ ID NO: 614 - DNA, 43 bp, synthetic construct
  misc_feature 1..43: note = Synthetic oligonucleotide
  source 1..43: mol_type = other DNA
gaccacggcggcggtaaatcgcgccatggatatgcgctgttgc

SEQ ID NO: 615 - DNA, 43 bp, synthetic construct
  misc_feature 1..43: note = Synthetic oligonucleotide
  source 1..43: mol_type = other DNA
gaccacggcggcggtaaatacggccatggatatgcgctgttgc

SEQ ID NO: 616 - DNA, 43 bp, synthetic construct
  misc_feature 1..43: note = Synthetic oligonucleotide
  source 1..43: mol_type = other DNA
gaccacggcggcggtaaatatcgccatggatatgcgctgttgc

SEQ ID NO: 617 - DNA, 44 bp, synthetic construct
  misc_feature 1..44: note = Synthetic oligonucleotide
  source 1..44: mol_type = other DNA
cagaccacggcggcggtacgcttcgccatggatatgcgctgttg

SEQ ID NO: 618 - DNA, 44 bp, synthetic construct
  misc_feature 1..44: note = Synthetic oligonucleotide
  source 1..44: mol_type = other DNA
cagaccacggcggcggtaacgttcgccatggatatgcgctgttg

SEQ ID NO: 619 - DNA, 44 bp, synthetic construct
  misc_feature 1..44: note = Synthetic oligonucleotide
  source 1..44: mol_type = other DNA
cagaccacggcggcggtactcttcgccatggatatgcgctgttg

SEQ ID NO: 620 - DNA, 41 bp, synthetic construct
  misc_feature 1..41: note = Synthetic oligonucleotide
  source 1..41: mol_type = other DNA
gggcagcttggtccgccatcgcgtttccacgagcctccgct

SEQ ID NO: 621 - DNA, 41 bp, synthetic construct
  misc_feature 1..41: note = Synthetic oligonucleotide
  source 1..41: mol_type = other DNA
gggcagcttggtccgccattttgtttccacgagcctccgct

SEQ ID NO: 622 - DNA, 41 bp, synthetic construct
  misc_feature 1..41: note = Synthetic oligonucleotide
  source 1..41: mol_type = other DNA
gggcagcttggtccgccatctcgtttccacgagcctccgct

SEQ ID NO: 623 - DNA, 40 bp, synthetic construct
  misc_feature 1..40: note = Synthetic oligonucleotide
  source 1..40: mol_type = other DNA
gccgccttacgggcagccgcgtccgccatacggtttccac

SEQ ID NO: 624 - DNA, 40 bp, synthetic construct
  misc_feature 1..40: note = Synthetic oligonucleotide
  source 1..40: mol_type = other DNA
gccgccttacgggcagcacggtccgccatacggtttccac

SEQ ID NO: 625 - DNA, 40 bp, synthetic construct
  misc_feature 1..40: note = Synthetic oligonucleotide
  source 1..40: mol_type = other DNA
gccgccttacgggcagcctcgtccgccatacggtttccac

SEQ ID NO: 626 - DNA, 42 bp, synthetic construct
  misc_feature 1..42: note = Synthetic oligonucleotide
  source 1..42: mol_type = other DNA
gtctctgtgatcgccgccttcgcggcagcttggtccgccata

SEQ ID NO: 627 - DNA, 42 bp, synthetic construct
  misc_feature 1..42: note = Synthetic oligonucleotide
  source 1..42: mol_type = other DNA
gtctctgtgatcgccgcctttttggcagcttggtccgccata

SEQ ID NO: 628 - DNA, 42 bp, synthetic construct
  misc_feature 1..42: note = Synthetic oligonucleotide
  source 1..42: mol_type = other DNA
gtctctgtgatcgccgccttctcggcagcttggtccgccata

SEQ ID NO: 629 - DNA, 50 bp, synthetic construct
  misc_feature 1..50: note = Synthetic oligonucleotide
  source 1..50: mol_type = other DNA
atttgctgaaagaaggtcaacgttggcgtactgatgcgcgtaaggagacc

SEQ ID NO: 630 - DNA, 50 bp, synthetic construct
  misc_feature 1..50: note = Synthetic oligonucleotide
  source 1..50: mol_type = other DNA
ggtctccttacgcgcatcagtacgccaacgttgaccttctttcagcaaat

SEQ ID NO: 631 - DNA, 50 bp, synthetic construct
  misc_feature 1..50: note = Synthetic oligonucleotide
  source 1..50: mol_type = other DNA
gggattaagccacatattcgtcgcttgcgtgaccaggggatcttggtccc

SEQ ID NO: 632 - DNA, 50 bp, synthetic construct
  misc_feature 1..50: note = Synthetic oligonucleotide
  source 1..50: mol_type = other DNA
gggaccaagatcccctggtcacgcaagcgacgaatatgtggcttaatccc

SEQ ID NO: 633 - DNA, 25 bp, synthetic construct
  misc_feature 1..25: note = Synthetic oligonucleotide
  source 1..25: mol_type = other DNA
gtcgagtatctcagaaaagaagaca

SEQ ID NO: 634 - DNA, 22 bp, synthetic construct
  misc_feature 1..22: note = Synthetic oligonucleotide
  source 1..22: mol_type = other DNA
ctcggatgtaggaagtttcacc

SEQ ID NO: 635 - DNA, 24 bp, synthetic construct
  misc_feature 1..24: note = Synthetic oligonucleotide
  misc_feature 1: note = 5' SUN
  misc_feature 10..11: note = ZEN molecule
  misc_feature 24: note = 3' IABkFQ
  source 1..24: mol_type = other DNA
atgccctgcgtaaactggatgaca

SEQ ID NO: 636 - DNA, 2055 bp, synthetic construct
  misc_feature 1..2055: note = Synthetic oligonucleotide
  source 1..2055: mol_type = other DNA
atgactttaaatattgaggatgagcatcgtttacatgagacatcaaaagaacccgacgtgagcttagggtcaacgtggctttctgacttcccccaggcgtgggcggagactggcggaatggggttagctgtccgccaagcaccgttgatcatcccgttaaaggcaacgtctacacctgtctctatcaaacagtaccccatgagtcaagaggcccgcctggggattaagccacatattcagcgcttgctggaccaggggatcttggtcccatgtcaatctccgtggaacaccccccttctgcccgtgaaaaagccaggtacaaacgattatcgtccagttcaagatcttcgcgaggtcaacaaacgcgtagaagacatccatccgactgtacctaatccttataatctgttatcaggcctgcccccatcgcaccaatggtatacagtattagacttgaaagacgcgttcttttgcctgcgtctgcacccaacgtctcagccgctgtttgcgttcgaatggcgtgatcctgaaatgggaatttcgggtcagttaacctggactcgtctgccccagggctttaaaaacagccccacattgttcgatgaagcacttcaccgtgacttagcagacttccgtatccaacacccagacttaattctgttacagtatgttgacgaccttttgttggcggcaacgtctgaacttgactgtcagcaaggcacacgcgcgttattacaaacgttaggtaacttaggatatcgtgcgtccgcgaaaaaggcgcaaatttgtcaaaaacaggtaaagtaccttgggtatttgctgaaagaaggtcaacgttggctgactgaagcgcgtaaggagaccgtaatggggcagcctacgcctaagacgccacgccagttgcgtgaatttttgggcacagcgggattctgtcgtttatggattcctgggttcgctgaaatggctgcacccctgtaccccttaacaaaaacagggacgcttttcaactgggggccagaccagcaaaaggcgtatcaggagatcaaacaagctttgttgaccgcacccgcgttgggtcttccggatttaaccaagccctttgagctgttcgttgatgaaaaacagggatatgcaaaaggagtattaacccaaaagttaggcccgtggcgtcgccctgttgcttacttgagtaaaaaattggatcctgtcgcagcaggatggccaccgtgcttgcgtatggtcgcggcaattgccgttttgacaaaggatgcaggtaagttgacgatgggtcaacccttagtaatcttggctccacatgctgtagaagcgttagtaaagcagcccccagaccgctggctttctaatgcgcgcatgacccactatcaggcgcttctgcttgatacggatcgtgtacaatttggaccagttgtagctttgaatccagctactttgcttccccttccagaagaaggacttcagcacaattgtttagatattctggccgaggcacatgggacgcgccctgatttgacggatcagccactgcctgatgccgaccatacatggtatactggcggcagtagtcttcttcaagaggggcaacgcaaggcgggagcagccgtcactacggagaccgaagttatctgggccaaagcgttacccgcgggaacatccgcgcaacgtgcacagttaatcgctctgacacaggccctgaagatggcagagggcaaaaagttgaatgtctacaccaactcacgttatgcttttgcaacagcgcatatccatggcgaaatttaccgccgccgtggtctgctgactagtgagggtaaggaaattaaaaataaagatgagattcttgcgttgttaaaagctttattcttaccaaaacgcctttcgatcattcattgcccggggcatcaaaagggtcactcagcggaggctcgtggaaaccgtatggcggaccaagctgcccgtaaggcggcgatcacagagaccccggatacatcaacgctgttgatcgaaaacagctctccctacactagcgagcatttttaa

SEQ ID NO: 637 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 638 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSRKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 639 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 640 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIRRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 641 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPRLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 642 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 643 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPAQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 644 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSKKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 645 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSMKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 646 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSIEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 647 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSKEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 648 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIHRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 649 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIIRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 650 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLKPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 651 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLNPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 652 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTMARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 653 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTWARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 654 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSRKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 655 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPRLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 656 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 657 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIRRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 658 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPAQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 659 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSRKQYPMSQEARLGIKPHIQRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 660 - protein, 683 aa, synthetic construct
  REGION 1..683: note = RTase mutant
MTLNIEDEHRLHETSKEPDVSLGSTWLSDFPQAWAETGGMGLAVRQAPLIIPLKATSTPVSRKQYPMSREARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEH

SEQ ID NO: 661 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSRKQYPMSQEARLGIKPHIRRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 662 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSRKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPAQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 663 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIQRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 664 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 665 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPAQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 666 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 667 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPAQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 668 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIRRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPAQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 669 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIQRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 670 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSQEARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 671 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 672 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 673 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 674 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLKPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 675 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLNPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 676 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSIEARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 677 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSKEARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 678 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIHRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 679 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIIRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 680 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTMARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 681 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTWARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 682 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSKKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 683 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSMKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 684 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSIKQYPMSIEARLGIKPHIHRLLDQGILVPCQSPWNTPLKPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTMARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 685 - protein, 684 aa, synthetic construct
  REGION 1..684: note = RTase mutant
MTLNIEDEHRLHETSKEPDVSLGSTWLSDFPQAWAETGGMGLAVRQAPLIIPLKATSTPVSMKQYPMSIEARLGIKPHIHRLLDQGILVPCQSPWNTPLKPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTMARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 686 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLRILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 687 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHEHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 688 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRELREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 689 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGELFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 690 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWRTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 691 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWRTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 692 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIQRLRDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 693 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLRDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHIHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 694 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRELREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHEHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 695 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLRDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRELREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHEHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 696 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRELREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLRILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHEHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 697 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLRDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRELREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLRILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHEHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 698 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLRDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRELREFLGTAGFCRLWIPGFAEMAAPLYPLTKTGELFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHEHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 699 - protein, 683 aa, synthetic construct
  REGION 1..683: note = RTase mutant
TLNIEDEHRLHETSKEPDVSLGSTWLSDFPQAWAETGGMGLAVRQAPLIIPLKATSTPVSIKQYPMSREARLGIKPHIRRLRDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRELREFLGTAGFCRLWIPGFAEMAAPLYPLTKTGELFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLRILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHEHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 700 - DNA, 49 bp, synthetic construct; identical to SEQ ID NO: 489
  misc_feature 1..49: note = Synthetic Oligonucleotide
  source 1..49: mol_type = other DNA

SEQ ID NO: 701 - DNA, 49 bp, synthetic construct; identical to SEQ ID NO: 606
  misc_feature 1..49: note = Synthetic Oligonucleotide
  source 1..49: mol_type = other DNA

SEQ ID NO: 702 - DNA, 48 bp, synthetic construct; identical to SEQ ID NO: 496
  misc_feature 1..48: note = Synthetic Oligonucleotide
  source 1..48: mol_type = other DNA

SEQ ID NO: 703 - DNA, 48 bp, synthetic construct; identical to SEQ ID NO: 613
  misc_feature 1..48: note = Synthetic Oligonucleotide
  source 1..48: mol_type = other DNA

SEQ ID NO: 704 - DNA, 45 bp, synthetic construct; identical to SEQ ID NO: 452
  misc_feature 1..45: note = Synthetic Oligonucleotide
  source 1..45: mol_type = other DNA

SEQ ID NO: 705 - DNA, 45 bp, synthetic construct; identical to SEQ ID NO: 568
  misc_feature 1..45: note = Synthetic Oligonucleotide
  source 1..45: mol_type = other DNA

SEQ ID NO: 706 - DNA, 47 bp, synthetic construct
  misc_feature 1..47: note = Synthetic Oligonucleotide
  source 1..47: mol_type = other DNA
gattaagccacatattcagcgcttgtatgaccaggggatcttggtcc

SEQ ID NO: 707 - DNA, 47 bp, synthetic construct
  misc_feature 1..47: note = Synthetic Oligonucleotide
  source 1..47: mol_type = other DNA
ggaccaagatcccctggtcatacaagcgctgaatatgtggcttaatc

SEQ ID NO: 708 - DNA, 47 bp, synthetic construct
  misc_feature 1..47: note = Synthetic Oligonucleotide
  source 1..47: mol_type = other DNA
tgctgaaagaaggtcaacgttggatcactgaagcgcgtaaggagacc

SEQ ID NO: 709 - DNA, 47 bp, synthetic construct
  misc_feature 1..47: note = Synthetic Oligonucleotide
  source 1..47: mol_type = other DNA
ggtctccttacgcgcttcagtgatccaacgttgaccttctttcagca

SEQ ID NO: 710 - DNA, 49 bp, synthetic construct
  misc_feature 1..49: note = Synthetic Oligonucleotide
  source 1..49: mol_type = other DNA
agttgacgatgggtcaacccttaaacatcttggctccacatgctgtaga

SEQ ID NO: 711 - DNA, 49 bp, synthetic construct
  misc_feature 1..49: note = Synthetic Oligonucleotide
  source 1..49: mol_type = other DNA
tctacagcatgtggagccaagatgtttaagggttgacccatcgtcaact

SEQ ID NO: 712 - DNA, 48 bp, synthetic construct
  misc_feature 1..48: note = Synthetic Oligonucleotide
  source 1..48: mol_type = other DNA
cgttatgcttttgcaacagcgcattggcatggcgaaatttaccgccgc

SEQ ID NO: 713 - DNA, 48 bp, synthetic construct
  misc_feature 1..48: note = Synthetic Oligonucleotide
  source 1..48: mol_type = other DNA
gcggcggtaaatttcgccatgccaatgcgctgttgcaaaagcataacg

SEQ ID NO: 714 - DNA, 52 bp, synthetic construct
  misc_feature 1..52: note = Synthetic Oligonucleotide
  source 1..52: mol_type = other DNA
gccagttgcgtgaatttttgggcaaagcgggattctgtcgtttatggattcc

SEQ ID NO: 715 - DNA, 52 bp, synthetic construct
  misc_feature 1..52: note = Synthetic Oligonucleotide
  source 1..52: mol_type = other DNA
ggaatccataaacgacagaatcccgctttgcccaaaaattcacgcaactggc

SEQ ID NO: 716 - DNA, 2055 bp, synthetic construct; identical to SEQ ID NO: 636
  misc_feature 1..2055: note = Synthetic Oligonucleotide
  source 1..2055: mol_type = other DNA

SEQ ID NO: 717 - protein, 684 aa, synthetic construct; identical to SEQ ID NO: 637
  REGION 1..684: note = Synthetic Peptide

SEQ ID NO: 718 - DNA, 2052 bp, synthetic construct
  misc_feature 1..2052: note = Synthetic Oligonucleotide
  source 1..2052: mol_type = other DNA
atgactttaaatattgaggatgagcatcgtttacatgagacatcaaaagaacccgacgtgagcttagggtcaacgtggctttctgacttcccccaggcgtgggcggagactggcggaatggggttagctgtccgccaagcaccgttgatcatcccgttaaaggcaacgtctacacctgtctctatcaaacagtaccccatgagtcgtgaggcccgcctggggattaagccacatattcgtcgcttgctggaccaggggatcttggtcccatgtcaatctccgtggaacaccccccttcgtcccgtgaaaaagccaggtacaaacgattatcgtccagttcaagatcttcgcgaggtcaacaaacgcgtagaagacatccatccgactgtacctaatccttataatctgttatcaggcctgcccccatcgcaccaatggtatacagtattagacttgaaagacgcgttcttttgcctgcgtctgcacccaacgtctcagccgctgtttgcgttcgaatggcgtgatcctgaaatgggaatttcgggtcagttaacctggactcgtctgccccagggctttaaaaacagccccacattgttcgatgaagcacttcaccgtgacttagcagacttccgtatccaacacccagacttaattctgttacagtatgttgacgaccttttgttggcggcaacgtctgaacttgactgtcagcaaggcacacgcgcgttattacaaacgttaggtaacttaggatatcgtgcgtccgcgaaaaaggcgcaaatttgtcaaaaacaggtaaagtaccttgggtatttgctgaaagaaggtcaacgttggctgactgatgcgcgtaaggagaccgtaatggggcagcctacgcctaagacgccacgcgaattgcgtgaatttttgggcacagcgggattctgtcgtttatggattcctgggttcgctgaaatggctgcacccctgtaccccttaacaaaaacagggacgcttttcaactgggggccagaccagcaaaaggcgtatcaggagatcaaacaagctttgttgaccgcacccgcgttgggtcttccggatttaaccaagccctttgagctgttcgttgatgaaaaacagggatatgcaaaaggagtattaacccaaaagttaggcccgtggcgtcgccctgttgcttacttgagtaaaaaattggatcctgtcgcagcaggatggccaccgtgcttgcgtatggtcgcggcaattgccgttttgacaaaggatgcaggtaagttgacgatgggtcaacccttacgtatcttggctccacatgctgtagaagcgttagtaaagcagcccccagaccgctggctttctaatgcgcgcatgacccactatcaggcgcttctgcttgatacggatcgtgtacaatttggaccagttgtagctttgaatccagctactttgcttccccttccagaagaaggacttcagcacaattgtttagatattctggccgaggcacatgggacgcgccctgatttgacggatcagccactgcctgatgccgaccatacatggtatactggcggcagtagtcttcttcaagaggggcaacgcaaggcgggagcagccgtcactacggagaccgaagttatctgggccaaagcgttacccgcgggaacatccgcgcaacgtgcacagttaatcgctctgacacaggccctgaagatggcagagggcaaaaagttgaatgtctacaccaactcacgttatgcttttgcaacagcgcattggcatggcgaaatttaccgccgccgtggtctgctgactagtgagggtaaggaaattaaaaataaagatgagattcttgcgttgttaaaagctttattcttaccaaaacgcctttcgatcattcattgcccggggcatcaaaagggtcactcagcggaggctcgtggaaaccgtatggcggaccaagctgcccgtaaggcggcgatcacagagaccccggatacatcaacgctgttgatcgaaaacagctctccctacactagcgagcatttt

SEQ ID NO: 719 - protein, 684 aa, synthetic construct
  REGION 1..684: note = Synthetic Peptide
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRELREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLRILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHWHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 720 - DNA, 2052 bp, synthetic construct
  misc_feature 1..2052: note = Synthetic Oligonucleotide
  source 1..2052: mol_type = other DNA
atgactttaaatattgaggatgagcatcgtttacatgagacatcaaaagaacccgacgtgagcttagggtcaacgtggctttctgacttcccccaggcgtgggcggagactggcggaatggggttagctgtccgccaagcaccgttgatcatcccgttaaaggcaacgtctacacctgtctctatcaaacagtaccccatgagtcgtgaggcccgcctggggattaagccacatattcgtcgcttgctggaccaggggatcttggtcccatgtcaatctccgtggaacaccccccttcgtcccgtgaaaaagccaggtacaaacgattatcgtccagttcaagatcttcgcgaggtcaacaaacgcgtagaagacatccatccgactgtacctaatccttataatctgttatcaggcctgcccccatcgcaccaatggtatacagtattagacttgaaagacgcgttcttttgcctgcgtctgcacccaacgtctcagccgctgtttgcgttcgaatggcgtgatcctgaaatgggaatttcgggtcagttaacctggactcgtctgccccagggctttaaaaacagccccacattgttcgatgaagcacttcaccgtgacttagcagacttccgtatccaacacccagacttaattctgttacagtatgttgacgaccttttgttggcggcaacgtctgaacttgactgtcagcaaggcacacgcgcgttattacaaacgttaggtaacttaggatatcgtgcgtccgcgaaaaaggcgcaaatttgtcaaaaacaggtaaagtaccttgggtatttgctgaaagaaggtcaacgttggattactgatgcgcgtaaggagaccgtaatggggcagcctacgcctaagacgccacgcgaattgcgtgaatttttgggcacagcgggattctgtcgtttatggattcctgggttcgctgaaatggctgcacccctgtaccccttaacaaaaacagggacgcttttcaactgggggccagaccagcaaaaggcgtatcaggagatcaaacaagctttgttgaccgcacccgcgttgggtcttccggatttaaccaagccctttgagctgttcgttgatgaaaaacagggatatgcaaaaggagtattaacccaaaagttaggcccgtggcgtcgccctgttgcttacttgagtaaaaaattggatcctgtcgcagcaggatggccaccgtgcttgcgtatggtcgcggcaattgccgttttgacaaaggatgcaggtaagttgacgatgggtcaacccttacgtatcttggctccacatgctgtagaagcgttagtaaagcagcccccagaccgctggctttctaatgcgcgcatgacccactatcaggcgcttctgcttgatacggatcgtgtacaatttggaccagttgtagctttgaatccagctactttgcttccccttccagaagaaggacttcagcacaattgtttagatattctggccgaggcacatgggacgcgccctgatttgacggatcagccactgcctgatgccgaccatacatggtatactggcggcagtagtcttcttcaagaggggcaacgcaaggcgggagcagccgtcactacggagaccgaagttatctgggccaaagcgttacccgcgggaacatccgcgcaacgtgcacagttaatcgctctgacacaggccctgaagatggcagagggcaaaaagttgaatgtctacaccaactcacgttatgcttttgcaacagcgcattggcatggcgaaatttaccgccgccgtggtctgctgactagtgagggtaaggaaattaaaaataaagatgagattcttgcgttgttaaaagctttattcttaccaaaacgcctttcgatcattcattgcccggggcatcaaaagggtcactcagcggaggctcgtggaaaccgtatggcggaccaagctgcccgtaaggcggcgatcacagagaccccggatacatcaacgctgttgatcgaaaacagctctccctacactagcgagcatttt

SEQ ID NO: 721 - protein, 684 aa, synthetic construct
  REGION 1..684: note = Synthetic Peptide
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWITDARKETVMGQPTPKTPRELREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLRILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHWHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 722 - DNA, 2052 bp, synthetic construct
  misc_feature 1..2052: note = Synthetic Oligonucleotide
  source 1..2052: mol_type = other DNA
atgactttaaatattgaggatgagcatcgtttacatgagacatcaaaagaacccgacgtgagcttagggtcaacgtggctttctgacttcccccaggcgtgggcggagactggcggaatggggttagctgtccgccaagcaccgttgatcatcccgttaaaggcaacgtctacacctgtctctatcaaacagtaccccatgagtcgtgaggcccgcctggggattaagccacatattcgtcgcttgtatgaccaggggatcttggtcccatgtcaatctccgtggaacaccccccttcgtcccgtgaaaaagccaggtacaaacgattatcgtccagttcaagatcttcgcgaggtcaacaaacgcgtagaagacatccatccgactgtacctaatccttataatctgttatcaggcctgcccccatcgcaccaatggtatacagtattagacttgaaagacgcgttcttttgcctgcgtctgcacccaacgtctcagccgctgtttgcgttcgaatggcgtgatcctgaaatgggaatttcgggtcagttaacctggactcgtctgccccagggctttaaaaacagccccacattgttcgatgaagcacttcaccgtgacttagcagacttccgtatccaacacccagacttaattctgttacagtatgttgacgaccttttgttggcggcaacgtctgaacttgactgtcagcaaggcacacgcgcgttattacaaacgttaggtaacttaggatatcgtgcgtccgcgaaaaaggcgcaaatttgtcaaaaacaggtaaagtaccttgggtatttgctgaaagaaggtcaacgttggattactgatgcgcgtaaggagaccgtaatggggcagcctacgcctaagacgccacgcgaattgcgtgaatttttgggcacagcgggattctgtcgtttatggattcctgggttcgctgaaatggctgcacccctgtaccccttaacaaaaacagggacgcttttcaactgggggccagaccagcaaaaggcgtatcaggagatcaaacaagctttgttgaccgcacccgcgttgggtcttccggatttaaccaagccctttgagctgttcgttgatgaaaaacagggatatgcaaaaggagtattaacccaaaagttaggcccgtggcgtcgccctgttgcttacttgagtaaaaaattggatcctgtcgcagcaggatggccaccgtgcttgcgtatggtcgcggcaattgccgttttgacaaaggatgcaggtaagttgacgatgggtcaacccttacgtatcttggctccacatgctgtagaagcgttagtaaagcagcccccagaccgctggctttctaatgcgcgcatgacccactatcaggcgcttctgcttgatacggatcgtgtacaatttggaccagttgtagctttgaatccagctactttgcttccccttccagaagaaggacttcagcacaattgtttagatattctggccgaggcacatgggacgcgccctgatttgacggatcagccactgcctgatgccgaccatacatggtatactggcggcagtagtcttcttcaagaggggcaacgcaaggcgggagcagccgtcactacggagaccgaagttatctgggccaaagcgttacccgcgggaacatccgcgcaacgtgcacagttaatcgctctgacacaggccctgaagatggcagagggcaaaaagttgaatgtctacaccaactcacgttatgcttttgcaacagcgcattggcatggcgaaatttaccgccgccgtggtctgctgactagtgagggtaaggaaattaaaaataaagatgagattcttgcgttgttaaaagctttattcttaccaaaacgcctttcgatcattcattgcccggggcatcaaaagggtcactcagcggaggctcgtggaaaccgtatggcggaccaagctgcccgtaaggcggcgatcacagagaccccggatacatcaacgctgttgatcgaaaacagctctccctacactagcgagcatttt

SEQ ID NO: 723 - protein, 684 aa, synthetic construct
  REGION 1..684: note = Synthetic Peptide
MTLNIEDEHRLHETSKEPDVSLGSTWLSDFPQAWAETGGMGLAVRQAPLIIPLKATSTPVSIKQYPMSREARLGIKPHIRRLYDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWITDARKETVMGQPTPKTPRELREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLRILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHWHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 724 - DNA, 2052 bp, synthetic construct
  misc_feature 1..2052: note = Synthetic Oligonucleotide
  source 1..2052: mol_type = other DNA
atgactttaaatattgaggatgagcatcgtttacatgagacatcaaaagaacccgacgtgagcttagggtcaacgtggctttctgacttcccccaggcgtgggcggagactggcggaatggggttagctgtccgccaagcaccgttgatcatcccgttaaaggcaacgtctacacctgtctctatcaaacagtaccccatgagtcgtgaggcccgcctggggattaagccacatattcgtcgcttgtatgaccaggggatcttggtcccatgtcaatctccgtggaacaccccccttcgtcccgtgaaaaagccaggtacaaacgattatcgtccagttcaagatcttcgcgaggtcaacaaacgcgtagaagacatccatccgactgtacctaatccttataatctgttatcaggcctgcccccatcgcaccaatggtatacagtattagacttgaaagacgcgttcttttgcctgcgtctgcacccaacgtctcagccgctgtttgcgttcgaatggcgtgatcctgaaatgggaatttcgggtcagttaacctggactcgtctgccccagggctttaaaaacagccccacattgttcgatgaagcacttcaccgtgacttagcagacttccgtatccaacacccagacttaattctgttacagtatgttgacgaccttttgttggcggcaacgtctgaacttgactgtcagcaaggcacacgcgcgttattacaaacgttaggtaacttaggatatcgtgcgtccgcgaaaaaggcgcaaatttgtcaaaaacaggtaaagtaccttgggtatttgctgaaagaaggtcaacgttggattactgatgcgcgtaaggagaccgtaatggggcagcctacgcctaagacgccacgcgaattgcgtgaatttttgggcaaagcgggattctgtcgtttatggattcctgggttcgctgaaatggctgcacccctgtaccccttaacaaaaacagggacgcttttcaactgggggccagaccagcaaaaggcgtatcaggagatcaaacaagctttgttgaccgcacccgcgttgggtcttccggatttaaccaagccctttgagctgttcgttgatgaaaaacagggatatgcaaaaggagtattaacccaaaagttaggcccgtggcgtcgccctgttgcttacttgagtaaaaaattggatcctgtcgcagcaggatggccaccgtgcttgcgtatggtcgcggcaattgccgttttgacaaaggatgcaggtaagttgacgatgggtcaacccttaaacatcttggctccacatgctgtagaagcgttagtaaagcagcccccagaccgctggctttctaatgcgcgcatgacccactatcaggcgcttctgcttgatacggatcgtgtacaatttggaccagttgtagctttgaatccagctactttgcttccccttccagaagaaggacttcagcacaattgtttagatattctggccgaggcacatgggacgcgccctgatttgacggatcagccactgcctgatgccgaccatacatggtatactggcggcagtagtcttcttcaagaggggcaacgcaaggcgggagcagccgtcactacggagaccgaagttatctgggccaaagcgttacccgcgggaacatccgcgcaacgtgcacagttaatcgctctgacacaggccctgaagatggcagagggcaaaaagttgaatgtctacaccaactcacgttatgcttttgcaacagcgcattggcatggcgaaatttaccgccgccgtggtctgctgactagtgagggtaaggaaattaaaaataaagatgagattcttgcgttgttaaaagctttattcttaccaaaacgcctttcgatcattcattgcccggggcatcaaaagggtcactcagcggaggctcgtggaaaccgtatggcggaccaagctgcccgtaaggcggcgatcacagagaccccggatacatcaacgctgttgatcgaaaacagctctccctacactagcgagcatttt

SEQ ID NO: 725 - protein, 684 aa, synthetic construct
  REGION 1..684: note = Synthetic Peptide
MTLNIEDEHRLHETSKEPDVSLGSTWLSDFPQAWAETGGMGLAVRQAPLIIPLKATSTPVSIKQYPMSREARLGIKPHIRRLYDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWITDARKETVMGQPTPKTPRELREFLGKAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLNILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHWHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 726 - DNA, 2052 bp, synthetic construct
  misc_feature 1..2052: note = Synthetic Oligonucleotide
  source 1..2052: mol_type = other DNA
atgactttaaatattgaggatgagcatcgtttacatgagacatcaaaagaacccgacgtgagcttagggtcaacgtggctttctgacttcccccaggcgtgggcggagactggcggaatggggttagctgtccgccaagcaccgttgatcatcccgttaaaggcaacgtctacacctgtctctatcaaacagtaccccatgagtcgtgaggcccgcctggggattaagccacatattcgtcgcttgctggaccaggggatcttggtcccatgtcaatctccgtggaacaccccccttcgtcccgtgaaaaagccaggtacaaacgattatcgtccagttcaagatcttcgcgaggtcaacaaacgcgtagaagacatccatccgactgtacctaatccttataatctgttatcaggcctgcccccatcgcaccaatggtatacagtattagacttgaaagacgcgttcttttgcctgcgtctgcacccaacgtctcagccgctgtttgcgttcgaatggcgtgatcctgaaatgggaatttcgggtcagttaacctggactcgtctgccccagggctttaaaaacagccccacattgttcgatgaagcacttcaccgtgacttagcagacttccgtatccaacacccagacttaattctgttacagtatgttgacgaccttttgttggcggcaacgtctgaacttgactgtcagcaaggcacacgcgcgttattacaaacgttaggtaacttaggatatcgtgcgtccgcgaaaaaggcgcaaatttgtcaaaaacaggtaaagtaccttgggtatttgctgaaagaaggtcaacgttggctgactgatgcgcgtaaggagaccgtaatggggcagcctacgcctaagacgccacgcgaattgcgtgaatttttgggcaaagcgggattctgtcgtttatggattcctgggttcgctgaaatggctgcacccctgtaccccttaacaaaaacagggacgcttttcaactgggggccagaccagcaaaaggcgtatcaggagatcaaacaagctttgttgaccgcacccgcgttgggtcttccggatttaaccaagccctttgagctgttcgttgatgaaaaacagggatatgcaaaaggagtattaacccaaaagttaggcccgtggcgtcgccctgttgcttacttgagtaaaaaattggatcctgtcgcagcaggatggccaccgtgcttgcgtatggtcgcggcaattgccgttttgacaaaggatgcaggtaagttgacgatgggtcaacccttacgtatcttggctccacatgctgtagaagcgttagtaaagcagcccccagaccgctggctttctaatgcgcgcatgacccactatcaggcgcttctgcttgatacggatcgtgtacaatttggaccagttgtagctttgaatccagctactttgcttccccttccagaagaaggacttcagcacaattgtttagatattctggccgaggcacatgggacgcgccctgatttgacggatcagccactgcctgatgccgaccatacatggtatactggcggcagtagtcttcttcaagaggggcaacgcaaggcgggagcagccgtcactacggagaccgaagttatctgggccaaagcgttacccgcgggaacatccgcgcaacgtgcacagttaatcgctctgacacaggccctgaagatggcagagggcaaaaagttgaatgtctacaccaactcacgttatgcttttgcaacagcgcatgaacatggcgaaatttaccgccgccgtggtctgctgactagtgagggtaaggaaattaaaaataaagatgagattcttgcgttgttaaaagctttattcttaccaaaacgcctttcgatcattcattgcccggggcatcaaaagggtcactcagcggaggctcgtggaaaccgtatggcggaccaagctgcccgtaaggcggcgatcacagagaccccggatacatcaacgctgttgatcgaaaacagctctccctacactagcgagcatttt

SEQ ID NO: 727 - protein, 684 aa, synthetic construct
  REGION 1..684: note = Synthetic Peptide
MTLNIEDEHRLHETSKEPDVSLGSTWLSDFPQAWAETGGMGLAVRQAPLIIPLKATSTPVSIKQYPMSREARLGIKPHIRRLLDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTDARKETVMGQPTPKTPRELREFLGKAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLRILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHEHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 728 - DNA, 2052 bp, synthetic construct
  misc_feature 1..2052: note = Synthetic Oligonucleotide
  source 1..2052: mol_type = other DNA
atgactttaaatattgaggatgagcatcgtttacatgagacatcaaaagaacccgacgtgagcttagggtcaacgtggctttctgacttcccccaggcgtgggcggagactggcggaatggggttagctgtccgccaagcaccgttgatcatcccgttaaaggcaacgtctacacctgtctctatcaaacagtaccccatgagtcgtgaggcccgcctggggattaagccacatattcgtcgcttgtatgaccaggggatcttggtcccatgtcaatctccgtggaacaccccccttcgtcccgtgaaaaagccaggtacaaacgattatcgtccagttcaagatcttcgcgaggtcaacaaacgcgtagaagacatccatccgactgtacctaatccttataatctgttatcaggcctgcccccatcgcaccaatggtatacagtattagacttgaaagacgcgttcttttgcctgcgtctgcacccaacgtctcagccgctgtttgcgttcgaatggcgtgatcctgaaatgggaatttcgggtcagttaacctggactcgtctgccccagggctttaaaaacagccccacattgttcgatgaagcacttcaccgtgacttagcagacttccgtatccaacacccagacttaattctgttacagtatgttgacgaccttttgttggcggcaacgtctgaacttgactgtcagcaaggcacacgcgcgttattacaaacgttaggtaacttaggatatcgtgcgtccgcgaaaaaggcgcaaatttgtcaaaaacaggtaaagtaccttgggtatttgctgaaagaaggtcaacgttggattactgatgcgcgtaaggagaccgtaatggggcagcctacgcctaagacgccacgcgaattgcgtgaatttttgggcacagcgggattctgtcgtttatggattcctgggttcgctgaaatggctgcacccctgtaccccttaacaaaaacagggacgcttttcaactgggggccagaccagcaaaaggcgtatcaggagatcaaacaagctttgttgaccgcacccgcgttgggtcttccggatttaaccaagccctttgagctgttcgttgatgaaaaacagggatatgcaaaaggagtattaacccaaaagttaggcccgtggcgtcgccctgttgcttacttgagtaaaaaattggatcctgtcgcagcaggatggccaccgtgcttgcgtatggtcgcggcaattgccgttttgacaaaggatgcaggtaagttgacgatgggtcaacccttacgtatcttggctccacatgctgtagaagcgttagtaaagcagcccccagaccgctggctttctaatgcgcgcatgacccactatcaggcgcttctgcttgatacggatcgtgtacaatttggaccagttgtagctttgaatccagctactttgcttccccttccagaagaaggacttcagcacaattgtttagatattctggccgaggcacatgggacgcgccctgatttgacggatcagccactgcctgatgccgaccatacatggtatactggcggcagtagtcttcttcaagaggggcaacgcaaggcgggagcagccgtcactacggagaccgaagttatctgggccaaagcgttacccgcgggaacatccgcgcaacgtgcacagttaatcgctctgacacaggccctgaagatggcagagggcaaaaagttgaatgtctacaccaactcacgttatgcttttgcaacagcgcatgaacatggcgaaatttaccgccgccgtggtctgctgactagtgagggtaaggaaattaaaaataaagatgagattcttgcgttgttaaaagctttattcttaccaaaacgcctttcgatcattcattgcccggggcatcaaaagggtcactcagcggaggctcgtggaaaccgtatggcggaccaagctgcccgtaaggcggcgatcacagagaccccggatacatcaacgctgttgatcgaaaacagctctccctacactagcgagcatttt

SEQ ID NO: 729 - protein, 684 aa, synthetic construct
  REGION 1..684: note = Synthetic Peptide
MTLNIEDEHRLHETSKEPDVSLGSTWLSDFPQAWAETGGMGLAVRQAPLIIPLKATSTPVSIKQYPMSREARLGIKPHIRRLYDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWITDARKETVMGQPTPKTPRELREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLRILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHEHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 730 - DNA, 2052 bp, synthetic construct
  misc_feature 1..2052: note = Synthetic Oligonucleotide
  source 1..2052: mol_type = other DNA
atgactttaaatattgaggatgagcatcgtttacatgagacatcaaaagaacccgacgtgagcttagggtcaacgtggctttctgacttcccccaggcgtgggcggagactggcggaatggggttagctgtccgccaagcaccgttgatcatcccgttaaaggcaacgtctacacctgtctctatcaaacagtaccccatgagtcgtgaggcccgcctggggattaagccacatattcgtcgcttgtatgaccaggggatcttggtcccatgtcaatctccgtggaacaccccccttcgtcccgtgaaaaagccaggtacaaacgattatcgtccagttcaagatcttcgcgaggtcaacaaacgcgtagaagacatccatccgactgtacctaatccttataatctgttatcaggcctgcccccatcgcaccaatggtatacagtattagacttgaaagacgcgttcttttgcctgcgtctgcacccaacgtctcagccgctgtttgcgttcgaatggcgtgatcctgaaatgggaatttcgggtcagttaacctggactcgtctgccccagggctttaaaaacagccccacattgttcgatgaagcacttcaccgtgacttagcagacttccgtatccaacacccagacttaattctgttacagtatgttgacgaccttttgttggcggcaacgtctgaacttgactgtcagcaaggcacacgcgcgttattacaaacgttaggtaacttaggatatcgtgcgtccgcgaaaaaggcgcaaatttgtcaaaaacaggtaaagtaccttgggtatttgctgaaagaaggtcaacgttggattactgatgcgcgtaaggagaccgtaatggggcagcctacgcctaagacgccacgcgaattgcgtgaatttttgggcaaagcgggattctgtcgtttatggattcctgggttcgctgaaatggctgcacccctgtaccccttaacaaaaacagggacgcttttcaactgggggccagaccagcaaaaggcgtatcaggagatcaaacaagctttgttgaccgcacccgcgttgggtcttccggatttaaccaagccctttgagctgttcgttgatgaaaaacagggatatgcaaaaggagtattaacccaaaagttaggcccgtggcgtcgccctgttgcttacttgagtaaaaaattggatcctgtcgcagcaggatggccaccgtgcttgcgtatggtcgcggcaattgccgttttgacaaaggatgcaggtaagttgacgatgggtcaacccttacgtatcttggctccacatgctgtagaagcgttagtaaagcagcccccagaccgctggctttctaatgcgcgcatgacccactatcaggcgcttctgcttgatacggatcgtgtacaatttggaccagttgtagctttgaatccagctactttgcttccccttccagaagaaggacttcagcacaattgtttagatattctggccgaggcacatgggacgcgccctgatttgacggatcagccactgcctgatgccgaccatacatggtatactggcggcagtagtcttcttcaagaggggcaacgcaaggcgggagcagccgtcactacggagaccgaagttatctgggccaaagcgttacccgcgggaacatccgcgcaacgtgcacagttaatcgctctgacacaggccctgaagatggcagagggcaaaaagttgaatgtctacaccaactcacgttatgcttttgcaacagcgcatgaacatggcgaaatttaccgccgccgtggtctgctgactagtgagggtaaggaaattaaaaataaagatgagattcttgcgttgttaaaagctttattcttaccaaaacgcctttcgatcattcattgcccggggcatcaaaagggtcactcagcggaggctcgtggaaaccgtatggcggaccaagctgcccgtaaggcggcgatcacagagaccccggatacatcaacgctgttgatcgaaaacagctctccctacactagcgagcatttt

SEQ ID NO: 731 - protein, 684 aa, synthetic construct
  REGION 1..684: note = Synthetic Peptide
MTLNIEDEHRLHETSKEPDVSLGSTWLSDFPQAWAETGGMGLAVRQAPLIIPLKATSTPVSIKQYPMSREARLGIKPHIRRLYDQGILVPCQSPWNTPLRPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWITDARKETVMGQPTPKTPRELREFLGKAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLRILAPHAVEALVKQPPDRWLSNARMTHYQALLLDTDRVQFGPVVALNPATLLPLPEEGLQHNCLDILAEAHGTRPDLTDQPLPDADHTWYTGGSSLLQEGQRKAGAAVTTETEVIWAKALPAGTSAQRAQLIALTQALKMAEGKKLNVYTNSRYAFATAHEHGEIYRRRGLLTSEGKEIKNKDEILALLKALFLPKRLSIIHCPGHQKGHSAEARGNRMADQAARKAAITETPDTSTLLIENSSPYTSEHF

SEQ ID NO: 732 - protein, 16 aa, synthetic construct
  REGION 1..16: note = Synthetic Peptide
RDRNKNNDRRKAKENE

SEQ ID NO: 733 - protein, 22 aa, synthetic construct
  REGION 1..22: note = Synthetic Peptide
DPDNEAYEMPSEEGYQDYEPEA

SEQ ID NO: 734 - protein, 42 aa, synthetic construct
  REGION 1..42: note = Synthetic Peptide
QLGKNEEGAPQEGILEDMPVDPDNEAYEMPSEEGYQDYEPEA

SEQ ID NO: 735 - protein, 40 aa, synthetic construct
  REGION 1..40: note = Synthetic Peptide
EGMEEGEFSEAREDLAALEKDYEEVGAEGGDDEDDEGEEY

SEQ ID NO: 736 - protein, 50 aa, synthetic construct
  REGION 1..50: note = Synthetic Peptide
EGMDEMEFTEAESNMNDLVSEYQQYQDATADEEGDYEDEEEGEYQQEEEY

SEQ ID NO: 737 - protein, 114 aa, synthetic construct
  REGION 1..114: note = Synthetic Peptide
IDAAREEFLADNPGIDAEDANVQQFNAQKYVLQDGDIMWQVEFFADEGEEGECLPMLSGEAAQSVFDGDYDEIEIRQEWQEENTLHEWDEGEFQLEPPLDTEEGRAAADEWDER

SEQ ID NO: 738 - protein, 137 aa, synthetic construct
  REGION 1..137: note = Synthetic Peptide
ANPEQLEEQREETRLIIEELLEDGSDPDALYTIEHHLSADDLETLEKAAVEAFKLGYEVTDPEELEVEDGDIVICCDILSECALNADLIDAQVEQLMTLAEKFDVEYDGWGTYFEDPNGEDGDDEDFVDEDDDGVRH

SEQ ID NO: 739 - protein, 182 aa, synthetic construct
  REGION 1..182: note = Synthetic Peptide
DIVDSDQIEDIIQMINDMGIQVMEEAPDADDLMLAENTADEDAAEAAAQVLSSVESEIGRTTDPVRMYMREMGTVELLTREGEIDIAKRIEDGINQVQCSVAEYPEAITYLLEQYDRVEAEEARLSDLITGFVDPNAEEDLAPTATHVGSELSQEDLDDDEDEDEEDGDDDSADDDNSIDPE

SEQ ID NO: 740 - protein, 19 aa, synthetic construct
  REGION 1..19: note = Synthetic Peptide
PNGEDGDDEDFVDEDDDGV

SEQ ID NO: 741 - protein, 102 aa, synthetic construct
  REGION 1..102: note = Synthetic Peptide
MTTATFSRHVERSDLPLLVDFWAPCGPCKMMAPQFQQAAHQLEPTIRLAKVNIEAEPHLAAQFGIRSIPTLALFQGGREIARQAGVMGAQDIVRWTSTQVGR

SEQ ID NO: 742 - protein, 45 aa, synthetic construct
  REGION 1..45: note = Synthetic Peptide
KKDQLGKNEEGAPQEGILEDMPVDPDNEAYEMPSEEGYQDYEPEA

SEQ ID NO: 743 - protein, 13 aa, synthetic construct
  REGION 1..13: note = Synthetic Peptide
NEEGAPQEGILED

SEQ ID NO: 744 - protein, 13 aa, synthetic construct
  REGION 1..13: note = Synthetic Peptide
NDMPVDPDNEAYE

SEQ ID NO: 745 - protein, 22 aa, synthetic construct
  REGION 1..22: note = Synthetic Peptide
DPDNEAYEMPSEEGYQDYEPEA

SEQ ID NO: 746 - protein, 11 aa, synthetic construct
  REGION 1..11: note = Synthetic Peptide
EEGYQDYEPEA

SEQ ID NO: 747 - protein, 26 aa, synthetic construct
  REGION 1..26: note = Synthetic Peptide
DMSDEELEKKLTQYSEMDQEFVKQMI

SEQ ID NO: 748 - protein, 21 aa, synthetic construct
  REGION 1..21: note = Synthetic Peptide
SGSGTTTTTTTSTTTGGTDPT

SEQ ID NO: 749 - protein, 76 aa, synthetic construct
  REGION 1..76: note = Synthetic Peptide
VFTATTTLVPTKLETFPLDVLVNTAAEDLPRGVDPSRKENHLSDEDFKAVFGMTRSAFANLPLWKQQNLKKEKGLF

SEQ ID NO: 750 - protein, 35 aa, synthetic construct [sequence omitted]

SEQ ID NO: 751 - protein, 27 aa, synthetic construct
  REGION 1..27: note = Synthetic Peptide
GYIPEAPRDGQAYVRKDGEWVLLSTFL

SEQ ID NO: 752 - protein, 184 aa, synthetic construct
  REGION 1..184: note = Synthetic Peptide
TNVTFTMSGGTGDADLYVRAGSKPTSTTYDCRPYKGGNSEECSIDSPTAGTYHVMLRGYSAYSGVSLVGNITGGSTGGGSGTPQAGGGTVSDITANAGQWKHYTLDVPAGMANFTVTTSGGTGDADLFVKFGSQPTSSSYDCRPYKNGNAETCTFSNPQAGTWHLSVNAYQTFSGLTLSGQYQP

SEQ ID NO: 753 - protein, 67 aa, synthetic construct
  REGION 1..67: note = Synthetic Peptide
TNVTFTMSGGTGDADLYVRAGSKPTSTTYDCRPYKGGNSEECSIDSPTAGTYHVMLRGYSAYSGVSL

SEQ ID NO: 754 - protein, 85 aa, synthetic construct
  REGION 1..85: note = Synthetic Peptide
AGQWKHYTLDVPAGMANFTVTTSGGTGDADLFVKFGSQPTSSSYDCRPYKNGNAETCTFSNPQAGTWHLSVNAYQTFSGLTLSGQ

SEQ ID NO: 755 - protein, 105 aa, synthetic construct
  REGION 1..105: note = Synthetic Peptide
NPGGNVLQNNVPVTGLGAATGAELNYTVAVPAGSSQLRVTISGGSGDADLYVRQGSAPTDTSYTCRPYLSGNSETCTINSPAAGTWYVRVKAYSTFSGVTLNAQY

SEQ ID NO: 756 - protein, 106 aa, synthetic construct
  REGION 1..106: note = Synthetic Peptide
SCGPVATPLTNKAAVGGLNGTAGSSRLYSFEAAAGKQLSVITYGGTGNVSVYIAQGREPSASDNDGKSTRPGTSETVRVNKPVAGTYYIKVVGEAAYNGVSILATQ

SEQ ID NO: 757 - protein, 217 aa, synthetic construct
  REGION 1..217: note = Synthetic Peptide
NPGGNVLQNNVPVTGLGAATGAELNYTVAVPAGSSQLRVTISGGSGDADLYVRQGSAPTDTSYTCRPYLSGNSETCTINSPAAGTWYVRVKAYSTFSGVTLNAQYEEPCTESCGPVATPLTNKAAVGGLNGTAGSSRLYSFEAAAGKQLSVITYGGTGNVSVYIAQGREPSASDNDGKSTRPGTSETVRVNKPVAGTYYIKVVGEAAYNGVSILATQ

SEQ ID NO: 758 - protein, 217 aa, synthetic construct
  REGION 1..217: note = Synthetic Peptide
SCGPVATPLTNKAAVGGLNGTAGSSRLYSFEAAAGKQLSVITYGGTGNVSVYIAQGREPSASDNDGKSTRPGTSETVRVNKPVAGTYYIKVVGEAAYNGVSILATQEEPCTENPGGNVLQNNVPVTGLGAATGAELNYTVAVPAGSSQLRVTISGGSGDADLYVRQGSAPTDTSYTCRPYLSGNSETCTINSPAAGTWYVRVKAYSTFSGVTLNAQY

SEQ ID NO: 759 - protein, 20 aa, synthetic construct
  REGION 1..20: note = Synthetic Peptide
SFDIRAFYLRLAEQLASLRP

SEQ ID NO: 760 - protein, 11 aa, synthetic construct
  REGION 1..11: note = Synthetic Peptide
REEKPSSAPSS

SEQ ID NO: 761 - protein, 13 aa, synthetic construct
  REGION 1..13: note = Synthetic Peptide
REEKPAVTAAPKK

SEQ ID NO: 762 - protein, 64 aa, synthetic construct
  REGION 1..64: note = Synthetic Peptide
MVTVKFKYKGEELEVDISKIKKVWRVGKMISFTYDDNGKTGRGAVSEKDAPKELLQMLEKSGKK